(19) United States
(12) Patent Application Publication
(10) Pub. No.: US 2013/0013219 A1
(43) Pub. Date: Jan. 10, 2013

(54) Determining Susceptibility To A Sudden Cardiac Event

(75) Inventors: Steven Rosenberg, Oakland, CA (US); Michael R. Elashoff, Redwood City, CA (US); John Lincoln Blanchard, Redwood City, CA (US); Susan Elizabeth Daniels, Mountain View, CA (US); James Alan Wingrove, Sunnyvale, CA (US); Amy Jo-Nell Sehnert, San Francisco, CA (US)

(73) Assignee: CARDIODX ,INC., Palo Alto, CA (US)

(21) Appl. No.: 13/635,018

(22) PCT Filed: Mar. 18, 2011
(86) PCT No.: PCT/US2011/029041

§ 371 Date: Sep. 14, 2012

Related U.S. Application Data
(60) Provisional application No. 61/315,748, filed on Mar. 19, 2010.

(51) Int. Cl. G06F 19/10; C12Q 1/68

(52) U.S. Cl. 702/19; 435/6.11

(57) ABSTRACT

Disclosed herein is a method of do terming the likelihood of a sudden cardiac event, such as an arrythmia, in a subject. Also disclosed is a method of determining whether a subject is at risk of a sudden cardiac event arid whether the subject would benefit from a treatment such as implantation of an ICD.

### CROSS REFERENCE TO RELATED APPLICATIONS

This application claims the benefit of U.S. Provisional Application No. 61/315,748, filed Mar. 19, 2010, the entire disclosure of which is hereby incorporated by reference in its entirety for all purposes.

### BACKGROUND

1. Field

This application is directed to the areas of bioinformatics and heart conditions. The teachings relate to diagnosis and treatment of heart conditions, such as sudden cardiac death.

2. Background Material

Heart failure (HF) affects 5 million Americans, with 550,000 new cases diagnosed and 250,000 deaths each year. Sudden cardiac events (SCE) due to ventricular arrhythmias (ventricular tachycardia, VT; and ventricular fibrillation, VF) is a serious and common problem in the developed world and accounts for half of all deaths in HF. These arrhythmias may be precipitated by a complex interaction of environmental, clinical, and genetic factors. While therapies such as implanted cardioverter defibrillators (ICD) show benefit in this population, the current measure used to recommend implant of a primary prevention ICD, low ejection fraction (EF) <35%, has significant limitations. When using low EF alone as an indication for ICD, the majority (˜75%) of patients implanted never receive life-saving benefit from the device while at the same time being exposed to the risks and complications of this expensive, invasive therapy. Furthermore, there is currently no clinically-accepted measure to identify the even larger population of patients at risk for SCE with EF >35% who could derive benefit from an ICD. Genetic markers associated with lethal ventricular arrhythmias provide an important tool to identify patients at highest risk who would most benefit from directed ICD therapy.

Susceptibility for SCE is multi-factorial. SCE in adults most often occurs in the setting of coronary artery disease (CAD), but also occurs in the setting of non-ischemic conditions and other disorders. Genetic markers associated with the phenotype of VT and/or VF in a HF population would provide unique insight into an individual's risk for SCE and is expected to be additive (or at least complementary) to other anatomic, disease-based clinical measures currently used to assess this risk.

The importance of the influence of genetics on this problem is growing through the following lines of evidence: 1) Family history of SCE is a well-known important risk factor and the heritable risk is well established. 2) Genetics of rare inherited SCE disorders are well described and common variants in these disease genes are hypothesized to play a potentially important role outside of families, and 3) recent genome-wide association (GWAS) studies have identified genetic markers associated with quantitative traits such as QT interval duration that may influence SCE risk in the general population.

Accounting for the underlying genetic pre-disposition for a lethal arrhythmic event is potentially both distinct and complementary to other measures used today. Current risk-stratification methods focus on measurable anatomic features of the heart (e.g., EF, scar mass, wall motion) and the cardiac conduction system (e.g., electrophysiologic characteristics) after the heart is damaged by ischemic or non-ischemic causes. Allelic variation among multiple interlinked pathways leading to the final anatomic phenotype may influence a wide-range or a small portion of the final complex phenotype by altering the initiating triggers, disease progression, and/or faulty electrical propagation that ends with SCE.

Therefore, the embodiments of the present teachings demonstrate significant progress in identifying markers for the accurate measurement of SCE risk in subjects along with methods of their use.

### SUMMARY

Disclosed herein is a method for predicting the likelihood of a sudden cardiac event (SCE) in a subject, comprising: obtaining a first dataset associated with a sample obtained from the subject, wherein the first dataset comprises data for a single nucleotide polymorphism (SNP) marker selected from Table 15; and analyzing the first dataset to determine the presence or absence of data for the SNP marker, wherein the presence of the SNP marker data is positively correlated or negatively correlated with the likelihood of SCE in the subject.

In some aspects, the SNP marker is rs17024266.

In some aspects, the first dataset comprises data for at least two, three, four, five, six, seven, eight, nine, ten, eleven, twelve, thirteen, fourteen, fifteen, sixteen, seventeen, eighteen, nineteen, twenty or more SNP markers selected from Table 15, and further comprising analyzing the first dataset to determine the presence or absence of data for the at least two, three, four, five, six, seven, eight, nine, ten, eleven, twelve, thirteen, fourteen, fifteen, sixteen, seventeen, eighteen, nineteen, twenty or more SNP markers selected from Table 15.

In some aspects, the method further includes determining the likelihood of SCE in the subject according to the relative number of positively correlated and negatively correlated SNP marker data present in the first dataset.

In some aspects, the method further includes determining the likelihood that the subject would benefit from implantation of an internal cardioverter defibrillator (ICD) based on the analysis. In some aspects, the SCE is a ventricular arrhythmia.

In some aspects, the SNP marker comprises at least one SNP marker selected from the group consisting of: rs17024266, rs1472929, rs17093751, rs6791277, rs4665719, rs12477891, rs5943590, rs101861.5, and rs10088053.

In some aspects, the likelihood of SCE in the subject is increased in the subject compared to a control. In some aspects, the control is a second dataset associated with a control sample, wherein the second dataset comprises data for a control wild-type marker at a specified locus rather than the SNP marker at that locus. In some aspects, the likelihood of SCE in the subject is not increased in the subject compared to a control.

In some aspects, the method further includes selecting a therapeutic regimen based on the analysis.

In some aspects, the data is genotyping data.

In some aspects, the method is implemented on one or more computers. In some aspects, the first dataset is obtained stored on a storage memory. In some aspects, obtaining the first dataset associated with the sample comprises obtaining the sample and processing the sample to experimentally determine the first dataset. In some aspects, obtaining the first dataset associated with the sample comprises receiving the first dataset directly or indirectly from a third party that has processed the sample to experimentally determine the first dataset. In some aspects, the data is obtained from a nucleotide-based assay.

In some aspects, the subject is a human subject.

In some aspects, the method further includes assessing a clinical factor in the subject; and combining the assessment with the analysis of the first dataset to predict the likelihood of SCE in the subject. In some aspects, the clinical factor comprises at least one clinical factor selected from the group consisting of age, gender, race, implant indication, prior pacing status, ICD presence, cardiac resynchronization therapy defibrillator (CRT-D) presence, total number of devices, device type, defibrillation thresholds performed, number of programming zones, heart failure (HF) etiology, HF onset, left ventricular ejection fraction (LVEF) at implant, New York Heart Association (NYHA) class, months from most recent myocardial infarction (MI) at implant, prior arrhythmia event in setting of MI or arthroscopic chondral osseous autograft transplantation (Cor procedure), diabetes status, Blood Urea Nitrogen (BUN), Cr, renal disease history, rhythm parameters to determine sinus v. non-sinus, heart rate, QRS duration prior to implant, left bundle branch block, systolic blood pressure, history of hypertension, smoking status, pulmonary disease, body mass index (BMI), family history of sudden cardiac death, B-type natriuretic peptide (BNP) levels, prior cardiac surgeries, medications, microvolt-level T-wave alternans (MTWA) result, and inducibility at electro-physiologic study (EPS).

Also described herein is a method for determining the likelihood of SCE in a subject, comprising: obtaining a sample from the subject, wherein the sample comprises a SNP marker selected from Table 15; contacting the sample with a reagent; generating a complex between the reagent and the SNP marker; detecting the complex to obtain a dataset associated with the sample, wherein the dataset comprises data for the SNP marker; and analyzing the dataset to determine the presence or absence of the SNP marker, wherein the presence of the marker is positively correlated or negatively correlated with the likelihood of SCE in the subject.

In some aspects, the SNP marker is rs17024266,

In some aspects, the first dataset comprises data for at least two, three, four, five, six, seven, eight, nine, ten, eleven, twelve, thirteen, fourteen, fifteen, sixteen, seventeen, eighteen, nineteen, twenty or more SNP markers selected from Table 15, and further comprising analyzing the first dataset to determine the presence or absence of data for the at least two, three, four, five, six, seven, eight, nine, ten, eleven, twelve, thirteen, fourteen, fifteen, sixteen, seventeen, eighteen, nineteen, twenty or more SNP markers selected from Table 15.

In some aspects, the method further includes determining the likelihood of SCE in the subject according to the relative number of positively correlated and negatively correlated SNP marker data present in the first dataset.

In some aspects, the method further includes determining the likelihood that the subject would benefit from implantation of an internal cardioverter defibrillator (ICD) based on the analysis. In some aspects, the SCE is a ventricular arrhythmia.

In some aspects, the SNP marker comprises at least one SNP marker selected from the group consisting of: rs17024266, rs1472929, rs17093751, rs6791277, rs4665719, rs12477891, rs5943590, rs1018615, and rs10088053.

In some aspects, the likelihood of SCE in the subject is increased in the subject compared to a control. In some aspects, the control is a second dataset associated with a control sample, wherein the second dataset comprises data for a control wild-type marker at a specified locus rather than the SNP marker at that locus. In some aspects, the likelihood of SCE in the subject is not increased in the subject compared to a control.

In some aspects, the method further includes selecting a therapeutic regimen based on the analysis.

In some aspects, the data is genotyping data.

In some aspects, the method is implemented on one or more computers. In some aspects, the data is obtained from a nucleotide-based assay.

In some aspects, the subject is a human subject.

In some aspects, the method further includes assessing a clinical factor in the subject; and combining the assessment with the analysis of the first dataset to predict the likelihood of SCE in the subject. In some aspects, the clinical factor comprises at least one clinical factor selected from the group consisting of age, gender, race, implant indication, prior pacing status, ICD presence, cardiac resynchronization therapy defibrillator (CRT-D) presence, total number of devices, device type, defibrillation thresholds performed, number of programming zones, heart failure (HF) etiology, HF onset, left ventricular ejection fraction (LVEF) at implant, New York Heart Association (NYHA) class, months from most recent myocardial infarction (MI) at implant, prior arrhythmia event in setting of or arthroscopic chondral osseous autograft transplantation (Cor procedure), diabetes status, Blood Urea Nitrogen (BUN), Cr, renal disease history, rhythm parameters to determine sinus v. non-sinus, heart rate, QRS duration prior to implant, left bundle branch block, systolic blood pressure, history of hypertension, smoking status, pulmonary disease, body mass index (BMI), family history of sudden cardiac death, B-type natriuretic peptide (BNP) levels, prior cardiac surgeries, medications, microvolt-level T-wave alternans (MTWA) result, and inducibility at electro-physiologic study (EPS).

Also described herein is a computer-implemented method for predicting the likelihood of SCE in a subject, comprising: storing, in a storage memory, a dataset associated with a first sample obtained from the subject, wherein the dataset comprises data for a SNP marker selected from Table 15; and analyzing, by a computer processor, the dataset to determine the presence or absence of the SNP marker, wherein the presence of the SNP marker is positively correlated or negatively correlated with the likelihood of SCE in the subject.

In some aspects, the SNP marker is rs17024266,

In some aspects, the first dataset comprises data for at least two, three, four, five, six, seven, eight, nine, ten, eleven, twelve, thirteen, fourteen, fifteen, sixteen, seventeen, eighteen, nineteen, twenty or more SNP markers selected from Table 15, and further comprising analyzing the first dataset to determine the presence or absence of data for the at least two, three, four, five, six, seven, eight, nine, ten, eleven, twelve, thirteen, fourteen, fifteen, sixteen, seventeen, eighteen, nineteen, twenty or more SNP markers selected from Table 15.

In some aspects, the method further includes determining the likelihood of SCE in the subject according to the relative number of positively correlated and negatively correlated SNP marker data present in the first dataset.

In some aspects, the method further includes determining the likelihood that the subject would benefit from implantation of an internal cardioverter defibrillator (ICD) based on the analysis. In some aspects, the SCE is a ventricular arrhythmia.

In some aspects, the SNP marker comprises at least one SNP marker selected from the group consisting of: rs17024266, rs1472929, rs17093751, rs6791277, rs4665719, rs12477891, rs5943590, rs1018615, and rs10088053.

In some aspects, the likelihood of SCE in the subject is increased in the subject compared to a control. In some aspects, the control is a second dataset associated with a control sample, wherein the second dataset comprises data for a control wild-type marker at a specified locus rather than the SNP marker at that locus. In some aspects, the likelihood of SCE in the subject is not increased in the subject compared to a control.

In some aspects, the method further includes selecting a therapeutic regimen based on the analysis.

In some aspects, the data is genotyping data.

In some aspects, the method is implemented on one or more computers. In some aspects, the first dataset is obtained stored on a storage memory. In some aspects, obtaining the first dataset associated with the sample comprises obtaining the sample and processing the sample to experimentally determine the first dataset. In some aspects, obtaining the first dataset associated with the sample comprises receiving the first dataset directly or indirectly from a third party that has processed the sample to experimentally determine the first dataset. In some aspects, the data is obtained from a nucleotide-based assay.

In some aspects, the subject is a human subject.

In some aspects, the method further includes assessing a clinical factor in the subject; and combining the assessment with the analysis of the first dataset to predict the likelihood of SCE in the subject. In some aspects, the clinical factor comprises at least one clinical factor selected from the group consisting of age, gender, race, implant indication, prior pacing status, ICD presence, cardiac resynchronization therapy defibrillator (CRT-D) presence, total number of devices, device type, defibrillation thresholds performed, number of programming zones, heart failure (HF) etiology, HF onset, left ventricular ejection fraction (LVEF) at implant, New York Heart Association (NYHA) class, months from most recent myocardial infarction (MI) at implant, prior arrhythmia event in setting of MI or arthroscopic chondral osseous autograft transplantation (Cor procedure), diabetes status, Blood Urea Nitrogen (BUN), Cr, renal disease history, rhythm parameters to determine sinus v. non-sinus, heart rate, QRS duration prior to implant, left bundle branch block, systolic blood pressure, history of hypertension, smoking status, pulmonary disease, body mass index (BMI), family history of sudden cardiac death, B-type natriuretic peptide (BNP) levels, prior cardiac surgeries, medications, microvolt-level T-wave alternans (MTWA) result, and inducibility at electro-physiologic study (EPS).

Also described herein is a system for predicting the likelihood of SCE in a subject, the system comprising: a storage memory for storing a dataset associated with a sample obtained from the subject, wherein the dataset comprises data for a SNP marker selected from Table 15; and a processor communicatively coupled to the storage memory for analyzing the dataset to determine the presence or absence of the SNP marker, wherein the presence of the SNP marker is positively correlated or negatively correlated with the likelihood of SCE in the subject.

In some aspects, the SNP marker is rs17024266.

In some aspects, the first dataset comprises data for at least two, three, four, five, six, seven, eight, nine, ten, eleven, twelve, thirteen, fourteen, fifteen, sixteen, seventeen, eighteen, nineteen, twenty or more SNP markers selected from Table 15, and further comprising analyzing the first dataset to determine the presence or absence of data for the at least two, three, four, five, six, seven, eight, nine, ten, eleven, twelve, thirteen, fourteen, fifteen, sixteen, seventeen, eighteen, nineteen, twenty or more SNP markers selected from Table 15.

In some aspects, the system further includes determining the likelihood of SCE in the subject according to the relative number of positively correlated and negatively correlated SNP marker data present in the first dataset.

In some aspects, the system further includes determining the likelihood that the subject would benefit from implantation of an internal cardioverter defibrillator (ICD) based on the analysis. In some aspects, the SCE is a ventricular arrhythmia.

In some aspects, the SNP marker comprises at least one SNP marker selected from the group consisting of: rs17024266, rs1472929, rs17093751, rs6791277, rs4665719, rs12477891, rs5943590, rs1018615, and rs10088053.

In some aspects, the likelihood of SCE in the subject is increased in the subject compared to a control. In some aspects, the control is a second dataset associated with a control sample, wherein the second dataset comprises data for a control wild-type marker at a specified locus rather than the SNP marker at that locus. In some aspects, the likelihood of SCE in the subject is not increased in the subject compared to a control.

In some aspects, the system further includes selecting a therapeutic regimen based on the analysis.

In some aspects, the data is genotyping data.

In some aspects, the first dataset is obtained stored on a storage memory. In some aspects, obtaining the first dataset associated with the sample comprises obtaining the sample and processing the sample to experimentally determine the first dataset. In some aspects, obtaining the first dataset associated with the sample comprises receiving the first dataset directly or indirectly from a third party that has processed the sample to experimentally determine the first dataset. In some aspects, the data is obtained from a nucleotide-based assay.

In some aspects, the subject is a human subject.

In some aspects, the system further includes assessing a clinical factor in the subject; and combining the assessment with the analysis of the first dataset to predict the likelihood of SCE in the subject. In some aspects, the clinical factor comprises at least one clinical factor selected from the group consisting of age, gender, race, implant indication, prior pacing status, ICD presence, cardiac resynchronization therapy defibrillator (CRT-D) presence, total number of devices, device type, defibrillation thresholds performed, number of programming zones, heart failure (HF) etiology, HF onset, left ventricular ejection fraction (LVEF) at implant, New York Heart Association (NYHA) class, months from most recent myocardial infarction (MI) at implant, prior arrhythmia event in setting of MI or arthroscopic chondral osseous autograft transplantation (Cor procedure), diabetes status, Blood Urea Nitrogen (BUN), Cr, renal disease history, rhythm parameters to determine sinus v. non-sinus, heart rate, QRS duration prior to implant, left bundle branch block, systolic blood pressure, history of hypertension, smoking status, pulmonary disease, body mass index (BMI), family history of sudden cardiac death, B-type natriuretic peptide (BNP) levels, prior cardiac surgeries, medications, microvolt-level T-wave alternans (MTWA) result, and inducibility at electro-physiologic study (EPS).

Also described herein is a computer-readable storage medium storing computer executable program code, the program code comprising: program code for storing a dataset associated with a sample obtained from a subject, wherein the dataset comprises data for a SNP marker selected from Table 15; and program code for analyzing the dataset to determine the presence or absence of the SNP marker, wherein the presence of the SNP marker is positively correlated or negatively correlated with the likelihood of SCE in the subject.

Also described herein is a kit for use in predicting the likelihood of SCE in a subject, comprising: a set of reagents comprising a plurality of reagents for determining from a sample obtained from the subject data for a SNP marker selected from Table 15; and instructions for using the plurality of reagents to determine data from the sample. In some aspects, the instructions comprise instructions for conducting a nucleotide-based assay.

Also described herein is a kit for use in predicting the likelihood of SCE in a subject, comprising: a set of reagents consisting essentially of a plurality of reagents for determining from a sample obtained from the subject data for a SNP marker selected from Table 15; and instructions for using the plurality of reagents to determine data from the sample. In some aspects, the instructions comprise instructions for conducting a nucleotide-based assay.

### BRIEF DESCRIPTION OF THE DRAWINGS

FIG. 1 shows that 3.3% of SNPs ailed the applied SNP call rate based on a cutoff of 95%.

FIG. 2 is a deFinetti diagram that shows most of the tested SNPs out of equilibrium have a low SNP call rate <95%.

FIG. 3 is a cluster diagram of a representative example SNP (SNP_A-1859379).

FIG. 4 shows that the non-pseudo-autosomal SNPs on chromosome X show no such pathology.

FIG. 5 shows a gender determination plot.

FIG. 6 shows that subject gender was significantly associated with VT/VF time-to-event (TTE) in a Kaplan-Meier plot.

FIG. 7 is a Kaplan-Meier plot that shows there is no discernible association of high/low MADIT II score with VT/VF arrhythmia.

FIG. 8 shows that the individual components of the MADIT II score show no significant association, except for the NYHA class, which shows marginally-significant association.

FIG. 9 is a Kaplan-Meier plot showing no significant association of BUN level with VT/VF arrhythmia. FIG. 9 also shows that creatinine level has no discernible association with VT/VF arrhythmia.

FIG. 10 shows at diabetes status does not have a significant association with VT/VF arrhythmia.

FIG. 11 shows that primary geneset analyses shows no statistical significance.

FIG. 12 shows p-values of the secondary geneset analyses in the plot with the horizontal dashed-line showing the Bonferroni adjustment required to achieve significance for 414 tests. Two genes had significant association: CENPO and ADCY3.

FIG. 13 is a QQ normal plot that shows the null distribution from the permutation test fits a normal distribution for the CENPO gene.

FIG. 14 is a genotype cluster plot of the top hitting SNP (SNP_A-2053054) in the GWAS analyses.

FIG. 15 is a Kaplan-Meier plot showing differential survival between the different genotypes for SNP_A-2053054.

FIG. 16 shows a test of the Cox model fit that makes a proportional odds assumption and a gender plot.

FIG. 17 is a Manhattan plot showing the p-values for the SNPs on chromosome 4, which includes the top hitting SNPs. The red dashed-line at the top represents the conservative Bonferroni level required for genome-wide significance.

FIG. 18 is a plot showing the results of calculations for contiguous blocks and random blocks and for the several block sizes 100, 500, and 1000, and as a function of the percent cutoff. Each curve approaches 100% on the right. The right side values include the independent SNPs as well as the random noise.

FIG. 19 shows an estimated value of between 13% to 26% for the percentage of independent SNPs identified in the study.

### DETAILED DESCRIPTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Most of the words used in this specification have the meaning that would be attributed to those words by one skilled in the art. Words specifically defined in the specification have the meaning provided in the context of the present teachings as a whole, and as are typically understood by those skilled in the art. In the event that a conflict arises between an art-understood definition of a word or phrase and a definition of the word or phrase as specifically taught in this specification, the specification shall control.

It must be noted that, as used in the specification and the appended claims, the singular forms “a,” “an,” and “the” include plural referents unless the context clearly dictates otherwise.

Terms used in the claims and specification are defined as set forth below unless otherwise specified.

“Biomarker,” “biomarkers,” “marker” or “markers” refers to a sequence characteristic of a particular variant allele (i.e., polymorphic site) or wild-type allele. A marker can include any allele, including wild-types alleles, SNPs, microsatellites, insertions, deletions, duplications, and translocations. A marker can also include a peptide encoded by an allele comprising nucleic acids. A marker in the context of the present teachings encompasses, without limitation, cytokines, chemokines, growth factors, proteins, peptides, nucleic acids, oligonucleotides, and metabolites, together with their related metabolites, mutations, variants, polymorphisms, modifications, fragments, subunits, degradation products, elements, and other analytes or sample-derived measures. Markers can also include mutated proteins, mutated nucleic acids, variations in copy numbers and/or transcript variants. Markers also encompass non-blood borne factors and non-analyte physiological markers of health status, and/or other factors or markers not measured from samples biological samples such as bodily fluids), such as clinical parameters and traditional factors for clinical assessments. Markers can also include any indices that are calculated and/or created mathematically. Markers can also include combinations of any one or more of the foregoing measurements, including temporal trends and differences.

To “analyze” includes measurement and/or detection of data associated with a marker (such as, e.g., presence or absence of a SNP, allele, or constituent expression levels) in the sample (or, e.g., by obtaining a dataset reporting such measurements, as described below). In some aspects, an analysis can include comparing the measurement and/or detection against a measurement and/or detection in a sample or set of samples from the same subject or other control subject(s). The markers of the present teachings can be analyzed by any of various conventional methods known in the art.

A “subject” in the context of the present teachings is generally a mammal. The subject can be a patient. The term “mammal” as used herein includes but is not limited to a human, non-human primate, dog, cat, mouse, rat, cow, horse, and pig. Mammals other than humans can be advantageously used as subjects that represent animal models of inflammation. A subject can be male or female. A subject can be one who has been previously diagnosed or identified as having a sudden cardiac event. A subject can be one who has already undergone, or is undergoing, a therapeutic intervention for a sudden cardiac event. A subject can also be one who has not been previously diagnosed as having a sudden cardiac event; e.g., a subject can be one who exhibits one or more symptoms or risk factors for a sudden cardiac event, or a subject who does not exhibit symptoms or risk factors for a sudden cardiac event, or a subject who is asymptomatic for a sudden cardiac event.

A “sample” in the context of the present teachings refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including gingival crevicular fluid, bone marrow, cerebrospinal fluid (CSF), saliva, mucous, sputum, semen, sweat, urine, or any other bodily fluids. “Blood sample” can refer to whole blood or any fraction thereof, including blood cells, red blood cells, white blood cells or leucocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art.

A “dataset” is a set of data (e.g., numerical values) resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring, PCR, microarray, one or more primers, one or more probes, antibody binding, or ELISA. The phrase also encompasses receiving a set of data, e.g., from a third party that has processed the sample to experimentally determine the dataset. Additionally, the phrase encompasses mining data from at least one database or at least one publication or a combination of databases and publications.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject's clinical parameters based on a control.

A “prognosis” is a prediction as to the likely outcome of a disease. Prognostic estimates are useful in, e.g., determining an appropriate therapeutic regimen for a subject.

A “nucleotide-based assay” includes a nucleic acid binding assay capable of detecting a SNP, such as a hybridization assay that uses nucleic acid sequencing. Other examples of nucleotide-based assays include single base extensions (see, e.g., Kobayashi et al, Mol. Cell. Probes, 9:175-182, 1995); single-strand conformation polymorphism analysis, as described, e.g, in Orita et al., Proc. Nat. Acad. Sci. 86, 2766-2770 (1989), allele specific oligonucleotide hybridization (ASO) (e.g., Stoneking et al., Am. J. Hum. Genet. 48:70-382, 1991; Saiki et al., Nature 324, 163-166, 1986; EP 235,726; and WO 89/11548); and sequence-specific amplification or primer extension methods as described in, for example, WO 93/22456; U.S. Pat. Nos. 5,137,806; 5,595,890; 5,639,611; and U.S. Pat. No. 4,851,331; 5′-nuclease assays, as described in U.S. Pat. Nos. 5,210,015; 5,487,972; and 5,804,375; and Holland et al, 1988, Proc. Natl. Acad. Sci. USA 88:7276-7280. Other examples are described in U.S. Pat. Pub. 20110045469, herein incorporated by reference.

### Markers

The genome exhibits sequence variability between individuals at many locations in the genome; in other words, there are many polymorphic sites in a population. In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a disease or abnormal phenotype). Alleles that differ from the reference are referred to as “variant” alleles.

SNP nomenclature as reported herein refers to the official Reference SNP (rs) ID identification tag as assigned to each unique SNP by the National Center for Biotechnological Information (NCBI), as of the filing date of the instant specification and/or an application to which the instant specification claims priority. Further information can be found on the SNP database of the NCBI website.

A “haplotype” refers to a segment of a DNA strand that is characterized by a specific combination of two or more markers (e.g., alleles) arranged along the segment. In a certain embodiment, the haplotype can comprise two or more alleles, three or more alleles, four or more alleles, or five or more alleles. The term “susceptibility,” as described herein, encompasses at least increased susceptibility. Thus, particular markers and/or haplotypes of the invention may be characteristic of increased susceptibility of a sudden cardiac event, as characterized by a relative risk of greater than one compared to a control. Markers and/or haplotypes that confer increased susceptibility of a sudden cardiac event are furthermore considered to be “at-risk,” as they confer an increased risk of disease compared to a control.

A nucleotide position at which more than one sequence is possible in a population (either a natural population or a synthetic population, e.g., a library of synthetic molecules) is referred to herein as a “polymorphic site.” Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Alleles for SNP markers as referred to herein refer to the bases A, C, or T as they occur at the polymorphic site in the SNP assay employed. The person skilled in the art will realize that by assaying or reading the opposite strand, the complementary allele can in each case be measured. Thus, Coca polymorphic site containing an A/G polymorphism, the assay employed may either measure the percentage or ratio of the two bases possible, i.e., A and G. Alternatively, by designing an assay that determines the opposite strand on the DNA template, the percentage or ratio of the complementary bases T/C can be measured. Quantitatively (for example, in terms of relative risk), identical results would be obtained from measurement of either DNA strand (+strand or −strand). Polymorphic sites can allow for differences in sequences based on substitutions, insertions or deletions. For example, a polymorphic microsatellite has multiple small repeats of bases (such as CA repeats) at a particular site in which the number of repeat lengths varies in the general population. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence of interest. Alleles that differ from the reference are referred to as “variant” alleles. Variants can include changes that affect a polypeptide, e.g., a polypeptide encoded by a gene. These sequence differences, when compared to a reference nucleotide sequence, can include the insertion or deletion of a single nucleotide, or of more than one nucleotide. Such sequence differences may result in a frame shift; the change of at least one nucleotide, may result in a change in the encoded amino acid; the change of at least one nucleotide, may result in the generation of a premature stop codon; the deletion of several nucleotides, may result in a deletion of one or more amino acids encoded by the nucleotides; the insertion of one or several nucleotides, such as by unequal recombination or gene conversion, may result in an interruption of the coding sequence of a reading frame; duplication of all or a part of a sequence; transposition; or a rearrangement of a nucleotide sequence, as described in detail herein. Such sequence changes alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a sudden cardiac event or a susceptibility to a sudden cardiac event can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level in tumors. The polypeptide encoded by the reference nucleotide sequence is the “reference” polypeptide with a particular reference amino acid sequence, and polypeptides encoded by variant alleles are referred to as “variant” polypeptides with variant amino acid sequences.

A polymorphic microsatellite has multiple small repeats of bases that are 2-8 nucleotides in length (such as CA repeats) at a particular site, in which the number of repeat lengths varies in the general population. An indel is a common form of polymorphism comprising a small insertion or deletion that is typically only a few nucleotides long.

The haplotypes described herein can be a combination of various genetic markers, e.g., SNPs and microsatellites, having particular alleles at polymorphic sites. The haplotypes can comprise a combination of various genetic markers; therefore, detecting haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999)), PcR, LCR, Nested PCR and other techniques for nucleic acid amplification. These markers and SNPs can be identified in at-risk haplotypes. Certain methods of identifying relevant markers and SNPs include the use of linkage disequilibrium (LD) and/or LOD scores.

In certain methods described herein, an individual who is at-risk for a sudden cardiac event is an individual in whom an at-risk marker or haplotype is identified. In one aspect, the at-risk marker or haplotype is one that confers a significant increased risk (or susceptility) of a sudden cardiac event. In one embodiment, significance associated with a marker or haplotype is measured by a relative risk. In a further embodiment, the significance is measured by a percentage. In one embodiment, a significant increased risk is measured as a relative risk of at least about 1.2, including but not limited to: 1.2, 1.3, 1.4, 1.5, 1.6, 1.7, 1.8 and 1.9. In a further embodiment, a relative risk of at least 1.2 is significant. In a further embodiment, a relative risk of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%.

Thus, the term “susceptibility to a sudden cardiac event” indicates an increased risk or susceptility of a sudden cardiac event, by an amount that is significant, when a certain allele, marker, SNP or haplotype is present. It is understood however, that identifying whether an increased risk is medically significant may also depend on a variety of factors, including the specific disease, the marker or haplotype, and often, environmental factors.

An at-risk marker or haplotype in, or comprising portions of a gene, or in non-coding regions of the genome, is one where the marker or haplotype is more frequently present in an individual at risk for a sudden cardiac event (affected), compared to the frequency of its presence in a healthy individual (control), and wherein the presence of the marker or haplotype is indicative of susceptibility to a sudden cardiac event. As an example of a simple test for correlation would be a Fisher-exact test on a two by two table. Given a cohort of chromosomes the two by two table is constructed out of the number of chromosomes that include both of the markers or haplotypes, one of the markers or haplotypes but not the other and neither of the markers or haplotypes.

In certain aspects of the invention, at-risk marker or haplotype is an at-risk marker or haplotype within or near a gene, or in a non-coding region of the genome, that significantly correlates with a sudden cardiac event. In other aspects, an at-risk marker or haplotype comprises an at-risk marker or haplotype within or near a gene, or in a non-coding region of the genome, that significantly correlates with susceptibility to a sudden cardiac event.

Standard techniques for genotyping for the presence of SNPs and/or microsatellite markers can be used, such as fluorescent based techniques (Chen, et al., Genome Res. 9, 492 (1999)), PCR, LCR, Nested PCR and other techniques for nucleic acid amplification. In a preferred aspect, the method comprises assessing in an individual the presence or frequency of SNPs and/or microsatellites in, comprising portions of, a gene, wherein an excess or higher frequency of the SNPs and/or microsatellites compared to a healthy control individual is indicative that the individual is susceptible to a sudden cardiac event. Such SNPs and markers can form haplotypes that can be used as screening tools. These markers and SNPs can be identified in at-risk haploptypes. The presence of an at-risk haplotype is indicative of increased susceptibility to a sudden cardiac event, and therefore is indicative of an individual who falls within a target population for the treatment methods described herein.

### Nucleic Acids and Antibodies

Nucleic Acids, Portions and Variants

The nucleic acid molecules of the present invention can be RNA, for example, mRNA, or DNA, such as cDNA and genomic DNA. DNA molecules can be double-stranded or single-stranded; single-stranded RNA or DNA can be the coding, or sense, strand or the non-coding, or antisense strand. The nucleic acid molecule can include all or a portion of the coding sequence of the gene and can further comprise additional non-coding sequences such as introns and non-coding 3′ and 5′ sequences (including regulator sequences, for example).

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid molecule comprises at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb but not limited to 4 kb, 3 kb, 2 kb, 1 kb, 0.5 kb or 0.1 kb of nucleotides which flank the nucleic acid molecule in the genomic DNA of the cell from which the nucleic acid molecule is derived.

An isolated nucleic acid molecule can include a nucleic acid molecule or nucleic acid sequence that is synthesized chemically or by recombinant means. Such isolated nucleic acid molecules are useful as probes for isolating homologous sequences (e.g., from other mammalian species), for gene mapping (e.g., by in situ hybridization with chromosomes), or for detecting expression of the gene in tissue (e.g., human tissue), such as by Northern or Southern blot analysis.

Nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one aspect, the invention includes variants described herein that hybridize under high stringency hybridization conditions (e.g., for selective hybridization) to a nucleotide sequence encoding an amino acid sequence or a polymorphic variant thereof.

Such nucleic acid molecules can be detected and/or isolated by specific hybridization (e.g., under high stringency conditions). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 90%, 95%). For example, certain high stringency conditions can be used which distinguish perfectly complementary nucleic acids from those of less complementarity, “High stringency conditions,” “moderate stringency conditions” and “low stringency conditions,” as well as methods for nucleic acid hybridizations are explained on pages 2.10.1-2.10.16 and pages 6.3.1-6.3.6 in Current Protocols in Molecular Biology (Ausubel, F. et al., “Current Protocols in Molecular Biology”, John Wiley & Sons, (1998)), and in Kraus, M. and Aaronson, S., Methods Enzymol., 200:546-556 (1991), incorporated herein, by reference.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions ×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain aspects, the length of a sequence aligned for comparison purposes is at least 30%, for example, at least 40%, in certain aspects at least 60%, and in other aspects at least 70%, 80%, 90% or 95% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, word or can be varied (e.g., W=5 or W=20).

The present invention also provides isolated nucleic acid molecules that contain a fragment or portion that hybridizes under highly stringent conditions to a nucleotide sequence or the complement of such a sequence, and also provides isolated nucleic acid molecules that contain a fragment or portion that hybridizes under highly stringent conditions to a nucleotide sequence encoding an amino acid sequence or polymorphic variant thereof. The nucleic acid fragments of the invention are at least about 15, preferably at least about 18, 20, 23 or 25 nucleotides, and can be 30, 40, 50, 100, 200 or more nucleotides in length.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al., Science 254:1497-1500 (1991).

A probe or primer comprises a region of nucleotide sequence that hybridizes to at least about 15, for example about 20-25, and in certain aspects about 40, 50 or 75, consecutive nucleotides of a nucleic acid molecule comprising a contiguous nucleotide sequence of or polymorphic variant thereof. In other aspects, a probe or primer comprises 100 or fewer nucleotides, in certain aspects from 6 to 50 nucleotides, for example from 12 to 30 nucleotides. In other aspects, the probe or primer is at least 70% identical to the contiguous nucleotide sequence or to the complement of the contiguous nucleotide sequence, for example at least 80% identical, in certain aspects at least 90% identical, and in other aspects at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The nucleic acid molecules of the invention can be identified and isolated using standard molecular biology techniques and the sequence information provided herein. For example, nucleic acid molecules can be amplified and isolated by the polymerase chain reaction (PCR) using synthetic oligonucleotide primers designed based on the sequence of a nucleic acid sequence of interest or the complement of such a sequence, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis et al., Academic Press, San Diego, Calif., 1990); Manila et al., Nucl. Acids Res. 19: 4967 (1991); Eckert et al., PCR Methods and Applications 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci, USA 87:1874 (1990)) and nucleic acid based sequence amplification (NASBA). The tatter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled, for example, radiolabeled, and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Additionally, fluorescence methods are also available for analyzing nucleic acids (Chen et al., Genome Res. 9, 492 (1999)) and polypeptides. Using these or similar methods, the polypeptide and the DNA encoding the polypeptide can be isolated, sequenced and further characterized.

The nucleic acid sequences can also be used to compare with endogenous DNA sequences in patients to identify one or more of the disorders, and as probes, such as to hybridize and discover related DNA sequences or to subtract out known sequences from a sample. The nucleic acid sequences can further be used to derive primers for genetic fingerprinting. Portions or fragments of the nucleotide sequences identified herein (and the corresponding complete gene sequences) can be used in numerous ways, such as polynucleotide reagents. For example, these sequences can be used to (i) map their respective genes on a chromosome; and, thus, locate gene regions associated with genetic disease; (ii) identify an individual from a minute biological sample (tissue typing); and (iii) aid in forensic identification of a biological sample. The nucleic acid sequences can additionally be used as reagents in the screening and/or diagnostic assays described herein, and can also be included as components of kits (e.g., reagent kits) for use in the screening and/or diagnostic assays described herein.

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies which hind to altered or to non-altered (native) polypeptide, means for amplification of nucleic acids comprising a nucleic acid or for a portion of, or means for analyzing the nucleic acid sequence of a nucleic acid or for analyzing the amino acid sequence of a polypeptide as described herein, etc. The primers can be designed using portions of the nucleic acids flanking SNPs that are indicative of a sudden cardiac event.

Antibodies

Polyclonal antibodies and/or monoclonal antibodies that specifically bind one form of the gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)2 fragments which can be generated by treating the antibody with an enzyme such as pepsin. The invention provides polyclonal and monoclonal antibodies that bind to a polypeptide of the invention. The term “monoclonal antibody” or “monoclonal antibody composition,” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human cell hybridoma technique (Kozbor et al., Immunol. Today 4: 72 (1983)), the EBV-hybridoma technique (Cole et al., Monoclonal Antibodies and Cancer Therapy, Alan R. Liss, 1985, pp. 77-96) or trioma techniques. The technology for producing hybridomas is well known (see generally Current Protocols in Immunology (1994) Coligan et al., (eds.) John Wiley & Sons, Inc., New York, N.Y.), Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well-known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies; A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649 and U.S. Pat. No. 4,946,778 and No. 5,260,203, the disclosures of which are incorporated by reference.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide, Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

### Detection Assays

Nucleic acids, probes, primers, and antibodies such as those described herein can be used in a variety of methods of diagnosis of a susceptibility to a sudden cardiac event (e.g., an arrhythmia), as well as in kits (e.g., useful for diagnosis of a susceptibility to a sudden cardiac event). Similarly, the nucleic acids, probes, primers, and antibodies described herein can be used in methods of diagnosis of a protection against a sudden cardiac event, and also in kits. In one aspect, the kit comprises primers that can be used to amplify the markers of interest.

In one aspect of the invention, diagnosis of a susceptibility to a sudden cardiac event is made by detecting a polymorphism in a nucleic acid as described herein. The polymorphism can be a change in a nucleic acid, such as the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such change may be present in a single gene. Such sequence changes can cause a difference in the polypeptide encoded by a nucleic acid. For example, if the difference is a frame shift change, the frame shift can result in a change in the encoded amino acids, and/or can result in the generation of a premature stop codon, causing generation of a truncated polypeptide. Alternatively, a polymorphism associated with a disease or condition or a susceptibility to a disease or condition associated with a nucleic acid can be a synonymous alteration in one or more nucleotides (i.e., an alteration that does not result in a change in the polypeptide encoded by a nucleic acid). Such a polymorphism may alter splicing sites, affect the stability or transport of mRNA, or otherwise affect the transcription or translation of the gene.

In some aspects, a nucleotide-based assay is used to detect a SNP.

In a method of diagnosing a susceptibility to a sudden cardiac event, hybridization methods, such as Southern analysis, Northern analysis, or in situ hybridizations, can be used (see Current Protocols in Molecular Biology, Ausubel, F. et al., eds, John Wiley & Sons, including all supplements through 1999). For example, a biological sample (a “test sample”) from a test subject (the “test individual”) of genomic DNA, RNA, or cDNA, is obtained from an individual (RNA and cDNA can only be used for exonic markers), such as an individual suspected of having, being susceptible to or predisposed for, or carrying a defect for, a sudden cardiac event. The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in a nucleic acid is present, and/or to determine which splicing variant(s) encoded by the nucleic acid is present. The presence of the polymorphism or splicing variant(s) can be indicated by hybridization of the gene in the genomic DNA, RNA, or cDNA to a nucleic acid probe, A “nucleic acid probe,” as used herein, can be a DNA probe or an RNA probe; the nucleic acid probe can contain, for example, at least one polymorphism in a nucleic acid and/or contain a nucleic acid encoding a particular splicing variant of a nucleic acid. The probe can be any of the nucleic acid molecules described above (e.g., the gene or nucleic acid, a fragment, a vector comprising the gene or nucleic acid, a probe or primer, etc.).

To diagnose a susceptibility to a sudden cardiac event, a hybridization sample can be formed by contacting the test sample containing a nucleic acid with at least one nucleic acid probe. A probe for detecting mRNA or genomic DNA can be a labeled nucleic acid probe capable of hybridizing to mRNA or genomic DNA sequences. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA genomic DNA.

The hybridization sample is maintained under conditions that are sufficient to allow specific hybridization of the nucleic acid probe to a nucleic acid, “Specific hybridization,” as used herein, indicates exact hybridization (e.g., with no mismatches). Specific hybridization can be performed under high stringency conditions or moderate stringency conditions, for example, as described above. In a particularly preferred aspect, the hybridization conditions for specific hybridization are high stringency.

Specific hybridization, if present, is then detected using standard methods. If specific hybridization occurs between the nucleic acid probe and nucleic acid in the test sample, then the nucleic acid has the polymorphism, or is the splicing variant, that is present in the nucleic acid probe. More than one nucleic acid probe can also be used concurrently in this method. Specific hybridization of any one of the nucleic acid probes is indicative of a polymorphism in the nucleic acid, or of the presence of a particular splicing variant encoding the nucleic acid and can be diagnostic for a susceptibility to a sudden cardiac event.

In Northern analysis (see Current Protocols in Molecular Biology, Ausubel, F. et al., eds., John Wiley & Sons.) hybridization methods can be used to identify the presence of a polymorphism or a particular splicing variant, associated with a susceptibility to a sudden cardiac event or associated with a decreased susceptibility to a sudden cardiac event. For Northern analysis, a test sample of RNA is obtained from the individual by appropriate means. Specific hybridization of a nucleic acid probe to RNA from the individual is indicative of a polymorphism in a nucleic acid, or of the presence of a particular splicing variant encoded by a nucleic acid and is therefore diagnostic for the susceptibility to a sudden cardiac event. For representative examples of use of nucleic acid probes, see, for example, U.S. Pat. Nos. 5,288,611 and 4,851,330, both of which are herein incorporated by reference.

Alternatively, a peptide nucleic acid (PNA) probe can be used instead of a nucleic acid probe in the hybridization methods. PNA is a DNA mimic having a peptide-like, inorganic backbone, such as N-(2-aminoethyl) glycine units, with an organic base (A, G, C, T or U) attached to the glycine nitrogen via a methylene carbonyl linker (see, for example, Nielsen, P. E. et al., Bioconjugate Chemistry 5, American Chemical Society, p. 1 (1994). The PNA probe can be designed to specifically hybridize to a nucleic acid. Hybridization of the PNA probe to a nucleic acid can be diagnostic for a susceptibility to a sudden cardiac event.

In another method of the invention, alteration analysis by restriction digestion can be used to detect an alteration in the gene, if the alteration (mutation) or polymorphism in the gene results in the creation or elimination of a restriction site. A test sample containing genomic DNA is obtained from the individual. Polymerase chain reaction (PCR) can be used to amplify a nucleic acid (and, if necessary, the flanking sequences) in the test sample of genomic DNA from the test individual. RFLP analysis is conducted as described (see Current Protocols in Molecular Biology). The digestion pattern of the relevant DNA fragment indicates the presence or absence of the alteration or polymorphism in the nucleic acid, and therefore indicates the presence or absence a susceptibility to a sudden cardiac event.

Sequence analysis can also be used to detect specific polymorphisms in a nucleic acid. A test sample of DNA or RNA is obtained from the test individual. PCR or other appropriate methods can be used to amplify the gene or nucleic acid, and/or its flanking sequences, if desired. The sequence of a nucleic acid, or a fragment of the nucleic acid, or cDNA, or fragment of the cDNA, or mRNA, or fragment of the mRNA, is determined, using standard methods. The sequence of the nucleic acid, nucleic acid fragment, cDNA, cDNA fragment, mRNA, or mRNA fragment is compared with the known nucleic acid sequence of the gene or cDNA or mRNA, as appropriate. The presence of a polymorphism in a nucleic acid indicates that the individual has a susceptibility to a sudden cardiac event.

Allele-specific oligonucleotides can also be used to detect the presence of a polymorphism in a nucleic acid, through the use of dot-blot hybridization of amplified oligonucleotides with allele-specific oligonucleotide (ASO) probes (see, for example, Saiki, R. et al., Nature 324:163-166 (1986)). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to a nucleic acid, and, in the context of the instant invention, that contains a polymorphism associated with a susceptibility to a sudden cardiac event. An allele-specific oligonucleotide probe that is specific for particular polymorphisms in a nucleic acid can be prepared, using standard methods (see Current Protocols in Molecular Biology). To identify polymorphisms in the gene that are associated with a sudden cardiac event, a test sample of DNA is Obtained from the individual. PCR can be used to amplify all or a fragment of a nucleic acid and its flanking sequences. The DNA containing the amplified nucleic acid (or fragment of the gene or nucleic acid) is dot-blotted, using standard methods (see Current Protocols in Molecular Biology), and the blot is contacted with the oligonucleotide probe. The presence of specific hybridization of the probe to the amplified nucleic acid is then detected. Hybridization of an allele-specific oligonucleotide probe to DNA from the individual is indicative of a polymorphism in the nucleic acid, and is therefore indicative of susceptibility to a sudden cardiac event.

The invention further provides allele-specific oligonucleotides that hybridize to the reference or variant allele of a gene or nucleic acid comprising a single nucleotide polymorphism or to the complement thereof. These oligonucleotides can be probes or primers.

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer, which hybridizes at a distal site. Amplification proceeds from the two primers, resulting in a detectable product, which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′ end, or in the middle), the Tm could be increased considerably.

In another aspect, arrays of oligonucleotide probes that are complementary to target nucleic acid sequence segments from an individual can be used to identify polymorphisms in a nucleic acid. For example, in one aspect, an oligonucleotide array can be used. Oligonucleotide arrays typically comprise a plurality of different oligonucleotide probes that are coupled to a surface of a substrate in different known locations. These oligonucleotide arrays have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991), Pirrung et at, U.S. Pat. No. 5,143,854 (see also PCT Application No. WO 90/15070) and Fodor et al., PCT Publication No. WO 92/10092 and U.S. Pat. No. 5,424,186, the entire teachings of which are incorporated by reference herein, Techniques for the synthesis of these arrays using mechanical synthesis methods are described in, e.g., U.S. Pat. No. 5,384,261; the entire teachings are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., published PCT Application Nos. WO 92/10092 and WO 95/11995, and U.S. Pat. No. 5,424,186, the entire teachings of which are incorporated by reference herein. In brief a target nucleic acid sequence that includes one or more previously identified polymorphic markers is amplified by well-known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detecting a single polymorphism, arrays can include multiple detection blocks, and thus be capable of analyzing multiple, specific polymorphisms. In alternative aspects, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional uses of oligonucleotide arrays for polymorphism detection can be found, for example, in U.S. Pat. Nos. 5,858,659 and 5,837,832, the entire teachings of which are incorporated by reference herein. Other methods of nucleic acid analysis can be used to detect polymorphisms in a sudden cardiac event gene or variants encoded by a sudden cardiac event-associated gene. Representative methods include direct manual sequencing (Church and Gilbert, Proc. Natl. Acad. Sci. USA 81:1991-1995 (1988); Sanger, F. et al., Proc. Natl. Acad. Sci, USA 74:5463-5467 (1977); Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al., Proc. Natl. Acad. Sci. USA 86:232-236 (1989)), mobility shift analysis (Orita, M. et al., Proc. Natl. Acad. Sci, USA 86:2766-2770 (1989)), restriction enzyme analysis (Haven et Cell 15:25 (1978); Geever, et al., Proc. Natl. Acad. Sci. USA 78:5081 (1980); heteroduplex analysis; chemical mismatch cleavage (CMC) (Cotton et al., Proc, Natl. Acad. Sci. USA 85:4397-4401 (1985)); RNase protection assays (Myers, R. M. et al., Science 230:1242 (1985)); use of polypeptides which recognize nucleotide mismatches, such as E. coli mutS protein; allele-specific PCR, for example.

In one aspect of the invention, diagnosis of a susceptibility to a sudden cardiac event, can also be made by expression analysis by quantitative PCR (kinetic thermal cycling). This technique, utilizing TaqMan assays, can assess the presence of an alteration in the expression or composition of the polypeptide encoded by a nucleic acid or splicing variants encoded by a nucleic acid. TaqMan probes can also be used to allow the identification of polymorphisms and whether a patient is homozygous or heterozygous. Further, the expression of the variants can be quantified as physically or functionally different.

In another aspect of the invention, diagnosis of a susceptibility to a sudden cardiac event can be made by examining expression and/or composition of a polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by a nucleic acid, or for the presence of a particular variant encoded by a nucleic acid. An alteration in expression of a polypeptide encoded by a nucleic acid can be, for example, an alteration in the quantitative polypeptide expression (i.e., the amount of polypeptide produced); an alteration in the composition of a polypeptide encoded by a nucleic acid is an alteration in the qualitative polypeptide expression (e.g., expression of an altered polypeptide or of a different splicing variant). In a preferred aspect, diagnosis of a susceptibility to a sudden cardiac event can be made by detecting a particular splicing variant encoded by that nucleic acid, or a particular pattern of splicing variants.

Both such alterations (quantitative and qualitative) can also be present. The term “alteration” in the polypeptide expression or composition, as used herein, refers to an alteration in expression or composition in a test sample, as compared with the expression or composition of polypeptide by a nucleic acid in a control sample. A control sample is a sample that corresponds to the test sample (e.g., is from the same type of cells), and is from an individual who is not affected by a susceptibility to a sudden cardiac event. An alteration in the expression or composition of the polypeptide in the test sample, as compared with the control sample, is indicative of a susceptibility to a sudden cardiac event. Similarly, the presence of one or more different splicing variants in the test sample, or the presence of significantly different amounts of different splicing variants in the test sample, as compared with the control sample, is indicative of a susceptibility to a sudden cardiac event. Various means of examining expression or composition of the polypeptide encoded by a nucleic acid can be used, including: spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly Chapter 10). For example, in one aspect, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling a DNA probe with biotin such that it can be detected with fluorescently labeled streptavidin.

Western blotting analysis, using an antibody as described above that specifically binds to a polypeptide encoded by an altered nucleic acid or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered nucleic acid, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for a susceptibility to a sudden cardiac event, as is the presence (or absence) of particular splicing variants encoded by the nucleic acid.

In one aspect of this method, the level or amount of polypeptide encoded by a nucleic acid in a test sample is compared with the level or amount of the polypeptide encoded by the nucleic acid in a control sample. A level or amount of the polypeptide in the test sample that is higher or tower than the level or amount of the polypeptide in the control sample, such that the difference is statistically significant, is indicative of an alteration in the expression of the polypeptide encoded by the nucleic acid, and is diagnostic for a susceptibility to a sudden cardiac event. Alternatively, the composition of the polypeptide encoded by a nucleic acid in a test sample is compared with the composition of the polypeptide encoded by the nucleic acid in a control sample (e.g., the presence of different splicing variants). A difference in the composition of the polypeptide in the test sample, as compared with the composition of the polypeptide in the control sample, is diagnostic for a susceptibility to a sudden cardiac event. In another aspect, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to a sudden cardiac event.

The same methods can conversely be used to identify the presence of a difference when compared to a control (disease) sample. A difference from the control can be indicative of a protective allele against a sudden cardiac event.

In addition, one of skill will also understand that the above described methods can also generally be used to detect markers that do not include a polyporphism.

### Diagnostic and Genetic Tests and Methods

As described herein, certain markers and haplotypes comprising such markers are found to be useful for determination of susceptibility to a sudden cardiac event—i.e., they are found to be useful for diagnosing a susceptibility to a sudden cardiac event. Examples of methods for determining which markers are particularly useful in the determination of susceptibility to a sudden cardiac event are described in more detail in the Examples section below. Particular markers and haplotypes can be found more frequently in individuals with a sudden cardiac event than in individuals without a sudden cardiac event. Therefore, these markers and haplotypes can have predictive value for detecting a sudden cardiac event, or a susceptibility to a sudden cardiac event, in an individual. The haplotypes and markers described herein can be, in some cases, a combination of various genetic markers, e.g., SNPs and microsatellites. Therefore, detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

The knowledge about a genetic variant that confers a risk of developing a sudden cardiac event offers the opportunity to apply a genetic-test to distinguish between individuals with increased risk of developing the disease (i.e., carriers of the at-risk variant) and those with decreased risk of developing the disease (i.e., carriers of the protective variant). The core values of genetic testing, for individuals belonging to both of the above mentioned groups, are the possibilities of being able to diagnose the disease at an early stage and provide information to the clinician about prognosis/aggressiveness of the disease in order to be able to apply the most appropriate treatment. For example, the application of a genetic test for a sudden cardiac event can provide an opportunity for the detection of the disease at an earlier stage which may lead to the application of therapeutic measures at an earlier stage, and thus can minimize the deleterious effects of the symptoms and serious health consequences conferred by a sudden cardiac event.

Also described herein is a method for predicting the likelihood of a sudden cardiac event in a subject comprising a plurality of SNPs. In some aspects, the subject's genome comprises a plurality of SNPs shown in Table 15. In some aspects, the method includes weighting each positively correlated SNP and each negatively correlated SNP in Table 15 equally and predicting the likelihood of a sudden cardiac event based on the relative number of positively correlated and negatively correlated SNPs present in the subject. For example, if the subject comprises a greater number of positively correlated SNPs than negatively correlated SNPs then the subject has an increased likelihood of experiencing a sudden cardiac event.

### Clinical Factors

In some embodiments, one or more clinical factors in a subject can be assessed. In some embodiments, assessment of one or more clinical factors in a subject can be combined with a marker analysis in the subject to identify risk and/or susceptibility of SCE in the subject.

Various clinical factors are generally known to one of ordinary skill in the art to be associated with sudden cardiac events. In some embodiments, clinical factors known to one of ordinary skill in the art to be associated with a sudden cardiac event, such as an arrhythmia, can include age, gender, race, implant indication, prior pacing status, ICD presence, cardiac resynchronization therapy defibrillator (CRT-D) presence, total number of devices, device type, defibrillation thresholds performed, number of programming zones, heart failure (HF) etiology, HF onset, left ventricular ejection fraction (LVEF) at implant, New York Heart Association (NYHA) class, months from most recent myocardial infarction (MI) at implant, prior arrhythmia event in setting of MI or arthroscopic chondral osseous autograft transplantation (Cor procedure), diabetes status, Blood Urea Nitrogen (BUN), Cr, renal disease history, rhythm parameters to determine sinus v. non-sinus, heart rate, QRS duration prior to implant, left bundle branch block, systolic blood pressure, history of hypertension, smoking status, pulmonary disease, body mass index (BMI), family history of sudden cardiac death, B-type natriuretic peptide (BNP) levels, prior cardiac surgeries, medications, microvolt-level T-wave alternans (MTWA) result, and/or inducibility at electro-physiologic study (EPS).

See “A comparison of antiarrhythmic-drug therapy with implantable defibrillators in patients resuscitated from near-fatal ventricular arrhythmias. The Antiarrhythmics versus Implantable Defibrillators (AVID) Investigators.” N Engl J Med 1997; 337:1576-83; Bardy G H, Lee K L, Mark D B, et al. Amiodarone or an implantable cardioverter-defibrillator for congestive heart failure. N Engl J Med 2005; 352:225-37; Buxton A L, Lee K L, Fisher J D, Josephson M E, Prystowsky E N, Hafley G. A randomized study of the prevention of sudden death in patients with coronary artery disease. Multicenter Unsustained Tachycardia Trial Investigators. N Engl J Med 1999; 341:1882-90; Moss A J, Zareba W, Hall W J et al. Prophylactic implantation of a defibrillator in patients with myocardial infarction and reduced ejection fraction. N J Med 2002; 346:877-83; Kraaier K, Verhorst P M, van Dessel P F, Wilde A A, Scholten M F. Towards a better risk stratification for sudden cardiac death in patients with structural heart disease. Neth Heart J 2009; 17:101-6; Patel J B, Koplan B A. ICD Implantation in Patients With Ischemic Left Ventricular Dysfunction. Curr Treat Options Cardiovasc Med 2009; 11:3-9; Buxton A E, Lee K L, Hafley G E, et al. Limitations of ejection fraction for prediction of sudden death risk in patients with coronary artery disease: lessons from the MUSTT study. J Am Coll Cardiol 2007; 50: 1150-7; Cygankiewicz I, Gillespie J, Zareba W et al. Predictors of long-term mortality in Multicenter Automatic Defibrillator Implantation Trial II (MADIT II) patients with implantable cardioverter-defibrillators. Heart Rhythm 2009; 6:468-73; Levy W C, Lee K L, Hellkamp A S et al. Maximizing survival benefit with primary prevention implantable cardioverter-defibrillator therapy in a heart failure population. Circulation 2009; 120:835-42; Levy W C, Mozaffarian D, Linker D T et al. The Seattle Heart. Failure Model: prediction of survival in heart failure. Circulation 2006; 113:1424-33; Vazquez R, Bayes-Genis A, Cygankiewicz I et at. The MUSIC Risk score: a simple method for predicting mortality in ambulatory patients with chronic heart failure. Eur Heart J 2009; 30:1088-96; Chow T, Kereiakes D J, Onufer et al. Does microvolt T-wave alternans testing predict ventricular tachyarrhythmias in patients with ischemic cardiomyopathy and prophylactic defibrillators? The MASTER (Microvolt T Wave Alternans Testing for Risk Stratification of Post-Myocardial Infarction Patients) trial. J Am Coll Cardiol 2008; 52:1607-15; Costantini O, Hohnloser S H, Kirk M M et al. The ABCD (Alternans Before Cardioverter Defibrillator) Trial: strategies using I-wave alternans to improve efficiency of sudden cardiac death prevention. J Am Coll Cardiol 2009; 53:471-9; Blangy H, Sadoul N, Dousset B et al. Serum BNP, hs-C-reactive protein, procollagen to assess the risk of ventricular tachycardia in ICD recipients after myocardial infarction. Europace 2007; 9:724-9; Verma A, Kilicaslan F, Martin D O et al. Preimplantation B-type natriuretic peptide concentration is an independent predictor of future appropriate implantable defibrillator therapies. Heart 2006; 92:190-5; Wazni O M, Martin D O, Marrouche N F et al. Plasma B-type natriuretic peptide levels predict postoperative atrial fibrillation in patients undergoing cardiac surgery. Circulation 2004; 110:124-7; Dekker L R, Bezzina C R, Henriques J P et al. Familial sudden death is an important risk factor for primary ventricular fibrillation: a case-control study in acute myocardial infarction patients. Circulation 2006; 114:1140-5; Jouven X, Desnos M, Guerot C, Ducimetiere P. Predicting sudden death in the population: the Paris Prospective Study I. Circulation 1999; 99:1978-83; Brodine W N, Tung R T, Lee J K et al. Effects of beta-blockers on implantable cardioverter defibrillator therapy and survival in the patients with ischemic cardiomyopathy (from the Multicenter Automatic Defibrillator Implantation Trial-II), Am J Cardiol 2005; 96:691-5; Coleman C I, Kluger J, Bhavnani S et al. Association between statin use and mortality in patients with implantable cardioverter-defibrillators and left ventricular systolic dysfunction. Heart Rhythm 2008; 5:507-10.

All of the above cited references are herein incorporated by reference in their entirety for all purposes.

### Linkage Disequilibrium and Informative Gene Groups

Linkage disequilibrium refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at greater than expected frequencies are then said to be in “linkage disequilibrium.” The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern.

In addition to the allelic patterns described above, as described herein, one of skill in the art can readily identify other alleles (including polymorphisms and mutations) that are in linkage disequilibrium with an allele associated with a disease or disorder. For example, a nucleic acid sample from a first group of subjects without a particular disorder can be collected, as well as DNA from a second group of subjects with the disorder. The nucleic acid sample can then be compared to identify those alleles that are over-represented in the second group as compared with the first group, wherein such alleles are presumably associated with a disorder. Alternatively, alleles that are in linkage disequilibrium with an allele that is associated with the disorder can be identified, for example, by genotyping a large population and performing statistical analysis to determine which alleles appear more commonly together than expected. Preferably the group is chosen to be comprised of genetically related individuals. Genetically related individuals include individuals from the same race, the same ethnic group, or even the same family. As the degree of genetic relatedness between a control group and a test group increases, so does the predictive value of polymorphic alleles which are ever more distantly linked to a disease-causing allele. This is because less evolutionary time has passed to allow polymorphisms that are linked along a chromosome in a founder population to redistribute through genetic cross-over events. Thus race-specific, ethnic-specific, and even family-specific diagnostic genotyping assays can be developed to allow for the detection of disease alleles which arose at ever more recent times in human evolution, e.g., after divergence of the major human races, after the separation of human populations into distinct ethnic groups, and even within the recent history of a particular family line.

Linkage disequilibrium between two polymorphic markers or between one polymorphic marker and a disease-associated gene or mutation is a meta-stable state. Absent selective pressure or the sporadic linked reoccurrence of the underlying mutational events, the polymorphisms will eventually become disassociated by chromosomal recombination events and will thereby reach linkage equilibrium through the course of human evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring each generation. As a result, the more closely related two individuals are, the more likely it is that widely spaced polymorphisms may be co-inherited. Thus, for individuals related by common race, ethnicity or family, the reliability of ever more distantly spaced polymorphic loci can be relied upon as an indicator of inheritance of a linked disease-causing mutation.

In addition to the specific, exemplary markers or haplotypes identified in this application by name, accession number, SNP Reference number, or sequence, included within the scope of the invention are all operable markers and haplotypes and methods for their use to determine susceptibility to a SCE using numerical values of variant sequences having at least 90% or at least 95% or at least 97% or greater identity to the exemplified marker nucleotide sequences or haplotype nucleotide sequences or that encode proteins having sequences with at least 90% or at least 95% or at least 97% or greater identity to those encoded by the exemplified markers or haplotypes. The percentage of sequence identity may be determined using algorithms well known to those of ordinary skill in the art, including, BLASTn, and BLASTp, as described in Stephen F. Altschul et al., J. Mol. Biol. 215:403-410 (1990) and available at the National Center for Biotechnology information website maintained by the National Institutes of Health.

In accordance with an embodiment of the present invention, all operable markers or haplotypes and methods for their use in determining susceptibility to a SCE now known or later discovered to be highly correlated with the expression of an exemplary marker or haplotype can be used in addition to or in lieu of that exemplary marker or haplotype. Such highly correlated markers or haplotypes are contemplated to be within the literal scope of the claimed invention(s) or alternatively encompassed as equivalents to the exemplary markers or haplotypes. Identification of markers or haplotypes having numerical values that are highly correlated to those of the exemplary markers or haplotypes, and their use as a component for determining susceptibility to SCE is well within the level of ordinary skill in the art.

### Computer Implementation

In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

### Methods of Therapy

In another embodiment, methods can be employed for the treatment of a sudden cardiac event in subjects shown to be susceptible to SCEs through use of e.g., diagnostic methods disclosed herein. The term “treatment” as used herein, refers not only to ameliorating symptoms associated with a sudden cardiac event, but also preventing or delaying the onset of a sudden cardiac event; lessening the severity or frequency of symptoms of a sudden cardiac event; and/or also lessening the need for concomitant therapy with other drugs that ameliorate symptoms associated with a sudden cardiac event. In one aspect, the individual to be treated is an individual who is susceptible (at an increased risk) for a sudden cardiac event.

In some embodiments, methods can be employed for the treatment of other diseases or conditions associated with a sudden cardiac event. A therapeutic agent can be used both in methods of treatment of a sudden cardiac event, as well as in methods of treatment of other diseases or conditions associated with a sudden cardiac event.

In one embodiment, the methods of treatment can utilize implantation of a cardioverter defibrillator (ICD). The methods of treatment (prophylactic and/or therapeutic) can also utilize a therapeutic agent. The therapeutic agent(s) are administered in a therapeutically effective amount (i.e., an amount that is sufficient for “treatment,” as described above). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose response curves derived from in vitro or animal model test systems.

### Pharmaceutical Compositions

Methods for treatment of a sudden cardiac event in subjects shown to be susceptible to SCEs through use of the diagnostic methods are also encompassed. Said methods include administering a therapeutically-effective amount of therapeutic agent. A therapeutic agent can be formulated in pharmaceutical compositions. These compositions can comprise, in addition to one or more of the therapeutic agents, a pharmaceutically-acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability, Those of relevant skill in the art are well able to prepare suitable solutions using, for example, isotonic vehicles such as Sodium Chloride injection, Ringer's Injection, Lactated Ringer's Injection. Preservatives, stabilisers, buffers, antioxidants and/or other additives can be included, as required.

Whether it is a polypeptide, antibody, nucleic acid, small molecule or other pharmaceutically useful compound that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc, is within the responsibility of general practitioners and other medical doctors, and typically takes account of the disorder to be treated, the condition of the individual patient, the site of the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### EXAMPLES

Below are examples of specific embodiments of the invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of embodiments of the invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook et al., Molecular Cloning: A Laboratory Manual (2nd Edition, 1989); Methods in Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed (Plenum Press) Vols A and B (1992).

### Example 1

### Data and Quality Control (QC)

Subjects enrolled in the multicenter Diagnostic Investigation of Sudden Cardiac Event Risk (DISCERN) trial (ClinicalTrials.gov website ref. no. NCT00500708) served as the starting population for this study.

Data Collection and Reporting

Clinical Data

Clinical data came from the locked DISCERN DI data report exported from the DISCERN electronic case report form (eCRF) for n=680 experimental subjects. All subjects provided informed written consent for study participation under the DISCERN protocol approved by the Institutional Review Boards (IRBs) at the enrolling institutions. Clinical data were obtained through a combination of subject interview and abstraction from medical records and entered into the DISCERN electronic case report form (eCRF). Data monitoring (source data verification) was completed for ˜300 control subjects per the clinical monitoring plan. The clinical data is described in more detail below.

Event Data

For subjects who received device therapies (anti-tachycardia pacing (ATP) or shock), internal electrograms (IEGMs) were collected for adjudication of the event and categorization of the underlying treated rhythm. In the absence of retrievable IEGMs, clinical reports describing device therapies were used to adjudicate the event. All final event categories were determined by concordance of at least two independent, blinded readers or committee review. Event class, subject class, and event dates were provided for this analysis.

Biologic Samples

Blood samples for DNA isolation were drawn at enrollment, frozen and shipped/stored at CardioDx. A subset of the subjects had DNA extracted by an outside vendor (Gentris) and stored frozen at CardioDx.

DNA Samples

Genomic DNA was isolated from whole blood using an automated approach on the Hamilton Star (DNAdvance DNA Isolation Kit, Agencourt). The DNA was diluted to a concentration of 50 ng/μl and 1.2 ug was provided to the vendor, Expression Analysis (Durham, N.C.), for application on the Affymetrix human whole-genome 6.0 SNP array, Genotypes were determined based on array results provided by the vendor and the final experimental dataset determined.

The data QC was performed in two parts: the clinical data and the genotype data.

Clinical Data QC

At the analysis stage several inconsistencies were found over time, e.g., several samples had gender mismatches between the clinical and genetic information and several samples had primary prevention status inconsistencies. Samples with unresolved inconsistencies were deleted from further consideration. In order to reduce population structure only Caucasian subjects were chosen. A set of 658 subjects with complete genetic and clinical data were selected for further analysis, after excluding the inconsistent samples.

Genotype Data QC

The genotype data was generated by Expression Analysis (Durham, N.C.) using the Affymetrix SNP 6.0 platform as noted above. There were 667 DISCERN samples plus 8 identical controls. The SNP 6.0 platform contains genotype assays for 909,622 SNPs and 946,000 CNVs.

The genotypes were generated with the Birdseed algorithm version 2 by Expression Analysis and made available along with the cell files. For each sample the Birdseed output files contains for each SNP the genotype call, a confidence value for the genotype, and intensity values for each of the A and B alleles.

Three filters were applied.

Call Rates

A genotype is declared a NoCall when the confidence value is over the 0.1 threshold so a SNP assay fails when a NoCall is declared.

For a given sample, the sample call rate is the proportion of all SNPs successfully genotyped for that sample. For a given SNP, the SNP call rate is the proportion of all samples successfully genotyped for that SNP. The analysis plan imposes a passing sample call rate threshold of 80% and a passing SNP call rate of 95%.

The sample call rates and SNP call rates were calculated. One DISCERN sample had a call rate <80% and was excluded from further analysis (according to the analysis plan threshold).

The 8 replicated control samples had sample call rates 0.90<CR<0.95. The control sample was a pooled sample of males and females. This resulted in some mis-genotype clustering, as described below.

One DISCERN sample had a sample call rate=0.93 but the 665 (98.5%) DISCERN samples have sample call rate CR >0.95, which is within Affymetrix expectations.

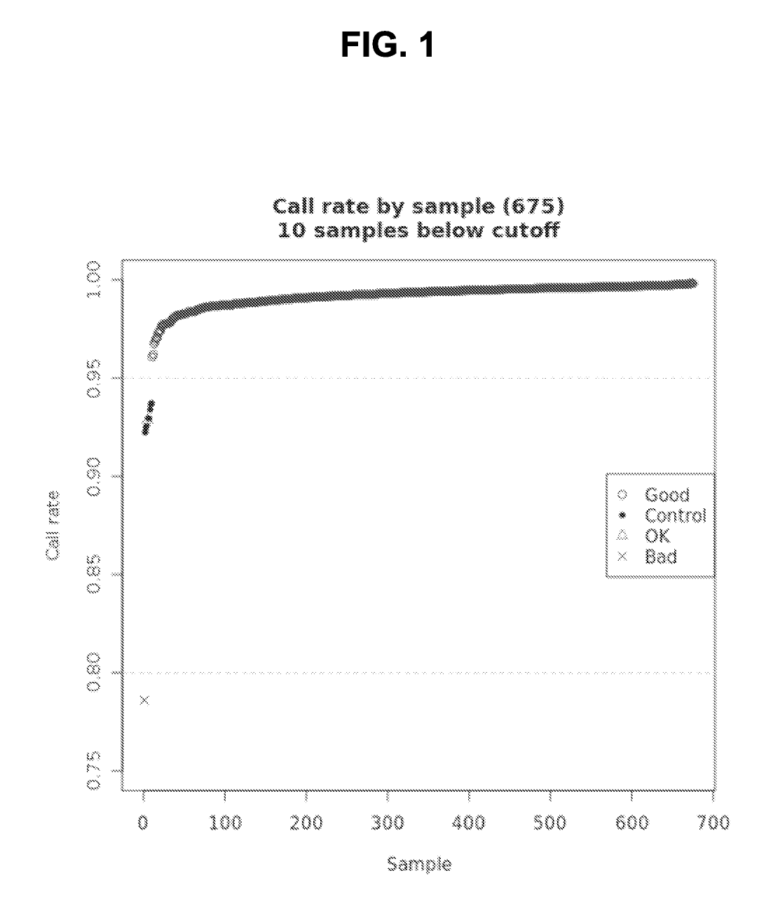

SNP call rates were calculated and a cutoff of 95% imposed resulting in 30,391 SNPs (3.3%), which is within Affymetrix expectations (FIG. 1).

Minor Allele Frequencies

The minor allele frequency was calculated for each SNP, a cutoff of 1% was imposed, with the result that 137,583 SNPs (15.1%) failed this cutoff. This was a large fraction of SNPs on the chip, but most of these SNPs have higher minor allele frequency in non-Caucasian populations. The minor allele frequencies obtained from the cohort were highly correlated (Pearson correlation=0.974) with the Caucasian minor-allele frequencies as reported by Affymetrix from the Caucasian HapMap sample set.

Hardy-Weinberg Equilibrium

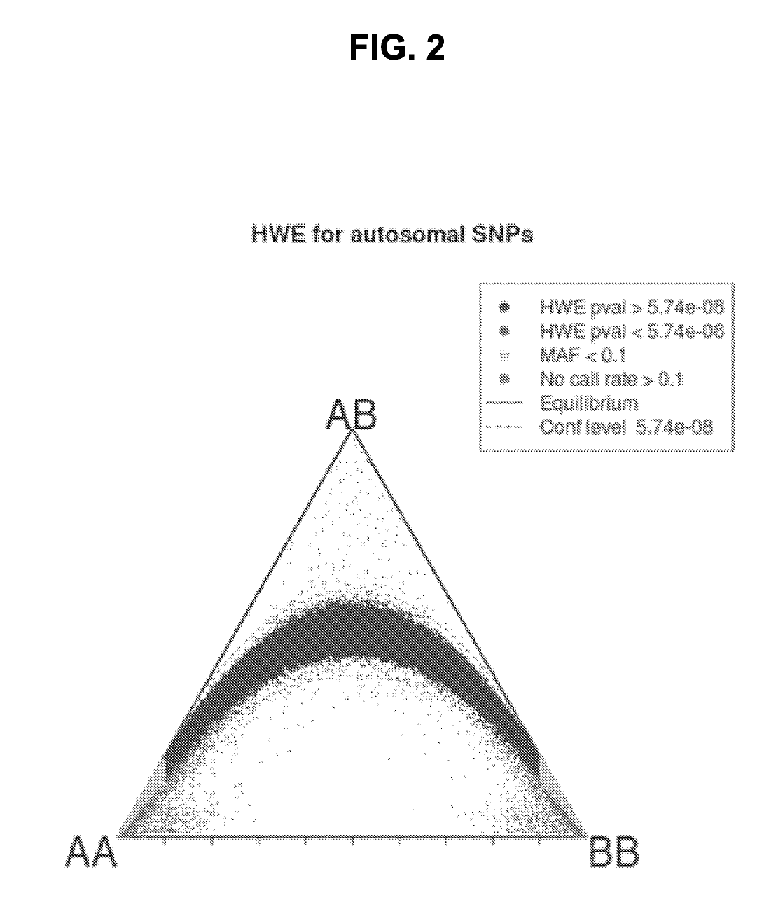

Hardy-Weinberg equilibrium (HWE) was calculated with an exact test for all autosomal and pseudo-autosomal SNPs. For non-pseudo-autosomal SNPs on chromosome X a modified chi-square test was used. This test combines the standard equilibrium model for females but includes the male genotypes, which are hemizygous, in the allele frequency estimates. SNPs on chromosome Y and mitochondria SNPs are hemizygous and were excluded. In the deFinetti diagram most of the SNPs out of equilibrium have a low SNP call rate <95% and were cut from further consideration (FIG. 2).

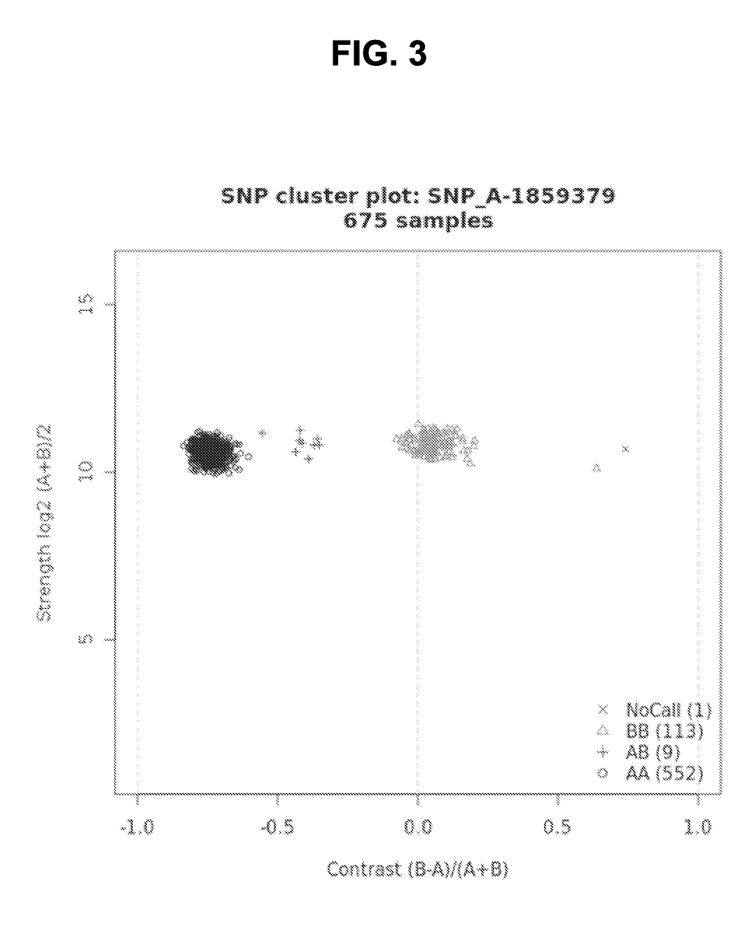

Among the remaining SNPs out of equilibrium with MAF>1, virtually no heterozygotes were a subset with mis-clustering likely due to the pooled replicate samples. This is evident from the deFinetti diagram at the bottom right and left corners (FIG. 2). The set of 8 replicates had an intermediate cluster that was declared heterozygotic by the clustering algorithm. In this case the true heterozygotes were declared minor allele homozygotes and equilibrium failed. The cluster diagram in FIG. 3 shows a representative example (SNP_A-1859379).

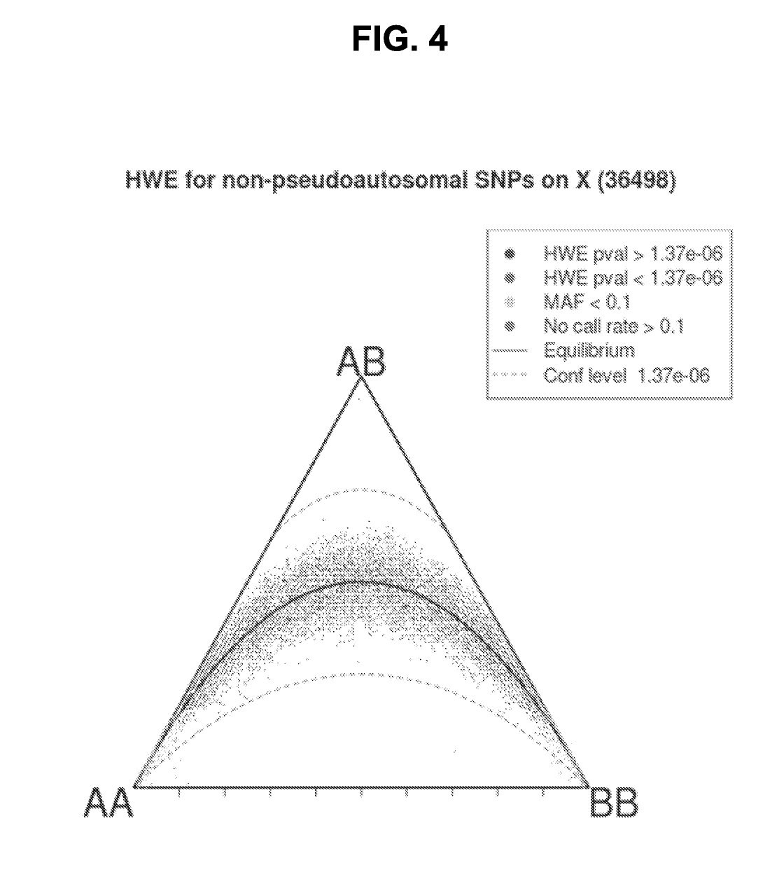

FIG. 4 shows that the non-pseudo-autosomal SNPs on chromosome X show no such pathology. The 89 SNPs with HWE p-value <1e−100 that show the worst disequilibrium were excluded.

Passing SNPs

The passing SNPs are those that survived the three filters: call rate, minor allele frequency, and HWE. The number of SNPs passing for further analysis was 748,158 out of a total of 909,622 SNPs on the chip.

Gender Determination

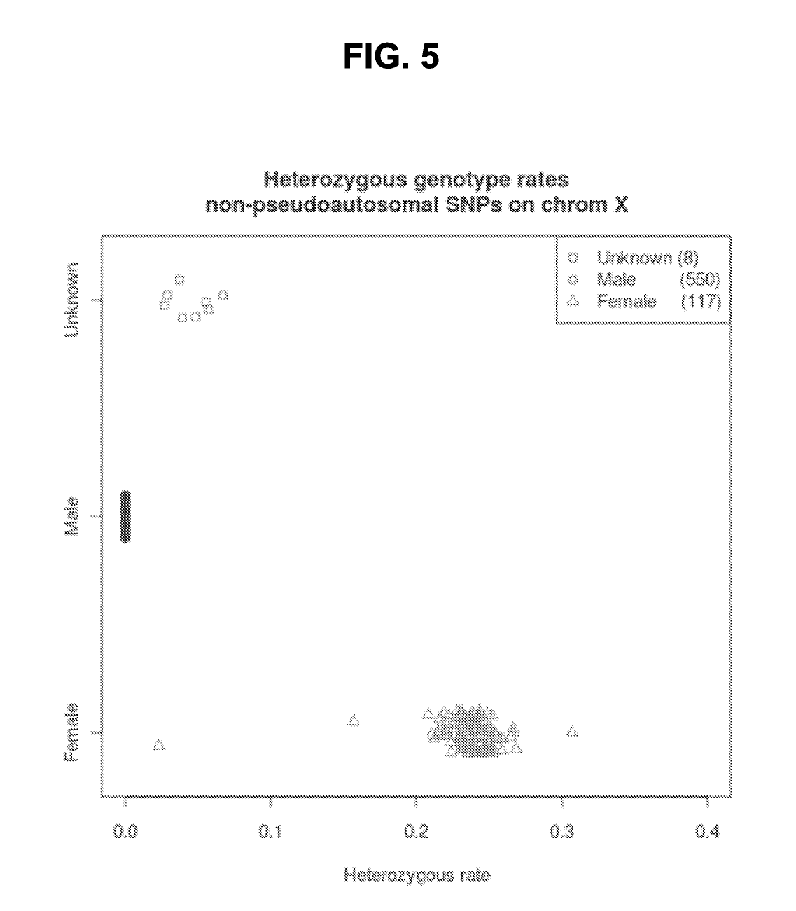

Only females can be heterozygotic at non-pseudoautosomal SNPs on chromosome X. Thus sample gender was inferred from the presence or absence of heterozygote genotypes non-pseudoautosomal SNPs on chromosome X. A female will have heterozygotic loci and males will not. From the plot (FIG. 5) one sample (on the lower left in green) was marked as female but lacks heterozygote loci and was inferred to be mate. The 8 samples (in the upper left corner in red) marked unknown are in an intermediate position (FIG. 5). These were the 8 replicated control samples that were pooled samples of males and females. This explains their intermediate position and illustrates that pooled samples result in incorrect genotypes.

Concordance

It was intended that the 8 replicated control samples would allow a concordance estimate of the genotype data set. The concordance of the replicate samples was 85.6%. This corresponds closely to that expected from their average sample call rate of 92.0%, which assuming random miscalls, gave an expect concordance of 92%*92%=86.6% The pooled nature of the control samples resulted in low call rates compared to the typical samples and so the controls are not completely representative of the typical samples. Thus the concordance of the controls is a low estimate of the true concordance of the data set. The average sample call rate excluding the failed sample and replicate samples is 99.2%. From this a concordance of 99.2%*99.2%=98.4% for the passing samples was estimated.

Clinical Data

Clinical data for each subject contains the categories:

age

gender

diabetes status

renal function

heart status

medications

The heart status fields were:

ejection fraction

NYHA class

sinus rhythm status

conduction problems

MI history

ECG measurements

The NYHA class status were not recorded for each subject.

Case Status and Time-to-Event

For each subject in the study, the time interval from the date of implant to the end of observation of the subject was called the total observation time of the subject. The phenotype of central interest in this study was ventricular tachycardia and fibrillation (VT/VF). Each subject had an event history recorded by their implant device. An expert panel adjudicated all potential events for each subject deciding in each case if a VT/VF event had occurred and recording the time. Each subject with an adjudicated VT/VF event was declared a case and the time interval from the date of implant to the first adjudicated event was called the tune-to-event. For subjects that are not cases their time-to-event measure was the same as the total Observation time. A subject that was not a case and had a total observation time of at least two years was called a control. Secondary prevention subjects have had a VT/VF event before implant surgery took place so they were classed as cases, but have no time-to-event measure.

Clinical Risk Factors for VT/VF

In this section the clinical covariates as risk factors for VT/VF is considered. It was also important to determine which clinical factors may be confounders for the genetic risk factor analysis performed in the sections below.

Statistical Model

We used a Cox proportional hazards model to test association of clinical covariates to VT/VF time-to-event data.

Time-to-event˜clinical covariates

where non-cases were censored.

Gender

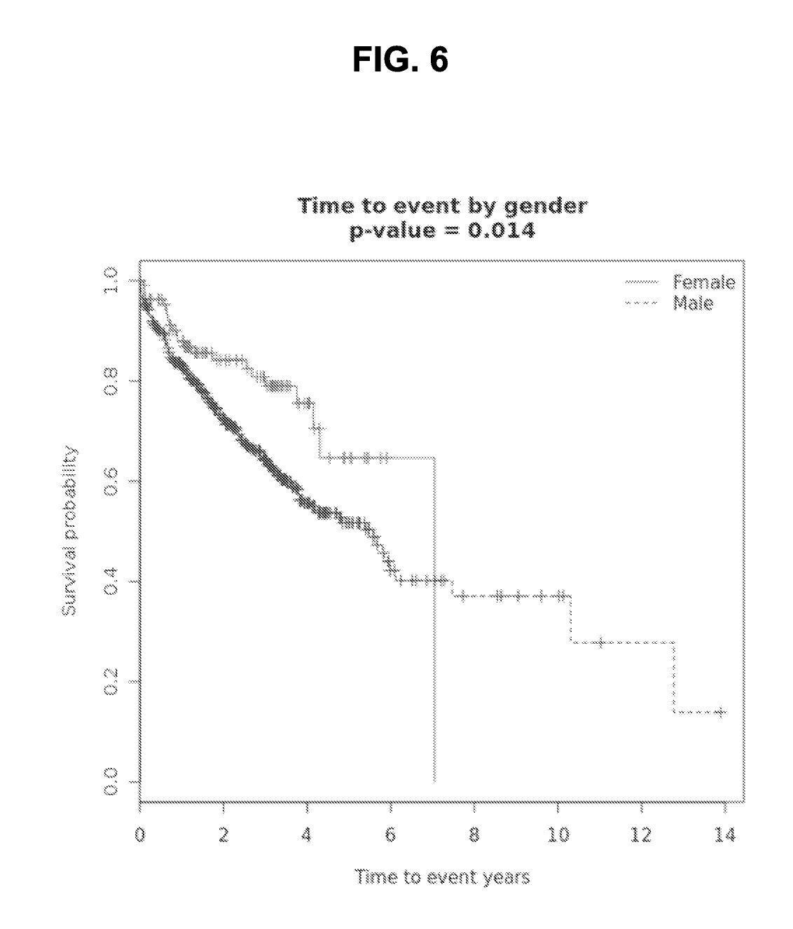

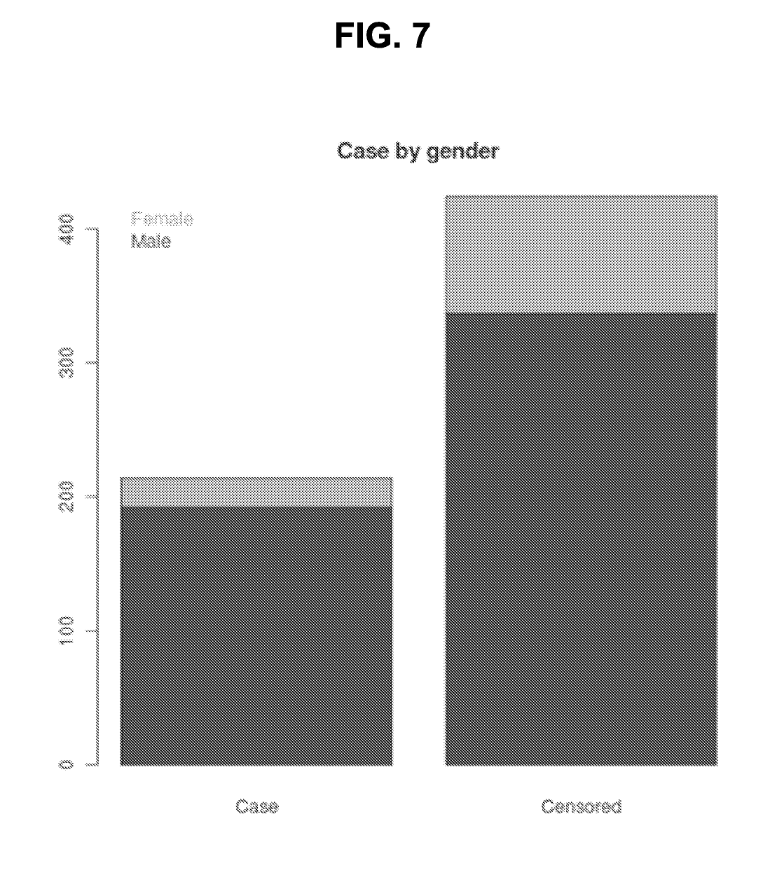

Subject gender was significantly associated with VT/VF time-to-event (TTE). This can be seen with the Kaplan-Meier plot of FIG. 6. This shows that the female subjects in the study survive longer than the males. This imbalance is also easily seen from the barplot of FIG. 7.

MADIT II Scores

The MADIT II score is the sum of five components: MADIT II score=non-sinus rhythm+age>65+NYHA class>2 (heart failure severity)+BUN level>28 (renal function)+diabetes.

The MADIT II score has known relation to patient survival from all causes. The Kaplan-Meier plot shows that there is no discernible association of high/low MADIT II score with VT/VF arrhythmia (FIG. 7).

Several components of the MADIT II score had incomplete data. The NYHA class was not recorded at time of implant for 34% of subjects. Of these, 14% had NYHA class recorded during follow-up and this was used. Another 10% were being prescribed loop diuretics, which was taken to indicate NYHA class >2, For the remaining 10% of subjects the NYHA class was imputed with a recursive partitioning algorithm.

The BUN level was not recorded for 21% of subjects. The missing values were imputed with a recursive partitioning algorithm. Missing BUN level measurements are correlated with good renal function, so in this case the attending physician may not have seen a need to order a BUN level test.

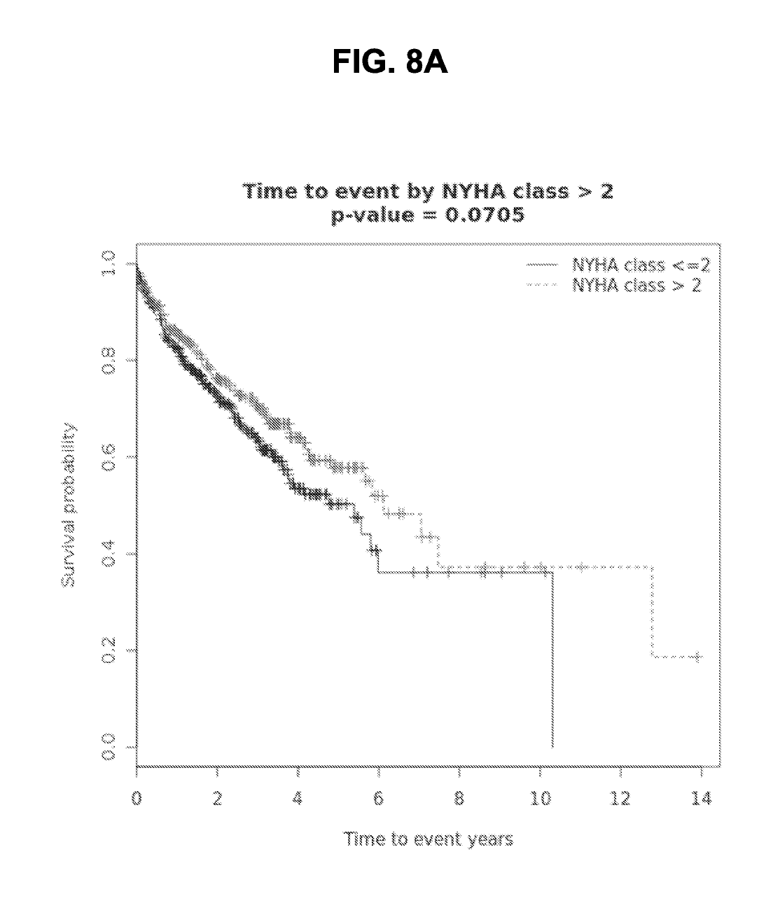

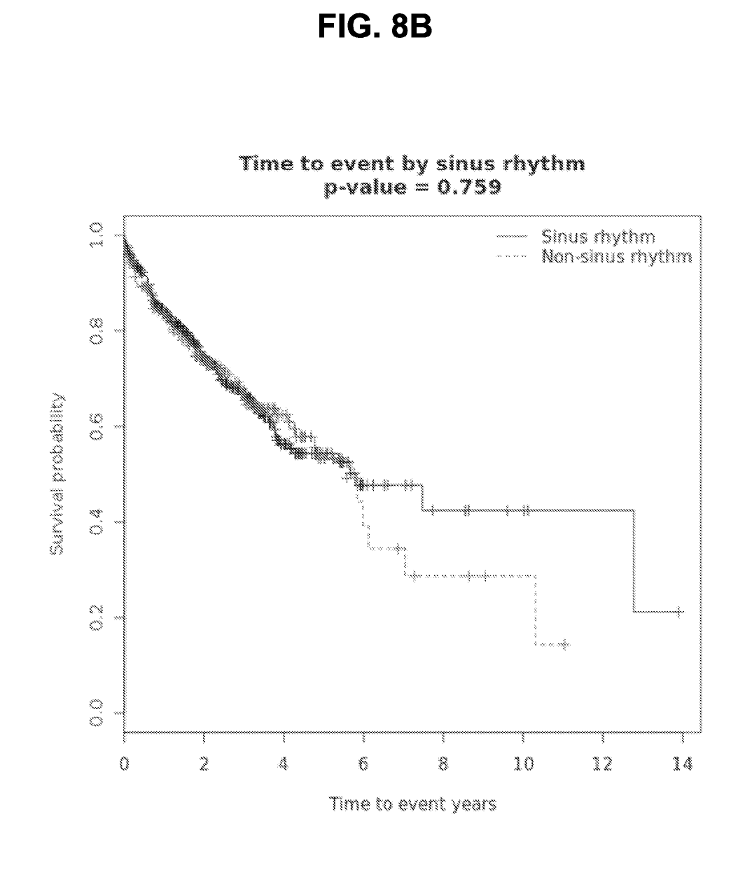

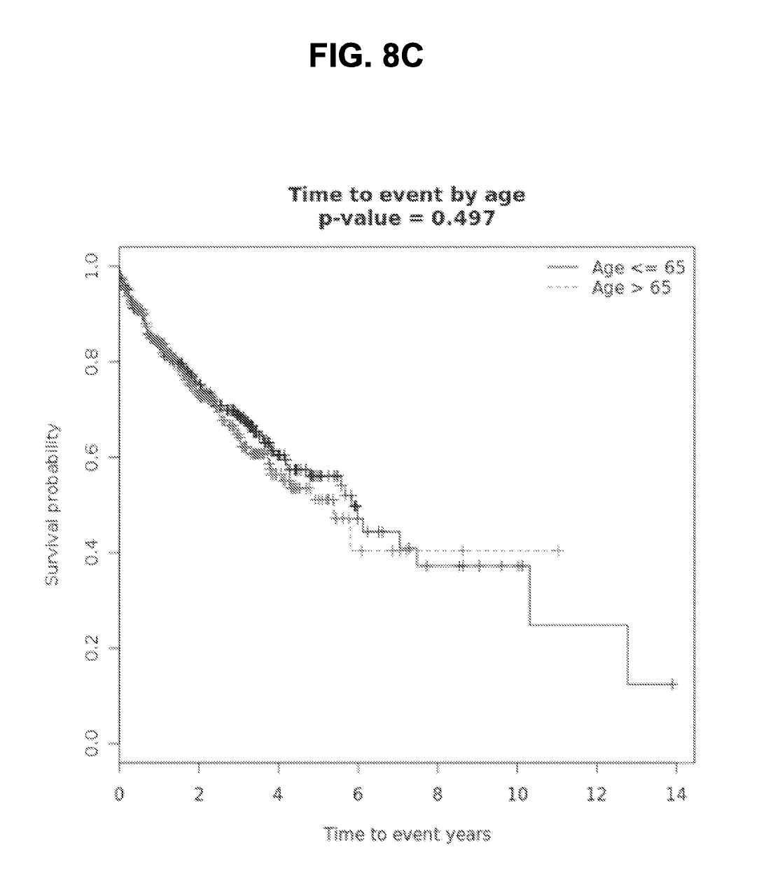

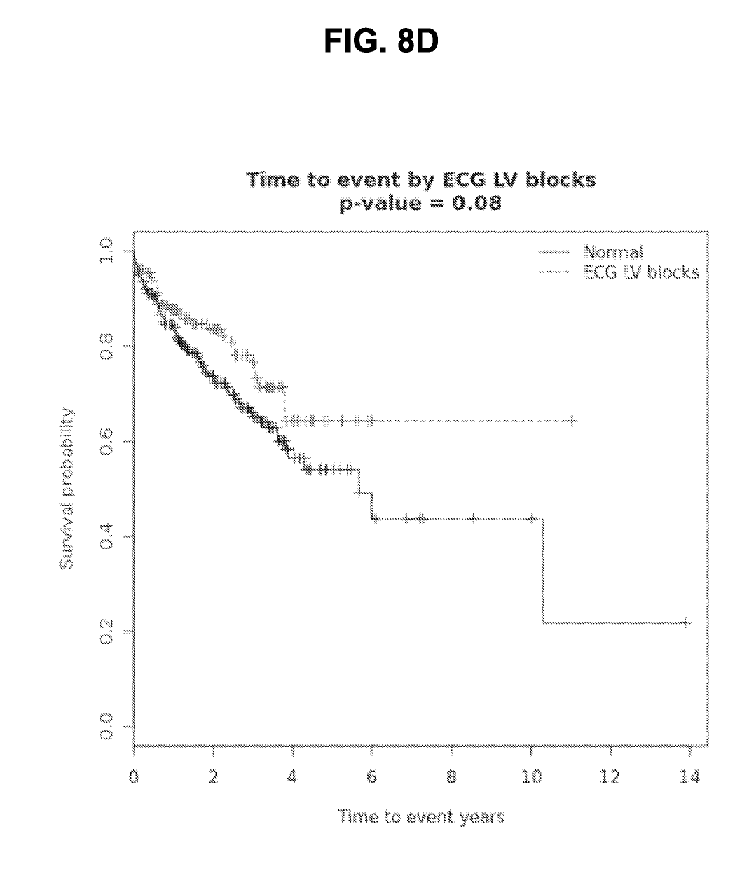

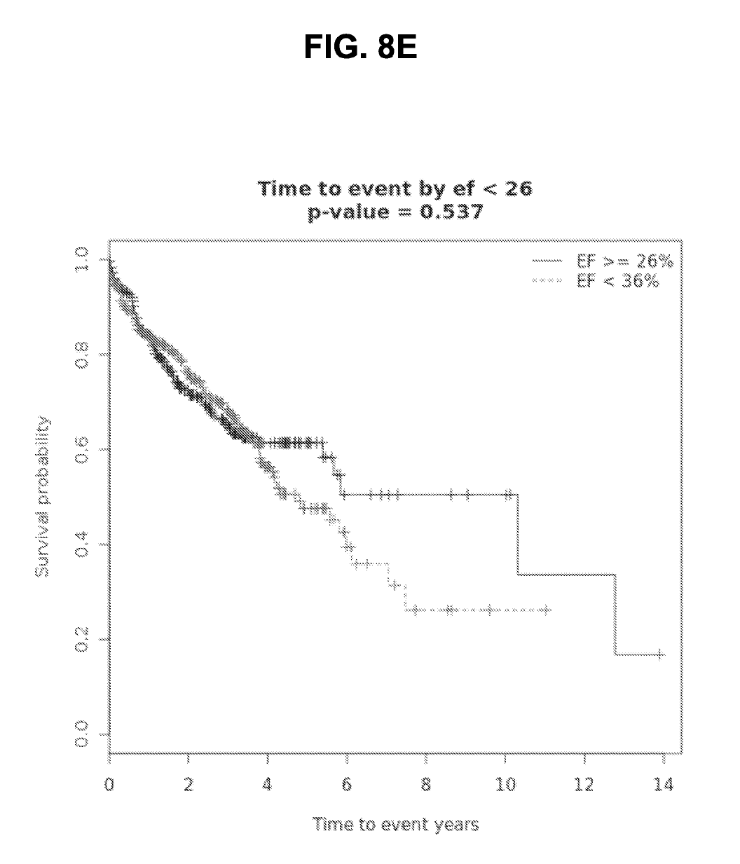

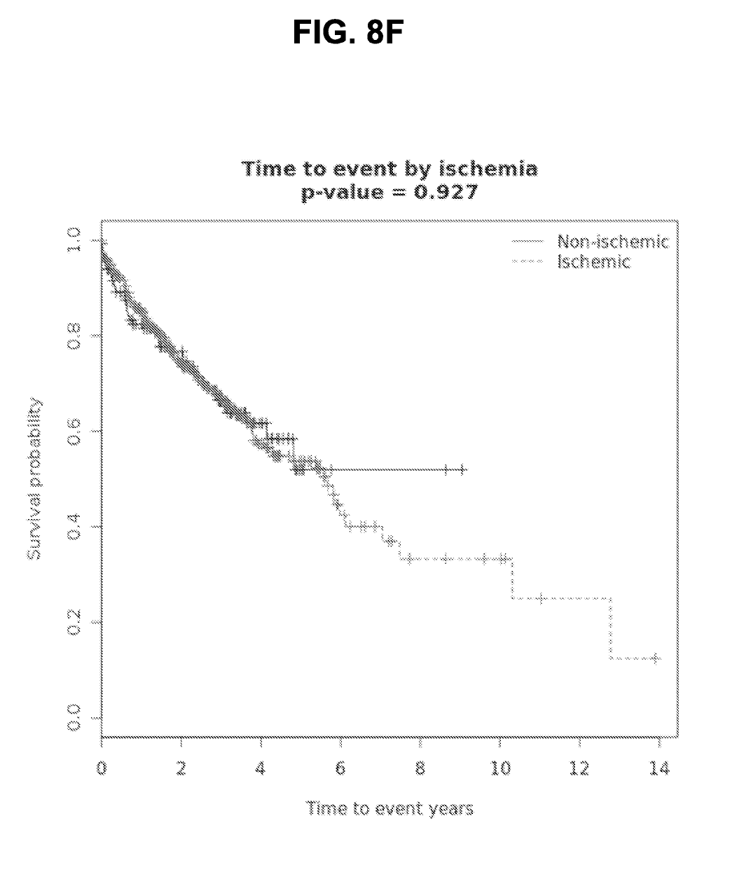

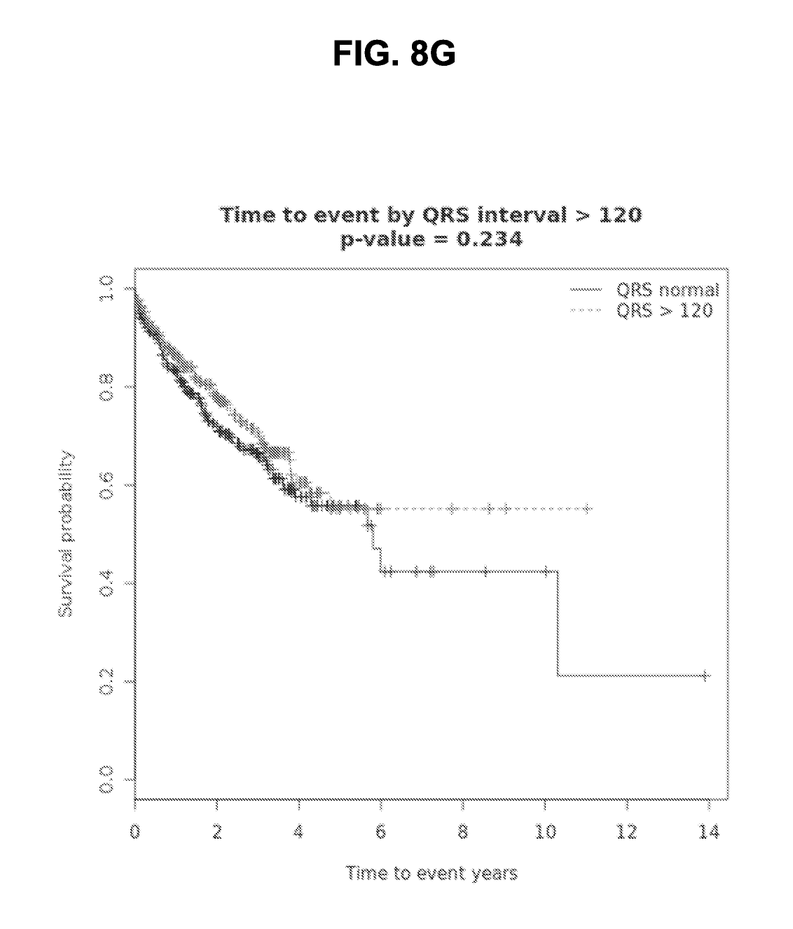

The individual components of the MADIT II score also showed no significant association, except for the NYHA class, which showed marginally significant association (FIG. 8).

The presence of ventricular conduction blocks versus no conduction block (left ventricular or otherwise) showed marginally significant association with VT/VF arrhythmia (FIG. 8). Age, ejection fraction, and ischemia showed no significant association (FIG. 8). The QRS interval, which has known genetic connections to arrhythmias, showed no significant association (FIG. 8).

Kidney Function

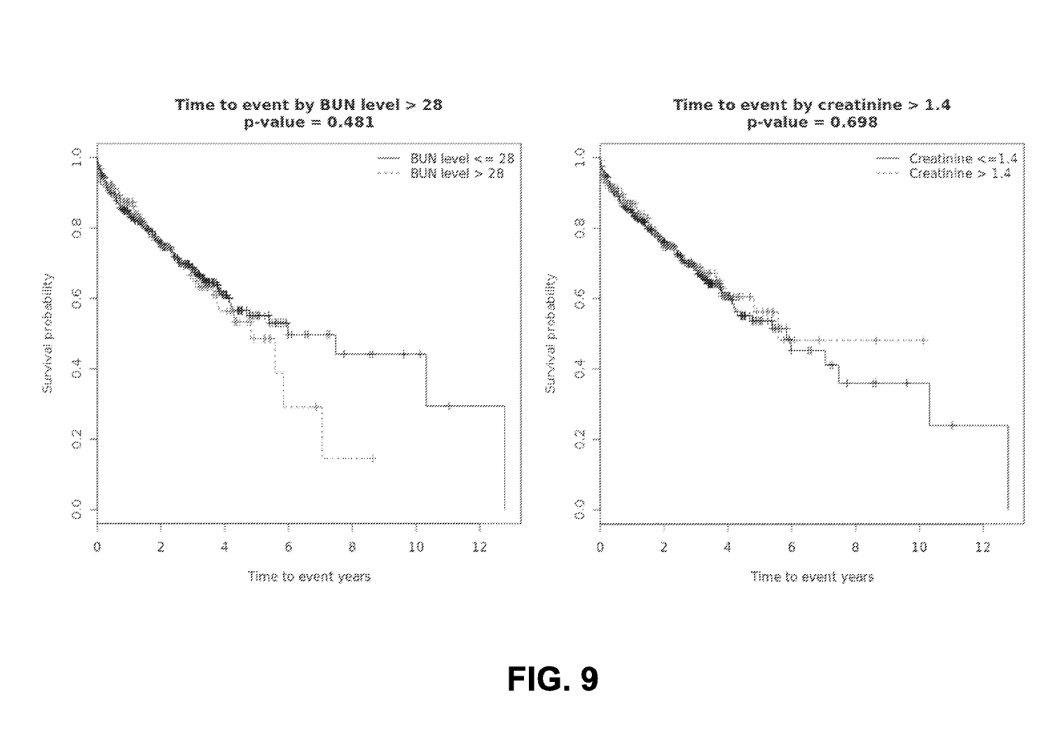

The blood urea nitrogen level (BUN) is an indicator of kidney function, where high BUN level indicates renal insufficiency. The Kaplan-Meier plot in FIG. 9 shows no significant association of BUN level with VT/VF arrhythmia. Creatinine level is also an indicator of kidney function and had no discernible association with VT/VF arrhythmia (FIG. 9).

Diabetes

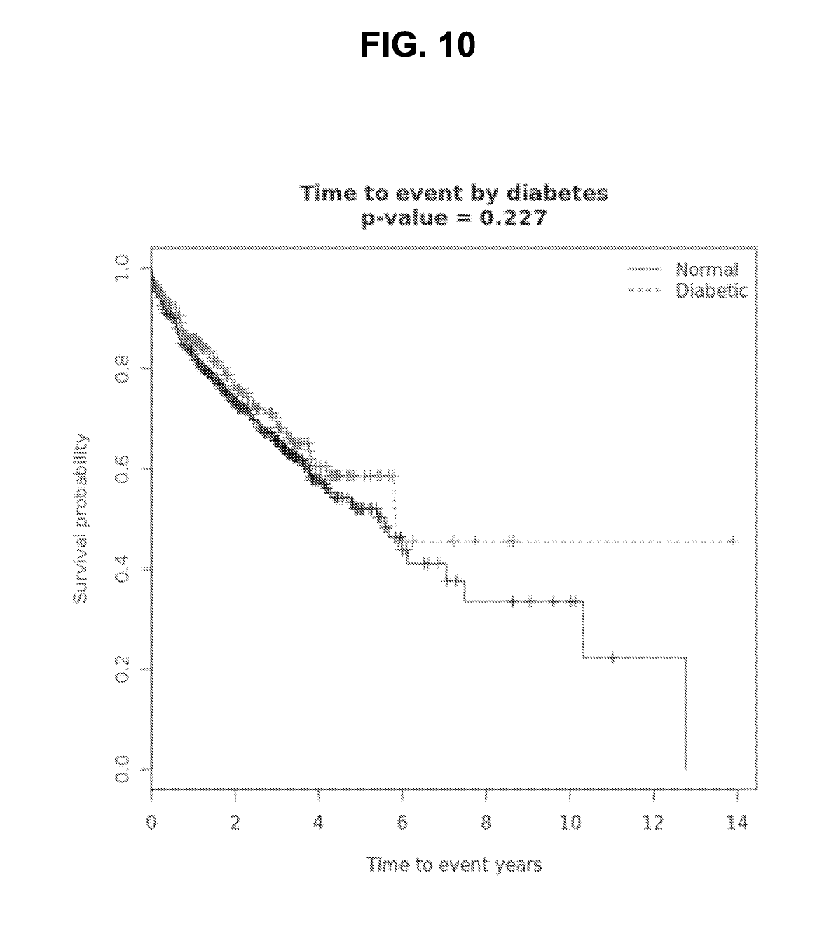

Diabetes status did not have a significant association with VT/VF arrhythmia (FIG. 10).

### Example 2

### Geneset Analysis

A geneset as used in this example is any collection of genes, such as genes in a pathway, whose combined action is expected to have association with a phenotype of interest. In the present study, we had SNP-based genotypes and connected SNPs to genes to carry out a geneset analysis. To do this we collected the SNPs near the genes of a geneset. Each gene had a number of annotated SNPs based on the distance of the SNP to the gene footprint or within overlapping LD bins. Thus each geneset resulted in a SNPset SNPs near the genes of the geneset. When a large SNPset contains only a few SNPs with actual association the signal-to-noise ratio may be too small to detect an association without more subjects. The strategy adopted to solve this was to choose a limited number of SNPs (e.g., from 10 to 100) for each gene in a geneset, rather than make all the SNPs available for each gene, which can result in very large SNPsets.

Genesets

The following genesets were compiled and contain a total of 414 genes TABLE 1-12):

| | | |
|---|---|---|
| 1. | Excitation-Contraction Coupling (Table 1) | (50) |
| 2. | Ion Channel genes (Table 2) | (43) |
| 3. | Ca++ handling and Ca++ dependent functions (Table 3) | (38) |
| 4. | Recently discovered loci (Table 4) | (8) |
| 5. | Gap junction and desmosomes (Table 5) | (10) |
| 6. | GPCRs and membrane receptors other (Table 6) | (11) |
| 7. | Transcription factors (Table 7) | (13) |
| 8. | Cytoskeletal and giant sarcomere proteins (Table 8) | (19) |
| 9. | Renin-Angiotensin-Aldosterone system (Table 9) | (5) |
| 10. | Mitochondrial/metabolic functions (Table 10) | (17) |
| 11. | Cardiac Calcium genes (Table 11) | (160) |
| 12. | Other genes (Table 12) | (123) |
| 13. | Arrhythmia genes (Table 13) | (304) |

Association Model

This statistical model is the same survival model as above with the addition of the gender covariate, which was seen to be associated with the VT/VF arrhythmia phenotype. That is, the Cox proportional hazards model.

Time-to-event˜gender˜gender+{geneset genotype derived data}

where non-cases are censored. The “geneset genotype derived data” were derived from the genotypes of the SNPs of a geneset by one of the several methods described below.

Minor Allele Count (MAC)

For each subject, we counted the number of minor alleles (MAC) among the SNPs of a geneset and checked this for association with VT/VF arrhythmia. In this case, the “geneset genotype derived data” were the minor allele counts for each subject. In this case we checked for association of the geneset with the survival model

Time-to-event˜gender+MAC

where non-cases are censored.

Signed Sum of Minor Alleles (SSUM)

This method is the same as above except we added minor alleles when protective and subtracted when deleterious. That is, each SNP of the geneset was checked individually for association with the model

Time-to-event˜gender+additive(genotype)

where non-cases are censored. We say the minor allele is protective when the association results in fewer arrhythmias. And that the minor allele is deleterious when the association results in more arrhythmias. The signed-sum of minor alleles (SSUM) is

SSUM=(sum of protective minor alleles)−(sum of deleterious minor alleles)

In this case we checked for association of the geneset with the survival model

Time-to-event˜gender+SSUM

where non-cases are censored.

Partial Least Squares (PLS)

In this method, we extracted the component of the genotype data that correlated with the case/control status of the subjects using the partial least squares (PLS) method. See “The pls package: principle components and partial least squares regression in R”, B-H Mevik and R. Wehrens, J. of Statistical Software, January 2007, vol 18, Issue 2. We checked this for association with VT/VF a arrhythmia with the Cox proportional hazards model adjusted for gender

Time-to-event˜gender+PLS component

where non-cases are censored.

Permutation Testing

Permutation testing is used for determining the p-values for all of the above geneset methods as the null distribution (the distribution of non-association) was unknown. This is computationally intensive, but in some situations there are alternatives, as illustrated in the examples below.

Primary Geneset Analyses

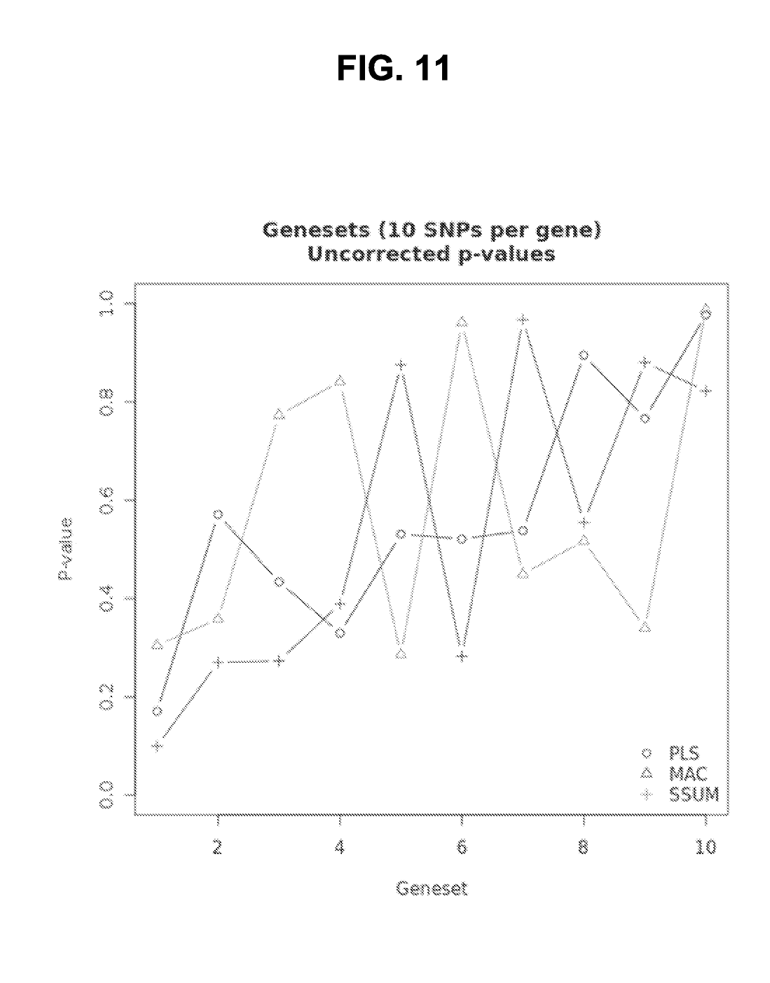

For each geneset with 10 SNPs per gene and all three methods were run with 10,000 permutations to determine p-values. As can be seen in the plot of FIG. 11, no result achieved statistical significance for any of the methods used.

Secondary Geneset Analyses

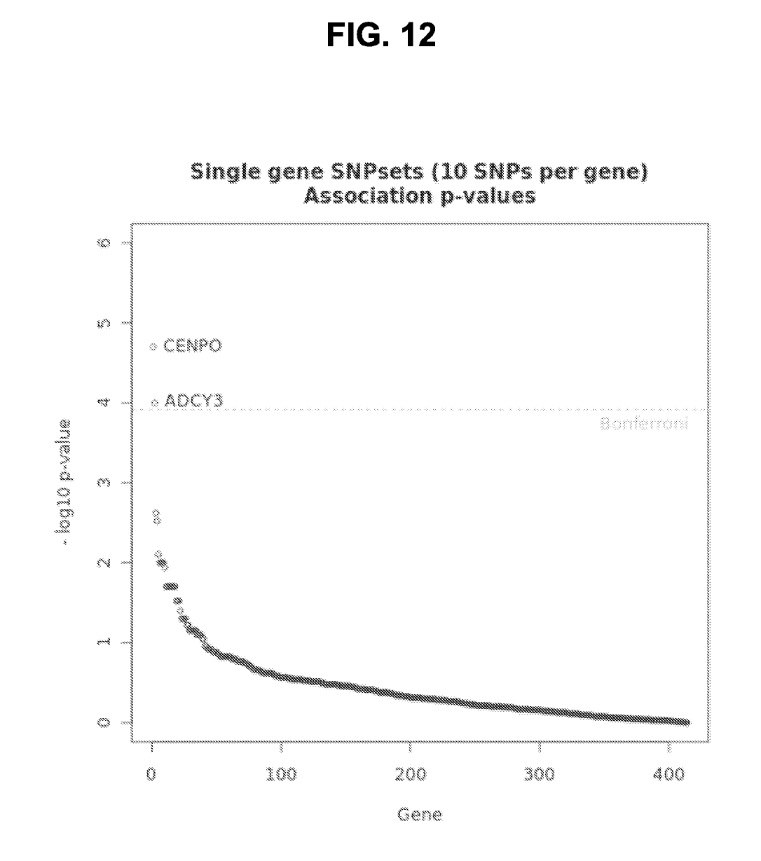

Each of the 414 genes were tested individually with 10 SNPs per gene with the PLS method and 1,000 permutations. The genes with the smallest p-values were run again with 50,000 permutations to obtain a more precise p-value estimation. The resulting p-values are shown in the plot with the horizontal dashed-line showing the Bonferroni adjustment required to achieve significance for 414 tests (FIG. 12). Two genes had significant association: CENPO and ADCY3. These genes are next to each other on the genome and possibly these associations are due to the same SNPs.

P-Value Calculations

Precise estimates of small p-values require more permutations (by the inverse square law.) An alternative is to fit a normal distribution on the null distribution (given by the permutation results) and calculate a z-score and a p-value. For the CENPO gene the QQ normal plot shows the null distribution from the permutation test fits a normal distribution (FIG. 13). A standard z-score calculation yields a p-value of 9.0e−6 with an adjusted p-value

adjusted p-value=414*9.0e−6=0.0037

### Example 3

### Genome-Wide Association Study (GWAS) Analysis

In the GWAS, or genome-wide association study, each SNP was tested individually for association with the VT/VF phenotype.

Statistical Model of Association

For each SNP, we tested if there is an association of time-to-event with genotype using the Cox proportional hazards model

Time-to-event˜gender+additive(genotype)

where non-cases are censored. The gender term is included as it is a possible confounder. This was the same as in the geneset analysis (above). Fitting this model to the data for a particular SNP yields a log hazard ratio and a p-value. The hazard ratio represents the differential hazard rate of having VT/VF arrhythmia from having one genotype versus another for this particular SNP. The p-value indicates the probability that this hazard ratio value occurred just by random (due to random sampling of the subjects in the study assuming the SNP is not associated with arrhythmia.) When the p-value is very small then it is inferred that the SNP is associated with arrhythmia. The results for all passing SNPs and for ischemic subjects only are shown in Table 14. The column definitions for Table 14 are shown below.

TABLE 14
Column Definitions

| | |
|---|---|
| pid | probeset ID (Affy SNP ID) |
| coef | log odds ratio of the genotype association |
| stderr | standard error of the log odds ratio |
| pval | p-value of the genotype association with time-to-event |
| | data |
| pval_holm | Holm correction of the p-value |
| pval_bonf | Bonfferoni correction of the p-value |
| pval_fdr | FDR (false discovery rate) for this size p-value |
| p_nc | proportion of NoCalls for this SNP |
| maf | minor allele frequency of this SNP |
| hwe | Hardy_Weinburg equilibrium p-value of this SNP |
| chr | chromosome containing the SNP |
| position | genomic position of the SNP |
| rsid | refSNP ID |
| npa_x | chrom X non-pseudoautosomal |
| odds_ratio | odds ratio |
| isc_coef | ischemic subset log odds ratio |
| isc_stderr | ischemic subset standard error |
| isc_pval | ischemic subset p-value |
| isc_pval_holm | ischemic subset Holm correction of the p-value |
| isc_pval_fdr | ischemic subset FDR |
| nyc_pval | pvalue of genotype association with NYHA class |
| ef_pval | pvalue of genotype association with ejection fraction |
| isc_nyc_pval | pvalue of genotype association with NYHA class for |
| | ischemic subjects only |
| isc_ef_pval | pvalue of genotype association with ejection fraction |
| | for ischemic subjects only |

From the adjusted p-value column (pval_holm) it is apparent that there is no single SNP with genome-wide significance. However, if a less conservative adjustment is made, the false discovery rate column (fdr) showed the top ten SNPs may have a Use discovery rate of 27% suggesting there is a true positive there. See next section.

Multiple Testing Adjustment

The p-value adjustment to account for multiple testing was performed with the Holm method and is given in the pval_holm column of Table 14. For the top hit, this is the same as the Bonferroni adjustment, which amounts to multiplying the p-value by 748,158 (the number of SNPs tested).

Adjusted p-value=7.96e−08*7.48e+5=0.060

This was not significant at the genome-wide level. But the number of SNPs (˜748 k) represents a conservative multiplication factor as all the SNPs are not independent, that is, their genotypes are correlated (as many SNPs cluster around genes and share LD bins.) We estimated the effective number of tests with a modified Gao method (see the next section). This method estimated that ˜13% to 20% of the SNPs represent independent tests for a multiplication factor of ˜748,000*0.15=112,000 to ˜748,000*0.26=194,000. Using this range of multiplication factors gives:

Adjusted p-value from

7.96e−08*1.12e+5=0.009

to

7.96e−08*1.94e+5=0.015

So the top hit (SNP_A-2053054) attained genome-wide significance using the less conservative multiple testing adjustment. But the next most significant hit only attained a level of 0.17 and was not significant at the genome level.

Genotype Cluster Plot

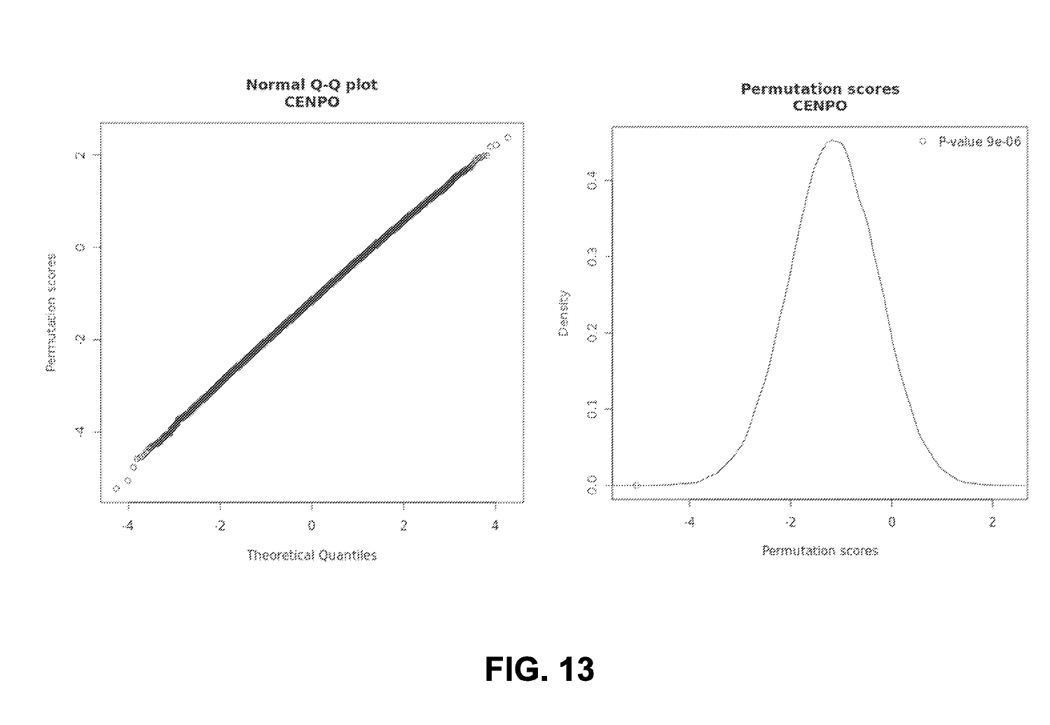

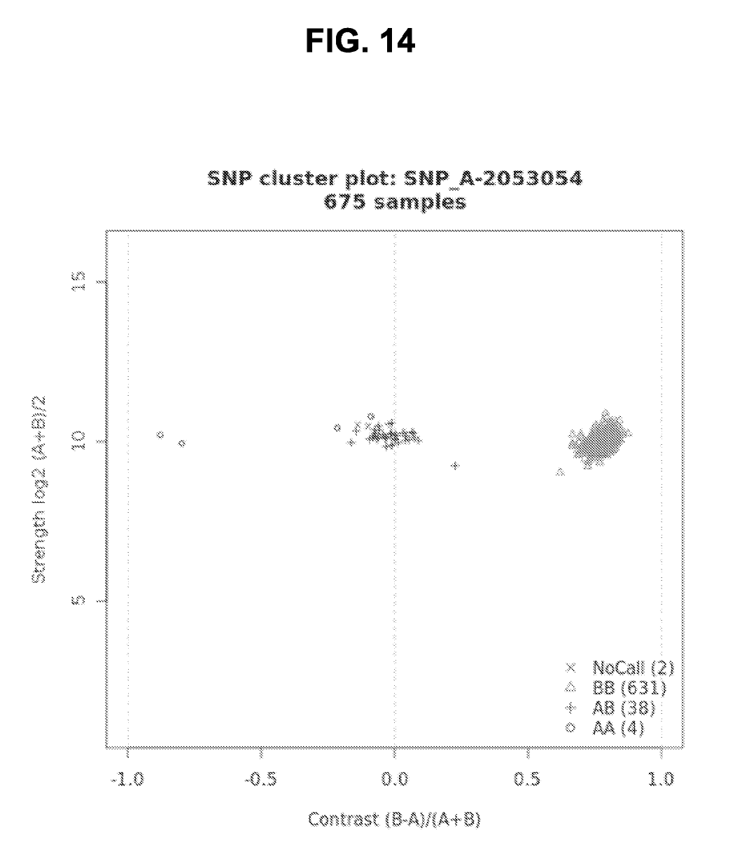

The genotype cluster plot of the top hitting SNP (SNP_A-2053054) is shown in FIG. 14.

Kaplan-Meier Plot

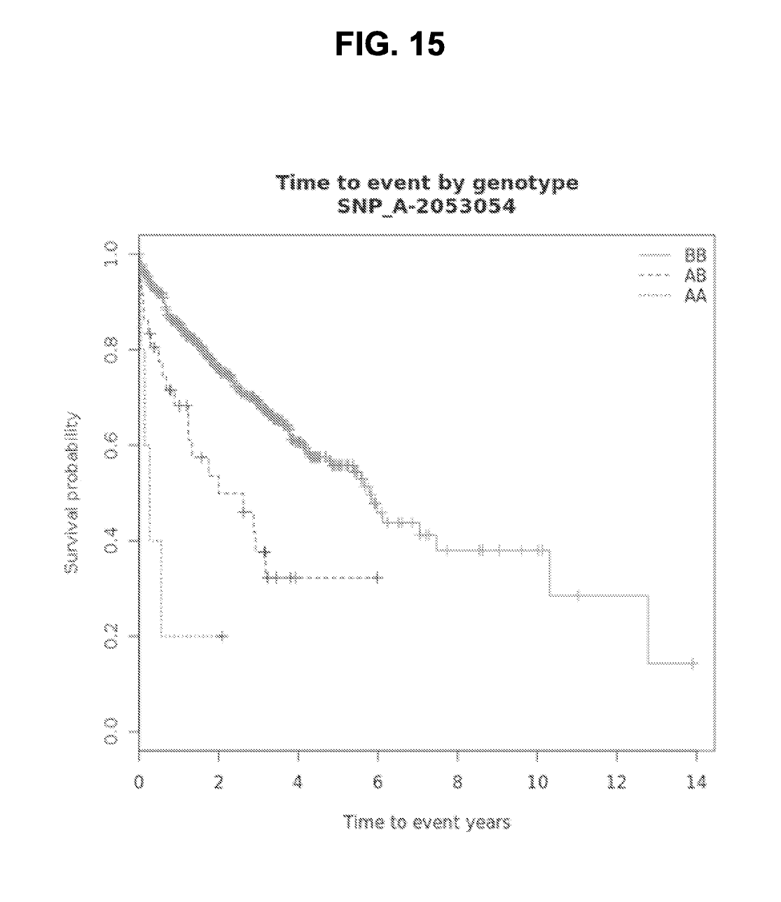

The Kaplan-Meier plot in FIG. 15 shows the differential survival between the different genotypes for SNP_A-2053054.

Proportional Odds Assumption

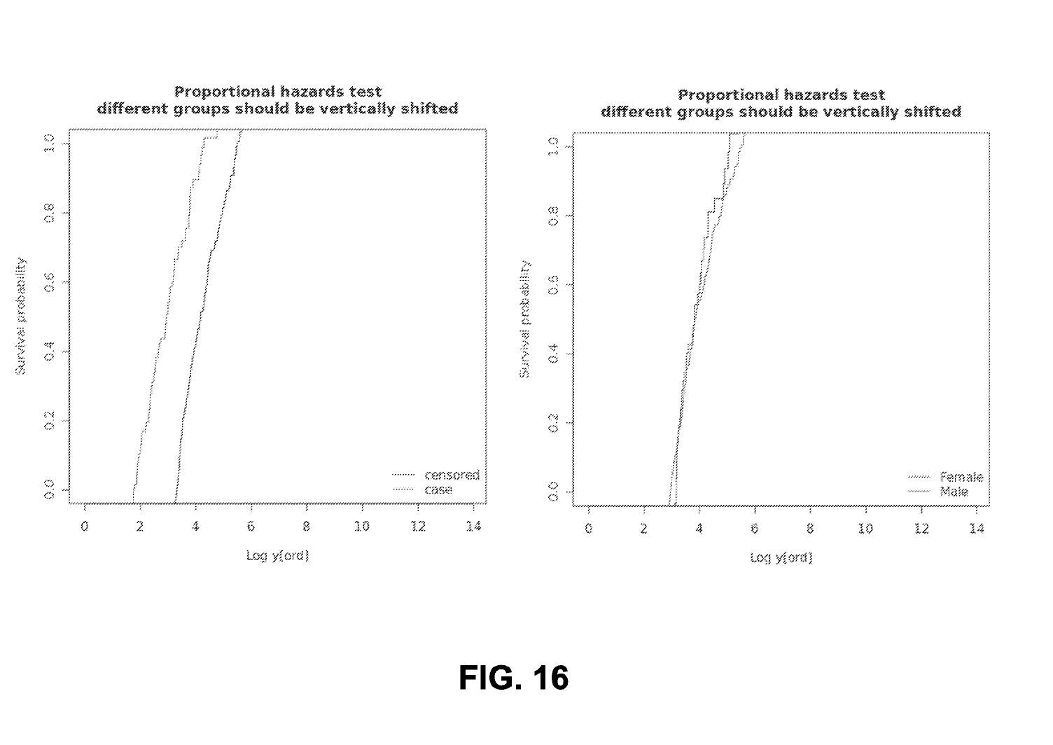

The Cox model fit makes a proportional odds assumption, which was tested in the plot of FIG. 16. When the two groups, cases and censored, are vertical shifts of each other then the proportional odds assumption holds very well, as in this case. The gender plot shows similar results (FIG. 16).

### Manhattan Plot

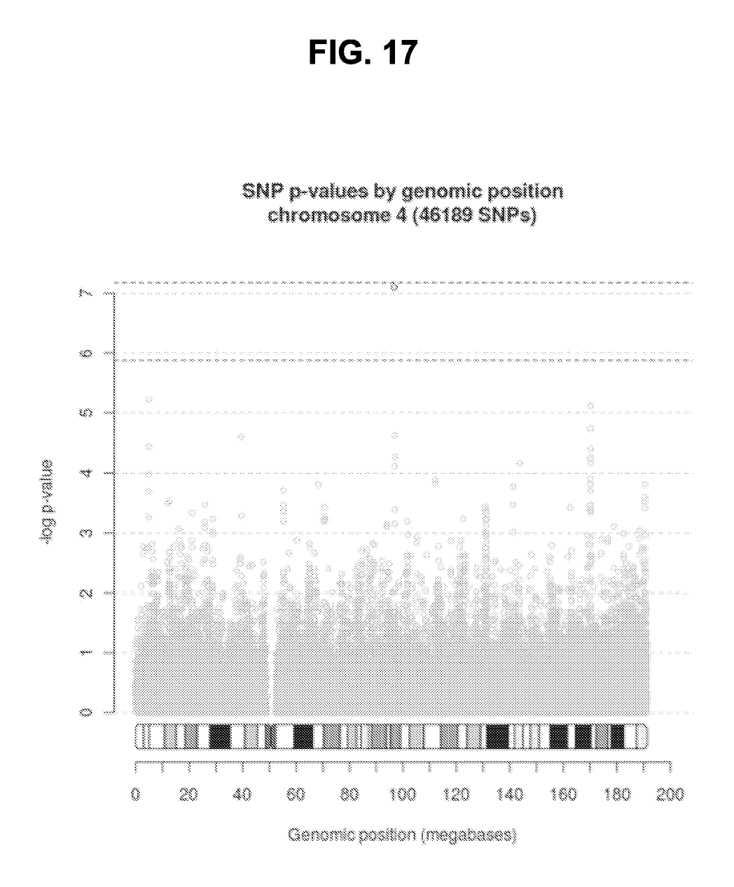

The Manhattan plot of FIG. 17 shows the p-values for the SNPs on chromosome 4, which includes the top hitting SNPs. The red dashed-line at the top represents the conservative Bonferroni level required for genome-wide significance.

Effective Number of Tests

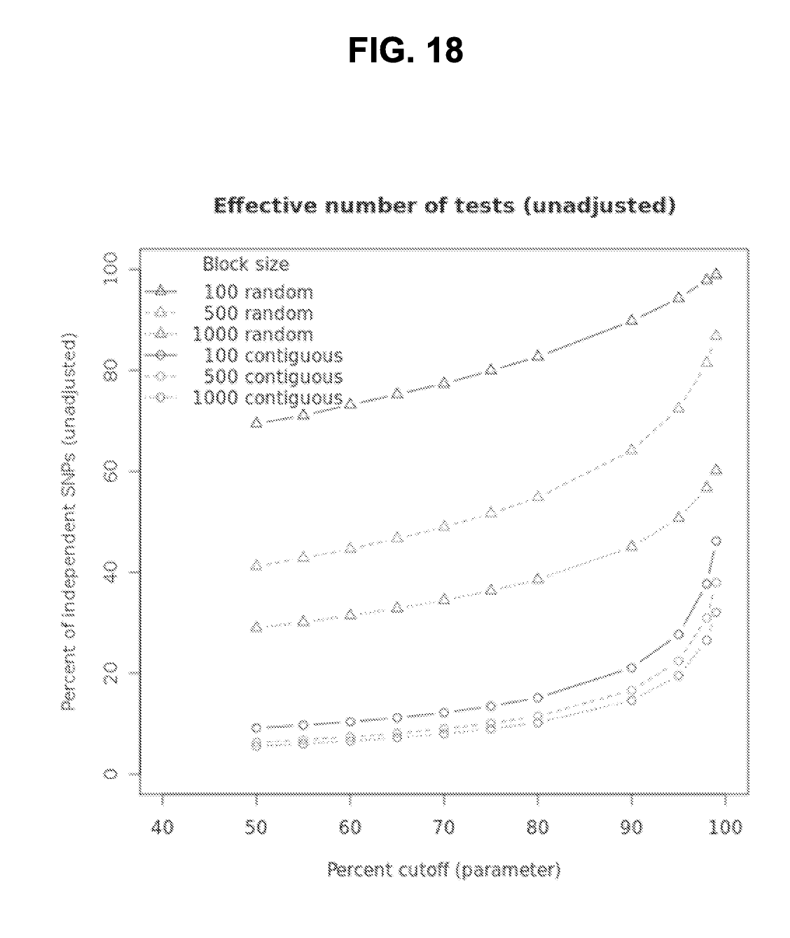

Briefly, the SNPs were partitioned into blocks of SNPs contiguous along the genome, for k=100, 500, and 1000. For each block of SNPs we formed the genotype matrix for the 658 passing samples. With this matrix we obtained the correlation matrix of SNP to SNP correlations. We obtained the list of singular values (eigenvalues) using the singular value decomposition (SVD) of the correlation matrix. The effective number of independent tests of a block of SNPs was the number of the largest singular values surpassing a fix proportion, given by a percent cutoff, of the total sum of singular values. The total effective number of tests was estimated by summing the values obtained from each block. To calibrate the method, a similar calculation was done with a random selection of SNP blocks that mirror the sizes of the contiguous SNP blocks. The plot in FIG. 18 shows the results of these calculations for contiguous blocks and random blocks and for the several block sizes 100, 500, and 1000, and as a function of the percent cutoff. Each curve approaches 100% on the right. The right side values include the independent SNPs as well as the random noise.

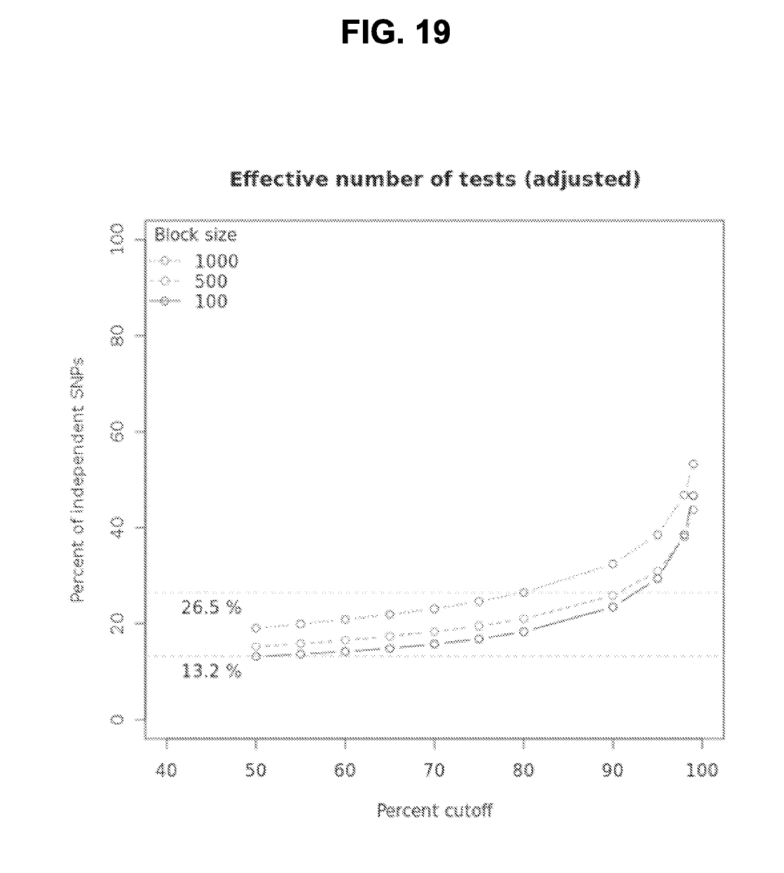

The random block results should represent the situation when the SNPs are nearly independent, as random SNPs are typically far from each other along the genome. But from the graph (FIG. 19) we see the curves for the random blocks have rather low values (e.g., not above 80%). We calibrated the contiguous block values by taking their proportion with respect to the random block values (divided the contiguous block values by the random block values for each cutoff value). From the following plot (FIG. 19) we estimated a value of anywhere from 13% to 26% for the percentage of independent SNPs.

### Example 4

### Analysis of Genes Located Near SNPs

The sympathetic and parasympathetic systems innervate the heart and are involved in controlling heart rate. In response to physical or mental stress, the sympathetic system is activated and norepinephrine (NE) is released. The released NE binds to beta-adrenergic receptors located on myocytes resulting in increased contractility. Compromised innervation of the heart by the sympathetic nervous system may be proarrhythmogenic and may lead to heart failure. Imaging studies have shown that aberrant sympathetic innervation is present in patients with Brugada's syndrome, a condition that leads to life-threatening ventricular tachyarrhythmias despite patients having what appear to be structurally normal hearts¹. In addition, mutations in the myocytic de-polarization/re-polarization pathways and contractile proteins have also been shown to be proarrhythmogenic^{2,3}.

We conducted a study (see Examples above) to identify genetic defects that are associated with increased firing rates of implantable cardiac defibrillator (ICD's); increased firing rates are indicative of increased susceptibility to arrhythmic events. The study investigated the association of ˜750,000 genetic markers (or single nucleotide polymorphisms, SNPs) for association with increased firing rates in a heart failure population in which all patients had an ICD. Using a false-discovery rated (FDR) cut-off, we identified 124 SNPs (Table 15) with an FDR less than 50%; these were derived from analyzing both the entire population as well as a subset of patients with ischemic heart failure. The 124 SNPs mapped to 68 distinct loci; 1 locus had no clear association with a nearby gene, 40 loci mapped to a single gene, 24 loci to two genes, and 3 loci mapped to 3 genes (Table 15). The SNPs shown in Table 15 are referred to by their Reference SNP ID, e.g. rs709932, as found on the NCBI SNP website on Mar. 17, 2010. For example, a query for rs12082124 on the NCBI SNP website on Mar. 17, 2010 returns the following information: rs 12082124 [Homo sapiens]GCAAAGGTAGAAAAACTCCTGAATTT[A/G]AAAGCACTAAACTAGGAGTCA GGCT (SEQ ID NO:1).

In order to better understand the biology of these top candidates, we used publically available data to further annotate the genes near the significant SNPs, in regards to their biologically function and pathways. Of the 69 clusters, 31 had genes (shown in BOLD below, also in Table 16) associated with them that were judged to have biologically relevant annotation based on the known biology around arrythmias.

Genes Involved in Neurogenesis and Cytoskeletal Rearrangement

Developmental defects can lead to improper neurogenesis and defective innervation. A number of the top SNPs are near genes that may be either involved in proper neuronal targeting and pathfinding (UNC5C)⁴, organization of the cytoskeleton in the growth cone (ARPC3, FRMD3, TANC2, TCP10L2)⁵⁻⁷, and transcriptional regulation of neural development (ZFHX3, ID4)^{8,9}. Interestingly, SNPs near ZFHX3 have recently been associated with increased likelihood of atrial fibrillation^{10,11}. PALLD encodes a cytoskeletal protein that is required for organizing the actin cytoskeleton¹². Knock-down of PPIA (cyclophilin A) in U2OS cells has been shown to disrupt F-actin structure. Biochemically PPIA bids N-WASP, which functions in the nucleation of actin via the Arp2/3 complex¹³.

MYLIP binds to the myosin regulatory light chain, which in turn protein regulates the activity of the actomyosin complex. Overexpression of MYLIP cDNA in PC12 cells has been shown to abrogate neurite outgrowth induced by nerve growth factor (NGF)¹⁴. SEMA6D, a semaphorin, has been shown to inhibit axonal extension of nerve growth factor differentiated PC12 cells, and also may a play a role in cardiac morphogenesis^{15,16}.

Genes Involved in Vesicle Transport and Vesicle Function

Vesicle transport in neurons is required for delivery of neurotransmitters such as norepinephrine (NE) to the synapse for subsequent release. Dynein is a complex of proteins which forms a molecular motor which moves vesicles along a molecular track composed of tubulin. DYNLR132 encodes one of the dynein light chains¹⁷. ACTR10 is a component of dynactin, a complex that binds to dynein and aids in bidirectional intracellular organelle transport¹⁸. NRSN2 is a neuronal protein that is found in the membranes of small vesicles and may play a role in vesicle transport¹⁹. STX18, a syntaxin, has been shown to be involved in membrane trafficking between the ER and Giolgi²⁰. ARL4C, an ADP-ribosylation factor, might modulate intracellular vesicular transport via interaction with microtubules²¹. SLC9A7 is expressed predominantly in the trans-Golgi network, and interacts with cytoskeletal components such as vimentin²².

Neuronal Adhesion

Adhesion molecules are required for the proper alignment of neurons and myocytes at the neuromuscular junction. CNTNAP2 is a member of the neurexin family which functions in the vertebrate nervous system as cell adhesion molecules and receptors, and may play a role in differentiation of the axon into distinct functional subdomains²³. NRXN1 is a neurexin which is involved in neuronal cell adhesion²⁴. LRRC7 is a protein that is found in the postsynaptic density in neurons and may function as a synaptic adhesion molecule²⁵. PCDH15 and PCDH9 are both members of the cadherin superfamily, which encode integral membrane proteins that mediate calcium-dependent cell-cell adhesion²⁶. LSAMP is a selective homophilic adhesion molecule that guides the development of specific patterns of neuronal connections²⁷. FYN is a well-characterized protein-tyrosine kinase which has been implicated in cell growth and survival. Recently FYN has been shown to negatively regulate synapse formation through inhibition of PTPRT, preventing its association with neuroligins²⁸.

Beta-Adrenergic Receptor Signaling and Modulation

Once released from the neuron into the synaptic cleft, NE binds to beta-adrenergic receptors to promote depolarization, and is also actively transported back into the neuron. UTRN is a protein that is located at the neuromuscular synapse and myotendinous junctions, where it participates in post-synaptic membrane maintenance and acetylcholine receptor clustering; as such is may play a role in the proper positioning of beta-AR's²⁹. ADCY3, an adenylate cyclase, has been shown to be stimulated by beta-adrenergic agonists and may play a role in beta-adrenergic signaling³⁰.

Upon binding by INE, beta-ARs are subjected to clathirin-pit mediated endocytosis as a mechanism to down-regulate NE signaling. ACVR1 biochemically interacts with AP2B1, one of the two large chain components of the assembly protein complex 2; AP2B1 has been shown to interact with beta-adrenergic receptors during endocytosis^{31,32}. ITSN2 is thought to regulate the formation of clathrin-coated vesicles and may play a role linking coated vesicles to the cytoskeleton through the Arp2/3 complex^{33,34}. ST13, a protein that interacts with Hsp70, has been shown to play a role in the internalization of G protein coupled receptors (GPCRs); as such it might play a role in the internalization of beta-adrenergic receptors³⁵.

NE is internalized back into the neuron through the sodium transporter SLC6A2. CACNA1D may form a molecular complex with SCL6A2 through its interaction with STX1A, a syntaxin that interacts with both proteins³¹.

Depolarization and Muscle Contraction.

CACNA1D is a component of a L-type voltage-dependent calcium channel, mutations in which are proarrhythmogenic³⁶. It has been shown that the activity of Ca2+ channels can be regulated by agents that disrupt or stabilize the cytoskeleton³⁷. Sadeghi et al have shown that both dystrophin and alpha-actinin colocalize with the L-type Ca2+ channel in mouse cardiac myocytes and to modulate channel function.

UTRN interacts with a number of components of the dystrophin-associated protein complex (DGC), which consists of dystrophin and several integral and peripheral membrane proteins, including dystroglycans, sarcoglycans, syntrophins and alpha- and beta-dystrobrevin. In the neuron, the DPC participates in macromolecular assemblies that anchor receptors to specialized sites within the membrane³⁹. SGCZ is part of the sarcoglycan complex, which is a component of the dystrophin-associated glycoprotein complex (DGC), which bridges the inner cytoskeleton and the extra-cellular matrix³⁹. MAST4, a microtubule associated serine/threonine kinase, may play a role in the DPC complex as an ortholog, MAST2, interacts with the syntrophin SNTB2³¹. Interestingly, all 4 orthologs (MAST1, 2, 3 and 4) bind to PTEN, a protein that negatively regulates intracellular levels of phosphatidylinositol-3,4,5-trisphosphate in cells and thus may play a role in Ca++ signaling in the heart³¹.

### APPENDIX A

Genes with Annotation by Homology

TANC1—TANC2

65% identical; neither protein has good literature annotation, however biochemically TANC1 interacts with:

SPTAN1—alpha spectrin

GRIN2B glutamate receptor, ionotropic, p value 0.000335

DLGAP1—discs, large (Drosophila) homolog-associated protein 1 (p value 0.00749, just missed 50% FDR cut-off)

ACTB—actin B

TCP10—TCP10L2

96% identical; neither protein has good literature annotation, however biochemically TCP10 interacts with:

PARD6A, PARD6B—involved in controlling neural migration

MAST2—MAST4

66% identical; all paralogs (MAST 1, 2, 3) bind PTEN, involved in Ca++ signaling; MAST2 also binds:

SNTB2—syntrophin, beta 2

DYNLL1—dynein, light chain, LC8-type 1

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

TABLE 1

| | | | | | | | Mutated or |
|---|---|---|---|---|---|---|---|
| | | | | | | | associated |
| Ensembl Gene | | Start Position | End Position | Transcript | | | with Human SCD |
| ID Ver 42 | Chromosome Name | (bp) | (bp) | count | HGNC Symbol | Gene Name | disorders |
| ENSG00000159251 | 15 | 32869724 | 32875181 | 1 | ACTC1 | actin, | x |
| | | | | | | alpha, | |
| | | | | | | cardiac | |
| | | | | | | muscle | |
| ENSG00000072110 | 14 | 68410793 | 68515747 | 1 | ACTN1 | actinin, | |
| | | | | | | alpha 1 | |
| ENSG00000184160 | 4 | 3738094 | 3740051 | | ADRA2C | adrenergic, | |
| | | | | | | alpha-2C-, | |
| | | | | | | receptor | |
| ENSG00000043591 | 10 | 115793796 | 115796657 | 2 | ADRB1 | adrenergic, | |
| | | | | | | beta-1-, | |
| | | | | | | receptor | |
| ENSG00000169252 | 5 | 148185001 | 148188447 | 1 | ADRB2 | adrenergic, | |
| | | | | | | beta-2-, | |
| | | | | | | receptor, | |
| | | | | | | surface | |
| ENSG00000188778 | 8 | 37939673 | 37943341 | 1 | ADRB3 | adrenergic, | |
| | | | | | | beta-3-, | |
| | | | | | | receptor | |
| ENSG00000173020 | 11 | 66790507 | 66810602 | 1 | ADRBK1 | adrenergic, | |
| | | | | | | beta, | |
| | | | | | | receptor | |
| | | | | | | kinase 1 | |
| ENSG00000100077 | 22 | 24290946 | 24449916 | | ADRBK2 | adrenergic, | |
| | | | | | | beta, | |
| | | | | | | receptor | |
| | | | | | | kinase 2 | |
| ADD | | | | | AKAP10 | A kinase | |
| | | | | | | (PRKA) | |
| | | | | | | anchor | |
| | | | | | | protein 10 | |
| ENSG00000170776 | 15 | 83578821 | 84093590 | 3 | AKAP13 | A kinase | |
| | | | | | | (PRKA) | |
| | | | | | | anchor | |
| | | | | | | protein 13 | |
| ENSG00000151320 | 14 | 31868274 | 32372018 | 1 | AKAP6 | A kinase | |
| | | | | | | (PRKA) | |
| | | | | | | anchor | |
| | | | | | | protein 6 | |
| ENSG00000127914 | 7 | 91408128 | 91577925 | 6 | AKAP9 | A kinase | x |
| | | | | | | (PRKA) | |
| | | | | | | anchor | |
| | | | | | | protein | |
| | | | | | | (yotiao) 9 | |
| ENSG00000198363 | 8 | 62578374 | 62789681 | 11 | ASPH | aspartate | |
| | | | | | | beta- | |
| | | | | | | hydroxylase; | |
| | | | | | | junctin | |
| | | | | | | included | |
| ENSG00000196296 | 16 | 28797310 | 28823331 | 1 | ATP2A1 | ATPase, | |
| | | | | | | Ca++ | |
| | | | | | | transporting, | |
| | | | | | | cardiac | |
| | | | | | | muscle, | |
| | | | | | | fast twitch 1 | |
| ENSG00000174437 | 12 | 109203815 | 109273278 | 3 | ATP2A2 | ATPase, | |
| | | | | | | Ca++ | |
| | | | | | | transporting, | |
| | | | | | | cardiac | |
| | | | | | | muscle, | |
| | | | | | | slow twitch 2 | |
| ENSG00000151067 | 12 | 2094650 | 2670626 | 5 | CACNA1C | calcium | x |
| | | | | | | channel, | |
| | | | | | | voltage- | |
| | | | | | | dependent, | |
| | | | | | | L type, | |
| | | | | | | alpha 1C | |
| | | | | | | subunit | |
| ENSG00000157388 | 3 | 53503723 | 53821112 | 2 | CACNA1D | calcium | |
| | | | | | | channel, | |
| | | | | | | voltage- | |
| | | | | | | dependent, | |
| | | | | | | L type, | |
| | | | | | | alpha 1D | |
| | | | | | | subunit | |
| ENSG00000153956 | 7 | 81417354 | 81910967 | 3 | CACNA2D1 | calcium | |
| | | | | | | channel, | |
| | | | | | | voltage- | |
| | | | | | | dependent, | |
| | | | | | | alpha | |
| | | | | | | 2/delta | |
| | | | | | | subunit 1 | |
| ENSG00000007402 | 3 | 50375237 | 50516032 | 2 | CACNA2D2 | calcium | |
| | | | | | | channel, | |
| | | | | | | voltage- | |
| | | | | | | dependent, | |
| | | | | | | alpha | |
| | | | | | | 2/delta | |
| | | | | | | subunit 2 | |
| ENSG00000157445 | 3 | 54131733 | 55083622 | 1 | CACNA2D3 | calcium | |
| | | | | | | channel, | |
| | | | | | | voltage- | |
| | | | | | | dependent, | |
| | | | | | | alpha | |
| | | | | | | 2/delta 3 | |
| | | | | | | subunit | |
| ENSG00000165995 | 10 | 18469612 | 18870797 | 9 | CACNB2 | calcium | |
| | | | | | | channel, | |
| | | | | | | voltage- | |
| | | | | | | dependent, | |
| | | | | | | beta 2 | |
| | | | | | | subunit | |
| ENSG00000167535 | 12 | 47498779 | 47508991 | 1 | CACNB3 | calcium | |
| | | | | | | channel, | |
| | | | | | | voltage- | |
| | | | | | | dependent, | |
| | | | | | | beta 3 | |
| | | | | | | subunit | |
| ENSG00000145349 | 4 | 114593022 | 114902177 | 4 | CAMK2D | calcium/ | |
| | | | | | | calmodulin- | |
| | | | | | | dependent | |
| | | | | | | protein | |
| | | | | | | kinase | |
| | | | | | | (CaM | |
| | | | | | | kinase) II | |
| | | | | | | delta | |
| ENSG00000077549 | 1 | 19537857 | 19684594 | 5 | CAPZB | Capping | |
| | | | | | | protein | |
| | | | | | | (actin | |
| | | | | | | filament) | |
| | | | | | | muscle Z- | |
| | | | | | | line, beta | |
| ENSG00000118729 | 1 | 116044151 | 116112925 | 1 | CASQ2 | calsequestrin | x |
| | | | | | | 2 (cardiac | |
| | | | | | | muscle) | |
| ENSG00000119782 | 2 | 24126075 | 24140055 | 4 | FKBP1B | FK506 | |
| | | | | | | binding | |
| | | | | | | protein 1B, | |
| | | | | | | 12.6 kDa | |
| ENSG00000114353 | 3 | 50239173 | 50271775 | 3 | GNAI2 | guanine | |
| | | | | | | nucleotide | |
| | | | | | | binding | |
| | | | | | | protein (G | |
| | | | | | | protein), | |
| | | | | | | alpha | |
| | | | | | | inhibiting | |
| | | | | | | activity | |
| | | | | | | polypeptide 2 | |
| ENSG00000111664 | 12 | 6820713 | 6826819 | 2 | GNB3 | guanine | |
| | | | | | | nucleotide | |
| | | | | | | binding | |
| | | | | | | protein (G | |
| | | | | | | protein), | |
| | | | | | | beta | |
| | | | | | | polypeptide 3 | |
| ENSG00000134571 | 11 | 47309527 | 47330806 | 1 | MYBPC3 | myosin | x |
| | | | | | | binding | |
| | | | | | | protein C, | |
| | | | | | | cardiac | |
| ENSG00000197616 | 14 | 22921038 | 22946665 | 2 | MYH6 | myosin, | |
| | | | | | | heavy | |
| | | | | | | polypeptide | |
| | | | | | | 6, cardiac | |
| | | | | | | muscle, | |
| | | | | | | alpha | |
| | | | | | | (cardio- | |
| | | | | | | myopathy, | |
| | | | | | | hypertrophic 1) | |
| ENSG00000092054 | 14 | 22951789 | 22974690 | 2 | MYH7 | myosin, | x |
| | | | | | | heavy | |
| | | | | | | polypeptide | |
| | | | | | | 7, cardiac | |
| | | | | | | muscle, | |
| | | | | | | beta | |
| ENSG00000111245 | 12 | 109833009 | 109842766 | 1 | MYL2 | myosin, | x |
| | | | | | | light | |
| | | | | | | polypeptide 2, | |
| | | | | | | regulatory, | |
| | | | | | | cardiac, | |
| | | | | | | slow | |
| ENSG00000160808 | 3 | 46874371 | 46879938 | 1 | MYL3 | myosin, | x |
| | | | | | | light | |
| | | | | | | polypeptide | |
| | | | | | | 3, alkali; | |
| | | | | | | ventricular, | |
| | | | | | | skeletal, | |
| | | | | | | slow | |
| | | | | | PDE4A | phospho- | |
| | | | | | | diesterase 4A | |
| ENSG00000113448 | 5 | 58305622 | 59320301 | 5 | PDE4D | phospho- | |
| | | | | | | diesterase 4D, | |
| | | | | | | cAMP- | |
| | | | | | | specific | |
| | | | | | | (phospho- | |
| | | | | | | diesterase | |
| | | | | | | E3 dunce | |
| | | | | | | homolog, | |
| | | | | | | Drosophila) | |
| ENSG00000198523 | 6 | 118976154 | 118988586 | 1 | PLN | phospholamban | x |
| ENSG00000072062 | 19 | 14063509 | 14089559 | 2 | PRKACA | protein | |
| | | | | | | kinase, | |
| | | | | | | cAMP- | |
| | | | | | | dependent, | |
| | | | | | | catalytic, | |
| | | | | | | alpha | |
| ENSG00000114302 | 3 | 48762099 | 48860274 | 2 | PRKAR2A | protein | |
| | | | | | | kinase, | |
| | | | | | | cAMP- | |
| | | | | | | dependent, | |
| | | | | | | regulatory, | |
| | | | | | | type II, | |
| | | | | | | alpha | |
| ENSG00000198626 | 1 | 235272128 | 236063911 | 3 | RYR2 | ryanodine | x |
| | | | | | | receptor 2 | |
| | | | | | | (cardiac) | |
| ENSG00000136450 | 17 | 53437651 | 53439593 | 2 | SFRS1 | splicing | |
| | | | | | | factor, | |
| | | | | | | arginine/ | |
| | | | | | | serine-rich 1 | |
| | | | | | | (splicing | |
| | | | | | | factor 2, | |
| | | | | | | alternate | |
| | | | | | | splicing | |
| | | | | | | factor) | |
| ENSG00000183023 | 2 | 40192790 | 40534188 | 5 | SLC8A1 | solute | |
| | | | | | | carrier | |
| | | | | | | family 8 | |
| | | | | | | (sodium/ | |
| | | | | | | calcium | |
| | | | | | | exchanger), | |
| | | | | | | member 1 | |
| ENSG00000118160 | 19 | 52623735 | 52666934 | 1 | SLC8A2 | solute | |
| | | | | | | carrier | |
| | | | | | | family 8 | |
| | | | | | | (sodium- | |
| | | | | | | calcium | |
| | | | | | | exchanger), | |
| | | | | | | member 2 | |
| ENSG00000090020 | 1 | 27297893 | 27366059 | 4 | SLC9A1 | solute | |
| | | | | | | carrier | |
| | | | | | | family 9 | |
| | | | | | | (sodium/ | |
| | | | | | | hydrogen | |
| | | | | | | exchanger), | |
| | | | | | | member 1 | |
| | | | | | | (antiporter, | |
| | | | | | | Na+/H+, | |
| | | | | | | amiloride | |
| | | | | | | sensitive) | |
| ENSG00000170290 | 11 | 107083319 | 107087992 | 1 | SLN | sarcolipin | |
| ENSG00000136842 | 9 | 99303742 | 99403357 | 2 | TMOD1 | tropomodulin 1 | |
| ENSG00000114854 | 3 | 52460158 | 52463098 | 1 | TNNC1 | troponin C | x |
| | | | | | | type 1 | |
| | | | | | | (slow) | |
| ENSG00000129991 | 19 | 60355014 | 60360496 | 1 | TNNI3 | troponin I | x |
| | | | | | | type 3 | |
| | | | | | | (cardiac) | |
| ENSG00000118194 | 1 | 199594759 | 199613431 | 10 | TNNT2 | troponin T | x |
| | | | | | | type 2 | |
| | | | | | | (cardiac) | |
| ENSG00000140416 | 15 | 61121891 | 61151164 | 7 | TPM1 | tropomyosin 1 | x |
| | | | | | | (alpha) | |
| ENSG00000186439 | 6 | 123579183 | 123999937 | 5 | TRDN | triadin | |

| | | | | Ion | | | |
|---|---|---|---|---|---|---|---|
| Ensembl Gene | Disease | Other | | (handling or | structural or | EG | |
| ID Ver 42 | Groupings | LOE | Organelle | dependence) | function | coupling | |
| ENSG00000159251 | HCM, | found in | | | myofilament | 1 | |
| | DCM | discovery | | | | | |
| | | HF v ctrl | | | | | |
| ENSG00000072110 | | | | | myofilament | 1 | |
| ENSG00000184160 | | | | Epi/NE | signaling, | 1 | low MAF |
| | | | | | sympathetic | | in whites |
| ENSG00000043591 | | | | Epi/NE | signaling, | 1 | |
| | | | | | sympathetic | | |
| ENSG00000169252 | | found in | | Epi/NE | signaling, | 1 | |
| | | discovery | | | sympathetic | | |
| | | HF v ctrl | | | | | |
| ENSG00000188778 | | least | | Epi/NE | signaling, | 1 | |
| | | described | | | sympathetic | | |
| ENSG00000173020 | | | | | phosphorylation | 1 | |
| ENSG00000100077 | | | | | phosphorylation | 1 | |
| ADD | | localization | | | | 1 | |
| | | of PKA | | | | | |
| ENSG00000170776 | | found in | | | phosphorylation | 1 | |
| | | discovery | | | | | |
| | | HF v ctrl | | | | | |
| ENSG00000151320 | | found in | | | phosphorylation | 1 | |
| | | discovery | | | | | |
| | | HF v ctrl | | | | | |
| ENSG00000127914 | LQT11 | | | | phosphorylation | 1 | |
| ENSG00000198363 | | transmembrane | SR | Ca++ | | 1 | |
| | | calsequestrin; | | | | | |
| | | colocalizes | | | | | |
| | | with the RYR | | | | | |
| | | and triadin | | | | | |
| ENSG00000196296 | | | SR | Ca++ | transmembrane | 1 | |
| | | | | | protein | | |
| ENSG00000174437 | | | SR | Ca++ | transmembrane | 1 | |
| | | | | | protein | | |
| ENSG00000151067 | LQT8 | found in | cell | Ca++ | | 1 | |
| | | discovery | membrane | | | | |
| | | HF v ctrl | | | | | |
| ENSG00000157388 | | found in | cell | Ca++ | | 1 | |
| | | discovery | membrane | | | | |
| | | HF v ctrl; | | | | | |
| | | Subunit | | | | | |
| | | of L-type | | | | | |
| | | calcium | | | | | |
| | | channel | | | | | |
| ENSG00000153956 | | found in | cell | Ca++ | | 1 | |
| | | discovery | membrane | | | | |
| | | HF v ctrl; | | | | | |
| | | Subunit: | | | | | |
| | | of L-type | | | | | |
| | | calcium | | | | | |
| | | channel | | | | | |
| ENSG00000007402 | | found in | cell | Ca++ | | 1 | |
| | | discovery | membrane | | | | |
| | | HF v ctrl; | | | | | |
| | | Subunit | | | | | |
| | | of L-type | | | | | |
| | | calcium | | | | | |
| | | channel | | | | | |
| ENSG00000157445 | | found in | cell | Ca++ | | 1 | |
| | | discovery | membrane | | | | |
| | | HF v ctrl; | | | | | |
| | | Subunit | | | | | |
| | | of L-type | | | | | |
| | | calcium | | | | | |
| | | channel | | | | | |
| ENSG00000165995 | | found in | cell | Ca++ | | 1 | |
| | | discovery | membrane | | | | |
| | | HF v ctrl; | | | | | |
| | | Subunit | | | | | |
| | | of L-type | | | | | |
| | | calcium | | | | | |
| | | channel | | | | | |
| ENSG00000167535 | | found in | cell | Ca++ | | 1 | |
| | | discovery | membrane | | | | |
| | | HF v ctrl; | | | | | |
| | | Subunit | | | | | |
| | | of L-type | | | | | |
| | | calcium | | | | | |
| | | channel | | | | | |
| ENSG00000145349 | | | | Ca++ | phosphorylation, | 1 | |
| | | | | | KEY | | |
| ENSG00000077549 | | | | | myofilament | 1 | |
| ENSG00000118729 | CPVT, | found in | SR | Ca++ | | 1 | |
| | recessive | discovery | | | | | |
| | | HF v ctrl | | | | | |
| ENSG00000119782 | | assoc | SR | Ca++ | | 1 | |
| | | with RYR | | | | | |
| ENSG00000114353 | | somatic | | | | 1 | |
| | | mutation | | | | | |
| | | and VT | | | | | |
| ENSG00000111664 | | | | | | 1 | |
| ENSG00000134571 | HCM | | | | myofilament | 1 | |
| ENSG00000197616 | | found in | | | myofilament | 1 | |
| | | discovery | | | | | |
| | | HF v ctrl | | | | | |
| ENSG00000092054 | HCM, | | | | myofilament | 1 | |
| | DCM | | | | | | |
| ENSG00000111245 | HCM | | | | myofilament | 1 | |
| ENSG00000160808 | HCM | | | | myofilament | 1 | |
| | | interacts | | | | 1 | |
| | | with | | | | | |
| | | AKAP6 | | | | | |
| ENSG00000113448 | | found in | SR | Ca++ | | 1 | |
| | | discovery | | | | | |
| | | HF v ctrl; | | | | | |
| | | assoc | | | | | |
| | | with RYR | | | | | |
| ENSG00000198523 | DCM | Found in | SR | Ca++ | | 1 | |
| | | QTGEN | | | | | |
| | | and | | | | | |
| | | QTSCD | | | | | |
| ENSG00000072062 | | CONFIRM | | Ca++ | phosphorylation, | 1 | |
| | | THIS | | | KEY | | |
| | | IS PKA | | | | | |
| ENSG00000114302 | | | | Ca++ | phosphorylation, | 1 | |
| | | | | | KEY | | |
| ENSG00000198626 | CPVT | found in | SR | Ca++ | | 1 | |
| | (exons 1- | discovery | | | | | |
| | 28, 37- | HF v ctrl; | | | | | |
| | 50, 75, | assoc | | | | | |
| | 83-105) | with | | | | | |
| | | lower | | | | | |
| | | SCA risk | | | | | |
| | | (AHA | | | | | |
| | | abstract) | | | | | |
| ENSG00000136450 | | regulates | | | splicing | 1 | |
| | | splicing | | | | | |
| | | of | | | | | |
| | | CAMK2D; | | | | | |
| | | deficiency | | | | | |
| | | causes | | | | | |
| | | severe | | | | | |
| | | EC | | | | | |
| | | coupling | | | | | |
| | | defects | | | | | |
| ENSG00000183023 | | | cell | Na+/Ca++ | membrane | 1 | |
| | | | membrane | | ion | | |
| | | | | | exchanger | | |
| ENSG00000118160 | | | cell | Na+/Ca++ | membrane | 1 | |
| | | | membrane | | ion | | |
| | | | | | exchanger | | |
| ENSG00000090020 | | | | Na+/H+ | membrane | 1 | |
| | | | | | ion | | |
| | | | | | exchanger | | |
| ENSG00000170290 | | interact | SR | | | 1 | |
| | | with PLN | | | | | |
| | | and | | | | | |
| | | ATP2A1 | | | | | |
| ENSG00000136842 | | found in | | | myofilament | 1 | |
| | | discovery | | | | | |
| | | HF v ctrl | | | | | |
| ENSG00000114854 | HCM | | | Ca++ | myofilament | 1 | |
| ENSG00000129991 | HCM | | | | myofilament | 1 | |
| ENSG00000118194 | HCM, | | | | myofilament | 1 | |
| | DCM | | | | | | |
| ENSG00000140416 | HCM | found in | | | myofilament | 1 | |
| | | discovery | | | | | |
| | | HF v ctrl | | | | | |
| ENSG00000186439 | | found in | SR | | | 1 | |
| | | discovery | | | | | |
| | | HF v ctrl; | | | | | |
| | | colocalizes | | | | | |
| | | with | | | | | |
| | | the RYR | | | | | |
| | | and | | | | | |
| | | junctin; | | | | | |
| | | skel m | | | | | |
| | | and | | | | | |
| | | cardiac | | | | | |
| | | isoforms | | | | | |

TABLE 2

| | | | | | | | Mutated or | | | | | | |
|---|---|---|---|---|---|---|---|---|---|---|---|---|---|
| | | | | | | | associated | | | | | | |
| | | | | | | | with | | | | | | |
| Ensembl | | Start | End | | | | Human | | | | Ion (handling | | |
| Gene ID | Chromosome | Position | Position | Transcript | HGNC | | SCD | Disease | | | or | | ion |
| Ver 42 | Name | (bp) | (bp) | count | Symbol | Gene Name | disorders | Groupings | Other LOE | Organelle | dependence) | structural | channels |
| ENSG00000130037 | 12 | 5023346 | 5026210 | 1 | KCNA5 | potassium | x | A fib | antiarrhythmic | | K+ | ion channel | 2 |
| | | | | | | voltage- | | | drug | | | | |
| | | | | | | gated | | | sensitivity | | | | |
| | | | | | | channel, | | | | | | | |
| | | | | | | shaker- | | | | | | | |
| | | | | | | related | | | | | | | |
| | | | | | | subfamily, | | | | | | | |
| | | | | | | member 5 | | | | | | | |
| ENSG00000175548 | 12 | 36996824 | 37001523 | 1 | ALG10B | asparagine- | | acquired | | | | ion channel | 2 |
| | | | | | | linked | | LQTS | | | | | |
| | | | | | | glycosylation | | | | | | | |
| | | | | | | 10 homolog | | | | | | | |
| | | | | | | B (yeast, | | | | | | | |
| | | | | | | alpha-1,2- | | | | | | | |
| | | | | | | glucosyltransferase) | | | | | | | |
| | | | | | | (KCR1) | | | | | | | |
| ENSG00000166257 | 11 | 123005107 | 1.23E+08 | 1 | SCN3B | sodium | x | Brugada | Leu10Pro | | Na+ | ion channel | 2 |
| | | | | | | channel, | | | | | | | |
| | | | | | | voltage- | | | | | | | |
| | | | | | | gated, type | | | | | | | |
| | | | | | | III, beta | | | | | | | |
| ENSG00000175538 | 11 | 73843536 | 73856186 | 1 | KCNE3 | potassium | x | Brugada | found in | | K+ | ion channel | 2 |
| | | | | | | voltage- | | Syndrome | discovery | | | | |
| | | | | | | gated | | | HF v ctrl; | | | | |
| | | | | | | channel, lsk- | | | hyperkalemic | | | | |
| | | | | | | related | | | periodic | | | | |
| | | | | | | family, | | | paralysis | | | | |
| | | | | | | member 3 | | | | | | | |
| ADD | | | | | GPD1L | glycerol-3- | x | Brugada, | site | | Na+ | | 2 |
| | | | | | | phosphate | | SIDS | homologous | | | | |
| | | | | | | dehydrogenase | | | to the | | | | |
| | | | | | | 1-Like | | | cardiac | | | | |
| | | | | | | | | | sodium | | | | |
| | | | | | | | | | channel | | | | |
| | | | | | | | | | SCN5A; | | | | |
| | | | | | | | | | Barry | | | | |
| | | | | | | | | | London | | | | |
| ENSG00000105711 | 19 | 40213374 | 40223192 | 1 | SCN1B | sodium | x | Brugadas | | | Na+ | ion channel | 2 |
| | | | | | | channel, | | and | | | | | |
| | | | | | | voltage- | | conduction | | | | | |
| | | | | | | gated, type I, | | defect | | | | | |
| | | | | | | beta | | | | | | | |
| ENSG00000069431 | 12 | 21845245 | 21985434 | 4 | ABCC9 | ATP-binding | x | DCM | found in | | K+ | receptor | |
| | | | | | | cassette, | | | discovery | | | | |
| | | | | | | sub-family C | | | HF v ctrl; | | | | |
| | | | | | | (CFTR/MRP), | | | assoc with | | | | |
| | | | | | | member 9 | | | K(ATP) | | | | |
| | | | | | | | | | channels | | | | |
| ENSG00000053918 | 11 | 2422797 | 2826915 | 4 | KCNQ1 | potassium | x | LQT1 | found in | | K+ | ion channel | 2 |
| | | | | | | voltage- | | | QTSCD | | | | |
| | | | | | | gated | | | and | | | | |
| | | | | | | channel, | | | QTGEN; | | | | |
| | | | | | | KQT-like | | | found in | | | | |
| | | | | | | subfamily, | | | discovery | | | | |
| | | | | | | member 1 | | | HF v ctrl | | | | |
| ENSG00000177098 | 11 | 117509302 | 1.18E+08 | 1 | SCN4B | sodium | x | LQT10 | | | Na+ | ion channel | 2 |
| | | | | | | channel, | | | | | | | |
| | | | | | | voltage- | | | | | | | |
| | | | | | | gated, type | | | | | | | |
| | | | | | | IV, beta | | | | | | | |
| ENSG00000055118 | 7 | 150272982 | 1.5E+08 | 3 | KCNH2 | potassium | x | LQT2 | found in | | K+ | ion channel | 2 |
| | | | | | | voltage- | | | QTSCD | | | | |
| | | | | | | gated | | | and | | | | |
| | | | | | | channel, | | | QTGEN | | | | |
| | | | | | | subfamily H | | | | | | | |
| | | | | | | (eag-related), | | | | | | | |
| | | | | | | member 2 | | | | | | | |
| ENSG00000183873 | 3 | 38564558 | 38666167 | 2 | SCN5A | sodium | x | LQT3, | found in | | Na+ | ion channel | 2 |
| | | | | | | channel, | | Brugadas | QTSCD | | | | |
| | | | | | | voltage- | | syndrome | and | | | | |
| | | | | | | gated, type | | | QTGEN | | | | |
| | | | | | | V, alpha | | | and assoc | | | | |
| | | | | | | (long QT | | | with SCA | | | | |
| | | | | | | syndrome 3) | | | risk (AHA | | | | |
| | | | | | | | | | abstract) | | | | |
| ENSG00000180509 | 21 | 34740858 | 34806443 | 1 | KCNE1 | potassium | x | LQT5 | found in | | K+ | ion channel | 2 |
| | | | | | | voltage- | | | QTGEN; | | | | |
| | | | | | | gated | | | found in | | | | |
| | | | | | | channel, lsk- | | | discovery | | | | |
| | | | | | | related | | | HF v ctrl | | | | |
| | | | | | | family, | | | | | | | |
| | | | | | | member 1 | | | | | | | |
| ENSG00000159197 | 21 | 34658193 | 34665307 | 1 | KCNE2 | potassium | x | LQT6 | | | K+ | ion channel | 2 |
| | | | | | | voltage- | | | | | | | |
| | | | | | | gated | | | | | | | |
| | | | | | | channel, lsk- | | | | | | | |
| | | | | | | related | | | | | | | |
| | | | | | | family, | | | | | | | |
| | | | | | | member 2 | | | | | | | |
| ENSG00000123700 | 17 | 65677271 | 65687755 | 1 | KCNJ2 | potassium | x | LQT7, CPVT | found in | | K+ | ion channel | 2 |
| | | | | | | inwardly- | | | QTSCD; | | | | |
| | | | | | | rectifying | | | found in | | | | |
| | | | | | | channel, | | | discovery | | | | |
| | | | | | | subfamily J, | | | HF v ctrl, | | | | |
| | | | | | | member 2 | | | and assoc | | | | |
| | | | | | | | | | with SCA | | | | |
| | | | | | | | | | risk (AHA | | | | |
| | | | | | | | | | abstract) | | | | |
| ENSG00000187486 | 11 | 17365042 | 17366214 | 1 | KCNJ11 | potassium | x | neonatal | | | K+ | ion channel | 2 |
| | | | | | | inwardly- | | diabetes, | | | | | |
| | | | | | | rectifying | | hyperinsuline | | | | | |
| | | | | | | channel, | | mic | | | | | |
| | | | | | | subfamily J, | | | | | | | |
| | | | | | | member 11 | | | | | | | |
| ENSG00000169432 | 2 | 166763060 | 1.67E+08 | 2 | SCN9A | sodium | x | pain | found in | neuroendocrine, | Na+ | ion channel | 2 |
| | | | | | | channel, | | syndromes, | discovery | smooth m | | | |
| | | | | | | voltage- | | seizure | HF v ctrl | | | | |
| | | | | | | gated, type | | disorders | | | | | |
| | | | | | | IX, alpha | | | | | | | |
| ADD | | | | | SCN10A | | x | PR interval, | new | | Na+ | ion channel | 2 |
| | | | | | | | | VF | findings | | | | |
| | | | | | | | | | AHA | | | | |
| ENSG00000138622 | 15 | 71400988 | 71448230 | 1 | HCN4 | hyperpolarization | x | SSS, | | | K+ | ion channel | 2 |
| | | | | | | activated | | Brugadas | | | | | |
| | | | | | | cyclic | | | | | | | |
| | | | | | | nucleotide- | | | | | | | |
| | | | | | | gated | | | | | | | |
| | | | | | | potassium | | | | | | | |
| | | | | | | channel 4 | | | | | | | |
| ADD | | | | | DPP6 | | x | VF (A. Wilde) | ncodes a | | K+ | | |
| | | | | | | | | | putative | | | | |
| | | | | | | | | | component | | | | |
| | | | | | | | | | of the | | | | |
| | | | | | | | | | transient | | | | |
| | | | | | | | | | outward | | | | |
| | | | | | | | | | current | | | | |
| ENSG00000164588 | 5 | 45297730 | 45731977 | 1 | HCN1 | hyperpolarization | | | found in | | K+ | ion channel | 2 |
| | | | | | | activated | | | discovery | | | | |
| | | | | | | cyclic | | | HF v ctrl | | | | |
| | | | | | | nucleotide- | | | | | | | |
| | | | | | | gated | | | | | | | |
| | | | | | | potassium | | | | | | | |
| | | | | | | channel 1 | | | | | | | |
| ENSG00000169282 | 3 | 157321095 | 1.58E+08 | 10 | KCNAB1 | potassium | | | found in | | K+ | ion channel | 2 |
| | | | | | | voltage- | | | discovery | | | | |
| | | | | | | gated | | | HF v ctrl | | | | |
| | | | | | | channel, | | | | | | | |
| | | | | | | shaker- | | | | | | | |
| | | | | | | related | | | | | | | |
| | | | | | | subfamily, | | | | | | | |
| | | | | | | beta member 1 | | | | | | | |
| ENSG00000069424 | 1 | 5974113 | 6083840 | 8 | KCNAB2 | potassium | | | found in | | K+ | ion channel | 2 |
| | | | | | | voltage- | | | discovery | | | | |
| | | | | | | gated | | | HF v ctrl | | | | |
| | | | | | | channel, | | | | | | | |
| | | | | | | shaker- | | | | | | | |
| | | | | | | related | | | | | | | |
| | | | | | | subfamily, | | | | | | | |
| | | | | | | beta member 2 | | | | | | | |
| ENSG00000120457 | 11 | 128266517 | 1.28E+08 | 1 | KCNJ5 | potassium | | | found in | | K+ | ion channel | 2 |
| | | | | | | inwardly- | | | discovery | | | | |
| | | | | | | rectifying | | | HF v ctrl | | | | |
| | | | | | | channel, | | | | | | | |
| | | | | | | subfamily J, | | | | | | | |
| | | | | | | member 5 | | | | | | | |
| ENSG00000135750 | 1 | 231816373 | 2.32E+08 | 3 | KCNK1 | potassium | | | found in | | K+ | ion channel | 2 |
| | | | | | | channel, | | | discovery | | | | |
| | | | | | | subfamily K, | | | HF v ctrl | | | | |
| | | | | | | member 1 | | | | | | | |
| ENSG00000182450 | 11 | 63815770 | 63828817 | 1 | KCNK4 | potassium | | | found in | | K+ | ion channel | 2 |
| | | | | | | channel, | | | discovery | | | | |
| | | | | | | subfamily K, | | | HF v ctrl | | | | |
| | | | | | | member 4 | | | | | | | |
| ENSG00000171385 | 1 | 112114807 | 1.12E+08 | 3 | KCND3 | potassium | | | found in | | K+ | ion channel | 2 |
| | | | | | | voltage- | | | discovery | | | | |
| | | | | | | gated | | | HF v ctrl; | | | | |
| | | | | | | channel, | | | repolarization | | | | |
| | | | | | | Shal-related | | | | | | | |
| | | | | | | subfamily, | | | | | | | |
| | | | | | | member 3 | | | | | | | |
| ENSG00000120049 | 10 | 103575721 | 1.04E+08 | 12 | KCNIP2 | Kv channel | | | ko mice | | K+ | ion channel | 2 |
| | | | | | | interacting | | | arrhythmias; | | | | |
| | | | | | | protein 2 | | | lto | | | | |
| ENSG00000184408 | 7 | 119701923 | 1.2E+08 | 1 | KCND2 | potassium | | | repolarization | | K+ | ion channel | 2 |
| | | | | | | voltage- | | | | | | | |
| | | | | | | gated | | | | | | | |
| | | | | | | channel, | | | | | | | |
| | | | | | | Shal-related | | | | | | | |
| | | | | | | subfamily, | | | | | | | |
| | | | | | | member 2 | | | | | | | |
| ENSG00000143105 | 1 | 110861396 | 1.11E+08 | 2 | KCNA10 | potassium | | | very little | | K+ | ion channel | 2 |
| | | | | | | voltage- | | | known | | | | |
| | | | | | | gated | | | | | | | |
| | | | | | | channel, | | | | | | | |
| | | | | | | shaker- | | | | | | | |
| | | | | | | related | | | | | | | |
| | | | | | | subfamily, | | | | | | | |
| | | | | | | member 10 | | | | | | | |
| ENSG00000074201 | 11 | 77004847 | 77026495 | 1 | CLNS1A | chloride | | | | | Cl− | ion channel | 2 |
| | | | | | | channel, | | | | | | | |
| | | | | | | nucleotide- | | | | | | | |
| | | | | | | sensitive, 1A | | | | | | | |
| ENSG00000099822 | 19 | 540893 | 568157 | 1 | HCN2 | hyperpolarization | | | | | K+ | ion channel | 2 |
| | | | | | | activated | | | | | | | |
| | | | | | | cyclic | | | | | | | |
| | | | | | | nucleotide- | | | | | | | |
| | | | | | | gated | | | | | | | |
| | | | | | | potassium | | | | | | | |
| | | | | | | channel 2 | | | | | | | |
| ENSG00000182255 | 11 | 29988341 | 29995064 | 1 | KCNA4 | potassium | | | | | K+ | ion channel | 2 |
| | | | | | | voltage- | | | | | | | |
| | | | | | | gated | | | | | | | |
| | | | | | | channel, | | | | | | | |
| | | | | | | shaker- | | | | | | | |
| | | | | | | related | | | | | | | |
| | | | | | | subfamily, | | | | | | | |
| | | | | | | member 4 | | | | | | | |
| ENSG00000151079 | 12 | 4789372 | 4791132 | 3 | KCNA6 | potassium | | | | | K+ | ion channel | 2 |
| | | | | | | voltage- | | | | | | | |
| | | | | | | gated | | | | | | | |
| | | | | | | channel, | | | | | | | |
| | | | | | | shaker- | | | | | | | |
| | | | | | | related | | | | | | | |
| | | | | | | subfamily, | | | | | | | |
| | | | | | | member 6 | | | | | | | |
| ENSG00000170049 | 17 | 7765902 | 7773478 | 2 | KCNAB3 | potassium | | | | | K+ | ion channel | 2 |
| | | | | | | voltage- | | | | | | | |
| | | | | | | gated | | | | | | | |
| | | | | | | channel, | | | | | | | |
| | | | | | | shaker- | | | | | | | |
| | | | | | | related | | | | | | | |
| | | | | | | subfamily, | | | | | | | |
| | | | | | | beta member 3 | | | | | | | |
| ENSG00000158445 | 20 | 47418353 | 47532591 | 1 | KCNB1 | potassium | | | | | K+ | ion channel | 2 |
| | | | | | | voltage- | | | | | | | |
| | | | | | | gated | | | | | | | |
| | | | | | | channel, | | | | | | | |
| | | | | | | Shab-related | | | | | | | |
| | | | | | | subfamily, | | | | | | | |
| | | | | | | member 1 | | | | | | | |
| ENSG00000176076 | X | 108753585 | 1.09E+08 | 2 | KCNE1L | KCNE1-like | | | | | K+ | ion channel | 2 |
| ENSG00000152049 | 2 | 223625171 | 2.24E+08 | 1 | KCNE4 | potassium | | | | | K+ | ion channel | 2 |
| | | | | | | voltage- | | | | | | | |
| | | | | | | gated | | | | | | | |
| | | | | | | channel, lsk- | | | | | | | |
| | | | | | | related | | | | | | | |
| | | | | | | family, | | | | | | | |
| | | | | | | member 4 | | | | | | | |
| ENSG00000184185 | 17 | 21220292 | 21260983 | 1 | KCNJ12 | potassium | | | | | K+ | ion channel | 2 |
| | | | | | | inwardly- | | | | | | | |
| | | | | | | rectifying | | | | | | | |
| | | | | | | channel, | | | | | | | |
| | | | | | | subfamily J, | | | | | | | |
| | | | | | | member 12 | | | | | | | |
| ENSG00000162989 | 2 | 155263339 | 1.55E+08 | 1 | KCNJ3 | potassium | | | | | K+ | ion channel | 2 |
| | | | | | | inwardly- | | | | | | | |
| | | | | | | rectifying | | | | | | | |
| | | | | | | channel, | | | | | | | |
| | | | | | | subfamily J, | | | | | | | |
| | | | | | | member 3 | | | | | | | |
| ENSG00000168135 | 22 | 37152278 | 37181149 | 1 | KCNJ4 | potassium | | | | | K+ | ion channel | 2 |
| | | | | | | inwardly- | | | | | | | |
| | | | | | | rectifying | | | | | | | |
| | | | | | | channel, | | | | | | | |
| | | | | | | subfamily J, | | | | | | | |
| | | | | | | member 4 | | | | | | | |
| ENSG00000121361 | 12 | 21809156 | 21819014 | 1 | KCNJ8 | potassium | | | | | K+ | ion channel | 2 |
| | | | | | | inwardly- | | | | | | | |
| | | | | | | rectifying | | | | | | | |
| | | | | | | channel, | | | | | | | |
| | | | | | | subfamily J, | | | | | | | |
| | | | | | | member 8 | | | | | | | |
| ENSG00000171303 | 2 | 26769123 | 26806207 | 1 | KCNK3 | potassium | | | | | K+ | ion channel | 2 |
| | | | | | | channel, | | | | | | | |
| | | | | | | subfamily K, | | | | | | | |
| | | | | | | member 3 | | | | | | | |
| ENSG00000099337 | 19 | 43502322 | 43511480 | 1 | KCNK6 | potassium | | | | | K+ | ion channel | 2 |
| | | | | | | channel, | | | | | | | |
| | | | | | | subfamily K, | | | | | | | |
| | | | | | | member 6 | | | | | | | |

TABLE 3

| | | | | | | | Mutated or | | | | | | |
|---|---|---|---|---|---|---|---|---|---|---|---|---|---|
| | | | | | | | associated | | | | | | |
| Ensembl | | Start | End | | | | with Human | | | | Ion (handling | structural | |
| Gene ID | Chromosome | Position | Position | Transcript | HGNC | Gene | SCD | Disease | Other | | or | or | EC |
| Ver 42 | Name | (bp) | (bp) | count | Symbol | Name | disorders | Groupings | LOE | Organelle | dependence) | function | coupling |
| ENSG00000163399 | 1 | 116717359 | 116754301 | 4 | ATP1A1 | ATPase, | | | role in | | | ATPase | |
| | | | | | | Na+/K+ | | | calcium | | | | |
| | | | | | | transporting, | | | signaling | | | | |
| | | | | | | alpha 1 | | | during | | | | |
| | | | | | | polypeptide | | | cardiac | | | | |
| | | | | | | | | | contraction | | | | |
| ENSG00000018625 | 1 | 158352172 | 158379996 | 2 | ATP1A2 | ATPase, | | | role in | | | ATPase | |
| | | | | | | Na+/K+ | | | calcium | | | | |
| | | | | | | transporting, | | | signaling | | | | |
| | | | | | | alpha 2 | | | during | | | | |
| | | | | | | (+) | | | cardiac | | | | |
| | | | | | | polypeptide | | | contraction | | | | |
| ENSG00000196296 | 16 | 28797310 | 28823331 | 1 | ATP2A1 | ATPase, | | | | SR | Ca++ | transmembrane | 1 |
| | | | | | | Ca++ | | | | | | protein | |
| | | | | | | transporting, | | | | | | | |
| | | | | | | cardiac | | | | | | | |
| | | | | | | muscle, | | | | | | | |
| | | | | | | fast | | | | | | | |
| | | | | | | twitch 1 | | | | | | | |
| ENSG00000174437 | 12 | 109203815 | 109273278 | 3 | ATP2A2 | ATPase, | | | | SR | Ca++ | transmembrane | 1 |
| | | | | | | Ca++ | | | | | | protein | |
| | | | | | | transporting, | | | | | | | |
| | | | | | | cardiac | | | | | | | |
| | | | | | | muscle, | | | | | | | |
| | | | | | | slow | | | | | | | |
| | | | | | | twitch 2 | | | | | | | |
| ENSG00000151067 | 12 | 2094650 | 2670626 | 5 | CACNA1C | calcium | x | LQT8 | found in | cell | Ca++ | | 1 |
| | | | | | | channel, | | | discovery | membrane | | | |
| | | | | | | voltage- | | | HF v | | | | |
| | | | | | | dependent, L | | | ctrl | | | | |
| | | | | | | type, | | | | | | | |
| | | | | | | alpha | | | | | | | |
| | | | | | | 1C | | | | | | | |
| | | | | | | subunit | | | | | | | |
| ENSG00000157388 | 3 | 53503723 | 53821112 | 2 | CACNA1D | calcium | | | found in | cell | Ca++ | | 1 |
| | | | | | | channel, | | | discovery | membrane | | | |
| | | | | | | voltage- | | | HF v | | | | |
| | | | | | | dependent, L | | | ctrl; | | | | |
| | | | | | | type, | | | Subunit | | | | |
| | | | | | | alpha | | | of L-type | | | | |
| | | | | | | 1D | | | calcium | | | | |
| | | | | | | subunit | | | channel | | | | |
| ENSG00000198216 | 1 | 179648918 | 180037339 | 6 | CACNA1E | calcium | | | | neuron, | Ca++ | | |
| | | | | | | channel, | | | | kidney, | | | |
| | | | | | | voltage- | | | | retina, | | | |
| | | | | | | dependent, | | | | spleen, | | | |
| | | | | | | alpha | | | | islet cells | | | |
| | | | | | | 1E | | | | | | | |
| | | | | | | subunit | | | | | | | |
| ENSG00000006283 | 17 | 45993820 | 46059541 | 6 | CACNA1G | calcium | | | found in | | Ca++ | | |
| | | | | | | channel, | | | discovery | | | | |
| | | | | | | voltage- | | | HF v | | | | |
| | | | | | | dependent, | | | ctrl; | | | | |
| | | | | | | alpha | | | subunit | | | | |
| | | | | | | 1G | | | of t-type | | | | |
| | | | | | | subunit | | | calcium | | | | |
| | | | | | | | | | channel, | | | | |
| | | | | | | | | | SA node | | | | |
| | | | | | | | | | cells | | | | |
| ENSG00000196557 | 16 | 1143739 | 1211772 | 2 | CACNA1H | calcium | | | | | Ca++ | | |
| | | | | | | channel, | | | | | | | |
| | | | | | | voltage- | | | | | | | |
| | | | | | | dependent, | | | | | | | |
| | | | | | | alpha | | | | | | | |
| | | | | | | 1H | | | | | | | |
| | | | | | | subunit | | | | | | | |
| ENSG00000153956 | 7 | 81417354 | 81910967 | 3 | CACNA2D1 | calcium | | | found in | | Ca++ | | 1 |
| | | | | | | channel, | | | discovery | | | | |
| | | | | | | voltage- | | | HF v | | | | |
| | | | | | | dependent, | | | ctrl; | | | | |
| | | | | | | alpha | | | Subunit | | | | |
| | | | | | | 2/delta | | | of L-type | | | | |
| | | | | | | subunit 1 | | | calcium | | | | |
| | | | | | | | | | channel | | | | |
| ENSG00000007402 | 3 | 50375237 | 50516032 | 2 | CACNA2D2 | calcium | | | found in | | Ca++ | | 1 |
| | | | | | | channel, | | | discovery | | | | |
| | | | | | | voltage- | | | HF v | | | | |
| | | | | | | dependent, | | | ctrl; | | | | |
| | | | | | | alpha | | | Subunit | | | | |
| | | | | | | 2/delta | | | of L-type | | | | |
| | | | | | | subunit 2 | | | calcium | | | | |
| | | | | | | | | | channel | | | | |
| ENSG00000157445 | 3 | 54131733 | 55083622 | 1 | CACNA2D3 | calcium | | | found in | cell | Ca++ | | 1 |
| | | | | | | channel, | | | discovery | membrane | | | |
| | | | | | | voltage- | | | HF v | | | | |
| | | | | | | dependent, | | | ctrl; | | | | |
| | | | | | | alpha | | | Subunit | | | | |
| | | | | | | 2/delta 3 | | | of L-type | | | | |
| | | | | | | subunit | | | calcium | | | | |
| | | | | | | | | | channel | | | | |
| ENSG00000151062 | 12 | 1771384 | 1898131 | 2 | CACNA2D4 | calcium | | | | | Ca++ | | |
| | | | | | | channel, | | | | | | | |
| | | | | | | voltage- | | | | | | | |
| | | | | | | dependent, | | | | | | | |
| | | | | | | alpha | | | | | | | |
| | | | | | | 2/delta | | | | | | | |
| | | | | | | subunit 4 | | | | | | | |
| ENSG00000067191 | 17 | 34583232 | 34607427 | 2 | CACNB1 | calcium | | | | | Ca++ | | |
| | | | | | | channel, | | | | | | | |
| | | | | | | voltage- | | | | | | | |
| | | | | | | dependent, | | | | | | | |
| | | | | | | beta 1 | | | | | | | |
| | | | | | | subunit | | | | | | | |
| ENSG00000165995 | 10 | 18469612 | 18870797 | 9 | CACNB2 | calcium | | | found in | | Ca++ | | 1 |
| | | | | | | channel, | | | discovery | | | | |
| | | | | | | voltage- | | | HF v | | | | |
| | | | | | | dependent, | | | ctrl; | | | | |
| | | | | | | beta 2 | | | Subunit | | | | |
| | | | | | | subunit | | | of L-type | | | | |
| | | | | | | | | | calcium | | | | |
| | | | | | | | | | channel | | | | |
| ENSG00000167535 | 12 | 47498779 | 47508991 | 1 | CACNB3 | calcium | | | found in | | Ca++ | | 1 |
| | | | | | | channel, | | | discovery | | | | |
| | | | | | | voltage- | | | HF v | | | | |
| | | | | | | dependent, | | | ctrl; | | | | |
| | | | | | | beta 3 | | | Subunit | | | | |
| | | | | | | subunit | | | of L-type | | | | |
| | | | | | | | | | calcium | | | | |
| | | | | | | | | | channel | | | | |
| ENSG00000182389 | 2 | S | 152663771 | 1 | CACNB4 | calcium | | | | | Ca++ | | |
| | | | | | | channel, | | | | | | | |
| | | | | | | voltage- | | | | | | | |
| | | | | | | dependent, | | | | | | | |
| | | | | | | beta 4 | | | | | | | |
| | | | | | | subunit | | | | | | | |
| ENSG00000198668 | 14 | 89933120 | 89944158 | | CALM1 | calmodulin 1 | | | | | | | |
| | | | | | | (phosphorylase | | | | | | | |
| | | | | | | kinase, | | | | | | | |
| | | | | | | delta) | | | | | | | |
| ENSG00000143933 | 2 | 47240736 | 47257140 | 1 | CALM2 | calmodulin 2 | | | | | | | |
| | | | | | | (phosphorylase | | | | | | | |
| | | | | | | kinase, | | | | | | | |
| | | | | | | delta) | | | | | | | |
| ENSG00000160014 | 19 | 51796352 | 51805878 | 1 | CALM3 | calmodulin 3 | | | | | | | |
| | | | | | | (phosphorylase | | | | | | | |
| | | | | | | kinase, | | | | | | | |
| | | | | | | delta) | | | | | | | |
| ENSG00000145349 | 4 | 114593022 | 114902177 | 4 | CAMK2D | calcium/ | | | | | Ca++ | phosphorylation, | 1 |
| | | | | | | calmodulin- | | | | | | KEY | |
| | | | | | | dependent | | | | | | | |
| | | | | | | protein | | | | | | | |
| | | | | | | kinase | | | | | | | |
| | | | | | | (CaM | | | | | | | |
| | | | | | | kinase) | | | | | | | |
| | | | | | | II delta | | | | | | | |
| ENSG00000108509 | 17 | 4812017 | 4831671 | 5 | CAMTA2 | calmodulin | | | | | | | |
| | | | | | | binding | | | | | | | |
| | | | | | | transcription | | | | | | | |
| | | | | | | activator 2 | | | | | | | |
| ENSG00000147044 | X | 41259131 | 41667660 | 8 | CASK | calcium/ | | | | | | | |
| | | | | | | calmodulin- | | | | | | | |
| | | | | | | dependent | | | | | | | |
| | | | | | | serine | | | | | | | |
| | | | | | | protein | | | | | | | |
| | | | | | | kinase | | | | | | | |
| | | | | | | (MAGUK | | | | | | | |
| | | | | | | family) | | | | | | | |
| ENSG00000118729 | 1 | 116044151 | 116112925 | 1 | CASQ2 | calsequestrin 2 | x | CPVT, | found in | SR | Ca++ | | 1 |
| | | | | | | (cardiac | | recessive | discovery | | | | |
| | | | | | | muscle) | | | HF v | | | | |
| | | | | | | | | | ctrl | | | | |
| ENSG00000119782 | 2 | 24126075 | 24140055 | 4 | FKBP1B | FK506 | | | assoc | SR | Ca++ | | 1 |
| | | | | | | binding | | | with RYR | | | | |
| | | | | | | protein | | | | | | | |
| | | | | | | 1B, | | | | | | | |
| | | | | | | 12.6 kDa | | | | | | | |
| ENSG00000172399 | 4 | 120276469 | 120328383 | 1 | MYOZ2 | myozenin 2 | | | Calsarcin | | | | |
| | | | | | | | | | 1; | | | | |
| | | | | | | | | | calcineurin- | | | | |
| | | | | | | | | | interacting | | | | |
| | | | | | | | | | protein | | | | |
| ENSG00000113448 | 5 | 58305622 | 59320301 | 5 | PDE4D | phosphodiesterase | | | found in | SR | Ca++ | | 1 |
| | | | | | | 4D, | | | discovery | | | | |
| | | | | | | cAMP- | | | HF v | | | | |
| | | | | | | specific | | | ctrl; | | | | |
| | | | | | | (phosphodiesterase | | | assoc | | | | |
| | | | | | | E3 | | | with RYR | | | | |
| | | | | | | dunce | | | | | | | |
| | | | | | | homolog, | | | | | | | |
| | | | | | | Drosophila) | | | | | | | |
| ENSG00000198523 | 6 | 118976154 | 118988586 | 1 | PLN | phospholamban | x | DCM | Found in | SR | Ca++ | | 1 |
| | | | | | | | | | QTGEN | | | | |
| | | | | | | | | | and | | | | |
| | | | | | | | | | QTSCD | | | | |
| ENSG00000138814 | 4 | 102163610 | 102487376 | 1 | PPP3CA | protein | | | found in | | | | |
| | | | | | | phosphatase 3 | | | discovery | | | | |
| | | | | | | (formerly | | | HF v | | | | |
| | | | | | | 2B), | | | ctrl | | | | |
| | | | | | | catalytic | | | | | | | |
| | | | | | | subunit, | | | | | | | |
| | | | | | | alpha | | | | | | | |
| | | | | | | isoform | | | | | | | |
| | | | | | | (calcineurin A | | | | | | | |
| | | | | | | alpha) | | | | | | | |
| ENSG00000114302 | 3 | 48762099 | 48860274 | 2 | PRKAR2A | protein | | | | | Ca++ | phosphorylation, | 1 |
| | | | | | | kinase, | | | | | | KEY | |
| | | | | | | cAMP- | | | | | | | |
| | | | | | | dependent, | | | | | | | |
| | | | | | | regulatory, | | | | | | | |
| | | | | | | type II, | | | | | | | |
| | | | | | | alpha | | | | | | | |
| ENSG00000154229 | 17 | 61729388 | 62237324 | 1 | PRKCA | protein | | | found in | | | | |
| | | | | | | kinase | | | discovery | | | | |
| | | | | | | C, | | | HF v | | | | |
| | | | | | | alpha | | | ctrl; | | | | |
| | | | | | | | | | fundamental | | | | |
| | | | | | | | | | regulator | | | | |
| | | | | | | | | | of | | | | |
| | | | | | | | | | cardiac | | | | |
| | | | | | | | | | contractility | | | | |
| | | | | | | | | | and | | | | |
| | | | | | | | | | Ca(2+) | | | | |
| | | | | | | | | | handling | | | | |
| | | | | | | | | | in | | | | |
| | | | | | | | | | myocytes | | | | |
| ENSG00000166501 | 16 | 23754823 | 24139358 | 2 | PRKCB1 | protein | | | found in | | | | |
| | | | | | | kinase | | | discovery | | | | |
| | | | | | | C, beta 1 | | | HF v | | | | |
| | | | | | | | | | ctrl | | | | |
| ENSG00000198626 | 1 | 235272128 | 236063911 | 3 | RYR2 | ryanodine | x | CPVT | found in | SR | Ca++ | | 1 |
| | | | | | | receptor 2 | | (exons 1-28, | discovery | | | | |
| | | | | | | (cardiac) | | 37-50, | HF v | | | | |
| | | | | | | | | 75, 83-105) | ctrl; | | | | |
| | | | | | | | | | assoc | | | | |
| | | | | | | | | | with | | | | |
| | | | | | | | | | lower | | | | |
| | | | | | | | | | SCA risk | | | | |
| | | | | | | | | | (AHA | | | | |
| | | | | | | | | | abstract) | | | | |
| ENSG00000136450 | 17 | 53437651 | 53439593 | 2 | SFRS1 | splicing | | | regulates | | | splicing | 1 |
| | | | | | | factor, | | | splicing | | | | |
| | | | | | | arginine/ | | | of | | | | |
| | | | | | | serine- | | | CAMK2D; | | | | |
| | | | | | | rich 1 | | | deficiency | | | | |
| | | | | | | (splicing | | | causes | | | | |
| | | | | | | factor | | | severe | | | | |
| | | | | | | 2, | | | EC | | | | |
| | | | | | | alternate | | | coupling | | | | |
| | | | | | | splicing | | | defects | | | | |
| | | | | | | factor) | | | | | | | |
| ENSG00000183023 | 2 | 40192790 | 40534188 | 5 | SLC8A1 | solute | | | | cell | Na+/Ca++ | membrane | 1 |
| | | | | | | carrier | | | | membrane | | ion | |
| | | | | | | family 8 | | | | | | exchanger | |
| | | | | | | (sodium/ | | | | | | | |
| | | | | | | calcium | | | | | | | |
| | | | | | | exchanger), | | | | | | | |
| | | | | | | member 1 | | | | | | | |
| ENSG00000118160 | 19 | 52623735 | 52666934 | 1 | SLC8A2 | solute | | | | cell | Na+/Ca++ | membrane | 1 |
| | | | | | | carrier | | | | membrane | | ion | |
| | | | | | | family 8 | | | | | | exchanger | |
| | | | | | | (sodium- | | | | | | | |
| | | | | | | calcium | | | | | | | |
| | | | | | | exchanger), | | | | | | | |
| | | | | | | member 2 | | | | | | | |
| ENSG00000170290 | 11 | 107083319 | 107087992 | 1 | SLN | sarcolipin | | | interact | SR | | | 1 |
| | | | | | | | | | with PLN | | | | |
| | | | | | | | | | and | | | | |
| | | | | | | | | | ATP2A1 | | | | |
| ENSG00000186439 | 6 | 123579183 | 123999937 | 5 | TRDN | triadin | | | found in | SR | | | 1 |
| | | | | | | | | | discovery | | | | |
| | | | | | | | | | HF v | | | | |
| | | | | | | | | | ctrl; | | | | |
| | | | | | | | | | colocalizes | | | | |
| | | | | | | | | | with | | | | |
| | | | | | | | | | the RYR | | | | |
| | | | | | | | | | and | | | | |
| | | | | | | | | | junctin; | | | | |
| | | | | | | | | | skel m | | | | |
| | | | | | | | | | and | | | | |
| | | | | | | | | | cardiac | | | | |
| | | | | | | | | | isoforms | | | | |

TABLE 4

| | | | | | | | Mutated or |
|---|---|---|---|---|---|---|---|
| Ensembl | | Start | End | | | | associated |
| Gene ID | Chromosome | Position | Position | Transcript | HGNC | Gene | with Human |
| Ver 42 | Name | (bp) | (bp) | count | Symbol | Name | SCD disorders |
| ENSG00000182533 | 3 | 8750253 | 8763451 | 2 | CAV3 | caveolin 3 | x |
| ENSG00000089250 | 12 | 116135362 | 116283965 | 3 | NOS1 | nitric oxide | |
| | | | | | | synthase 1 | |
| | | | | | | (neuronal) | |
| ENSG00000143153 | 1 | 167341559 | 167368584 | 3 | ATP1B1 | ATPase, | |
| | | | | | | Na+/K+ | |
| | | | | | | transporting, | |
| | | | | | | beta 1 | |
| | | | | | | polypeptide | |
| ADD | | | | | LITAF | | |
| ADD | | | | | GINS3 | | |
| ENSG00000198929 | 1 | 160306190 | 160604868 | 1 | NOS1AP | nitric oxide | |
| | | | | | | synthase 1 | |
| | | | | | | (neuronal) | |
| | | | | | | adaptor | |
| | | | | | | protein | |
| ADD | | | | | 9p21 | | |
| | | | | | markers | | |
| | | | | | 4p25 | | |
| | | | | | markers | | |

| Ensembl | | | | Ion (handling | |
|---|---|---|---|---|---|
| Gene ID | Disease | Other | | or | |
| Ver 42 | Groupings | LOE | Organelle | dependence) | structural |
| ENSG00000182533 | LQT9, HCM, | assoc | caveolae | variants alter | |
| | SIDS | with | | late Na+ | |
| | | dystrophin, | | current | |
| | | LGMD | | | |
| ENSG00000089250 | | found in | | | |
| | | discovery | | | |
| | | HF v | | | |
| | | ctrl | | | |
| ENSG00000143153 | | found in | | Na+/K+ | ATPase |
| | | discovery | | | |
| | | HF v | | | |
| | | ctrl; | | | |
| | | found in | | | |
| | | QTSCD | | | |
| ADD | | found in | | | |
| | | QTGEN | | | |
| | | and | | | |
| | | QTSCD | | | |
| ADD | | found in | | | |
| | | QTGEN | | | |
| | | and | | | |
| | | QTSCD; | | | |
| | | Roden | | | |
| | | zfish | | | |
| ENSG00000198929 | | QTSCD, | | | |
| | | QTGEN, | | | |
| | | SCD, | | | |
| | | found in | | | |
| | | discovery | | | |
| | | HF v | | | |
| | | ctrl | | | |
| ADD | | | | | |

TABLE 5

| Ensembl | | Start | End | | | |
|---|---|---|---|---|---|---|
| Gene ID | Chromosome | Position | Position | Transcript | HGNC | |
| Ver 42 | Name | (bp) | (bp) | count | Symbol | Gene Name |
| | 17 | 37164412 | 37196476 | 1 | JUP | junction |
| | | | | | | plakoglobin |
| ENSG00000134755 | 18 | 26900005 | 26936375 | 2 | DSC2 | desmocollin 3 |
| | | | | | DSG2 | desmoglein |
| ENSG00000096696 | 6 | 7486869 | 7531945 | 1 | DSP | desmoplakin |
| ENSG00000057294 | 12 | 32834954 | 32941041 | 2 | PKP2 | plakophilin 2 |
| ENSG00000152661 | 6 | 121798487 | 121812571 | 1 | GJA1 | gap junction |
| | | | | | | protein, |
| | | | | | | alpha 1, |
| | | | | | | 43 kDa |
| | | | | | | (connexin |
| | | | | | | 43) |
| ENSG00000143140 | 1 | 145695517 | 145712066 | 2 | GJA5 | gap junction |
| | | | | | | protein, |
| | | | | | | alpha 5, |
| | | | | | | 40 kDa |
| | | | | | | (connexin |
| | | | | | | 40) |
| ENSG00000182963 | 17 | 40237146 | 40263707 | 1 | GJA7 | gap junction |
| | | | | | | protein, |
| | | | | | | alpha 7, |
| | | | | | | 45 kDa |
| | | | | | | (connexin |
| | | | | | | 45) |
| ENSG00000169562 | X | 70351769 | 70362091 | 3 | GJB1 | gap junction |
| | | | | | | protein, beta |
| | | | | | | 1, 32 kDa |
| | | | | | | (connexin |
| | | | | | | 32, Charcot- |
| | | | | | | Marie-Tooth |
| | | | | | | neuropathy, |
| | | | | | | X-linked) |
| ENSG00000149596 | 20 | 42173749 | 42249632 | 2 | JPH2 | junctophilin 2 |

| | Mutated or | | | | Ion | |
|---|---|---|---|---|---|---|
| Ensembl | associated | | | | (handling | |
| Gene ID | with Human | Disease | Other | | or | |
| Ver 42 | SCD disorders | Groupings | LOE | Organelle | dependence) | structural |
| | x | ARVC | found in | | | desmosomes |
| | | | discovery | | | |
| | | | HF v | | | |
| | | | ctrl; | | | |
| | | | adhering | | | |
| | | | junctions, | | | |
| | | | the | | | |
| | | | desmosomes | | | |
| | | | and the | | | |
| | | | intermediate | | | |
| | | | junctions | | | |
| ENSG00000134755 | x | ARVC | | | | desmosomes |
| | x | ARVC | | | | desmosomes |
| ENSG00000096696 | x | ARVC | | | | desmosomes |
| ENSG00000057294 | x | ARVC | | | | desmosomes |
| ENSG00000152661 | | | | | | gap junction |
| ENSG00000143140 | | | | | | gap junction |
| ENSG00000182963 | | | | | | gap junction |
| ENSG00000169562 | | | | | | gap junction |
| ENSG00000149596 | | | | | | junctional |
| | | | | | | complex |

TABLE 6

| Ensembl | | | | | | | Mutated or | | | | Ion (handling | structural |
|---|---|---|---|---|---|---|---|---|---|---|---|---|
| Gene ID | Chromosome | Start Position | End Position | Transcript | HGNC | Gene | associated with Human | Disease | Other | | or | or |
| Ver 42 | Name | (bp) | (bp) | count | Symbol | Name | SCD disorders | Groupings | LOE | Organelle | dependence) | function |
| ENSG00000163485 | 1 | 201326405 | 201403156 | 4 | ADORA1 | adenosine | | | activates | | | GPCR |
| | | | | | | A1 | | | adenosine | | | |
| | | | | | | receptor | | | receptors; | | | |
| | | | | | | | | | contractility | | | |
| ENSG00000128271 | 22 | 23153537 | 23168309 | 2 | ADORA2A | adenosine | | | activates | | | GPCR |
| | | | | | | A2a | | | adenosine | | | |
| | | | | | | receptor | | | receptors; | | | |
| | | | | | | | | | contractility | | | |
| ENSG00000170425 | 17 | 15788956 | 15819935 | 1 | ADORA2B | adenosine | | | activates | | | GPCR |
| | | | | | | A2b | | | adenosine | | | |
| | | | | | | receptor | | | receptors; | | | |
| | | | | | | | | | contractility | | | |
| ENSG00000121933 | 1 | 111827493 | 111908107 | 6 | ADORA3 | adenosine | | | activates | | | GPCR |
| | | | | | | A3 | | | adenosine | | | |
| | | | | | | receptor | | | receptors; | | | |
| | | | | | | | | | contractility | | | |
| ENSG00000120907 | 8 | 26661584 | 26778839 | 12 | ADRA1A | adrenergic, | | | found in | symp NS | Epi/NE | GPCR |
| | | | | | | alpha-1A-, | | | discovery | | | |
| | | | | | | receptor | | | HF v ctrl | | | |
| ENSG00000170214 | 5 | 159276318 | 159332595 | 1 | ADRA1B | adrenergic, | | | | symp NS | Epi/NE | GPCR |
| | | | | | | alpha-1B-, | | | | | | |
| | | | | | | receptor | | | | | | |
| ENSG00000171873 | 20 | 4149329 | 4177659 | 1 | ADRA1D | adrenergic, | | | | symp NS | Epi/NE | GPCR |
| | | | | | | alpha-1D-, | | | | | | |
| | | | | | | receptor | | | | | | |
| ENSG00000150594 | 10 | 112826911 | 112830655 | 2 | ADRA2A | adrenergic, | | | | symp NS | Epi/NE | GPCR |
| | | | | | | alpha-2A-, | | | | | | |
| | | | | | | receptor | | | | | | |
| ENSG00000181210 | 2 | 96202419 | 96203762 | | ADRA2B | adrenergic, | | | | symp NS | Epi/NE | GPCR |
| | | | | | | alpha-2B-, | | | | | | |
| | | | | | | receptor | | | | | | |
| ENSG00000133019 | 1 | 237859012 | 238145373 | 2 | CHRM3 | cholinergic | | | Cardiac?? | | Ach | signaling, |
| | | | | | | receptor, | | | | | | parasymp |
| | | | | | | muscarinic 3 | | | | | | |
| ENSG00000103546 | 16 | 54248057 | 54296685 | 3 | SLC6A2 | solute | | | | | | Norepi |
| | | | | | | carrier | | | | | | transporter |
| | | | | | | family 6 | | | | | | |
| | | | | | | (neurotransmitter | | | | | | |
| | | | | | | transporter, | | | | | | |
| | | | | | | noradrenalin), | | | | | | |
| | | | | | | member 2 | | | | | | |

TABLE 7

| | | | | | | | Mutated or | | | | | | | | |
|---|---|---|---|---|---|---|---|---|---|---|---|---|---|---|---|
| | | | | | | | associated | | | | Ion | | | | |
| Ensembl | | Start | End | Tran- | | | with Human | | | | (handling | structural | | | tran- |
| Gene ID | Chromosome | Position | Position | script | HGNC | | SCD | Disease | Other | | or | or | EC | ion | scription |
| Ver 42 | Name | (bp) | (bp) | count | Symbol | Gene Name | disorders | Groupings | LOE | Organelle | dependence) | function | coupling | channels | factors |
| ENSG00000068305 | 15 | 97923712 | 98074131 | 3 | MEF2A | MADS box | x | CAD, MI | found in | nucleus | | | | | 4 |
| | | | | | | transcription | | | discovery | | | | | | |
| | | | | | | enhancer | | | HF v | | | | | | |
| | | | | | | factor 2, | | | ctrl: | | | | | | |
| | | | | | | polypeptide A | | | Topol | | | | | | |
| | | | | | | (myocyte | | | gene | | | | | | |
| | | | | | | enhancer | | | | | | | | | |
| | | | | | | factor 2A) | | | | | | | | | |
| ENSG00000129170 | 11 | 19160154 | 19180177 | 1 | CSRP3 | cysteine and | x | DCM, HCM | involved | | | | | | |
| | | | | | | glycine-rich | | | in | | | | | | |
| | | | | | | protein 3 | | | myogenesis | | | | | | |
| | | | | | | (cardiac LIM | | | | | | | | | |
| | | | | | | protein) | | | | | | | | | |
| ADD | | | | | PITX2 | | x | AF | | | | | | | |
| ENSG00000183072 | 5 | 172591744 | 172594868 | 1 | NKX2-5 | NK2 | x | ASD, | | nucleus | | | | | 4 |
| | | | | | | transcription | | conduction | | | | | | | |
| | | | | | | factor related, | | defect, and | | | | | | | |
| | | | | | | locus 5 | | other CHD | | | | | | | |
| | | | | | | (Drosophila) | | | | | | | | | |
| ENSG00000089225 | 12 | 113276119 | 113330630 | 3 | TBX5 | T-box 5 | x | ASD | | nucleus | | | | | 4 |
| ENSG00000105866 | 7 | 21434214 | 21520674 | 1 | SP4 | Sp4 | | | mouse | nucleus | | | | | 4 |
| | | | | | | transcription | | | model | | | | | | |
| | | | | | | factor | | | SCD/VF | | | | | | |
| ENSG00000180733 | 8 | 48812794 | 48813235 | 1 | CEBPD | CCAAT/ | | | | | | | | | |
| | | | | | | enhancer | | | | | | | | | |
| | | | | | | binding | | | | | | | | | |
| | | | | | | protein | | | | | | | | | |
| | | | | | | (C/EBP), | | | | | | | | | |
| | | | | | | delta | | | | | | | | | |
| ENSG00000136574 | 8 | 11599122 | 11654920 | 3 | GATA4 | GATA | | | | nucleus | | | | | 4 |
| | | | | | | binding | | | | | | | | | |
| | | | | | | protein 4 | | | | | | | | | |
| ENSG00000108840 | 17 | 39509647 | 39556540 | 2 | HDAC5 | histone | | | | nucleus | | | | | 4 |
| | | | | | | deacetylase 5 | | | | | | | | | |
| ENSG00000081189 | 5 | 88051922 | 88214818 | 2 | MEF2C | MADS box | | | | nucleus | | | | | 4 |
| | | | | | | transcription | | | | | | | | | |
| | | | | | | enhancer | | | | | | | | | |
| | | | | | | factor 2, | | | | | | | | | |
| | | | | | | polypeptide C | | | | | | | | | |
| | | | | | | (myocyte | | | | | | | | | |
| | | | | | | enhancer | | | | | | | | | |
| | | | | | | factor 2C) | | | | | | | | | |
| ENSG00000101096 | 20 | 49441083 | 49592665 | 2 | NFATC2 | nuclear factor | | | | nucleus | | | | | 4 |
| | | | | | | of activated | | | | | | | | | |
| | | | | | | T-cells, | | | | | | | | | |
| | | | | | | cytoplasmic, | | | | | | | | | |
| | | | | | | calcineurin- | | | | | | | | | |
| | | | | | | dependent 2 | | | | | | | | | |
| ENSG00000171786 | 1 | 158603481 | 158609262 | 1 | NHLH1 | nescient helix | | | | nucleus | | | | | 4 |
| | | | | | | loop helix 1 | | | | | | | | | |
| ENSG00000108064 | 10 | 59814788 | 59828987 | 2 | TFAM | transcription | | | | | | | | | |
| | | | | | | factor A, | | | | | | | | | |
| | | | | | | mitochondrial | | | | | | | | | |

TABLE 8

| | | | | | | | Mutated or | | | | | |
|---|---|---|---|---|---|---|---|---|---|---|---|---|
| Ensembl | | | | | | | associated | | | | Ion | |
| Gene ID | Chromosome | Start Position | End Position | Transcript | HGNC | | with Human SCD | Disease | | | (handling or | structural or |
| Ver 42 | Name | (bp) | (bp) | count | Symbol | Gene Name | disorders | Groupings | Other LOE | Organelle | dependence) | function |
| ENSG00000145362 | 4 | 114190319 | 114524334 | 4 | ANK2 | ankyrin 2, | x | LQT4 | assoc with | | | peripheral |
| | | | | | | neuronal | | | lower SCA | | | membrane |
| | | | | | | | | | risk (AHA | | | |
| | | | | | | | | | abstract) | | | |
| ADD | | | | | LDB3 | | x | DCM, non- | Cypher/ZASP, | | | |
| | | | | | | | | compaction | cytoskeletal | | | |
| | | | | | | | | | assembly; | | | |
| | | | | | | | | | interacts | | | |
| | | | | | | | | | with MYOZ | | | |
| ENSG00000168028 | 3 | 39423208 | 39429034 | 1 | RPSA | ribosomal | x | ARVC | Laminin | | | cytoskeletal |
| | | | | | | protein SA | | | receptor | | | |
| | | | | | | (LAMR1) | | | | | | |
| ENSG00000198947 | X | 31047257 | 33267479 | 15 | DMD | dystrophin | x | DCM, | | | | cytoskeletal |
| | | | | | | (muscular- | | muscular | | | | |
| | | | | | | dystrophy, | | dystrophy | | | | |
| | | | | | | Duchenne and | | | | | | |
| | | | | | | Becker types) | | | | | | |
| ENSG00000160789 | 1 | 154318993 | 154376504 | 9 | LMNA | lamin A/C | x | DCM | | | | cytoskeletal |
| ENSG00000101400 | 20 | 31459424 | 31495359 | 1 | SNTA1 | syntrophin, | x | LQT12 | | | | cytoskeletal |
| | | | | | | alpha 1 | | | | | | |
| | | | | | | (dystrophin- | | | | | | |
| | | | | | | associated | | | | | | |
| | | | | | | protein A1, | | | | | | |
| | | | | | | 59 kDa, acidic | | | | | | |
| | | | | | | component) | | | | | | |
| ENSG00000155657 | 2 | 179099985 | 179380394 | 12 | TTN | titin | x | HCM, DCM, | | | | sarcomere |
| | | | | | | | | muscular | | | | |
| | | | | | | | | dystrophy | | | | |
| ENSG00000148677 | 10 | 92661833 | 92671013 | 1 | ANKRD1 | ankyrin repeat | | | CARP, | | | sarcomere |
| | | | | | | domain 1 | | | colocalized | | | |
| | | | | | | (cardiac | | | with titin | | | |
| | | | | | | muscle) | | | | | | |
| ENSG00000115414 | 2 | 215933409 | 216009041 | 10 | FN1 | fibronectin 1 | | | found in | | | ECM, |
| | | | | | | | | | discovery | | | connective |
| | | | | | | | | | HF v ctrl | | | |
| ENSG00000170624 | 5 | 155686334 | 156125623 | 1 | SGCD | sarcoglycan, | | | found in | | | cytoskeletal |
| | | | | | | delta (35 kDa | | | discovery | | | |
| | | | | | | dystrophin- | | | HF v ctrl | | | |
| | | | | | | associated | | | | | | |
| | | | | | | glycoprotein) | | | | | | |
| ENSG00000151150 | 10 | 61458165 | 61819494 | 6 | ANK3 | ankyrin 3, | | | found in | | | peripheral |
| | | | | | | node of | | | discovery | | | membrane |
| | | | | | | Ranvier | | | HF v ctrl; | | | |
| | | | | | | (ankyrin G) | | | associates | | | |
| | | | | | | | | | with SCN5A | | | |
| ENSG00000134769 | 18 | 30327279 | 30725341 | 6 | DTNA | dystrobrevin, | | | found in | | | cytoskeletal |
| | | | | | | alpha | | | discovery | | | |
| | | | | | | | | | HF v ctrl; | | | |
| | | | | | | | | | component | | | |
| | | | | | | | | | of the | | | |
| | | | | | | | | | dystrophin- | | | |
| | | | | | | | | | associated | | | |
| | | | | | | | | | protein | | | |
| | | | | | | | | | complex | | | |
| | | | | | | | | | (DPC) | | | |
| ENSG00000137076 | 9 | 35687336 | 35722369 | 6 | TLN1 | talin 1 | | | found in | | | cytoskeletal |
| | | | | | | | | | discovery | | | |
| | | | | | | | | | HF v ctrl; | | | |
| | | | | | | | | | links | | | |
| | | | | | | | | | vinculin to | | | |
| | | | | | | | | | the integrins, | | | |
| | | | | | | | | | and, thus, | | | |
| | | | | | | | | | the cytoskeleton | | | |
| | | | | | | | | | to extracellular | | | |
| | | | | | | | | | matrix (ECM) | | | |
| | | | | | | | | | receptors | | | |
| ENSG00000154358 | 1 | 226462454 | 226633198 | 9 | OBSCN | obscurin, | | | obscurin | | | sarcomere |
| | | | | | | cytoskeletal | | | and titin | | | |
| | | | | | | calmodulin and | | | coassemble | | | |
| | | | | | | titin-interacting | | | during | | | |
| | | | | | | RhoGEF | | | myofibrillogenesis | | | |
| ENSG00000175084 | 2 | 219991343 | 219999705 | 2 | DES | desmin | | | | | | cytoskeletal |
| ENSG00000172164 | 8 | 121619297 | 121893264 | 1 | SNTB1 | syntrophin, | | | | | | cytoskeletal |
| | | | | | | beta 1 | | | | | | |
| | | | | | | (dystrophin- | | | | | | |
| | | | | | | associated | | | | | | |
| | | | | | | protein A1, | | | | | | |
| | | | | | | 59 kDa, basic | | | | | | |
| | | | | | | component 1) | | | | | | |
| ENSG00000168807 | 16 | 67778533 | 67892379 | 2 | SNTB2 | syntrophin, | | | | | | cytoskeletal |
| | | | | | | beta 2 | | | | | | |
| | | | | | | (dystrophin- | | | | | | |
| | | | | | | associated | | | | | | |
| | | | | | | protein A1, | | | | | | |
| | | | | | | 59 kDa, basic | | | | | | |
| | | | | | | component 2) | | | | | | |
| ENSG00000173991 | 17 | 35073966 | 35076326 | 1 | TCAP | titin-cap | | | | | | sarcomere |
| | | | | | | (telethonin) | | | | | | |
| ENSG00000035403 | 10 | 75427878 | 75549924 | 2 | VCL | vinculin | | | | | | cytoskeletal |

TABLE 9

| | | Start | End | | | |
|---|---|---|---|---|---|---|
| Ensembl Gene ID | Chromosome | Position | Position | Transcript | HGNC | |
| Ver 42 | Name | (bp) | (bp) | count | Symbol | Gene Name |
| ENSG00000135744 | 1 | 228904892 | 228916666 | 1 | AGT | angiotensinogen |
| | | | | | | (serpin |
| | | | | | | peptidase |
| | | | | | | inhibitor, clade |
| | | | | | | A, member 8) |
| ENSG00000151623 | 4 | 149219370 | 149582973 | 4 | NR3C2 | nuclear receptor |
| | | | | | | subfamily 3, |
| | | | | | | group C, |
| | | | | | | member 2 |
| ENSG00000092009 | 14 | 24044552 | 24047311 | 2 | CMA1 | chymase 1, |
| | | | | | | mast cell |
| ENSG00000159640 | 17 | 58908166 | 58938721 | 2 | ACE | angiotensin I |
| | | | | | | converting |
| | | | | | | enzyme |
| | | | | | | (peptidyl- |
| | | | | | | dipeptidase A) 1 |
| ENSG00000144891 | 3 | 149898355 | 149943478 | 1 | AGTR1 | angiotensin II |
| | | | | | | receptor, type 1 |

| | Mutated or | | | | |
|---|---|---|---|---|---|
| | associated with | | | | |
| Ensembl Gene ID | Human SCD | Disease | Other | | structural or |
| Ver 42 | disorders | Groupings | LOE | Organelle | function |
| ENSG00000135744 | | CAD, AF, | found in | | neurohormonal |
| | | HTN | discovery | | |
| | | | HF v ctrl | | |
| ENSG00000151623 | | | found in | | aldosterone |
| | | | discovery | | receptor |
| | | | HF v ctrl | | |
| ENSG00000092009 | | | works | | neurohormonal |
| | | | like ACE | | |
| | | | in heart | | |
| ENSG00000159640 | | | | | neurohormonal |
| ENSG00000144891 | | | | | neurohormonal |

TABLE 10

| | | | | | | | Mutated or | | | | | |
|---|---|---|---|---|---|---|---|---|---|---|---|---|
| | | | | | | | associated | | | | | |
| Ensembl | | Start | End | | | | with Human | | | | Ion | |
| Gene ID Ver | Chromosome | Position | Position | Transcript | HGNC | | SCD | Disease | | | (handling or | structural or |
| 42 | Name | (bp) | (bp) | count | Symbol | Gene Name | disorders | Groupings | Other LOE | Organelle | dependence) | function |
| ENSG00000106617 | 7 | 150884960 | 151204728 | 1 | PRKAG2 | protein kinase, | x | HCM | found in | | | |
| | | | | | | AMP-activated, | | | discovery HF v | | | |
| | | | | | | gamma 2 non- | | | ctrl; metabolic | | | |
| | | | | | | catalytic | | | stress-sensing | | | |
| | | | | | | subunit | | | protein kinase; | | | |
| | | | | | | | | | critical role in | | | |
| | | | | | | | | | regulating | | | |
| | | | | | | | | | cellular | | | |
| | | | | | | | | | glucose and | | | |
| | | | | | | | | | fatty acid | | | |
| | | | | | | | | | metabolic | | | |
| | | | | | | | | | pathways | | | |
| ENSG00000074582 | 2 | 219231772 | 219236399 | 1 | BCS1L | BCS1-like | x | mitochondrial | | mitochondria | | |
| | | | | | | (yeast) | | complex III | | | | |
| | | | | | | | | deficiency | | | | |
| ENSG00000014919 | 10 | 101461591 | 101482413 | 2 | COX15 | COX15 | x | infantile HCM | | | | |
| | | | | | | homolog, | | | | | | |
| | | | | | | cytochrome c | | | | | | |
| | | | | | | oxidase | | | | | | |
| | | | | | | assembly | | | | | | |
| | | | | | | protein (yeast) | | | | | | |
| ENSG00000110536 | 11 | 47543464 | 47562690 | 3 | NDUFS3 | NADH | x | Leigh | | mitochondria | | |
| | | | | | | dehydrogenase | | syndrome | | | | |
| | | | | | | (ubiquinone) | | | | | | |
| | | | | | | Fe—S protein 3, | | | | | | |
| | | | | | | 30 kDa (NADH- | | | | | | |
| | | | | | | coenzyme Q | | | | | | |
| | | | | | | reductase) | | | | | | |
| ENSG00000073578 | 5 | 271356 | 309815 | 3 | SDHA | succinate | x | Leigh | | mitochondria | | |
| | | | | | | dehydrogenase | | syndrome | | | | |
| | | | | | | complex, | | | | | | |
| | | | | | | subunit A, | | | | | | |
| | | | | | | flavoprotein | | | | | | |
| | | | | | | (Fp) | | | | | | |
| ENSG00000148290 | 9 | 135208431 | 135213182 | 1 | SURF1 | surfeit 1 | x | Leigh | | mitochondria | | assembly |
| | | | | | | | | syndrome | | | | factor for COX |
| ENSG00000164258 | 5 | 52892226 | 53014925 | 2 | NDUFS4 | NADH | | | found in | mitochondria | | |
| | | | | | | dehydrogenase | | | discovery HF v | | | |
| | | | | | | (ubiquinone) | | | ctrl | | | |
| | | | | | | Fe—S protein 4, | | | | | | |
| | | | | | | 18 kDa (NADH- | | | | | | |
| | | | | | | coenzyme Q | | | | | | |
| | | | | | | reductase) | | | | | | |
| ENSG00000006695 | 17 | 13913444 | 14052712 | 1 | COX10 | COX10 | | | found in | mitochondria | | |
| | | | | | | homolog, | | | discovery HF v | | | |
| | | | | | | cytochrome c | | | ctrl; rs2230355 | | | |
| | | | | | | oxidase | | | | | | |
| | | | | | | assembly | | | | | | |
| | | | | | | protein, heme | | | | | | |
| | | | | | | A: | | | | | | |
| | | | | | | farnesyltransferase | | | | | | |
| | | | | | | (yeast) | | | | | | |
| ENSG00000179142 | 8 | 143988983 | 143996261 | 1 | CYP11B2 | cytochrome | | | | mitochondria | | |
| | | | | | | P450, family | | | | | | |
| | | | | | | 11, subfamily | | | | | | |
| | | | | | | B, polypeptide 2 | | | | | | |
| ENSG00000091140 | 7 | 107318847 | 107347645 | 1 | DLD | dihydrolipoamide | | | | | | |
| | | | | | | dehydrogenase | | | | | | |
| | | | | | | (E3 | | | | | | |
| | | | | | | component of | | | | | | |
| | | | | | | pyruvate | | | | | | |
| | | | | | | dehydrogenase | | | | | | |
| | | | | | | complex, 2- | | | | | | |
| | | | | | | oxo-glutarate | | | | | | |
| | | | | | | complex, | | | | | | |
| | | | | | | branched | | | | | | |
| | | | | | | chain keto acid | | | | | | |
| | | | | | | dehydrogenase | | | | | | |
| | | | | | | complex) | | | | | | |
| ENSG00000115286 | 19 | 1334883 | 1346583 | 3 | NDUFS7 | NADH | | | | mitochondria | | |
| | | | | | | dehydrogenase | | | | | | |
| | | | | | | (ubiquinone) | | | | | | |
| | | | | | | Fe—S protein 7, | | | | | | |
| | | | | | | 20 kDa (NADH- | | | | | | |
| | | | | | | coenzyme Q | | | | | | |
| | | | | | | reductase) | | | | | | |
| ENSG00000110717 | 11 | 67554670 | 67560686 | 1 | NDUFS8 | NADH | | | | mitochondria | | |
| | | | | | | dehydrogenase | | | | | | |
| | | | | | | (ubiquinone) | | | | | | |
| | | | | | | Fe—S protein 8, | | | | | | |
| | | | | | | 23 kDa (NADH- | | | | | | |
| | | | | | | coenzyme Q | | | | | | |
| | | | | | | reductase) | | | | | | |
| ENSG00000167792 | 11 | 67130974 | 67136581 | 1 | NDUFV1 | NADH | | | | mitochondria | | |
| | | | | | | dehydrogenase | | | | | | |
| | | | | | | (ubiquinone) | | | | | | |
| | | | | | | flavoprotein 1, | | | | | | |
| | | | | | | 51 kDa | | | | | | |
| ENSG00000131828 | X | 19271968 | 19289724 | 5 | PDHA1 | pyruvate | | | | mitochondria | | multienzyme |
| | | | | | | dehydrogenase | | | | | | |
| | | | | | | (lipoamide) | | | | | | |
| | | | | | | alpha 1 | | | | | | |
| ENSG00000151729 | 4 | 186301392 | 186305418 | 1 | SLC25A4 | solute carrier | | | | mitochondria | | |
| | | | | | | family 25 | | | | | | |
| | | | | | | (mitochondrial | | | | | | |
| | | | | | | carrier; | | | | | | |
| | | | | | | adenine | | | | | | |
| | | | | | | nucleotide | | | | | | |
| | | | | | | translocator), | | | | | | |
| | | | | | | member 4 | | | | | | |
| ENSG00000112096 | 6 | 160020138 | 160034343 | 3 | SOD2 | superoxide | | | | mitochondria | | |
| | | | | | | dismutase 2, | | | | | | |
| | | | | | | mitochondrial | | | | | | |
| ENSG00000073905 | 5 | 133335506 | 133368723 | 1 | VDAC1 | voltage- | | | | mitochondria | | |
| | | | | | | dependent | | | | | | |
| | | | | | | anion channel 1 | | | | | | |

TABLE 11

| | current | | |
|---|---|---|---|
| Gene Symbol | set | notes | |
| ADCY1 | | brain, CNS | adenylate cyclase |
| ADCY2 | | | adenylate cyclase |
| ADCY3 | | | adenylate cyclase |
| ADCY4 | | | adenylate cyclase |
| ADCY5 | | | adenylate cyclase |
| ADCY6 | | | adenylate cyclase |
| ADCY7 | | | adenylate cyclase |
| ADCY8 | | | adenylate cyclase |
| ADCY9 | | | adenylate cyclase |
| ADRA1A | 6 | | |
| ADRA1B | 6 | | |
| ADRA1D | 6 | | |
| ADRB1 | 1 | | |
| ADRB2 | 1 | | |
| ADRB3 | 1 | | |
| ANXA6 | | | annexin |
| ARRB1 | | | arrestin |
| ARRB2 | | | arrestin |
| ATP1A1 | 3 | | |
| ATP1A2 | 3 | | |
| ATP1A4 | | | Na/K ATPase |
| ATP1B1 | | | Na/K ATPase |
| ATP1B2 | | | Na/K ATPase |
| ATP1B3 | | | Na/K ATPase |
| ATP2A1 | 1 | | |
| ATP2A2 | 1, 3 | | |
| ATP2A3 | | | |
| ATP2B1 | | | |
| ATP2B2 | | | |
| ATP2B3 | | | |
| CACNA1A | 1, 3 | | |
| CACNA1B | | | |
| CACNA1C | 1, 3 | | |
| CACNA1D | 1, 3 | | |
| CACNA1E | | | |
| CACNA1S | | | |
| CACNB1 | 3 | | |
| CACNB2 | 3 | | |
| CACNB3 | 3 | | |
| CACNB4 | 3 | | |
| CALM1 | 3 | | |
| CALM2 | 3 | | |
| CALM3 | 3 | | |
| CALR | | | calreticulin |
| CAMK1 | | | |
| CAMK2A | | | |
| CAMK2B | | | |
| CAMK2D | 1, 3 | | |
| CAMK2G | | | |
| CAMK4 | | | |
| CAMTA2 | 3 | | |
| CASQ1 | no set | skel m | |
| CASQ2 | 1, 3 | | |
| CASK | 3 | | |
| CHRM1 | | | |
| CHRM2 | | | |
| CHRM3 | 6 | | |
| CHRM4 | | | |
| CHRM5 | | | |
| FKBP1B | 3 | | |
| FXYD2 | | | |
| GJA1 | 5 | | gap junction |
| GJA12 | | | gap junction |
| GJA4 | | | gap junction |
| GJA5 | 5 | | gap junction |
| GJA7 | 5 | | gap junction |
| GJB1 | 5 | | gap junction |
| GJB2 | | | gap junction |
| GJB3 | | | gap junction |
| GJB4 | | | gap junction |
| GJB5 | | | gap junction |
| GJB6 | | | gap junction |
| GNA11 | | | G protein |
| GNAI2 | 1 | | G protein |
| GNAI3 | | | G protein |
| GNAO1 | | | G protein |
| GNAQ | | | G protein |
| GNAZ | | | G protein |
| GNB1 | | | G protein |
| GNB2 | | | G protein |
| GNB3 | 1 | | G protein |
| GNB4 | | | G protein |
| GNB5 | | | G protein |
| GNG12 | | | G protein |
| GNG13 | | | G protein |
| GNG2 | | | G protein |
| GNG3 | | | G protein |
| GNG4 | | | G protein |
| GNG5 | | | G protein |
| GNG7 | | | G protein |
| GNGT1 | | | G protein |
| GRK4 | | | G prot receptor kinase |
| GRK5 | | | G prot receptor kinase |
| GRK6 | | | G prot receptor kinase |
| ITPR1 | no set | CNS | |
| ITPR2 | no set | | found in our HF v discovery |
| ITPR3 | | | |
| KCNB1 | 2 | | |
| KCNJ3 | 2 | | |
| KCNJ5 | 2 | | |
| MGC11266 | | | |
| MYCBP | 1 | | |
| MYOZ2 | 3 | | |
| NME7 | | | |
| PDE4D | 3 | | |
| PEA15 | | | |
| PKIA | | | protein kinase |
| PKIB | | | protein kinase |
| PKIG | | | protein kinase |
| PLCB3 | | | phospholipase C |
| PLN | 1, 3 | | |
| PPP3CA | 3 | | |
| PRKACA | 1, 3 | | protein kinases |
| PRKACB | | | protein kinases |
| PRKAR1A | | | protein kinases |
| PRKAR1B | | | protein kinases |
| PRKAR2A | 1, 3 | | protein kinases |
| PRKAR2B | | | protein kinases |
| PRKCA | 3 | | protein kinases |
| PRKCB1 | 3 | | protein kinases |
| PRKCD | | | protein kinases |
| PRKCE | | | protein kinases |
| PRKCG | | | protein kinases |
| PRKCH | | | protein kinases |
| PRKCQ | | | protein kinases |
| PRKCZ | | | protein kinases |
| PRKD1 | | | protein kinases |
| RGS1 | | | regulator of G prot signaling |
| RGS10 | | | regulator of G prot signaling |
| RGS11 | | | regulator of G prot signaling |
| RGS14 | | | regulator of G prot signaling |
| RGS16 | | | regulator of G prot signaling |
| RGS17 | | | regulator of G prot signaling |
| RGS18 | | | regulator of G prot signaling |
| RGS19 | | | regulator of G prot signaling |
| RGS2 | | | regulator of G prot signaling |
| RGS20 | | | regulator of G prot signaling |
| RGS3 | | | regulator of G prot signaling |
| RGS4 | | | regulator of G prot signaling |
| RGS5 | | | regulator of G prot signaling |
| RGS6 | | | regulator of G prot signaling |
| RGS7 | | | regulator of G prot signaling |
| RGS9 | | | regulator of G prot signaling |
| RYR1 | no set | skel m | |
| RYR2 | 1, 3 | | |
| RYR3 | | | |
| SARA1 | | | |
| SFN | | | stratifin |
| SFRS1 | 3 | | |
| SLC8A1 | 1, 3 | | |
| SLC8A2 | 1, 3 | | |
| SLC8A3 | | | |
| SLC9A1 | 1 | | |
| SLN | 3 | | |
| TRDN | 3 | | |
| USP5 | | | |
| YWHAB | | brain | MONOOXYGENASE |
| | | | ACTIVATION |
| | | | PROTEIN |
| YWHAH | | brain | MONOOXYGENASE |
| | | | ACTIVATION |
| | | | PROTEIN |
| YWHAQ | | T cells | MONOOXYGENASE |
| | | | ACTIVATION |
| | | | PROTEIN |
| YWHAQ /// | | | |
| MIB1 | | | |

TABLE 12

| | | | | | | | Mutated or | | | | | |
|---|---|---|---|---|---|---|---|---|---|---|---|---|
| | | | | | | | associated | | | | | |
| Ensembl | Chromo- | Start | End | | | | with Human | | | | Ion | |
| Gene ID Ver | some | Position | Position | Transcript | HGNC | | SCD | Disease | Other | | (handling or | structural or |
| 42 | Name | (bp) | (bp) | count | Symbol | Gene Name | disorders | Groupings | LOE | Organelle | dependence) | function |
| ENSG00000158022 | 1 | 26250382 | 26266711 | 1 | TRIM63 | tripartite motif- | | | | | | ? |
| | | | | | | containing 63 | | | | | | |
| NOT FOUND | | | | | SERPINE1 | serpin peptidase | | | | | | ? |
| | | | | | | inhibitor, clade E | | | | | | |
| | | | | | | (nexin, | | | | | | |
| | | | | | | plasminogen | | | | | | |
| | | | | | | activator inhibitor | | | | | | |
| | | | | | | type 1), member 1 | | | | | | |
| NOT FOUND | | | | | GP1BB | glycoprotein lb | | | | | | ? |
| | | | | | | (platelet), beta | | | | | | |
| | | | | | | polypeptide | | | | | | |
| ENSG00000169564 | 2 | 70168090 | 70169766 | 1 | PCBP1 | poly(rC) binding | | | | | | ? |
| | | | | | | protein 1 | | | | | | |
| ENS000000168610 | 17 | 37718869 | 37794039 | 5 | STAT3 | signal transducer | | | | | | acute phase |
| | | | | | | and activator of | | | | | | response |
| | | | | | | transcription 3 | | | | | | |
| | | | | | | (acute-phase | | | | | | |
| | | | | | | response factor) | | | | | | |
| ENSG00000169418 | 1 | 151917737 | 151933092 | 2 | NPR1 | natriuretic peptide | | | | | | ANP receptor |
| | | | | | | receptor | | | | | | |
| | | | | | | A/guanylate | | | | | | |
| | | | | | | cyclase A | | | | | | |
| | | | | | | (atrionatriuretic | | | | | | |
| | | | | | | peptide receptor | | | | | | |
| | | | | | | A) | | | | | | |
| ENSG00000130522 | 19 | 18252251 | 18253294 | 1 | JUND | jun D proto- | | | broad | | | AP1 |
| | | | | | | oncogene | | | functions, | | | transcription |
| | | | | | | | | | non- | | | factor |
| | | | | | | | | | cardiac | | | |
| ENSG00000164305 | 4 | 185785845 | 185807623 | 2 | CASP3 | caspase 3, | | | | | | apoptosis |
| | | | | | | apoptosis-related | | | | | | |
| | | | | | | cysteine peptidase | | | | | | |
| ENSG00000064012 | 2 | 201806426 | 201860677 | 9 | CASP8 | caspase 8, | | | | | | apoptosis |
| | | | | | | apoptosis-related | | | | | | |
| | | | | | | cysteine peptidase | | | | | | |
| ENSG00000002330 | 11 | 63793878 | 63808740 | 1 | BAD | BCL2-antagonist | | | | | | apoptosis |
| | | | | | | of cell death | | | | | | |
| ENSG00000087088 | 19 | 54149929 | 54156864 | 5 | BAX | BCL2-associated | | | | | | apoptosis |
| | | | | | | X protein | | | | | | |
| ENSG00000188389 | 2 | 242440711 | 242449731 | 2 | PDCD1 | programmed cell | | | autoimmune | | | apoptosis |
| | | | | | | death 1 | | | DCM, | | | |
| | | | | | | | | | mice | | | |
| ENSG00000171552 | 20 | 29715916 | 29774366 | 4 | BCL2L1 | BCL2-like 1 | | | | | | apoptosis |
| ENSG00000120937 | 1 | 11840108 | 11841575 | 2 | NPPB | natriuretic peptide | | | | | | BNP |
| | | | | | | precursor B | | | | | | |
| ENSG00000108691 | 17 | 29606409 | 29608329 | 1 | CCL2 | chemokine (C-C | | | | | | chemokines |
| | | | | | | motif) ligand 2 | | | | | | |
| ENSG00000161570 | 17 | 31222613 | 31231490 | 1 | CCL5 | chemokine (C-C | | | | | | chemokines |
| | | | | | | motif) ligand 5 | | | | | | |
| ENSG00000131187 | 3 | 5 | 176761747 | 1.77E+08 | F12 | coagulation factor | | | | | | clotting |
| | | | | | | XII (Hageman | | | | | | |
| | | | | | | factor) | | | | | | |
| ENSG00000124491 | 2 | 6 | 6089317 | 6265901 | F13A1 | coagulation factor | | | found in | | | clotting |
| | | | | | | XIII, A1 | | | discovery | | | |
| | | | | | | polypeptide | | | HF v | | | |
| | | | | | | | | | ctrl | | | |
| ENSG00000180210 | 3 | 11 | 46697331 | 46717631 | F2 | coagulation factor | | | | | | clotting |
| | | | | | | II (thrombin) | | | | | | |
| ENSG00000117525 | 2 | 1 | 94767369 | 94779944 | F3 | coagulation factor | | | | | | clotting |
| | | | | | | III (thromboplastin, | | | | | | |
| | | | | | | tissue factor) | | | | | | |
| ENSG00000198734 | 3 | 1 | 167750028 | 1.68E+08 | F5 | coagulation factor | | | | | | clotting |
| | | | | | | V (proaccelerin, | | | | | | |
| | | | | | | labile factor) | | | | | | |
| ENSG00000057593 | 2 | 13 | 112808106 | 1.13E+08 | F7 | coagulation factor | | | | | | clotting |
| | | | | | | VII (serum | | | | | | |
| | | | | | | prothrombin | | | | | | |
| | | | | | | conversion | | | | | | |
| | | | | | | accelerator) | | | | | | |
| ENSG00000171564 | 1 | 4 | 155703596 | 1.56E+08 | FGB | fibrinogen beta | | | | | | clotting |
| | | | | | | chain | | | | | | |
| ENSG00000108821 | 17 | 45616456 | 45633992 | 1 | COL1A1 | collagen, type I, | | | | | | collagens |
| | | | | | | alpha 1 | | | | | | |
| ENSG00000168542 | 2 | 189547344 | 189585717 | 2 | COL3A1 | collagen, type III, | | | | | | collagens |
| | | | | | | alpha 1 (Ehlers- | | | | | | |
| | | | | | | Danios syndrome | | | | | | |
| | | | | | | type IV, autosomal | | | | | | |
| | | | | | | dominant) | | | | | | |
| ENSG00000171497 | 4 | 159849730 | 159864002 | 1 | PPID | peptidylprolyl | | | | | | cyclophilin |
| | | | | | | isomerase D | | | | | | |
| | | | | | | (cyclophilin D) | | | | | | |
| ENSG00000204490 | 6 | 31651314 | 31654092 | 1 | TNF | tumor necrosis | | | | | | cytokine |
| | | | | | | factor (TNF | | | | | | |
| | | | | | | superfamily, | | | | | | |
| | | | | | | member 2) | | | | | | |
| ENSG00000150281 | 16 | 30815429 | 30822381 | 1 | CTF1 | cardiotrophin 1 | | | induces | | | cytokine. growth |
| | | | | | | | | | myocyte | | | factor |
| | | | | | | | | | hypertrophy, | | | |
| | | | | | | | | | signals | | | |
| | | | | | | | | | through | | | |
| | | | | | | | | | gp130 | | | |
| ENSG00000117594 | 1 | 207926133 | 207974918 | 3 | HSD11B1 | hydroxysteroid | | | | | | dehydrogenase |
| | | | | | | (11-beta) | | | | | | |
| | | | | | | dehydrogenase 1 | | | | | | |
| ENSG00000142871 | 1 | 85819005 | 85822233 | 2 | CYR61 | cysteine-rich, | | | | | | ECM signaling |
| | | | | | | angiogenic | | | | | | |
| | | | | | | inducer, 61 | | | | | | |
| ENSG00000140564 | 15 | 89212889 | 89227691 | 1 | FURIN | furin (paired basic | | | | | | enzyme |
| | | | | | | amino acid | | | | | | |
| | | | | | | cleaving enzyme) | | | | | | |
| ENSG00000177000 | 4 | 1 | 11768367 | 11788702 | MTHFR | 5,10- | | homocysteinuria | | | | enzyme |
| | | | | | | methylenetetrahydrofolate | | | | | | |
| | | | | | | reductase | | | | | | |
| | | | | | | (NADPH) | | | | | | |
| ENSG00000146070 | 6 | 46779897 | 46811389 | 2 | PLA2G7 | phospholipase A2. | | role in CAD | Lp- | | | enzyme |
| | | | | | | group VII (platelet- | | | PLA2 | | | |
| | | | | | | activating factor | | | | | | |
| | | | | | | acetylhydrolase, | | | | | | |
| | | | | | | plasma) | | | | | | |
| ENSG00000088832 | 20 | 1297625 | 1321806 | 4 | FKBP1A | FK506 binding | | | FKBP1 | | | FK506BP |
| | | | | | | protein 1A, 12 kDa | | | B more | | | |
| | | | | | | | | | important | | | |
| ENSG00000152413 | 5 | 78707505 | 78788599 | 2 | HOMER1 | homer homolog 1 | | | enriched | CNS | | glutamate |
| | | | | | | (Drosophila) | | | at | | | binding protein |
| | | | | | | | | | excitatory | | | |
| | | | | | | | | | synapses | | | |
| ENSG00000138685 | 4 | 123967313 | 124038840 | 1 | FGF2 | fibroblast growth | | | found in | | | growth factor |
| | | | | | | factor 2 (basic) | | | discovery | | | |
| | | | | | | | | | HF v | | | |
| | | | | | | | | | ctrl | | | |
| ENSG00000177885 | 17 | 70825753 | 70913384 | 2 | GRB2 | growth factor | | | | | | growth factor |
| | | | | | | receptor-bound | | | | | | |
| | | | | | | protein 2 | | | | | | |
| ENSG00000017427 | 12 | 101313809 | 101398471 | 2 | IGF1 | insulin-like growth | | | | | | growth factor |
| | | | | | | factor 1 | | | | | | |
| | | | | | | (somatomedin C) | | | | | | |
| ENSG00000170962 | 11 | 103283131 | 103540317 | 1 | PDGFD | platelet derived | | | | | | growth factor |
| | | | | | | growth factor D | | | | | | |
| ENSG00000112715 | 6 | 43845924 | 43862202 | 8 | VEGFA | vascular | | | | | | growth factor |
| | | | | | | endothelial growth | | | | | | |
| | | | | | | factor A | | | | | | |
| ENSG00000136238 | 7 | 6380651 | 6410120 | 2 | RAC1 | ras-related C3 | | | found in | | | GTP binding |
| | | | | | | botulinum toxin | | | discovery | | | protein |
| | | | | | | substrate 1 (rho | | | HF v | | | |
| | | | | | | family, small GTP | | | ctrl; | | | |
| | | | | | | binding protein | | | possibly | | | |
| | | | | | | Rac1) | | | involved | | | |
| | | | | | | | | | in | | | |
| | | | | | | | | | hypertrophic | | | |
| | | | | | | | | | response | | | |
| ENSG00000109971 | 11 | 122433411 | 122438054 | 1 | HSPA8 | heat shock 70 kDa | | | | | | heat shock |
| | | | | | | protein 8 | | | | | | proteins |
| ENSG00000004776 | 19 | 40937336 | 40939799 | 1 | HSPB6 | heat shock | | | | | | heat shock |
| | | | | | | protein, alpha- | | | | | | proteins |
| | | | | | | crystallin-related, | | | | | | |
| | | | | | | B6 | | | | | | |
| ENSG00000109846 | 11 | 111284560 | 111287704 | 1 | CRYAB | crystallin, alpha B | x | desmin | | | | heat shock |
| | | | | | | | | related | | | | proteins |
| | | | | | | | | myopatht, | | | | |
| | | | | | | | | cataracts | | | | |
| ENSG00000148926 | 11 | 10283172 | 10285491 | 1 | ADM | adrenomedullin | | | | | | hormone |
| ENSG00000172270 | 19 | 462896 | 534492 | 3 | BSG | basigin (Ok blood | | | | | | immunoglobulin |
| | | | | | | group) | | | | | | |
| ENSG00000132693 | 1 | 157948703 | 157951003 | 5 | CRP | C-reactive protein, | | | | | | inflammation |
| | | | | | | pentraxin-related | | | | | | |
| ENSG00000164171 | 1 | 5 | 52321014 | 52423805 | ITGA2 | integrin, alpha 2 | | | | | | integrins |
| | | | | | | (CD49B, alpha 2 | | | | | | |
| | | | | | | subunit of VLA-2 | | | | | | |
| | | | | | | receptor) | | | | | | |
| ENSG00000147166 | X | 70438309 | 70441946 | 1 | ITGB1BP2 | integrin beta 1 | | | | | | integrins |
| | | | | | | binding protein | | | | | | |
| | | | | | | (melusin) 2 | | | | | | |
| ENSG00000056345 | 1 | 17 | 42686207 | 42745076 | ITGB3 | integrin, beta 3 | | | | | | integrins |
| | | | | | | (platelet | | | | | | |
| | | | | | | glycoprotein IIIa, | | | | | | |
| | | | | | | antigen CD61) | | | | | | |
| ENSG00000111537 | 12 | 66834816 | 66839790 | 1 | IFNG | interferon, gamma | | | | | | interferon |
| ENSG00000137462 | 4 | 154842102 | 154846301 | 1 | TLR2 | toll-like receptor 2 | | | | | | interleukin-like |
| | | | | | | | | | | | | receptor |
| ENSG00000136634 | 1 | 205007570 | 205012462 | 1 | IL10 | interleukin 10 | | | | | | interleukins |
| ENSG00000125538 | 2 | 113303808 | 113310827 | 1 | IL1B | interleukin 1, beta | | | | | | interleukins |
| ENSG00000113520 | 5 | 132037272 | 132046267 | 4 | IL4 | interleukin 4 | | | | | | interleukins |
| ENSG00000136244 | 7 | 22732028 | 22738091 | 1 | IL6 | interleukin 6 | | | | | | interleukins |
| | | | | | | (interferon, beta 2) | | | | | | |
| ENSG00000134352 | 5 | 55266680 | 55326529 | 8 | IL6ST | interleukin 6 signal | | | | | | interleukins |
| | | | | | | transducer (gp130, | | | | | | |
| | | | | | | oncostatin M | | | | | | |
| | | | | | | receptor) | | | | | | |
| ENSG00000109572 | 4 | 170778297 | 170878731 | 2 | CLCN3 | chloride channel 3 | | | expressed | | Cl− | ion channel |
| | | | | | | | | | in | | | |
| | | | | | | | | | brain | | | |
| | | | | | | | | | and | | | |
| | | | | | | | | | neurons | | | |
| NOT FOUND | | | | | CLNS1B | chloride channel, | | | not in | | Cl− | ion channel |
| | | | | | | nucleotide- | | | OMIM | | | |
| | | | | | | sensitive, 1B | | | | | | |
| ENSG00000144285 | 2 | 166553919 | 166638395 | 3 | SCN1A | sodium channel, | x | generalized | | neuron, skel m | Na+ | ion channel |
| | | | | | | voltage-gated, | | epilepsy with | | | | |
| | | | | | | type I, alpha | | febrile | | | | |
| | | | | | | | | seizures, | | | | |
| | | | | | | | | myoclonic | | | | |
| | | | | | | | | epilesy | | | | |
| ENSG00000151704 | 11 | 128213125 | 128242478 | 2 | KCNJ1 | potassium | x | Bartter | | kidney | K+ | ion channel |
| | | | | | | inwardly-rectifying | | syndrome | | | | |
| | | | | | | channel, subfamily | | | | | | |
| | | | | | | J, member 1 | | | | | | |
| ENSG00000111262 | 12 | 4890806 | 4892293 | 1 | KCNA1 | potassium voltage- | | myokymia | | skel m | K+ | ion channel |
| | | | | | | gated channel, | | (rippling of | | | | |
| | | | | | | shaker-related | | muscles) and | | | | |
| | | | | | | subfamily, member | | episodic | | | | |
| | | | | | | 1 (episodic ataxia | | ataxia | | | | |
| | | | | | | with myokymia) | | | | | | |
| ENSG00000149575 | 11 | 117538729 | 117552546 | 1 | SCN2B | sodium channel, | | | | neurons | Na+ | ion channel |
| | | | | | | voltage-gated, | | | | | | |
| | | | | | | type II, beta | | | | | | |
| ENSG00000153253 | 2 | 165652286 | 165768799 | 4 | SCN3A | sodium channel, | | | | neuron, skel m | Na+ | ion channel |
| | | | | | | voltage-gated, | | | | | | |
| | | | | | | type III, alpha | | | | | | |
| ENSG00000007314 | 17 | 59369646 | 59404010 | 1 | SCN4A | sodium channel, | x | hyperkalemic | | skel m | Na+ | ion channel |
| | | | | | | voltage-gated, | | periodic | | | | |
| | | | | | | type IV, alpha | | paralysis, | | | | |
| | | | | | | | | myotonias, | | | | |
| | | | | | | | | myasthenia | | | | |
| ENSG00000082701 | 3 | 121028238 | 121295954 | 2 | GSK3B | glycogen synthase | | | | | | kinase |
| | | | | | | kinase 3 beta | | | | | | |
| ENSG00000096968 | 9 | 4975245 | 5118183 | 1 | JAK2 | Janus kinase 2 (a | | | | | | kinase |
| | | | | | | protein tyrosine | | | | | | |
| | | | | | | kinase) | | | | | | |
| ENSG00000142208 | 14 | 104306734 | 104333125 | 1 | AKT1 | v-akt murine | | | found in | | | kinase |
| | | | | | | thymoma viral | | | discovery | | | |
| | | | | | | oncogene | | | HF v | | | |
| | | | | | | homolog 1 | | | ctrl | | | |
| ENSG00000115641 | 2 | 105343717 | 105421392 | 4 | FHL2 | four and a half LIM | | | not | | | LIM protein |
| | | | | | | domains 2 | | | essential | | | |
| | | | | | | | | | for | | | |
| | | | | | | | | | cardiac | | | |
| | | | | | | | | | development | | | |
| | | | | | | | | | and | | | |
| | | | | | | | | | function | | | |
| ENSG00000005893 | X | 119446367 | 119487189 | 3 | LAMP2 | lysosomal- | x | HCM, Danon | | | | lysosomal |
| | | | | | | associated | | disease | | | | membrane |
| | | | | | | membrane protein 2 | | | | | | protein |
| ENSG00000065559 | 17 | 11864866 | 11987865 | 1 | MAP2K4 | mitogen-activated | | | | | | MAPKs |
| | | | | | | protein kinase | | | | | | |
| | | | | | | kinase 4 | | | | | | |
| ENSG00000095015 | 5 | 56147216 | 56225472 | 1 | MAP3K1 | mitogen-activated | | | | | | MAPKs |
| | | | | | | protein kinase | | | | | | |
| | | | | | | kinase kinase 1 | | | | | | |
| ENSG00000197442 | 6 | 136919878 | 137155349 | 3 | MAP3K5 | mitogen-activated | | | | | | MAPKs |
| | | | | | | protein kinase | | | | | | |
| | | | | | | kinase kinase 5 | | | | | | |
| ENSG00000100030 | 22 | 20446873 | 20551730 | 1 | MAPK1 | mitogen-activated | | | | | | MAPKs |
| | | | | | | protein kinase 1 | | | | | | |
| ENSG00000112062 | 6 | 36103551 | 36186513 | 3 | MAPK14 | mitogen-activated | | | | | | MAPKs |
| | | | | | | protein kinase 14 | | | | | | |
| ENSG00000196611 | 11 | 102165861 | 102174099 | 1 | MMP1 | matrix | | | | | | MMPs |
| | | | | | | metallopeptidase 1 | | | | | | |
| | | | | | | (interstitial | | | | | | |
| | | | | | | collagenase) | | | | | | |
| ENSG00000137745 | 11 | 102318937 | 102331672 | 2 | MMP13 | matrix | | | | | | MMPs |
| | | | | | | metallopeptidase | | | | | | |
| | | | | | | 13 (collagenase 3) | | | | | | |
| ENSG00000157227 | 14 | 22375676 | 22385088 | 1 | MMP14 | matrix | | | found in | | | MMPs |
| | | | | | | metallopeptidase | | | discovery | | | |
| | | | | | | 14 (membrane- | | | HF v | | | |
| | | | | | | inserted) | | | ctrl | | | |
| ENSG00000087245 | 16 | 54070589 | 54098101 | 1 | MMP2 | matrix | | | | | | MMPs |
| | | | | | | metallopeptidase 2 | | | | | | |
| | | | | | | (gelatinase A, | | | | | | |
| | | | | | | 72 kDa gelatinase, | | | | | | |
| | | | | | | 72 kDa type IV | | | | | | |
| | | | | | | collagenase) | | | | | | |
| ENSG00000149968 | 11 | 102211738 | 102219552 | 1 | MMP3 | matrix | | | | | | MMPs |
| | | | | | | metallopeptidase 3 | | | | | | |
| | | | | | | (stromelysin 1, | | | | | | |
| | | | | | | progelatinase) | | | | | | |
| ENSG00000100985 | 20 | 44070954 | 44078607 | 1 | MMP9 | matrix | | | | | | MMPs |
| | | | | | | metallopeptidase 9 | | | | | | |
| | | | | | | (gelatinase B, | | | | | | |
| | | | | | | 92 kDa gelatinase, | | | | | | |
| | | | | | | 92 kDa type IV | | | | | | |
| | | | | | | collagenase) | | | | | | |
| ENSG00000080815 | 14 | 72672915 | 72756862 | 4 | PSEN1 | presenilin 1 | x | DCM, | | | | multi-function |
| | | | | | | (Alzheimer | | Alzheimer's | | | | |
| | | | | | | disease 3) | | | | | | |
| ENSG00000137808 | 15 | 67094125 | 67136516 | 2 | NOX5 | NADPH oxidase, | | | functions | | | NADPH oxidase |
| | | | | | | EF-hand calcium | | | as a | | | |
| | | | | | | binding domain 5 | | | H+ | | | |
| | | | | | | | | | channel | | | |
| | | | | | | | | | in a | | | |
| | | | | | | | | | Ca(2+)- | | | |
| | | | | | | | | | dependent | | | |
| | | | | | | | | | manner | | | |
| ENSG00000182687 | 17 | 71582479 | 71585168 | 1 | GALR2 | galanin receptor 2 | | | | | | neuropeptide |
| ENSG00000139133 | 12 | 34066483 | 34072501 | 1 | ALG10A | asparagine-linked | | | | | | not in NCBI or |
| | | | | | | glycosylation 10 | | | | | | OMIM |
| | | | | | | homolog (yeast, | | | | | | |
| | | | | | | alpha-1,2- | | | | | | |
| | | | | | | glucosyltransferase) | | | | | | |
| ENSG00000158125 | 2 | 31410691 | 31491117 | 2 | XDH | xanthine | x | xanthanurias | | | | oxidative |
| | | | | | | dehydrogenase | | | | | | metabolism |
| ENSG00000172531 | 11 | 66922228 | 66925978 | 3 | PPP1CA | protein | | | | | | phosphatases |
| | | | | | | phosphatase 1, | | | | | | |
| | | | | | | catalytic subunit, | | | | | | |
| | | | | | | alpha isoform | | | | | | |
| ENSG00000135447 | 12 | 53257439 | 53268723 | 2 | PPP1R1A | protein | | | | | | phosphatases |
| | | | | | | phosphatase 1, | | | | | | |
| | | | | | | regulatory | | | | | | |
| | | | | | | (inhibitor) subunit | | | | | | |
| | | | | | | 1A | | | | | | |
| ENSG00000108819 | 17 | 45567695 | 45582873 | 1 | PPP1R9B | protein | | | | | | phosphatases |
| | | | | | | phosphatase 1, | | | | | | |
| | | | | | | regulatory subunit | | | | | | |
| | | | | | | 9B, spinophilin | | | | | | |
| ENSG00000156475 | 5 | 145949265 | 146415783 | 2 | PPP2R2B | protein | | | | | | phosphatases |
| | | | | | | phosphatase 2 | | | | | | |
| | | | | | | (formerly 2A), | | | | | | |
| | | | | | | regulatory subunit | | | | | | |
| | | | | | | B (PR 52), beta | | | | | | |
| | | | | | | isoform | | | | | | |
| ENSG00000073711 | 3 | 137167257 | 137349423 | 2 | PPP2R3A | protein | | | | | | phosphatases |
| | | | | | | phosphatase 2 | | | | | | |
| | | | | | | (formerly 2A), | | | | | | |
| | | | | | | regulatory subunit | | | | | | |
| | | | | | | B″, alpha | | | | | | |
| ENSG00000188386 | 9 | 103393718 | 103397104 | 2 | PPP3R2 | protein | | | | | | phosphatases |
| | | | | | | phosphatase 3 | | | | | | |
| | | | | | | (formerly 2B), | | | | | | |
| | | | | | | regulatory subunit | | | | | | |
| | | | | | | B, beta isoform | | | | | | |
| ENSG00000180817 | 10 | 71632592 | 71663196 | 2 | PPA1 | pyrophosphatase | | | | | | phosphatases |
| | | | | | | (inorganic) 1 | | | | | | |
| ENSG00000179295 | 12 | 111340919 | 111432099 | 1 | PTPN11 | protein tyrosine | x | HCM, | | | | phosphatases |
| | | | | | | phosphatase, non- | | Noonan syndr | | | | |
| | | | | | | receptor type 11 | | | | | | |
| | | | | | | (Noonan | | | | | | |
| | | | | | | syndrome 1) | | | | | | |
| ENSG00000112293 | 6 | 24536384 | 24597829 | 2 | GPLD1 | glycosylphosphatidylinositol | | | | | | phospholipase |
| | | | | | | specific | | | | | | |
| | | | | | | phospholipase D1 | | | | | | |
| ENSG00000135047 | 9 | 89530254 | 89536127 | 3 | CTSL | cathepsin L | | implicated in | | | | protease |
| | | | | | | | | pathologic | | | | |
| | | | | | | | | processes | | | | |
| | | | | | | | | including | | | | |
| | | | | | | | | myofibril | | | | |
| | | | | | | | | necrosis in | | | | |
| | | | | | | | | myopathies | | | | |
| | | | | | | | | and in MI | | | | |
| ENSG00000150995 | 3 | 4510136 | 4863432 | 4 | ITPR1 | inositol 1,4,5- | | | | CNS | | receptor |
| | | | | | | triphosphate | | | | | | |
| | | | | | | receptor, type 1 | | | | | | |
| ENSG00000123104 | 12 | 26381609 | 26877347 | 2 | ITPR2 | inositol 1,4,5- | | | found in | | | receptor |
| | | | | | | triphosphate | | | discovery | | | |
| | | | | | | receptor, type 2 | | | HF v | | | |
| | | | | | | | | | ctrl | | | |
| ENSG00000113594 | 5 | 38510823 | 38631253 | 1 | LIFR | leukemia inhibitory | | | found in | | | receptor |
| | | | | | | factor receptor | | | discovery | | | |
| | | | | | | alpha | | | HF v | | | |
| | | | | | | | | | ctrl | | | |
| ENSG00000138095 | 2 | 43968391 | 44076648 | 3 | LRPPRC | leucine-rich PPR- | x | Leigh | | | | regulatory |
| | | | | | | motif containing | | syndrome | | | | protein |
| ENSG00000135486 | 12 | 52960755 | 52965297 | 2 | HNRPA1 | heterogeneous | | | | | | ribonucleoprotein |
| | | | | | | nuclear | | | | | | |
| | | | | | | ribonucleoprotein | | | | | | |
| | | | | | | A1 | | | | | | |
| ENSG00000165119 | 9 | 85772818 | 85785339 | 8 | HNRPK | heterogeneous | | | | | | ribonucleoprotein |
| | | | | | | nuclear | | | | | | |
| | | | | | | ribonucleoprotein K | | | | | | |
| ENSG00000133216 | 1 | 22910045 | 23114405 | 4 | EPHB2 | EPH receptor B2 | | | | | | RTK |
| ENSG00000118785 | 4 | 89115890 | 89123592 | 3 | SPP1 | secreted | | | involved | | | secreted protein |
| | | | | | | phosphoprotein 1 | | | in the | | | |
| | | | | | | (osteopontin, bone | | | regulation | | | |
| | | | | | | sialoprotein I, early | | | of | | | |
| | | | | | | T-lymphocyte | | | cardiac | | | |
| | | | | | | activation 1) | | | remodeling | | | |
| ENSG00000175387 | 18 | 43618435 | 43711221 | 2 | SMAD2 | SMAD family | | | | | | signaling |
| | | | | | | member 2 | | | | | | |
| ENSG00000166949 | 15 | 65145249 | 65274586 | 1 | SMAD3 | SMAD family | | | found in | | | signaling |
| | | | | | | member 3 | | | discovery | | | |
| | | | | | | | | | HF v | | | |
| | | | | | | | | | ctrl; | | | |
| | | | | | | | | | signaling | | | |
| | | | | | | | | | TGFbeta | | | |
| ENSG00000141646 | 18 | 46810611 | 46860142 | 1 | SMAD4 | SMAD family | | | | | | signaling |
| | | | | | | member 4 | | | | | | |
| ENSG00000164056 | 4 | 124537406 | 124544357 | 1 | SPRY1 | sprouty homolog | | | | | | signaling |
| | | | | | | 1, antagonist of | | | | | | |
| | | | | | | FGF signaling | | | | | | |
| | | | | | | (Drosophila) | | | | | | |
| ENSG00000166068 | 15 | 36331808 | 36433526 | 1 | SPRED1 | sprouty-related, | | | | | | signaling |
| | | | | | | EVH1 domain | | | | | | |
| | | | | | | containing 1 | | | | | | |
| ENSG00000104936 | 19 | 50965579 | 50977469 | 6 | DMPK | dystrophia | x | myotonic | | | | skel m, brain |
| | | | | | | myotonica-protein | | dystrophy | | | | |
| | | | | | | kinase | | | | | | |
| ENSG00000196218 | 19 | 43616180 | 43770012 | 5 | RYR1 | ryanodine receptor | | | | | | skeletal m |
| | | | | | | 1 (skeletal) | | | | | | |
| ENSG00000143318 | 1 | 158426970 | 158438300 | 2 | CASQ1 | calsequestrin 1 | | | | | | skeletal m |
| | | | | | | (fast-twitch, | | | | | | |
| | | | | | | skeletal muscle) | | | | | | |
| ENSG00000161547 | 17 | 72241796 | 72244837 | 3 | SFRS2 | splicing factor, | | | | | | splicing factor |
| | | | | | | arginine/serine- | | | | | | |
| | | | | | | rich 2 | | | | | | |
| ENSG00000105329 | 19 | 46528254 | 46551628 | 1 | TGFB1 | transforming | | | | | | TGFbeta |
| | | | | | | growth factor, beta | | | | | | |
| | | | | | | 1 (Camurati- | | | | | | |
| | | | | | | Engelmann | | | | | | |
| | | | | | | disease) | | | | | | |
| ENSG00000102265 | X | 47326634 | 47331132 | 4 | TIMP1 | TIMP | | | | | | TIMPs |
| | | | | | | metallopeptidase | | | | | | |
| | | | | | | inhibitor 1 | | | | | | |
| ENSG00000035862 | 17 | 74360658 | 74433067 | 1 | TIMP2 | TIMP | | | | | | TIMPs |
| | | | | | | metallopeptidase | | | | | | |
| | | | | | | inhibitor 2 | | | | | | |
| ENSG00000100234 | 22 | 31526802 | 31589025 | 2 | TIMP3 | TIMP | | | | | | TIMPs |
| | | | | | | metallopeptidase | | | | | | |
| | | | | | | inhibitor 3 (Sorsby | | | | | | |
| | | | | | | fundus dystrophy, | | | | | | |
| | | | | | | pseudoinflammatory) | | | | | | |
| ENSG00000157150 | 3 | 12169578 | 12175851 | 1 | TIMP4 | TIMP | | | | | | TIMPs |
| | | | | | | metallopeptidase | | | | | | |
| | | | | | | inhibitor 4 | | | | | | |
| ENSG00000109320 | 4 | 103641518 | 103757506 | 1 | NFKB1 | nuclear factor of | | CAD, | | | | transcription |
| | | | | | | kappa light | | inflammation | | | | factor |
| | | | | | | polypeptide gene | | | | | | |
| | | | | | | enhancer in B- | | | | | | |
| | | | | | | cells 1 (p105) | | | | | | |
| ENSG00000049247 | 1 | 7825731 | 7836161 | 3 | UTS2 | urotensin 2 | | | secreted | | | vasoactive |
| | | | | | | | | | protein | | | peptide |
| | | | | | | | | | with | | | |
| | | | | | | | | | vasoactive | | | |
| | | | | | | | | | properties; | | | |
| | | | | | | | | | altered | | | |
| | | | | | | | | | expression | | | |
| | | | | | | | | | in | | | |
| | | | | | | | | | HF | | | |
| ENSG00000078401 | 6 | 12398582 | 12405413 | 1 | EDN1 | endothelin 1 | | | | | | vasoconstrictor |
| | | | | | | | | | | | | peptide |
| ENSG00000106125 | 7 | 30917993 | 30931656 | 3 | AQP1 | aquaporin 1 | | | | | | water channel |
| | | | | | | (Colton blood | | | | | | |
| | | | | | | group) | | | | | | |
| ENSG00000145740 | 5 | 68425839 | 68462648 | 2 | SLC30A5 | solute carrier | | | involved | | | zinc transporter |
| | | | | | | family 30 (zinc | | | in | | | |
| | | | | | | transporter), | | | maintenance | | | |
| | | | | | | member 5 | | | of | | | |
| | | | | | | | | | the | | | |
| | | | | | | | | | cells | | | |
| | | | | | | | | | involved | | | |
| | | | | | | | | | in the | | | |
| | | | | | | | | | cardiac | | | |
| | | | | | | | | | conduction | | | |
| | | | | | | | | | system | | | |

TABLE 13

| | Chromo- | Start | End | | | |
|---|---|---|---|---|---|---|
| Ensembl Gene ID | some | Position | Position | Transcript | HGNC | |
| Ver 42 | Name | (bp) | (bp) | count | Symbol | Gene Name |
| ENSG00000002330 | 11 | 63793878 | 63808740 | 1 | BAD | BCL2-antagonist of cell death |
| ENSG00000004776 | 19 | 40937336 | 40939799 | 1 | HSPB6 | heat shock protein, alpha-crystallin-related, B6 |
| ENSG00000005893 | X | 119446367 | 119487189 | 3 | LAMP2 | lysosomal-associated membrane protein 2 |
| ENSG00000006283 | 17 | 45993820 | 46059541 | 6 | CACNA1G | calcium channel, voltage-dependent, alpha 1G subunit |
| ENSG00000006695 | 17 | 13913444 | 14052712 | 1 | COX10 | COX10 homolog, cytochrome c oxidase assembly |
| | | | | | | protein, heme A: farnesyltransferase (yeast) |
| ENSG00000007314 | 17 | 59369646 | 59404010 | 1 | SCN4A | sodium channel, voltage-gated, type IV, alpha |
| ENSG00000007402 | 3 | 50375237 | 50516032 | 2 | CACNA2D2 | calcium channel, voltage-dependent, alpha 2/delta subunit 2 |
| ENSG00000014919 | 10 | 101461591 | 101482413 | 2 | COX15 | COX15 homolog, cytochrome c oxidase assembly |
| | | | | | | protein (yeast) |
| ENSG00000017427 | 12 | 101313809 | 101398471 | 2 | IGF1 | insulin-like growth factor 1 (somatomedin C) |
| ENSG00000018625 | 1 | 158352172 | 158379996 | 2 | ATP1A2 | ATPase, Na+/K+ transporting, alpha 2 (+) polypeptide |
| ENSG00000035403 | 10 | 75427878 | 75549924 | 2 | VCL | vinculin |
| ENSG00000035862 | 17 | 74360658 | 74433067 | 1 | TIMP2 | TIMP metallopeptidase inhibitor 2 |
| ENSG00000043591 | 10 | 115793796 | 115796657 | 2 | ADRB1 | adrenergic, beta-1-, receptor |
| ENSG00000049247 | 1 | 7825731 | 7836161 | 3 | UTS2 | urotensin 2 |
| ENSG00000053918 | 11 | 2422797 | 2826915 | 4 | KCNQ1 | potassium voltage-gated channel, KQT-like subfamily, |
| | | | | | | member 1 |
| ENSG00000055118 | 7 | 150272982 | 150306121 | 3 | KCNH2 | potassium voltage-gated channel, subfamily H |
| | | | | | | (eag-related), member 2 |
| ENSG00000056345 | 1 | 17 | 42686207 | 42745076 | ITGB3 | integrin, beta 3 (platelet glycoprotein IIIa, antigen CD61) |
| ENSG00000057294 | 12 | 32834954 | 32941041 | 2 | PKP2 | plakophilin 2 |
| ENSG00000057593 | 2 | 13 | 112808106 | 112822996 | F7 | coagulation factor VII (serum prothrombin |
| | | | | | | conversion accelerator) |
| ENSG00000064012 | 2 | 201806426 | 201860677 | 9 | CASP8 | caspase 8, apoptosis-related cysteine peptidase |
| ENSG00000065559 | 17 | 11864866 | 11987865 | 1 | MAP2K4 | mitogen-activated protein kinase kinase 4 |
| ENSG00000067191 | 17 | 34583232 | 34607427 | 2 | CACNB1 | calcium channel, voltage-dependent, beta 1 subunit |
| ENSG00000068305 | 15 | 97923712 | 98074131 | 3 | MEF2A | MADS box transcription enhancer factor 2, polypeptide A |
| | | | | | | (myocyte enhancer factor 2A) |
| ENSG00000069424 | 1 | 5974113 | 6083840 | 8 | KCNAB2 | potassium voltage-gated channel, shaker-related subfamily, |
| | | | | | | beta member 2 |
| ENSG00000069431 | 12 | 21845245 | 21985434 | 4 | ABCC9 | ATP-binding cassette, sub-family C (CFTR/MRP), |
| | | | | | | member 9 |
| ENSG00000072062 | 19 | 14063509 | 14089559 | 2 | PRKACA | protein kinase, cAMP-dependent, catalytic, alpha |
| ENSG00000072110 | 14 | 68410793 | 68515747 | 1 | ACTN1 | actinin, alpha 1 |
| ENSG00000073578 | 5 | 271356 | 309815 | 3 | SDHA | succinate dehydrogenase complex, subunit A, |
| | | | | | | flavoprotein (Fp) |
| ENSG00000073711 | 3 | 137167257 | 137349423 | 2 | PPP2R3A | protein phosphatase 2 (formerly 2A), |
| | | | | | | regulatory subunit B”, alpha |
| ENSG00000073905 | 5 | 133335506 | 133368723 | 1 | VDAC1 | voltage-dependent anion channel 1 |
| ENSG00000074201 | 11 | 77004847 | 77026495 | 1 | CLNS1A | chloride channel, nucleotide-sensitive, 1A |
| ENSG00000074582 | 2 | 219231772 | 219236399 | 1 | BCS1L | BCS1-like (yeast) |
| ENSG00000077549 | 1 | 19537857 | 19684594 | 5 | CAPZB | Capping protein (actin filament) muscle Z-line, beta |
| ENSG00000078401 | 6 | 12398582 | 12405413 | 1 | EDN1 | endothelin 1 |
| ENSG00000080815 | 14 | 72672915 | 72756862 | 4 | PSEN1 | presenilin 1 (Alzheimer disease 3) |
| ENSG00000081189 | 5 | 88051922 | 88214818 | 2 | MEF2C | MADS box transcription enhancer factor 2, polypeptide C |
| | | | | | | (myocyte enhancer factor 2C) |
| ENSG00000082701 | 3 | 121028238 | 121295954 | 2 | GSK3B | glycogen synthase kinase 3 beta |
| ENSG00000087088 | 19 | 54149929 | 54156864 | 5 | BAX | BCL2-associated X protein |
| ENSG00000087245 | 16 | 54070589 | 54098101 | 1 | MMP2 | matrix metallopeptidase 2 (gelatinase A, 72 kDa |
| | | | | | | gelatinase, 72 kDa type IV collagenase) |
| ENSG00000088832 | 20 | 1297625 | 1321806 | 4 | FKBP1A | FK506 binding protein 1A, 12 kDa |
| ENSG00000089225 | 12 | 113276119 | 113330630 | 3 | TBX5 | T-box 5 |
| ENSG00000089250 | 12 | 116135362 | 116283965 | 3 | NOS1 | nitric oxide synthase 1 (neuronal) |
| ENSG00000090020 | 1 | 27297893 | 27366059 | 4 | SLC9A1 | solute carrier family 9 (sodium/hydrogen exchanger), |
| | | | | | | member 1 (antiporter, Na+/H+, amiloride sensitive) |
| ENSG00000091140 | 7 | 107318847 | 107347645 | 1 | DLD | dihydrolipoamide dehydrogenase (E3 component of |
| | | | | | | pyruvate dehydrogenase complex, 2-oxo-glutarate complex, |
| | | | | | | branched chain keto acid dehydrogenase complex) |
| ENSG00000092009 | 14 | 24044552 | 24047311 | 2 | CMA1 | chymase 1, mast cell |
| ENSG00000092054 | 14 | 22951789 | 22974690 | 2 | MYH7 | myosin, heavy polypeptide 7, cardiac muscle, beta |
| ENSG00000095015 | 5 | 56147216 | 56225472 | 1 | MAP3K1 | mitogen-activated protein kinase kinase kinase 1 |
| ENSG00000096696 | 6 | 7486869 | 7531945 | 1 | DSP | desmoplakin |
| ENSG00000096968 | 9 | 4975245 | 5118183 | 1 | JAK2 | Janus kinase 2 (a protein tyrosine kinase) |
| ENSG00000099337 | 19 | 43502322 | 43511480 | 1 | KCNK6 | potassium channel, subfamily K, member 6 |
| ENSG00000099822 | 19 | 540893 | 568157 | 1 | HCN2 | hyperpolarization activated cyclic nucleotide-gated |
| | | | | | | potassium channel 2 |
| ENSG00000100030 | 22 | 20446873 | 20551730 | 1 | MAPK1 | mitogen-activated protein kinase 1 |
| ENSG00000100077 | 22 | 24290946 | 24449916 | 1 | ADRBK2 | adrenergic, beta, receptor kinase 2 |
| ENSG00000100234 | 22 | 31526802 | 31589025 | 2 | TIMP3 | TIMP metallopeptidase inhibitor 3 |
| | | | | | | (Sorsby fundus dystrophy, pseudoinflammatory) |
| ENSG00000100985 | 20 | 44070954 | 44078607 | 1 | MMP9 | matrix metallopeptidase 9 (gelatinase B, 92 kDa gelatinase, |
| | | | | | | 92 kDa type IV collagenase) |
| ENSG00000101096 | 20 | 49441083 | 49592665 | 2 | NFATC2 | nuclear factor of activated T-cells, cytoplasmic, |
| | | | | | | calcineurin-dependent 2 |
| ENSG00000101400 | 20 | 31459424 | 31495359 | 1 | SNTA1 | syntrophin, alpha 1 (dystrophin-associated protein A1, |
| | | | | | | 59 kDa, acidic component) |
| ENSG00000102265 | X | 47326634 | 47331132 | 4 | TIMP1 | TIMP metallopeptidase inhibitor 1 |
| ENSG00000103546 | 16 | 54248057 | 54296685 | 3 | SLC6A2 | solute carrier family 6 (neurotransmitter transporter, |
| | | | | | | noradrenalin), member 2 |
| ENSG00000104936 | 19 | 50965579 | 50977469 | 6 | DMPK | dystrophia myotonica-protein kinase |
| ENSG00000105329 | 19 | 46528254 | 46551628 | 1 | TGFB1 | transforming growth factor, beta 1 |
| | | | | | | (Camurati-Engelmann disease) |
| ENSG00000105711 | 19 | 40213374 | 40223192 | 1 | SCN1B | sodium channel, voltage-gated, type I, beta |
| ENSG00000105866 | 7 | 21434214 | 21520674 | 1 | SP4 | Sp4 transcription factor |
| ENSG00000106125 | 7 | 30917993 | 30931656 | 3 | AQP1 | aquaporin 1 (Colton blood group) |
| ENSG00000106617 | 7 | 150884960 | 151204728 | 1 | PRKAG2 | protein kinase, AMP-activated, |
| | | | | | | gamma 2 non-catalytic subunit |
| ENSG00000108064 | 10 | 59814788 | 59828987 | 2 | TFAM | transcription factor A, mitochondrial |
| ENSG00000108509 | 17 | 4812017 | 4831671 | 5 | CAMTA2 | calmodulin binding transcription activator 2 |
| ENSG00000108691 | 17 | 29606409 | 29608329 | 1 | CCL2 | chemokine (C-C motif) ligand 2 |
| ENSG00000108819 | 17 | 45567695 | 45582873 | 1 | PPP1R9B | protein phosphatase 1, regulatory subunit 9B, spinophilin |
| ENSG00000108821 | 17 | 45616456 | 45633992 | 1 | COL1A1 | collagen, type I, alpha 1 |
| ENSG00000108840 | 17 | 39509647 | 39556540 | 2 | HDAC5 | histone deacetylase 5 |
| ENSG00000109320 | 4 | 103641518 | 103757506 | 1 | NFKB1 | nuclear factor of kappa light polypeptide gene |
| | | | | | | enhancer in B-cells 1 (p105) |
| ENSG00000109572 | 4 | 170778297 | 170878731 | 2 | CLCN3 | chloride channel 3 |
| ENSG00000109846 | 11 | 111284560 | 111287704 | 1 | CRYAB | crystallin, alpha B |
| ENSG00000109971 | 11 | 122433411 | 122438054 | 1 | HSPA8 | heat shock 70 kDa protein 8 |
| ENSG00000110536 | 11 | 47543464 | 47562690 | 3 | NDUFS3 | NADH dehydrogenase (ubiquinone) |
| | | | | | | Fe—S protein 3, 30 kDa (NADH-coenzyme Q reductase) |
| ENSG00000110717 | 11 | 67554670 | 67560686 | 1 | NDUFS8 | NADH dehydrogenase (ubiquinone) |
| | | | | | | Fe—S protein 8, 23 kDa (NADH-coenzyme Q reductase) |
| ENSG00000111245 | 12 | 109833009 | 109842766 | 1 | MYL2 | myosin, light polypeptide 2, regulatory, cardiac, slow |
| ENSG00000111262 | 12 | 4890806 | 4892293 | 1 | KCNA1 | potassium voltage-gated channel, shaker-related subfamily, |
| | | | | | | member 1 (episodic ataxia with myokymia) |
| ENSG00000111537 | 12 | 66834816 | 66839790 | 1 | IFNG | interferon, gamma |
| ENSG00000111664 | 12 | 6820713 | 6826819 | 2 | GNB3 | guanine nucleotide binding protein (G protein), |
| | | | | | | beta polypeptide 3 |
| ENSG00000112062 | 6 | 36103551 | 36186513 | 3 | MAPK14 | mitogen-activated protein kinase 14 |
| ENSG00000112096 | 6 | 160020138 | 160034343 | 3 | SOD2 | superoxide dismutase 2, mitochondrial |
| ENSG00000112293 | 6 | 24536384 | 24597829 | 2 | GPLD1 | glycosylphosphatidylinositol specific phospholipase D1 |
| ENSG00000112715 | 6 | 43845924 | 43862202 | 8 | VEGFA | vascular endothelial growth factor A |
| ENSG00000113448 | 5 | 58305622 | 59320301 | 5 | PDE4D | phosphodiesterase 4D, cAMP-specific (phosphodiesterase |
| | | | | | | E3 dunce homolog, Drosophila) |
| ENSG00000113520 | 5 | 132037272 | 132046267 | 4 | IL4 | interleukin 4 |
| ENSG00000113594 | 5 | 38510823 | 38631253 | 1 | LIFR | leukemia inhibitory factor receptor alpha |
| ENSG00000114302 | 3 | 48762099 | 48860274 | 2 | PRKAR2A | protein kinase, cAMP-dependent, regulatory, type II, alpha |
| ENSG00000114353 | 3 | 50239173 | 50271775 | 3 | GNAI2 | guanine nucleotide binding protein (G protein), |
| | | | | | | alpha inhibiting activity polypeptide 2 |
| ENSG00000114854 | 3 | 52460158 | 52463098 | 1 | TNNC1 | troponin C type 1 (slow) |
| ENSG00000115286 | 19 | 1334883 | 1346583 | 3 | NDUFS7 | NADH dehydrogenase(ubiquinone) Fe—S protein 7, |
| | | | | | | 20 kDa (NADH-coenzyme Q reductase) |
| ENSG00000115414 | 2 | 215933409 | 216009041 | 10 | FN1 | fibronectin 1 |
| ENSG00000115641 | 2 | 105343717 | 105421392 | 4 | FHL2 | four and a half LIM domains 2 |
| ENSG00000117525 | 2 | 1 | 94767369 | 94779944 | F3 | coagulation factor III (thromboplastin, tissue factor) |
| ENSG00000117594 | 1 | 207926133 | 207974918 | 3 | HSD11B1 | hydroxysteroid (11-beta) dehydrogenase 1 |
| ENSG00000118160 | 19 | 52623735 | 52666934 | 1 | SLC8A2 | solute carrier family 8 (sodium-calcium exchanger), |
| | | | | | | member 2 |
| ENSG00000118194 | 1 | 199594759 | 199613431 | 10 | TNNT2 | troponin T type 2 (cardiac) |
| ENSG00000118729 | 1 | 116044151 | 116112925 | 1 | CASQ2 | calsequestrin 2 (cardiac muscle) |
| ENSG00000118785 | 4 | 89115890 | 89123592 | 3 | SPP1 | secreted phosphoprotein 1 (osteopontin, bone sialoprotein |
| | | | | | | I, early T-lymphocyte activation 1) |
| ENSG00000119782 | 2 | 24126075 | 24140055 | 4 | FKBP1B | FK506 binding protein 1B, 12.6 kDa |
| ENSG00000120049 | 10 | 103575721 | 103593667 | 12 | KCNIP2 | Kv channel interacting protein 2 |
| ENSG00000120457 | 11 | 128266517 | 128293159 | 1 | KCNJ5 | potassium inwardly-rectifying channel, |
| | | | | | | subfamily J, member 5 |
| ENSG00000120907 | 8 | 26661584 | 26778839 | 12 | ADRA1A | adrenergic, alpha-1A-, receptor |
| ENSG00000120937 | 1 | 11840108 | 11841575 | 2 | NPPB | natriuretic peptide precursor B |
| ENSG00000121361 | 12 | 21809156 | 21819014 | 1 | KCNJ8 | potassium inwardly-rectifying channel, |
| | | | | | | subfamily J, member 8 |
| ENSG00000121933 | 1 | 111827493 | 111908107 | 6 | ADORA3 | adenosine A3 receptor |
| ENSG00000123104 | 12 | 26381609 | 26877347 | 2 | ITPR2 | inositol 1,4,5-triphosphate receptor, type 2 |
| ENSG00000123700 | 17 | 65677271 | 65687755 | 1 | KCNJ2 | potassium inwardly-rectifying channel, |
| | | | | | | subfamily J, member 2 |
| ENSG00000124491 | 2 | 6 | 6089317 | 6265901 | F13A1 | coagulation factor XIII, A1 polypeptide |
| ENSG00000125538 | 2 | 113303808 | 113310827 | 1 | IL1B | interleukin 1, beta |
| ENSG00000127914 | 7 | 91408128 | 91577925 | 6 | AKAP9 | A kinase (PRKA) anchor protein (yotiao) 9 |
| ENSG00000128271 | 22 | 23153537 | 23168309 | 2 | ADORA2A | adenosine A2a receptor |
| ENSG00000129170 | 11 | 19160154 | 19180177 | 1 | CSRP3 | cysteine and glycine-rich protein 3 (cardiac LIM protein) |
| ENSG00000129991 | 19 | 60355014 | 60360496 | 1 | TNNI3 | troponin I type 3 (cardiac) |
| ENSG00000130037 | 12 | 5023346 | 5026210 | 1 | KCNA5 | potassium voltage-gated channel, shaker-related |
| | | | | | | subfamily, member 5 |
| ENSG00000130522 | 19 | 18252251 | 18253294 | 1 | JUND | jun D proto-oncogene |
| ENSG00000131187 | 3 | 5 | 176761747 | 176769183 | F12 | coagulation factor XII (Hageman factor) |
| ENSG00000131828 | X | 19271968 | 19289724 | 5 | PDHA1 | pyruvate dehydrogenase (lipoamide) alpha 1 |
| ENSG00000132693 | 1 | 157948703 | 157951003 | 5 | CRP | C-reactive protein, pentraxin-related |
| ENSG00000133019 | 1 | 237859012 | 238145373 | 2 | CHRM3 | cholinergic receptor, muscarinic 3 |
| ENSG00000133216 | 1 | 22910045 | 23114405 | 4 | EPHB2 | EPH receptor B2 |
| ENSG00000134352 | 5 | 55266680 | 55326529 | 8 | IL6ST | interleukin 6 signal transducer |
| | | | | | | (gp130, oncostatin M receptor) |
| ENSG00000134571 | 11 | 47309527 | 47330806 | 1 | MYBPC3 | myosin binding protein C, cardiac |
| ENSG00000134755 | 18 | 26900005 | 26936375 | 2 | DSC2 | desmocollin 3 |
| ENSG00000134769 | 18 | 30327279 | 30725341 | 6 | DTNA | dystrobrevin, alpha |
| ENSG00000135047 | 9 | 89530254 | 89536127 | 3 | CTSL | cathepsin L |
| ENSG00000135447 | 12 | 53257439 | 53268723 | 2 | PPP1R1A | protein phosphatase 1, regulatory (inhibitor) subunit 1A |
| ENSG00000135486 | 12 | 52960755 | 52965297 | 2 | HNRPA1 | heterogeneous nuclear ribonucleoprotein A1 |
| ENSG00000135744 | 1 | 228904892 | 228916666 | 1 | AGT | angiotensinogen (serpin peptidase inhibitor, |
| | | | | | | clade A, member 8) |
| ENSG00000135750 | 1 | 231816373 | 231874881 | 3 | KCNK1 | potassium channel, subfamily K, member 1 |
| ENSG00000136238 | 7 | 6380651 | 6410120 | 2 | RAC1 | ras-related C3 botulinum toxin substrate 1 (rho family, |
| | | | | | | small GTP binding protein Rac1) |
| ENSG00000136244 | 7 | 22732028 | 22738091 | 1 | IL6 | interleukin 6 (interferon, beta 2) |
| ENSG00000136450 | 17 | 53437651 | 53439593 | 2 | SFRS1 | splicing factor, arginine/serine-rich 1 |
| | | | | | | (splicing factor 2, alternate splicing factor) |
| ENSG00000136574 | 8 | 11599122 | 11654920 | 3 | GATA4 | GATA binding protein 4 |
| ENSG00000136634 | 1 | 205007570 | 205012462 | 1 | IL10 | interleukin 10 |
| ENSG00000136842 | 9 | 99303742 | 99403357 | 2 | TMOD1 | tropomodulin 1 |
| ENSG00000137076 | 9 | 35687336 | 35722369 | 6 | TLN1 | talin 1 |
| ENSG00000137462 | 4 | 154842102 | 154846301 | 1 | TLR2 | toll-like receptor 2 |
| ENSG00000137745 | 11 | 102318937 | 102331672 | 2 | MMP13 | matrix metallopeptidase 13 (collagenase 3) |
| ENSG00000137808 | 15 | 67094125 | 67136516 | 2 | NOX5 | NADPH oxidase, EF-hand calcium binding domain 5 |
| ENSG00000138095 | 2 | 43968391 | 44076648 | 3 | LRPPRC | leucine-rich PPR-motif containing |
| ENSG00000138622 | 15 | 71400988 | 71448230 | 1 | HCN4 | hyperpolarization activated cyclic nucleotide-gated |
| | | | | | | potassium channel 4 |
| ENSG00000138685 | 4 | 123967313 | 124038840 | 1 | FGF2 | fibroblast growth factor 2 (basic) |
| ENSG00000138814 | 4 | 102163610 | 102487376 | 1 | PPP3CA | protein phosphatase 3 (formerly 2B), catalytic subunit, |
| | | | | | | alpha isoform (calcineurin A alpha) |
| ENSG00000139133 | 12 | 34066483 | 34072501 | 1 | ALG10A | asparagine-linked glycosylation 10 homolog |
| | | | | | | (yeast, alpha-1,2-glucosyltransferase) |
| ENSG00000140416 | 15 | 61121891 | 61151164 | 7 | TPM1 | tropomyosin 1 (alpha) |
| ENSG00000140564 | 15 | 89212889 | 89227691 | 1 | FURIN | furin (paired basic amino acid cleaving enzyme) |
| ENSG00000141646 | 18 | 46810611 | 46860142 | 1 | SMAD4 | SMAD family member 4 |
| ENSG00000142208 | 14 | 104306734 | 104333125 | 1 | AKT1 | v-akt murine thymoma viral oncogene homolog 1 |
| ENSG00000142871 | 1 | 85819005 | 85822233 | 2 | CYR61 | cysteine-rich, angiogenic inducer, 61 |
| ENSG00000143105 | 1 | 110861396 | 110862983 | 2 | KCNA10 | potassium voltage-gated channel, shaker-related |
| | | | | | | subfamily, member 10 |
| ENSG00000143140 | 1 | 145695517 | 145712066 | 2 | GJA5 | gap junction protein, alpha 5, 40 kDa (connexin 40) |
| ENSG00000143153 | 1 | 167341559 | 167368584 | 3 | ATP1B1 | ATPase, Na+/K+ transporting, beta 1 polypeptide |
| ENSG00000143318 | 1 | 158426970 | 158438300 | 2 | CASQ1 | calsequestrin 1 (fast-twitch, skeletal muscle) |
| ENSG00000143933 | 2 | 47240736 | 47257140 | 1 | CALM2 | calmodulin 2 (phosphorylase kinase, delta) |
| ENSG00000144285 | 2 | 166553919 | 166638395 | 3 | SCN1A | sodium channel, voltage-gated, type I, alpha |
| ENSG00000144891 | 3 | 149898355 | 149943478 | 1 | AGTR1 | angiotensin II receptor, type 1 |
| ENSG00000145349 | 4 | 114593022 | 114902177 | 4 | CAMK2D | calcium/calmodulin-dependent protein kinase |
| | | | | | | (CaM kinase) II delta |
| ENSG00000145362 | 4 | 114190319 | 114524334 | 4 | ANK2 | ankyrin 2, neuronal |
| ENSG00000145740 | 5 | 68425839 | 68462648 | 2 | SLC30A5 | solute carrier family 30 (zinc transporter), member 5 |
| ENSG00000146070 | 6 | 46779897 | 46811389 | 2 | PLA2G7 | phospholipase A2, group VII (platelet-activating |
| | | | | | | factor acetylhydrolase, plasma) |
| ENSG00000147044 | X | 41259131 | 41667660 | 8 | CASK | calcium/calmodulin-dependent serine protein kinase |
| | | | | | | (MAGUK family) |
| ENSG00000147166 | X | 70438309 | 70441946 | 1 | ITGB1BP2 | integrin beta 1 binding protein (melusin) 2 |
| ENSG00000148290 | 9 | 135208431 | 135213182 | 1 | SURF1 | surfeit 1 |
| ENSG00000148677 | 10 | 92661833 | 92671013 | 1 | ANKRD1 | ankyrin repeat domain 1 (cardiac muscle) |
| ENSG00000148926 | 11 | 10283172 | 10285491 | 1 | ADM | adrenomedullin |
| ENSG00000149575 | 11 | 117538729 | 117552546 | 1 | SCN2B | sodium channel, voltage-gated, type II, beta |
| ENSG00000149596 | 20 | 42173749 | 42249632 | 2 | JPH2 | junctophilin 2 |
| ENSG00000149968 | 11 | 102211738 | 102219552 | 1 | MMP3 | matrix metallopeptidase 3 (stromelysin 1, progelatinase) |
| ENSG00000150281 | 16 | 30815429 | 30822381 | 1 | CTF1 | cardiotrophin 1 |
| ENSG00000150594 | 10 | 112826911 | 112830655 | 2 | ADRA2A | adrenergic, alpha-2A-, receptor |
| ENSG00000150995 | 3 | 4510136 | 4863432 | 4 | ITPR1 | inositol 1,4,5-triphosphate receptor, type 1 |
| ENSG00000151062 | 12 | 1771384 | 1898131 | 2 | CACNA2D4 | calcium channel, voltage-dependent, alpha 2/delta subunit 4 |
| ENSG00000151067 | 12 | 2094650 | 2670626 | 5 | CACNA1C | calcium channel, voltage-dependent, L type, |
| | | | | | | alpha 1C subunit |
| ENSG00000151079 | 12 | 4789372 | 4791132 | 3 | KCNA6 | potassium voltage-gated channel, shaker-related |
| | | | | | | subfamily, member 6 |
| ENSG00000151150 | 10 | 61458165 | 61819494 | 6 | ANK3 | ankyrin 3, node of Ranvier (ankyrin G) |
| ENSG00000151320 | 14 | 31868274 | 32372018 | 1 | AKAP6 | A kinase (PRKA) anchor protein 6 |
| ENSG00000151623 | 4 | 149219370 | 149582973 | 4 | NR3C2 | nuclear receptor subfamily 3, group C, member 2 |
| ENSG00000151704 | 11 | 128213125 | 128242478 | 2 | KCNJ1 | potassium inwardly-rectifying channel, |
| | | | | | | subfamily J, member 1 |
| ENSG00000151729 | 4 | 186301392 | 186305418 | 1 | SLC25A4 | solute carrier family 25 (mitochondrial carrier; |
| | | | | | | adenine nucleotide translocator), member 4 |
| ENSG00000152049 | 2 | 223625171 | 223626872 | 1 | KCNE4 | potassium voltage-gated channel, lsk-related family, |
| | | | | | | member 4 |
| ENSG00000152413 | 5 | 78707505 | 78788599 | 2 | HOMER1 | homer homolog 1 (Drosophila) |
| ENSG00000152661 | 6 | 121798487 | 121812571 | 1 | GJA1 | gap junction protein, alpha 1, 43 kDa (connexin 43) |
| ENSG00000153253 | 2 | 165652286 | 165768799 | 4 | SCN3A | sodium channel, voltage-gated, type III, alpha |
| ENSG00000153956 | 7 | 81417354 | 81910967 | 3 | CACNA2D1 | calcium channel, voltage-dependent, alpha 2/delta subunit 1 |
| ENSG00000154229 | 17 | 61729388 | 62237324 | 1 | PRKCA | protein kinase C, alpha |
| ENSG00000154358 | 1 | 226462454 | 226633198 | 9 | OBSCN | obscurin, cytoskeletal calmodulin and |
| | | | | | | titin-interacting RhoGEF |
| ENSG00000155657 | 2 | 179099985 | 179380394 | 12 | TTN | titin |
| ENSG00000156475 | 5 | 145949265 | 146415783 | 2 | PPP2R2B | protein phosphatase 2 (formerly 2A), |
| | | | | | | regulatory subunit B (PR 52), beta isoform |
| ENSG00000157150 | 3 | 12169578 | 12175851 | 1 | TIMP4 | TIMP metallopeptidase inhibitor 4 |
| ENSG00000157227 | 14 | 22375676 | 22385088 | 1 | MMP14 | matrix metallopeptidase 14 (membrane-inserted) |
| ENSG00000157388 | 3 | 53503723 | 53821112 | 2 | CACNA1D | calcium channel, voltage-dependent, L type, |
| | | | | | | alpha 1D subunit |
| ENSG00000157445 | 3 | 54131733 | 55083622 | 1 | CACNA2D3 | calcium channel, voltage-dependent, alpha 2/delta 3 subunit |
| ENSG00000158022 | 1 | 26250382 | 26266711 | 1 | TRIM63 | tripartite motif-containing 63 |
| ENSG00000158125 | 2 | 31410691 | 31491117 | 2 | XDH | xanthine dehydrogenase |
| ENSG00000158445 | 20 | 47418353 | 47532591 | 1 | KCNB1 | potassium voltage-gated channel, Shab-related subfamily, |
| | | | | | | member 1 |
| ENSG00000159197 | 21 | 34658193 | 34665307 | 1 | KCNE2 | potassium voltage-gated channel, lsk-related |
| | | | | | | family, member 2 |
| ENSG00000159251 | 15 | 32869724 | 32875181 | 1 | ACTC1 | actin, alpha, cardiac muscle |
| ENSG00000159640 | 17 | 58908166 | 58938721 | 2 | ACE | angiotensin I converting enzyme (peptidyl-dipeptidase A) 1 |
| ENSG00000160014 | 19 | 51796352 | 51805878 | 1 | CALM3 | calmodulin 3 (phosphorylase kinase, delta) |
| ENSG00000160789 | 1 | 154318993 | 154376504 | 9 | LMNA | lamin A/C |
| ENSG00000160808 | 3 | 46874371 | 46879938 | 1 | MYL3 | myosin, light polypeptide 3, alkali; ventricular, |
| | | | | | | skeletal, slow |
| ENSG00000161547 | 17 | 72241796 | 72244837 | 3 | SFRS2 | splicing factor, arginine/serine-rich 2 |
| ENSG00000161570 | 17 | 31222613 | 31231490 | 1 | CCL5 | chemokine (C-C motif) ligand 5 |
| ENSG00000162989 | 2 | 155263339 | 155421260 | 1 | KCNJ3 | potassium inwardly-rectifying channel, |
| | | | | | | subfamily J, member 3 |
| ENSG00000163399 | 1 | 116717359 | 116754301 | 4 | ATP1A1 | ATPase, Na+/K+ transporting, alpha 1 polypeptide |
| ENSG00000163485 | 1 | 201326405 | 201403156 | 4 | ADORA1 | adenosine A1 receptor |
| ENSG00000164056 | 4 | 124537406 | 124544357 | 1 | SPRY1 | sprouty homolog 1, antagonist of FGF signaling |
| | | | | | | (Drosophila) |
| ENSG00000164171 | 1 | 5 | 52321014 | 52423805 | ITGA2 | integrin, alpha 2 (CD49B, alpha 2 subunit of |
| | | | | | | VLA-2 receptor) |
| ENSG00000164258 | 5 | 52892226 | 53014925 | 2 | NDUFS4 | NADH dehydrogenase (ubiquinone) Fe—S protein 4, |
| | | | | | | 18 kDa (NADH-coenzyme Q reductase) |
| ENSG00000164305 | 4 | 185785845 | 185807623 | 2 | CASP3 | caspase 3, apoptosis-related cysteine peptidase |
| ENSG00000164588 | 5 | 45297730 | 45731977 | 1 | HCN1 | hyperpolarization activated cyclic nucleotide- |
| | | | | | | gated potassium channel 1 |
| ENSG00000165119 | 9 | 85772818 | 85785339 | 8 | HNRPK | heterogeneous nuclear ribonucleoprotein K |
| ENSG00000165995 | 10 | 18469612 | 18870797 | 9 | CACNB2 | calcium channel, voltage-dependent, beta 2 subunit |
| ENSG00000166068 | 15 | 36331808 | 36433526 | 1 | SPRED1 | sprouty-related, EVH1 domain containing 1 |
| ENSG00000166257 | 11 | 123005107 | 123030165 | 1 | SCN3B | sodium channel, voltage-gated, type III, beta |
| ENSG00000166501 | 16 | 23754823 | 24139358 | 2 | PRKCB1 | protein kinase C, beta 1 |
| ENSG00000166949 | 15 | 65145249 | 65274586 | 1 | SMAD3 | SMAD family member 3 |
| ENSG00000167535 | 12 | 47498779 | 47508991 | 1 | CACNB3 | calcium channel, voltage-dependent, beta 3 subunit |
| ENSG00000167792 | 11 | 67130974 | 67136581 | 1 | NDUFV1 | NADH dehydrogenase (ubiquinone) flavoprotein 1, 51 kDa |
| ENSG00000168028 | 3 | 39423208 | 39429034 | 1 | RPSA | ribosomal protein SA (LAMR1) |
| ENSG00000168135 | 22 | 37152278 | 37181149 | 1 | KCNJ4 | potassium inwardly-rectifying channel, subfamily J, |
| | | | | | | member 4 |
| ENSG00000168542 | 2 | 189547344 | 189585717 | 2 | COL3A1 | collagen, type III, alpha 1 (Ehlers-Danlos |
| | | | | | | syndrome type IV, autosomal dominant) |
| ENSG00000168610 | 17 | 37718869 | 37794039 | 5 | STAT3 | signal transducer and activator of transcription |
| | | | | | | 3 (acute-phase response factor) |
| ENSG00000168807 | 16 | 67778533 | 67892379 | 2 | SNTB2 | syntrophin, beta 2 (dystrophin-associated |
| | | | | | | protein A1, 59 kDa, basic component 2) |
| ENSG00000169252 | 5 | 148185001 | 148188447 | 1 | ADRB2 | adrenergic, beta-2-, receptor, surface |
| ENSG00000169282 | 3 | 157321095 | 157739237 | 10 | KCNAB1 | potassium voltage-gated channel, |
| | | | | | | shaker-related subfamily, beta member 1 |
| ENSG00000169418 | 1 | 151917737 | 151933092 | 2 | NPR1 | natriuretic peptide receptor A/guanylate cyclase A |
| | | | | | | (atrionatriuretic peptide receptor A) |
| ENSG00000169432 | 2 | 166763060 | 166876560 | 2 | SCN9A | sodium channel, voltage-gated, type IX, alpha |
| ENSG00000169562 | X | 70351769 | 70362091 | 3 | GJB1 | gap junction protein, beta 1, 32 kDa (connexin 32, |
| | | | | | | Charcot-Marie-Tooth neuropathy, X-linked) |
| ENSG00000169564 | 2 | 70168090 | 70169766 | 1 | PCBP1 | poly(rC) binding protein 1 |
| ENSG00000170049 | 17 | 7765902 | 7773478 | 2 | KCNAB3 | potassium voltage-gated channel, shaker-related |
| | | | | | | subfamily, beta member 3 |
| ENSG00000170214 | 5 | 159276318 | 159332595 | 1 | ADRA1B | adrenergic, alpha-1B-, receptor |
| ENSG00000170290 | 11 | 107083319 | 107087992 | 1 | SLN | sarcolipin |
| ENSG00000170425 | 17 | 15788956 | 15819935 | 1 | ADORA2B | adenosine A2b receptor |
| ENSG00000170624 | 5 | 155686334 | 156125623 | 1 | SGCD | sarcoglycan, delta (35 kDa dystrophin- |
| | | | | | | associated glycoprotein) |
| ENSG00000170776 | 15 | 83578821 | 84093590 | 3 | AKAP13 | A kinase (PRKA) anchor protein 13 |
| ENSG00000170962 | 11 | 103283131 | 103540317 | 1 | PDGFD | platelet derived growth factor D |
| ENSG00000171303 | 2 | 26769123 | 26806207 | 1 | KCNK3 | potassium channel, subfamily K, member 3 |
| ENSG00000171385 | 1 | 112114807 | 112333300 | 3 | KCND3 | potassium voltage-gated channel, Shal-related |
| | | | | | | subfamily, member 3 |
| ENSG00000171497 | 4 | 159849730 | 159864002 | 1 | PPID | peptidylprolyl isomerase D (cyclophilin D) |
| ENSG00000171552 | 20 | 29715916 | 29774366 | 4 | BCL2L1 | BCL2-like 1 |
| ENSG00000171564 | 1 | 4 | 155703596 | 155711683 | FGB | fibrinogen beta chain |
| ENSG00000171786 | 1 | 158603481 | 158609262 | 1 | NHLH1 | nescient helix loop helix 1 |
| ENSG00000171873 | 20 | 4149329 | 4177659 | 1 | ADRA1D | adrenergic, alpha-1D-, receptor |
| ENSG00000172164 | 8 | 121619297 | 121893264 | 1 | SNTB1 | syntrophin, beta 1 (dystrophin-associated protein A1, |
| | | | | | | 59 kDa, basic component 1) |
| ENSG00000172270 | 19 | 462896 | 534492 | 3 | BSG | basigin (Ok blood group) |
| ENSG00000172399 | 4 | 120276469 | 120328383 | 1 | MYOZ2 | myozenin 2 |
| ENSG00000172531 | 11 | 66922228 | 66925978 | 3 | PPP1CA | protein phosphatase 1, catalytic subunit, alpha isoform |
| ENSG00000173020 | 11 | 66790507 | 66810602 | 1 | ADRBK1 | adrenergic, beta, receptor kinase 1 |
| ENSG00000173801 | 17 | 37164412 | 37196476 | 1 | JUP | junction plakoglobin |
| ENSG00000173991 | 17 | 35073966 | 35076326 | 1 | TCAP | titin-cap (telethonin) |
| ENSG00000174437 | 12 | 109203815 | 109273278 | 3 | ATP2A2 | ATPase, Ca++ transporting, cardiac muscle, slow twitch 2 |
| ENSG00000175084 | 2 | 219991343 | 219999705 | 2 | DES | desmin |
| ENSG00000175387 | 18 | 43618435 | 43711221 | 2 | SMAD2 | SMAD family member 2 |
| ENSG00000175538 | 11 | 73843536 | 73856186 | 1 | KCNE3 | potassium voltage-gated channel, lsk-related family, |
| | | | | | | member 3 |
| ENSG00000175548 | 12 | 36996824 | 37001523 | 1 | ALG10B | asparagine-linked glycosylation 10 homolog B |
| | | | | | | (yeast, alpha-1,2-glucosyltransferase) (KCR1) |
| ENSG00000176076 | X | 108753585 | 108755057 | 2 | KCNE1L | KCNE1-like |
| ENSG00000177000 | 4 | 1 | 11768367 | 11788702 | MTHFR | 5,10-methylenetetrahydrofolate reductase (NADPH) |
| ENSG00000177098 | 11 | 117509302 | 117528747 | 1 | SCN4B | sodium channel, voltage-gated, type IV, beta |
| ENSG00000177885 | 17 | 70825753 | 70913384 | 2 | GRB2 | growth factor receptor-bound protein 2 |
| ENSG00000179142 | 8 | 143988983 | 143996261 | 1 | CYP11B2 | cytochrome P450, family 11, subfamily B, polypeptide 2 |
| ENSG00000179295 | 12 | 111340919 | 111432099 | 1 | PTPN11 | protein tyrosine phosphatase, non-receptor type 11 |
| | | | | | | (Noonan syndrome 1) |
| ENSG00000180210 | 3 | 11 | 46697331 | 46717631 | F2 | coagulation factor II (thrombin) |
| ENSG00000180509 | 21 | 34740858 | 34806443 | 1 | KCNE1 | potassium voltage-gated channel, lsk-related |
| | | | | | | family, member 1 |
| ENSG00000180733 | 8 | 48812794 | 48813235 | 1 | CEBPD | CCAAT/enhancer binding protein (C/EBP), delta |
| ENSG00000180817 | 10 | 71632592 | 71663196 | 2 | PPA1 | pyrophosphatase (inorganic) 1 |
| ENSG00000181210 | 2 | 96202419 | 96203762 | | ADRA2B | adrenergic, alpha-2B-, receptor |
| ENSG00000182255 | 11 | 29988341 | 29995064 | 1 | KCNA4 | potassium voltage-gated channel, shaker-related |
| | | | | | | subfamily, member 4 |
| ENSG00000182389 | 2 | S | 152663771 | 1 | CACNB4 | calcium channel, voltage-dependent, beta 4 subunit |
| ENSG00000182450 | 11 | 63815770 | 63828817 | 1 | KCNK4 | potassium channel, subfamily K, member 4 |
| ENSG00000182533 | 3 | 8750253 | 8763451 | 2 | CAV3 | caveolin 3 |
| ENSG00000182687 | 17 | 71582479 | 71585168 | 1 | GALR2 | galanin receptor 2 |
| ENSG00000182963 | 17 | 40237146 | 40263707 | 1 | GJA7 | gap junction protein, alpha 7, 45 kDa (connexin 45) |
| ENSG00000183023 | 2 | 40192790 | 40534188 | 5 | SLC8A1 | solute carrier family 8 (sodium/calcium |
| | | | | | | exchanger), member 1 |
| ENSG00000183072 | 5 | 172591744 | 172594868 | 1 | NKX2-5 | NK2 transcription factor related, locus 5 (Drosophila) |
| ENSG00000183873 | 3 | 38564558 | 38666167 | 2 | SCN5A | sodium channel, voltage-gated, type V, alpha |
| | | | | | | (long QT syndrome 3) |
| ENSG00000184160 | 4 | 3738094 | 3740051 | | ADRA2C | adrenergic, alpha-2C-, receptor |
| ENSG00000184185 | 17 | 21220292 | 21260983 | 1 | KCNJ12 | potassium inwardly-rectifying channel, subfamily J, |
| | | | | | | member 12 |
| ENSG00000184408 | 7 | 119701923 | 120175148 | 1 | KCND2 | potassium voltage-gated channel, Shal-related |
| | | | | | | subfamily, member 2 |
| ENSG00000186439 | 6 | 123579183 | 123999937 | 5 | TRDN | triadin |
| ENSG00000187486 | 11 | 17365042 | 17366214 | 1 | KCNJ11 | potassium inwardly-rectifying channel, subfamily J, |
| | | | | | | member 11 |
| ENSG00000188386 | 9 | 103393718 | 103397104 | 2 | PPP3R2 | protein phosphatase 3 (formerly 2B), |
| | | | | | | regulatory subunit B, beta isoform |
| ENSG00000188389 | 2 | 242440711 | 242449731 | 2 | PDCD1 | programmed cell death 1 |
| ENSG00000188778 | 8 | 37939673 | 37943341 | 1 | ADRB3 | adrenergic, beta-3-, receptor |
| ENSG00000196218 | 19 | 43616180 | 43770012 | 5 | RYR1 | ryanodine receptor 1 (skeletal) |
| ENSG00000196296 | 16 | 28797310 | 28823331 | 1 | ATP2A1 | ATPase, Ca++ transporting, cardiac muscle, fast twitch 1 |
| ENSG00000196557 | 16 | 1143739 | 1211772 | 2 | CACNA1H | calcium channel, voltage-dependent, alpha 1H subunit |
| ENSG00000196611 | 11 | 102165861 | 102174099 | 1 | MMP1 | matrix metallopeptidase 1 (interstitial collagenase) |
| ENSG00000197442 | 6 | 136919878 | 137155349 | 3 | MAP3K5 | mitogen-activated protein kinase kinase kinase 5 |
| ENSG00000197616 | 14 | 22921038 | 22946665 | 2 | MYH6 | myosin, heavy polypeptide 6, cardiac muscle, alpha |
| | | | | | | (cardiomyopathy, hypertrophic 1) |
| ENSG00000198216 | 1 | 179648918 | 180037339 | 6 | CACNA1E | calcium channel, voltage-dependent, alpha 1E subunit |
| ENSG00000198363 | 8 | 62578374 | 62789681 | 11 | ASPH | aspartate beta-hydroxylase |
| ENSG00000198523 | 6 | 118976154 | 118988586 | 1 | PLN | phospholamban |
| ENSG00000198626 | 1 | 235272128 | 236063911 | 3 | RYR2 | ryanodine receptor 2 (cardiac) |
| ENSG00000198668 | 14 | 89933120 | 89944158 | | CALM1 | calmodulin 1 (phosphorylase kinase, delta) |
| ENSG00000198734 | 3 | 1 | 167750028 | 167822450 | F5 | coagulation factor V (proaccelerin, labile factor) |
| ENSG00000198929 | 1 | 160306190 | 160604868 | 1 | NOS1AP | nitric oxide synthase 1 (neuronal) adaptor protein |
| ENSG00000198947 | X | 31047257 | 33267479 | 15 | DMD | dystrophin (muscular dystrophy, Duchenne and |
| | | | | | | Becker types) |
| ENSG00000204490 | 6 | 31651314 | 31654092 | 1 | TNF | tumor necrosis factor (TNF superfamily, member 2) |
| NOT FOUND | | | | | CLNS1B | chloride channel, nucleotide-sensitive, 1B |
| NOT FOUND | | | | | GP1BB | glycoprotein Ib (platelet), beta polypeptide |
| NOT FOUND | | | | | SERPINE1 | serpin peptidase inhibitor, clade E (nexin, plasminogen |
| | | | | | | activator inhibitor type 1), member 1 |

TABLE 14

| pid | coef | stderr | pval | pval_holm | pval_bonf | pval_fdr | p_nc | maf | hwe |
|---|---|---|---|---|---|---|---|---|---|
| SNP_A- | −0.9697 | 0.1806 | 7.96e−08 | 0.0596 | 0.0596 | 0.0596 | 0.00304 | 0.0343 | 0.00511 |
| 2053054 | | | | | | | | | |
| SNP_A- | −0.3608 | 0.07674 | 2.58e−06 | 1 | 1 | 0.241 | 0.0228 | 0.241 | 0.215 |
| 8370399 | | | | | | | | | |
| SNP_A- | 0.9792 | 0.2108 | 3.4e−06 | 1 | 1 | 0.273 | 0 | 0.038 | 1 |
| 8631553 | | | | | | | | | |
| SNP_A- | 0.5797 | 0.1339 | 1.49e−05 | 1 | 1 | 0.451 | 0.00456 | 0.111 | 0.233 |
| 8285583 | | | | | | | | | |
| SNP_A- | −0.5908 | 0.137 | 1.62e−05 | 1 | 1 | 0.451 | 0 | 0.112 | 0.844 |
| 1854346 | | | | | | | | | |
| SNP_A- | −0.9818 | 0.2343 | 2.79e−05 | 1 | 1 | 0.461 | 0.00152 | 0.0104 | 0.961 |
| 8647043 | | | | | | | | | |
| SNP_A- | 0.542 | 0.1316 | 3.84e−05 | 1 | 1 | 0.465 | 0.00456 | 0.143 | 0.109 |
| 2183445 | | | | | | | | | |
| SNP_A- | 0.7006 | 0.1763 | 7.1e−05 | 1 | 1 | 0.539 | 0.0365 | 0.0536 | 0.699 |
| 8530310 | | | | | | | | | |
| SNP_A- | −0.6793 | 0.1741 | 9.56e−05 | 1 | 1 | 0.563 | 0.00304 | 0.0655 | 1 |
| 8456423 | | | | | | | | | |
| SNP_A- | 0.6486 | 0.1592 | 4.63e−05 | 1 | 1 | 0.487 | 0.00304 | 0.0716 | 0.133 |
| 8596066 | | | | | | | | | |
| SNP_A- | 0.5127 | 0.1291 | 7.14e−05 | 1 | 1 | 0.539 | 0 | 0.138 | 1 |
| 8582554 | | | | | | | | | |
| SNP_A- | −0.7061 | 0.1478 | 1.76e−06 | 1 | 1 | 0.241 | 0.00456 | 0.074 | 0.773 |
| 1967375 | | | | | | | | | |
| SNP_A- | −1.429 | 0.2992 | 1.77e−06 | 1 | 1 | 0.241 | 0.00152 | 0.0145 | 1 |
| 8366760 | | | | | | | | | |
| SNP_A- | 0.7065 | 0.147 | 1.54e−06 | 1 | 1 | 0.241 | 0.00456 | 0.0756 | 0.78 |
| 8478064 | | | | | | | | | |
| SNP_A- | −0.8792 | 0.1859 | 2.27e−06 | 1 | 1 | 0.241 | 0.0258 | 0.0484 | 0.39 |
| 8349850 | | | | | | | | | |
| SNP_A- | −0.4554 | 0.1244 | 0.000251 | 1 | 1 | 0.669 | 0.0167 | 0.237 | 0.277 |
| 8544970 | | | | | | | | | |
| SNP_A- | 0.628 | 0.1608 | 9.41e−05 | 1 | 1 | 0.563 | 0 | 0.0714 | 0.132 |
| 1988493 | | | | | | | | | |
| SNP_A- | 0.4471 | 0.1256 | 0.000371 | 1 | 1 | 0.69 | 0.00912 | 0.243 | 0.523 |
| 4265535 | | | | | | | | | |
| SNP_A- | −0.4396 | 0.1239 | 0.000388 | 1 | 1 | 0.69 | 0 | 0.248 | 0.835 |
| 2080370 | | | | | | | | | |
| SNP_A- | −0.4396 | 0.1239 | 0.000388 | 1 | 1 | 0.69 | 0 | 0.248 | 0.835 |
| 2092022 | | | | | | | | | |
| SNP_A- | −0.3714 | 0.1048 | 0.000392 | 1 | 1 | 0.69 | 0.0076 | 0.252 | 0.0487 |
| 2202302 | | | | | | | | | |
| SNP_A- | 0.4378 | 0.1234 | 0.000388 | 1 | 1 | 0.69 | 0.0182 | 0.254 | 0.678 |
| 1959929 | | | | | | | | | |
| SNP_A- | −0.4403 | 0.1239 | 0.00038 | 1 | 1 | 0.69 | 0.0076 | 0.249 | 0.676 |
| 8668446 | | | | | | | | | |
| SNP_A- | 0.4396 | 0.1239 | 0.000388 | 1 | 1 | 0.69 | 0.00304 | 0.248 | 0.835 |
| 8417654 | | | | | | | | | |
| SNP_A- | 0.3838 | 0.09517 | 5.51e−05 | 1 | 1 | 0.494 | 0 | 0.366 | 0.675 |
| 8386477 | | | | | | | | | |
| SNP_A- | −0.4256 | 0.1234 | 0.000562 | 1 | 1 | 0.722 | 0 | 0.247 | 0.916 |
| 1985257 | | | | | | | | | |
| SNP_A- | 0.4256 | 0.1234 | 0.000562 | 1 | 1 | 0.722 | 0.00304 | 0.246 | 0.753 |
| 1896426 | | | | | | | | | |
| SNP_A- | −0.7528 | 0.1626 | 3.64e−06 | 1 | 1 | 0.273 | 0.0167 | 0.0672 | 0.76 |
| 4272029 | | | | | | | | | |
| SNP_A- | −0.9908 | 0.2372 | 2.96e−05 | 1 | 1 | 0.461 | 0 | 0.0274 | 1 |
| 2168866 | | | | | | | | | |
| SNP_A- | −0.978 | 0.2373 | 3.76e−05 | 1 | 1 | 0.465 | 0 | 0.0281 | 1 |
| 1787940 | | | | | | | | | |
| SNP_A- | −0.8545 | 0.2022 | 2.38e−05 | 1 | 1 | 0.461 | 0 | 0.0327 | 0.147 |
| 4224892 | | | | | | | | | |
| SNP_A- | 0.5037 | 0.1061 | 2.06e−06 | 1 | 1 | 0.241 | 0 | 0.239 | 0.52 |
| 8470071 | | | | | | | | | |
| SNP_A- | −0.4588 | 0.1025 | 7.62e−06 | 1 | 1 | 0.368 | 0 | 0.34 | 0.794 |
| 1803248 | | | | | | | | | |
| SNP_A- | −0.4563 | 0.1015 | 6.93e−06 | 1 | 1 | 0.368 | 0 | 0.305 | 0.311 |
| 8565161 | | | | | | | | | |
| SNP_A- | 1.103 | 0.2468 | 7.84e−06 | 1 | 1 | 0.368 | 0.0137 | 0.0247 | 0.324 |
| 8351277 | | | | | | | | | |
| SNP_A- | −0.4254 | 0.1234 | 0.000564 | 1 | 1 | 0.722 | 0.00152 | 0.247 | 1 |
| 8668443 | | | | | | | | | |
| SNP_A- | 1.008 | 0.3107 | 0.00118 | 1 | 1 | 0.81 | 0 | 0.0137 | 1 |
| 8421072 | | | | | | | | | |
| SNP_A- | −0.3582 | 0.1046 | 0.000613 | 1 | 1 | 0.723 | 0.00152 | 0.254 | 0.0507 |
| 1842166 | | | | | | | | | |
| SNP_A- | −0.4625 | 0.1223 | 0.000156 | 1 | 1 | 0.601 | 0.00152 | 0.15 | 0.879 |
| 4220021 | | | | | | | | | |
| SNP_A- | 1.211 | 0.2522 | 1.56e−06 | 1 | 1 | 0.241 | 0.00456 | 0.0137 | 0.112 |
| 8299340 | | | | | | | | | |
| SNP_A- | −0.853 | 0.2267 | 0.000168 | 1 | 1 | 0.614 | 0 | 0.0304 | 1 |
| 2152929 | | | | | | | | | |
| SNP_A- | 0.7088 | 0.1825 | 0.000103 | 1 | 1 | 0.565 | 0 | 0.0479 | 1 |
| 1953240 | | | | | | | | | |
| SNP_A- | −0.8963 | 0.3249 | 0.00581 | 1 | 1 | 0.869 | 0.00608 | 0.0122 | 1 |
| 2002771 | | | | | | | | | |
| SNP_A- | 0.2738 | 0.09666 | 0.00461 | 1 | 1 | 0.869 | 0 | 0.362 | 0.152 |
| 2047393 | | | | | | | | | |
| SNP_A- | −1.058 | 0.2454 | 1.63e−05 | 1 | 1 | 0.451 | 0.00152 | 0.0251 | 0.338 |
| 8672704 | | | | | | | | | |
| SNP_A- | 0.4334 | 0.1028 | 2.47e−05 | 1 | 1 | 0.461 | 0.00152 | 0.423 | 0.381 |
| 8677651 | | | | | | | | | |
| SNP_A- | −0.9755 | 0.233 | 2.84e−05 | 1 | 1 | 0.461 | 0 | 0.0236 | 0.0457 |
| 8493887 | | | | | | | | | |
| SNP_A- | 0.4167 | 0.1014 | 3.98e−05 | 1 | 1 | 0.465 | 0 | 0.305 | 0.854 |
| 8490285 | | | | | | | | | |
| SNP_A- | −0.9344 | 0.2193 | 2.04e−05 | 1 | 1 | 0.461 | 0.0213 | 0.0179 | 1.77e−05 |
| 2166983 | | | | | | | | | |
| SNP_A- | 0.7738 | 0.1917 | 5.42e−05 | 1 | 1 | 0.494 | 0.00152 | 0.035 | 0.184 |
| 8658724 | | | | | | | | | |
| SNP_A- | 0.4345 | 0.09654 | 6.77e−06 | 1 | 1 | 0.368 | 0.0304 | 0.416 | 0.745 |
| 8378831 | | | | | | | | | |
| SNP_A- | −1 | 0.233 | 1.76e−05 | 1 | 1 | 0.451 | 0 | 0.0289 | 1 |
| 8296527 | | | | | | | | | |
| SNP_A- | −1.181 | 0.3525 | 0.000805 | 1 | 1 | 0.757 | 0 | 0.0114 | 1 |
| 1972641 | | | | | | | | | |
| SNP_A- | −1.447 | 0.3273 | 9.84e−06 | 1 | 1 | 0.388 | 0 | 0.0114 | 1 |
| 8405569 | | | | | | | | | |
| SNP_A- | 0.4402 | 0.1223 | 0.000318 | 1 | 1 | 0.67 | 0 | 0.15 | 1 |
| 2171537 | | | | | | | | | |
| SNP_A- | 0.3237 | 0.1167 | 0.00553 | 1 | 1 | 0.869 | 0 | 0.185 | 1 |
| 4252168 | | | | | | | | | |
| SNP_A- | 0.7064 | 0.2536 | 0.00535 | 1 | 1 | 0.869 | 0.0106 | 0.0276 | 1 |
| 8453740 | | | | | | | | | |
| SNP_A- | −0.5536 | 0.1363 | 4.89e−05 | 1 | 1 | 0.487 | 0 | 0.207 | 0.906 |
| 4252121 | | | | | | | | | |
| SNP_A- | 0.6841 | 0.1562 | 1.18e−05 | 1 | 1 | 0.42 | 0.0152 | 0.0633 | 1 |
| 2000347 | | | | | | | | | |
| SNP_A- | 0.5262 | 0.1163 | 6e−06 | 1 | 1 | 0.368 | 0.00304 | 0.168 | 0.674 |
| 8510071 | | | | | | | | | |
| SNP_A- | −0.4241 | 0.1166 | 0.000277 | 1 | 1 | 0.67 | 0 | 0.171 | 0.585 |
| 2083150 | | | | | | | | | |
| SNP_A- | −0.4241 | 0.1166 | 0.000277 | 1 | 1 | 0.67 | 0 | 0.171 | 0.585 |
| 4235811 | | | | | | | | | |
| SNP_A- | 0.3994 | 0.1084 | 0.00023 | 1 | 1 | 0.669 | 0.00304 | 0.351 | 0.494 |
| 8485648 | | | | | | | | | |
| SNP_A- | −0.3243 | 0.08004 | 5.08e−05 | 1 | 1 | 0.487 | 0.0137 | 0.204 | 0.426 |
| 8356840 | | | | | | | | | |
| SNP_A- | −0.4403 | 0.124 | 0.000382 | 1 | 1 | 0.69 | 0.0076 | 0.145 | 0.434 |
| 1834789 | | | | | | | | | |
| SNP_A- | −0.5352 | 0.1198 | 7.87e−06 | 1 | 1 | 0.368 | 0.00152 | 0.164 | 0.669 |
| 8642499 | | | | | | | | | |
| SNP_A- | 0.6073 | 0.1951 | 0.00185 | 1 | 1 | 0.812 | 0 | 0.0509 | 0.403 |
| 4205314 | | | | | | | | | |
| SNP_A- | −0.4182 | 0.09718 | 1.68e−05 | 1 | 1 | 0.451 | 0.00152 | 0.413 | 0.748 |
| 2152506 | | | | | | | | | |
| SNP_A- | −0.445 | 0.1347 | 0.000955 | 1 | 1 | 0.791 | 0.00152 | 0.112 | 0.844 |
| 8432970 | | | | | | | | | |
| SNP_A- | −0.4461 | 0.1087 | 4.09e−05 | 1 | 1 | 0.469 | 0 | 0.209 | 0.558 |
| 8548394 | | | | | | | | | |
| SNP_A- | −0.4188 | 0.09738 | 1.7e−05 | 1 | 1 | 0.451 | 0.00456 | 0.408 | 0.628 |
| 8681923 | | | | | | | | | |
| SNP_A- | 0.535 | 0.1252 | 1.93e−05 | 1 | 1 | 0.461 | 0.0122 | 0.147 | 1 |
| 8630612 | | | | | | | | | |
| SNP_A- | 0.6276 | 0.1442 | 1.34e−05 | 1 | 1 | 0.451 | 0 | 0.0775 | 0.58 |
| 4204345 | | | | | | | | | |
| SNP_A- | −0.4092 | 0.09948 | 3.9e−05 | 1 | 1 | 0.465 | 0.0304 | 0.433 | 0.126 |
| 8398578 | | | | | | | | | |
| SNP_A- | 0.4769 | 0.1082 | 1.05e−05 | 1 | 1 | 0.391 | 0 | 0.21 | 0.35 |
| 2243420 | | | | | | | | | |
| SNP_A- | 0.4061 | 0.09686 | 2.76e−05 | 1 | 1 | 0.461 | 0 | 0.412 | 0.872 |
| 2052179 | | | | | | | | | |
| SNP_A- | 0.4061 | 0.09686 | 2.76e−05 | 1 | 1 | 0.461 | 0 | 0.412 | 0.872 |
| 2059271 | | | | | | | | | |
| SNP_A- | 0.6998 | 0.1682 | 3.16e−05 | 1 | 1 | 0.465 | 0.00152 | 0.0548 | 1 |
| 2206221 | | | | | | | | | |
| SNP_A- | −0.404 | 0.09763 | 3.51e−05 | 1 | 1 | 0.465 | 0.00456 | 0.412 | 0.519 |
| 2262428 | | | | | | | | | |
| SNP_A- | 0.3975 | 0.09696 | 4.14e−05 | 1 | 1 | 0.469 | 0.00304 | 0.412 | 0.809 |
| 1864375 | | | | | | | | | |
| SNP_A- | −0.6269 | 0.1487 | 2.5e−05 | 1 | 1 | 0.461 | 0 | 0.189 | 1 |
| 1976890 | | | | | | | | | |
| SNP_A- | 0.4365 | 0.1053 | 3.38e−05 | 1 | 1 | 0.465 | 0.00152 | 0.368 | 0.801 |
| 4287784 | | | | | | | | | |
| SNP_A- | −0.4373 | 0.1052 | 3.22e−05 | 1 | 1 | 0.465 | 0.00304 | 0.37 | 0.867 |
| 1942320 | | | | | | | | | |
| SNP_A- | 0.4885 | 0.1176 | 3.24e−05 | 1 | 1 | 0.465 | 0 | 0.185 | 0.605 |
| 8456608 | | | | | | | | | |
| SNP_A- | 0.4549 | 0.1055 | 1.61e−05 | 1 | 1 | 0.451 | 0.0289 | 0.354 | 0.00562 |
| 8627377 | | | | | | | | | |
| SNP_A- | 0.4048 | 0.09589 | 2.43e−05 | 1 | 1 | 0.461 | 0 | 0.408 | 0.687 |
| 8366937 | | | | | | | | | |
| SNP_A- | −0.435 | 0.1055 | 3.71e−05 | 1 | 1 | 0.465 | 0 | 0.367 | 0.737 |
| 4273665 | | | | | | | | | |
| SNP_A- | −0.468 | 0.1163 | 5.68e−05 | 1 | 1 | 0.494 | 0 | 0.281 | 0.7 |
| 4195397 | | | | | | | | | |
| SNP_A- | −0.4406 | 0.09967 | 9.85e−06 | 1 | 1 | 0.388 | 0 | 0.486 | 0.938 |
| 2113673 | | | | | | | | | |
| AFFX- | −0.4406 | 0.09967 | 9.85e−06 | 1 | 1 | 0.388 | 0 | 0.486 | 0.938 |
| SNP_6891433 | | | | | | | | | |
| SNP_A- | −1.072 | 0.2663 | 5.66e−05 | 1 | 1 | 0.494 | 0.0365 | 0.0142 | 1 |
| 1965187 | | | | | | | | | |
| SNP_A- | −0.4025 | 0.09841 | 4.32e−05 | 1 | 1 | 0.482 | 0 | 0.467 | 0.938 |
| 1985390 | | | | | | | | | |
| SNP_A- | 0.4147 | 0.1009 | 3.97e−05 | 1 | 1 | 0.465 | 0.0365 | 0.386 | 0.0189 |
| 2199372 | | | | | | | | | |
| SNP_A- | −0.48 | 0.1162 | 3.61e−05 | 1 | 1 | 0.465 | 0.00152 | 0.167 | 0.483 |
| 2223920 | | | | | | | | | |
| SNP_A- | −0.4278 | 0.1048 | 4.45e−05 | 1 | 1 | 0.487 | 0.0076 | 0.364 | 0.673 |
| 2075251 | | | | | | | | | |
| SNP_A- | 0.4713 | 0.1161 | 4.95e−05 | 1 | 1 | 0.487 | 0 | 0.289 | 0.849 |
| 1965505 | | | | | | | | | |
| SNP_A- | 0.4148 | 0.09835 | 2.47e−05 | 1 | 1 | 0.461 | 0.00608 | 0.393 | 0.461 |
| 8603804 | | | | | | | | | |
| SNP_A- | −0.4013 | 0.09899 | 5.05e−05 | 1 | 1 | 0.487 | 0.0228 | 0.399 | 0.25 |
| 1962473 | | | | | | | | | |
| SNP_A- | −0.387 | 0.1251 | 0.00198 | 1 | 1 | 0.823 | 0.0243 | 0.154 | 0.0684 |
| 8613839 | | | | | | | | | |
| SNP_A- | −0.5053 | 0.1647 | 0.00215 | 1 | 1 | 0.835 | 0 | 0.0631 | 0.738 |
| 1957079 | | | | | | | | | |
| SNP_A- | −0.7168 | 0.2356 | 0.00235 | 1 | 1 | 0.836 | 0.00912 | 0.0284 | 1 |
| 8432286 | | | | | | | | | |
| SNP_A- | 0.4313 | 0.1071 | 5.64e−05 | 1 | 1 | 0.494 | 0 | 0.267 | 0.842 |
| 4288330 | | | | | | | | | |
| SNP_A- | 0.4313 | 0.1071 | 5.64e−05 | 1 | 1 | 0.494 | 0.00304 | 0.266 | 0.92 |
| 4300393 | | | | | | | | | |
| SNP_A- | −0.7276 | 0.1762 | 3.63e−05 | 1 | 1 | 0.465 | 0.00152 | 0.0609 | 1 |
| 8407616 | | | | | | | | | |
| SNP_A- | −0.4113 | 0.1012 | 4.78e−05 | 1 | 1 | 0.487 | 0 | 0.271 | 0.139 |
| 1949138 | | | | | | | | | |
| SNP_A- | −0.4552 | 0.1087 | 2.84e−05 | 1 | 1 | 0.461 | 0.00152 | 0.193 | 1 |
| 1908453 | | | | | | | | | |
| SNP_A- | 0.4069 | 0.09902 | 3.97e−05 | 1 | 1 | 0.465 | 0.00152 | 0.464 | 0.875 |
| 8596473 | | | | | | | | | |
| SNP_A- | 0.4498 | 0.1073 | 2.78e−05 | 1 | 1 | 0.461 | 0 | 0.177 | 0.143 |
| 2031097 | | | | | | | | | |
| SNP_A- | 0.2804 | 0.09888 | 0.00457 | 1 | 1 | 0.869 | 0.0076 | 0.371 | 0.616 |
| 2144434 | | | | | | | | | |
| SNP_A- | 0.2941 | 0.1179 | 0.0126 | 1 | 1 | 0.91 | 0 | 0.182 | 0.896 |
| 2110070 | | | | | | | | | |
| SNP_A- | −0.4243 | 0.1048 | 5.18e−05 | 1 | 1 | 0.49 | 0 | 0.393 | 1 |
| 1902860 | | | | | | | | | |
| SNP_A- | 0.7181 | 0.1707 | 2.58e−05 | 1 | 1 | 0.461 | 0 | 0.054 | 0.713 |
| 1868624 | | | | | | | | | |
| SNP_A- | 0.3953 | 0.09746 | 4.99e−05 | 1 | 1 | 0.487 | 0.00152 | 0.396 | 0.684 |
| 2153320 | | | | | | | | | |
| SNP_A- | −1.088 | 0.2551 | 1.99e−05 | 1 | 1 | 0.461 | 0.00608 | 0.0222 | 1 |
| 8569796 | | | | | | | | | |
| SNP_A- | −0.4345 | 0.1066 | 4.56e−05 | 1 | 1 | 0.487 | 0.00152 | 0.193 | 0.167 |
| 8352538 | | | | | | | | | |
| SNP_A- | 1.203 | 0.2895 | 3.23e−05 | 1 | 1 | 0.465 | 0.0334 | 0.0118 | 1 |
| 8479123 | | | | | | | | | |
| SNP_A- | −0.5304 | 0.1269 | 2.92e−05 | 1 | 1 | 0.461 | 0.0122 | 0.142 | 0.872 |
| 8582717 | | | | | | | | | |
| SNP_A- | 0.4471 | 0.1079 | 3.42e−05 | 1 | 1 | 0.465 | 0 | 0.255 | 0.918 |
| 8660563 | | | | | | | | | |
| SNP_A- | −1.249 | 0.2979 | 2.73e−05 | 1 | 1 | 0.461 | 0 | 0.0122 | 1 |
| 8532464 | | | | | | | | | |
| SNP_A- | 0.4107 | 0.1012 | 4.93e−05 | 1 | 1 | 0.487 | 0 | 0.421 | 0.0163 |
| 8611802 | | | | | | | | | |
| SNP_A- | −0.6448 | 0.2793 | 0.0209 | 1 | 1 | 0.936 | 0.00304 | 0.0244 | 1 |
| 8669637 | | | | | | | | | |
| SNP_A- | −0.4387 | 0.1023 | 1.81e−05 | 1 | 1 | 0.451 | 0.0137 | 0.442 | 0.0466 |
| 8662057 | | | | | | | | | |
| SNP_A- | −0.3694 | 0.2244 | 0.0997 | 1 | 1 | 0.966 | 0.00152 | 0.0457 | 1 |
| 1925576 | | | | | | | | | |
| SNP_A- | 0.397 | 0.09854 | 5.6e−05 | 1 | 1 | 0.494 | 0.00456 | 0.485 | 0.815 |
| 8399794 | | | | | | | | | |
| SNP_A- | 0.9333 | 0.2289 | 4.56e−05 | 1 | 1 | 0.487 | 0 | 0.0228 | 0.287 |
| 2054062 | | | | | | | | | |

| pid | chr | position | rsid | npa_x | odds_ratio | isc_coef | isc_stderr | isc_pval |
|---|---|---|---|---|---|---|---|---|
| SNP_A- | 4 | 96760067 | rs17024266 | FALSE | 0.379 | −1.064 | 0.2031 | 1.623e−07 |
| 2053054 | | | | | | | | |
| SNP_A- | X | 28977674 | rs5943590 | TRUE | 0.697 | −0.4079 | 0.08998 | 5.801e−06 |
| 8370399 | | | | | | | | |
| SNP_A- | 1 | 155572157 | rs1018615 | FALSE | 2.66 | 1.209 | 0.2724 | 9.096e−06 |
| 8631553 | | | | | | | | |
| SNP_A- | 9 | 82910311 | rs953188 | FALSE | 1.79 | 0.77 | 0.1563 | 8.399e−07 |
| 8285583 | | | | | | | | |
| SNP_A- | 9 | 82948468 | rs997020 | FALSE | 0.554 | −0.7411 | 0.1622 | 4.897e−06 |
| 1854346 | | | | | | | | |
| SNP_A- | X | 107557678 | rs7060905 | TRUE | 0.375 | −1.119 | 0.2371 | 2.371e−06 |
| 8647043 | | | | | | | | |
| SNP_A- | 9 | 82911335 | rs10867699 | FALSE | 1.72 | 0.7339 | 0.1583 | 3.568e−06 |
| 2183445 | | | | | | | | |
| SNP_A- | 3 | 149836430 | rs275697 | FALSE | 2.01 | 0.9483 | 0.208 | 5.121e−06 |
| 8530310 | | | | | | | | |
| SNP_A- | 13 | 94515499 | rs4148536 | FALSE | 0.507 | −0.9382 | 0.1922 | 1.057e−06 |
| 8456423 | | | | | | | | |
| SNP_A- | 6 | 166836302 | rs12524741 | FALSE | 1.91 | 0.7753 | 0.177 | 1.183e−05 |
| 8596066 | | | | | | | | |
| SNP_A- | 9 | 82979213 | rs2809841 | FALSE | 1.67 | 0.6885 | 0.1568 | 1.121e−05 |
| 8582554 | | | | | | | | |
| SNP_A- | 2 | 235029590 | rs1876715 | FALSE | 0.494 | −0.6633 | 0.1716 | 0.0001107 |
| 1967375 | | | | | | | | |
| SNP_A- | 3 | 21227105 | rs6791277 | FALSE | 0.24 | −1.356 | 0.3478 | 9.608e−05 |
| 8366760 | | | | | | | | |
| SNP_A- | 2 | 235017645 | rs1472929 | FALSE | 2.03 | 0.6691 | 0.1703 | 8.551e−05 |
| 8478064 | | | | | | | | |
| SNP_A- | 2 | 51881093 | rs12477891 | FALSE | 0.415 | −0.8571 | 0.2251 | 0.0001402 |
| 8349850 | | | | | | | | |
| SNP_A- | 1 | 217912907 | rs1856326 | FALSE | 0.634 | −0.7402 | 0.1607 | 4.092e−06 |
| 8544970 | | | | | | | | |
| SNP_A- | 6 | 166837330 | rs6934309 | FALSE | 1.87 | 0.7769 | 0.1769 | 1.127e−05 |
| 1988493 | | | | | | | | |
| SNP_A- | 1 | 217916196 | rs10779374 | FALSE | 1.56 | 0.7625 | 0.1636 | 3.15e−06 |
| 4265535 | | | | | | | | |
| SNP_A- | 1 | 217910815 | rs11118383 | FALSE | 0.644 | −0.7397 | 0.1609 | 4.264e−06 |
| 2080370 | | | | | | | | |
| SNP_A- | 1 | 217912636 | rs1856327 | FALSE | 0.644 | −0.7397 | 0.1609 | 4.264e−06 |
| 2092022 | | | | | | | | |
| SNP_A- | 6 | 167521338 | rs2345970 | FALSE | 0.69 | −0.5864 | 0.126 | 3.252e−06 |
| 2202302 | | | | | | | | |
| SNP_A- | 1 | 217905980 | rs10863478 | FALSE | 1.55 | 0.7527 | 0.1602 | 2.622e−06 |
| 1959929 | | | | | | | | |
| SNP_A- | 1 | 217914460 | rs10495133 | FALSE | 0.644 | −0.7407 | 0.1609 | 4.153e−06 |
| 8668446 | | | | | | | | |
| SNP_A- | 1 | 217914398 | rs10779373 | FALSE | 1.55 | 0.7397 | 0.1609 | 4.264e−06 |
| 8417654 | | | | | | | | |
| SNP_A- | 6 | 112042375 | rs6926543 | FALSE | 1.47 | 0.4776 | 0.1113 | 1.763e−05 |
| 8386477 | | | | | | | | |
| SNP_A- | 1 | 217909214 | rs1416000 | FALSE | 0.653 | −0.7397 | 0.1609 | 4.264e−06 |
| 1985257 | | | | | | | | |
| SNP_A- | 1 | 217907040 | rs10779368 | FALSE | 1.53 | 0.7397 | 0.1609 | 4.264e−06 |
| 1896426 | | | | | | | | |
| SNP_A- | 8 | 62928256 | rs10088053 | FALSE | 0.471 | −0.7385 | 0.1964 | 0.0001702 |
| 4272029 | | | | | | | | |
| SNP_A- | 3 | 21196407 | rs7648626 | FALSE | 0.371 | −1.112 | 0.2628 | 2.346e−05 |
| 2168866 | | | | | | | | |
| SNP_A- | 3 | 21196353 | rs6550568 | FALSE | 0.376 | −1.112 | 0.2628 | 2.346e−05 |
| 1787940 | | | | | | | | |
| SNP_A- | 4 | 96755517 | rs17024261 | FALSE | 0.425 | −0.9477 | 0.2272 | 3.032e−05 |
| 4224892 | | | | | | | | |
| SNP_A- | 2 | 24871364 | rs4665719 | FALSE | 1.65 | 0.473 | 0.1298 | 0.0002435 |
| 8470071 | | | | | | | | |
| SNP_A- | 4 | 169962988 | rs7654189 | FALSE | 0.632 | −0.49 | 0.1238 | 7.572e−05 |
| 1803248 | | | | | | | | |
| SNP_A- | 1 | 71125458 | rs1409981 | FALSE | 0.634 | −0.4879 | 0.1235 | 7.781e−05 |
| 8565161 | | | | | | | | |
| SNP_A- | 1 | 69458450 | rs12082124 | FALSE | 3.01 | 1.19 | 0.3058 | 0.0001003 |
| 8351277 | | | | | | | | |
| SNP_A- | 1 | 217909523 | rs1415282 | FALSE | 0.654 | −0.7141 | 0.1595 | 7.577e−06 |
| 8668443 | | | | | | | | |
| SNP_A- | 2 | 158389887 | rs16842126 | FALSE | 2.74 | 1.853 | 0.3679 | 4.727e−07 |
| 8421072 | | | | | | | | |
| SNP_A- | — | 785 | rs3119588 | FALSE | 0.699 | −0.5566 | 0.1256 | 9.402e−06 |
| 1842166 | | | | | | | | |
| SNP_A- | 6 | 19002215 | rs6917825 | FALSE | 0.63 | −0.601 | 0.1399 | 1.731e−05 |
| 4220021 | | | | | | | | |
| SNP_A- | 14 | 57375098 | rs17093751 | FALSE | 3.36 | 1.187 | 0.346 | 0.0006033 |
| 8299340 | | | | | | | | |
| SNP_A- | 4 | 141044042 | rs17050999 | FALSE | 0.426 | −1.181 | 0.2762 | 1.901e−05 |
| 2152929 | | | | | | | | |
| SNP_A- | 15 | 44672449 | rs1400412 | FALSE | 2.03 | 0.9163 | 0.2187 | 2.803e−05 |
| 1953240 | | | | | | | | |
| SNP_A- | 12 | 56765747 | rs2720185 | FALSE | 0.408 | −1.571 | 0.3346 | 2.653e−06 |
| 2002771 | | | | | | | | |
| SNP_A- | 4 | 88103790 | rs12651081 | FALSE | 1.31 | 0.5367 | 0.117 | 4.469e−06 |
| 2047393 | | | | | | | | |
| SNP_A- | 9 | 1801657 | rs10963396 | FALSE | 0.347 | −1.186 | 0.3023 | 8.737e−05 |
| 8672704 | | | | | | | | |
| SNP_A- | 14 | 96141802 | rs234605 | FALSE | 1.54 | 0.466 | 0.1215 | 0.000126 |
| 8677651 | | | | | | | | |
| SNP_A- | 20 | 35327104 | rs7267965 | FALSE | 0.377 | −1.158 | 0.2886 | 6.028e−05 |
| 8493887 | | | | | | | | |
| SNP_A- | 7 | 70213501 | rs886739 | FALSE | 1.52 | 0.4687 | 0.1197 | 9.031e−05 |
| 8490285 | | | | | | | | |
| SNP_A- | 5 | 65966059 | rs16895353 | FALSE | 0.393 | −0.9253 | 0.2447 | 0.000156 |
| 2166983 | | | | | | | | |
| SNP_A- | 4 | 96861645 | rs6814329 | FALSE | 2.17 | 0.827 | 0.2143 | 0.0001138 |
| 8658724 | | | | | | | | |
| SNP_A- | 2 | 24950456 | rs7567997 | FALSE | 1.54 | 0.4135 | 0.1141 | 0.0002896 |
| 8378831 | | | | | | | | |
| SNP_A- | 5 | 7917532 | rs16879248 | FALSE | 0.368 | −0.9826 | 0.2622 | 0.0001785 |
| 8296527 | | | | | | | | |
| SNP_A- | 3 | 53797359 | rs3774598 | FALSE | 0.307 | −1.725 | 0.3979 | 1.455e−05 |
| 1972641 | | | | | | | | |
| SNP_A- | 6 | 144656559 | rs7740792 | FALSE | 0.235 | −1.668 | 0.4594 | 0.0002812 |
| 8405569 | | | | | | | | |
| SNP_A- | 6 | 18969221 | rs4716312 | FALSE | 1.55 | 0.5844 | 0.1386 | 2.485e−05 |
| 2171537 | | | | | | | | |
| SNP_A- | 4 | 88076615 | rs1447993 | FALSE | 1.38 | 0.5764 | 0.13 | 9.33e−06 |
| 4252168 | | | | | | | | |
| SNP_A- | 4 | 82273150 | rs11723204 | FALSE | 2.03 | 1.222 | 0.2749 | 8.701e−06 |
| 8453740 | | | | | | | | |
| SNP_A- | 2 | 12593877 | rs13013085 | FALSE | 0.575 | −0.6388 | 0.1705 | 0.0001788 |
| 4252121 | | | | | | | | |
| SNP_A- | 10 | 45286428 | rs901683 | FALSE | 1.98 | 0.6551 | 0.179 | 0.0002523 |
| 2000347 | | | | | | | | |
| SNP_A- | 4 | 4871714 | rs4689946 | FALSE | 1.69 | 0.4842 | 0.1424 | 0.0006735 |
| 8510071 | | | | | | | | |
| SNP_A- | 16 | 10680908 | rs10221110 | FALSE | 0.654 | −0.5663 | 0.1355 | 2.916e−05 |
| 2083150 | | | | | | | | |
| SNP_A- | 16 | 10680325 | rs2719715 | FALSE | 0.654 | −0.5663 | 0.1355 | 2.916e−05 |
| 4235811 | | | | | | | | |
| SNP_A- | 22 | 47541093 | rs13056461 | FALSE | 1.49 | 0.56 | 0.1348 | 3.277e−05 |
| 8485648 | | | | | | | | |
| SNP_A- | X | 29021974 | rs2651175 | TRUE | 0.723 | −0.3479 | 0.09383 | 0.0002087 |
| 8356840 | | | | | | | | |
| SNP_A- | 6 | 18969437 | rs1360771 | FALSE | 0.644 | −0.5896 | 0.1413 | 3.013e−05 |
| 1834789 | | | | | | | | |
| SNP_A- | 16 | 78483602 | rs13330604 | FALSE | 0.586 | −0.4704 | 0.1449 | 0.001172 |
| 8642499 | | | | | | | | |
| SNP_A- | 17 | 58504151 | rs8072580 | FALSE | 1.84 | 0.9418 | 0.218 | 1.551e−05 |
| 4205314 | | | | | | | | |
| SNP_A- | 2 | 24984411 | rs6733224 | FALSE | 0.658 | −0.4139 | 0.1146 | 0.0003063 |
| 2152506 | | | | | | | | |
| SNP_A- | 16 | 10677661 | rs12925749 | FALSE | 0.641 | −0.6552 | 0.1533 | 1.919e−05 |
| 8432970 | | | | | | | | |
| SNP_A- | 13 | 65929499 | rs10507737 | FALSE | 0.64 | −0.4704 | 0.129 | 0.0002645 |
| 8548394 | | | | | | | | |
| SNP_A- | 2 | 24956950 | rs2033653 | FALSE | 0.658 | −0.4105 | 0.115 | 0.0003578 |
| 8681923 | | | | | | | | |
| SNP_A- | 8 | 14079214 | rs7840084 | FALSE | 1.71 | 0.5533 | 0.155 | 0.0003575 |
| 8630612 | | | | | | | | |
| SNP_A- | 2 | 235015475 | rs6743014 | FALSE | 1.87 | 0.5722 | 0.167 | 0.0006123 |
| 4204345 | | | | | | | | |
| SNP_A- | 2 | 24983966 | rs10200566 | FALSE | 0.664 | −0.4164 | 0.1166 | 0.0003531 |
| 8398578 | | | | | | | | |
| SNP_A- | 2 | 24592914 | rs1545255 | FALSE | 1.61 | 0.3977 | 0.1293 | 0.002098 |
| 2243420 | | | | | | | | |
| SNP_A- | 2 | 24984046 | rs10198275 | FALSE | 1.5 | 0.3973 | 0.1141 | 0.0004973 |
| 2052179 | | | | | | | | |
| SNP_A- | 2 | 24984820 | rs6545814 | FALSE | 1.5 | 0.3973 | 0.1141 | 0.0004973 |
| 2059271 | | | | | | | | |
| SNP_A- | 14 | 57374152 | rs2145489 | FALSE | 2.01 | 0.7099 | 0.2014 | 0.0004234 |
| 2206221 | | | | | | | | |
| SNP_A- | 2 | 24972389 | rs6545800 | FALSE | 0.668 | −0.3916 | 0.1148 | 0.0006473 |
| 2262428 | | | | | | | | |
| SNP_A- | 2 | 24985490 | rs11900505 | FALSE | 1.49 | 0.3967 | 0.1141 | 0.0005097 |
| 1864375 | | | | | | | | |
| SNP_A- | 4 | 39424918 | rs10517528 | FALSE | 0.534 | −0.5733 | 0.1719 | 0.000854 |
| 1976890 | | | | | | | | |
| SNP_A- | 19 | 61792033 | rs741252 | FALSE | 1.55 | 0.4229 | 0.1272 | 0.0008836 |
| 4287784 | | | | | | | | |
| SNP_A- | 19 | 61784980 | rs11084454 | FALSE | 0.646 | −0.4229 | 0.1272 | 0.0008836 |
| 1942320 | | | | | | | | |
| SNP_A- | 3 | 117233816 | rs9821040 | FALSE | 1.63 | 0.4632 | 0.1398 | 0.0009233 |
| 8456608 | | | | | | | | |
| SNP_A- | 6 | 16165496 | rs4716037 | FALSE | 1.58 | 0.4057 | 0.1269 | 0.001384 |
| 8627377 | | | | | | | | |
| SNP_A- | 2 | 24953832 | rs2384058 | FALSE | 1.5 | 0.369 | 0.1132 | 0.001118 |
| 8366937 | | | | | | | | |
| SNP_A- | 19 | 61787066 | rs4801343 | FALSE | 0.647 | −0.4207 | 0.1275 | 0.00967 |
| 4273665 | | | | | | | | |
| SNP_A- | 16 | 72446965 | rs10500575 | FALSE | 0.626 | −0.4723 | 0.1394 | 0.0007026 |
| 4195397 | | | | | | | | |
| SNP_A- | 19 | 18034603 | rs372889 | FALSE | 0.644 | −0.3285 | 0.1168 | 0.004928 |
| 2113673 | | | | | | | | |
| AFFX- | 19 | 18034603 | rs372889 | FALSE | 0.644 | −0.3285 | 0.1168 | 0.004928 |
| SNP_6891433 | | | | | | | | |
| SNP_A- | 2 | 144498321 | rs16823406 | FALSE | 0.342 | −1.045 | 0.3117 | 0.0007962 |
| 1965187 | | | | | | | | |
| SNP_A- | 6 | 22469455 | rs1205925 | FALSE | 0.669 | −0.3886 | 0.1202 | 0.001221 |
| 1985390 | | | | | | | | |
| SNP_A- | 2 | 24989124 | rs2384061 | FALSE | 1.51 | 0.3669 | 0.1176 | 0.00181 |
| 2199372 | | | | | | | | |
| SNP_A- | 4 | 4864223 | rs1907991 | FALSE | 0.619 | −0.4423 | 0.1429 | 0.001974 |
| 2223920 | | | | | | | | |
| SNP_A- | 19 | 61777581 | rs10421285 | FALSE | 0.652 | −0.4085 | 0.1265 | 0.001248 |
| 2075251 | | | | | | | | |
| SNP_A- | 2 | 157099822 | rs2568816 | FALSE | 1.6 | 0.421 | 0.133 | 0.001545 |
| 1965505 | | | | | | | | |
| SNP_A- | 2 | 24933274 | rs11675457 | FALSE | 1.51 | 0.3513 | 0.1165 | 0.00257 |
| 8603804 | | | | | | | | |
| SNP_A- | 2 | 24961701 | rs1865689 | FALSE | 0.669 | −0.3667 | 0.1163 | 0.001615 |
| 1962473 | | | | | | | | |
| SNP_A- | 20 | 273102 | rs6084145 | FALSE | 0.679 | −0.5859 | 0.1409 | 3.215e−05 |
| 8613839 | | | | | | | | |
| SNP_A- | 2 | 214668085 | rs11900000 | FALSE | 0.603 | −0.8506 | 0.204 | 3.041e−05 |
| 1957079 | | | | | | | | |
| SNP_A- | 6 | 119479680 | rs794258 | FALSE | 0.488 | −1.115 | 0.2676 | 3.078e−05 |
| 8432286 | | | | | | | | |
| SNP_A- | 4 | 169929444 | rs7679982 | FALSE | 1.54 | 0.4141 | 0.1324 | 0.001761 |
| 4288330 | | | | | | | | |
| SNP_A- | 4 | 169928846 | rs17708289 | FALSE | 1.54 | 0.4141 | 0.1324 | 0.001761 |
| 4300393 | | | | | | | | |
| SNP_A- | 9 | 85325008 | rs17086403 | FALSE | 0.483 | −0.6386 | 0.2157 | 0.003067 |
| 8407616 | | | | | | | | |
| SNP_A- | 2 | 24546313 | rs2165738 | FALSE | 0.663 | −0.3713 | 0.1233 | 0.00259 |
| 1949138 | | | | | | | | |
| SNP_A- | 12 | 9369404 | rs7302181 | FALSE | 0.634 | −0.3906 | 0.136 | 0.004073 |
| 1908453 | | | | | | | | |
| SNP_A- | 4 | 169963645 | rs11726774 | FALSE | 1.5 | 0.3411 | 0.119 | 0.004151 |
| 8596473 | | | | | | | | |
| SNP_A- | 12 | 9422322 | rs10492108 | FALSE | 1.57 | 0.3767 | 0.1343 | 0.005041 |
| 2031097 | | | | | | | | |
| SNP_A- | 11 | 107222310 | rs11212408 | FALSE | 1.32 | 0.4963 | 0.1197 | 3.373e−05 |
| 2144434 | | | | | | | | |
| SNP_A- | 4 | 88082794 | rs6836128 | FALSE | 1.34 | 0.5552 | 0.1311 | 2.29e−05 |
| 2110070 | | | | | | | | |
| SNP_A- | 6 | 22406678 | rs849877 | FALSE | 0.654 | −0.3612 | 0.1238 | 0.003525 |
| 1902860 | | | | | | | | |
| SNP_A- | 14 | 57497859 | rs17094008 | FALSE | 2.05 | 0.5926 | 0.2163 | 0.006142 |
| 1868624 | | | | | | | | |
| SNP_A- | 2 | 24954596 | rs2033655 | FALSE | 1.48 | 0.3292 | 0.115 | 0.004194 |
| 2153320 | | | | | | | | |
| SNP_A- | 9 | 1826746 | rs10116883 | FALSE | 0.337 | −0.9396 | 0.3517 | 0.007552 |
| 8569796 | | | | | | | | |
| SNP_A- | 12 | 9422128 | rs11050596 | FALSE | 0.648 | −0.366 | 0.1329 | 0.005877 |
| 8352538 | | | | | | | | |
| SNP_A- | 3 | 7805879 | rs7641662 | FALSE | 3.33 | 0.9576 | 0.3673 | 0.009134 |
| 8479123 | | | | | | | | |
| SNP_A- | 12 | 96773971 | rs12825850 | FALSE | 0.588 | −0.3971 | 0.1574 | 0.01166 |
| 8582717 | | | | | | | | |
| SNP_A- | 20 | 42750069 | rs7262172 | FALSE | 1.56 | 0.3304 | 0.1331 | 0.01303 |
| 8660563 | | | | | | | | |
| SNP_A- | 14 | 57272128 | rs1092014 | FALSE | 0.287 | −1.017 | 0.4186 | 0.01513 |
| 8532464 | | | | | | | | |
| SNP_A- | 10 | 54716153 | rs10824983 | FALSE | 1.51 | 0.3222 | 0.121 | 0.007762 |
| 8611802 | | | | | | | | |
| SNP_A- | 6 | 161410210 | rs3757020 | FALSE | 0.525 | −1.186 | 0.2839 | 2.934e−05 |
| 8669637 | | | | | | | | |
| SNP_A- | 4 | 169924575 | rs869396 | FALSE | 0.645 | −0.2639 | 0.1221 | 0.03075 |
| 8662057 | | | | | | | | |
| SNP_A- | 6 | 5665825 | rs1977059 | FALSE | 0.691 | −1.006 | 0.2381 | 2.376e−05 |
| 1925576 | | | | | | | | |
| SNP_A- | 7 | 147516396 | rs17170877 | FALSE | 1.49 | 0.3014 | 0.12 | 0.01202 |
| 8399794 | | | | | | | | |
| SNP_A- | 14 | 57309830 | rs1956681 | FALSE | 2.54 | 0.7331 | 0.3155 | 0.02015 |
| 2054062 | | | | | | | | |

| pid | isc_pval_holm | isc_pval_fdr | nyc_pval | ef_pval | isc_nyc_pval | isc_ef_pval |
|---|---|---|---|---|---|---|
| SNP_A- | 0.121426 | 0.121426 | 0.597 | 0.432 | 0.399 | 0.938 |
| 2053054 | | | | | | |
| SNP_A- | 1 | 0.206669 | 0.354 | 0.0728 | 0.534 | 0.0157 |
| 8370399 | | | | | | |
| SNP_A- | 1 | 0.270544 | 0.743 | 0.344 | 0.649 | 0.577 |
| 8631553 | | | | | | |
| SNP_A- | 0.628374 | 0.18575 | 0.687 | 0.334 | 0.908 | 0.531 |
| 8285583 | | | | | | |
| SNP_A- | 1 | 0.191565 | 0.519 | 0.229 | 0.879 | 0.591 |
| 1854346 | | | | | | |
| SNP_A- | 1 | 0.18575 | 0.975 | 0.488 | 0.801 | 0.922 |
| 8647043 | | | | | | |
| SNP_A- | 1 | 0.18575 | 0.654 | 0.226 | 0.912 | 0.614 |
| 2183445 | | | | | | |
| SNP_A- | 1 | 0.191565 | 0.257 | 0.119 | 0.399 | 0.0158 |
| 8530310 | | | | | | |
| SNP_A- | 0.790797 | 0.18575 | 0.488 | 0.405 | 0.975 | 0.297 |
| 8456423 | | | | | | |
| SNP_A- | 1 | 0.305196 | 0.733 | 0.222 | 0.344 | 0.17 |
| 8596066 | | | | | | |
| SNP_A- | 1 | 0.301132 | 0.9 | 0.0338 | 0.976 | 0.0847 |
| 8582554 | | | | | | |
| SNP_A- | 1 | 0.607431 | 0.155 | 0.561 | 0.97 | 0.634 |
| 1967375 | | | | | | |
| SNP_A- | 1 | 0.607431 | 0.193 | 0.177 | 0.142 | 0.0889 |
| 8366760 | | | | | | |
| SNP_A- | 1 | 0.607431 | 0.295 | 0.625 | 0.742 | 0.69 |
| 8478064 | | | | | | |
| SNP_A- | 1 | 0.611853 | 0.324 | 0.642 | 0.167 | 0.928 |
| 8349850 | | | | | | |
| SNP_A- | 1 | 0.18575 | 0.0448 | 0.499 | 0.133 | 0.395 |
| 8544970 | | | | | | |
| SNP_A- | 1 | 0.301132 | 0.812 | 0.258 | 0.344 | 0.21 |
| 1988493 | | | | | | |
| SNP_A- | 1 | 0.18575 | 0.0516 | 0.438 | 0.135 | 0.354 |
| 4265535 | | | | | | |
| SNP_A- | 1 | 0.18575 | 0.0382 | 0.486 | 0.097 | 0.339 |
| 2080370 | | | | | | |
| SNP_A- | 1 | 0.18575 | 0.0382 | 0.486 | 0.097 | 0.339 |
| 2092022 | | | | | | |
| SNP_A- | 1 | 0.18575 | 0.452 | 0.706 | 0.45 | 0.0339 |
| 2202302 | | | | | | |
| SNP_A- | 1 | 0.18575 | 0.0826 | 0.678 | 0.136 | 0.424 |
| 1959929 | | | | | | |
| SNP_A- | 1 | 0.18575 | 0.0366 | 0.577 | 0.0889 | 0.431 |
| 8668446 | | | | | | |
| SNP_A- | 1 | 0.18575 | 0.0382 | 0.486 | 0.097 | 0.339 |
| 8417654 | | | | | | |
| SNP_A- | 1 | 0.399696 | 0.753 | 0.474 | 0.825 | 0.59 |
| 8386477 | | | | | | |
| SNP_A- | 1 | 0.18575 | 0.0457 | 0.496 | 0.097 | 0.339 |
| 1985257 | | | | | | |
| SNP_A- | 1 | 0.18575 | 0.0457 | 0.496 | 0.097 | 0.339 |
| 1896426 | | | | | | |
| SNP_A- | 1 | 0.63668 | 0.43 | 0.622 | 0.826 | 0.0316 |
| 4272029 | | | | | | |
| SNP_A- | 1 | 0.455799 | 0.632 | 0.493 | 0.123 | 0.548 |
| 2168866 | | | | | | |
| SNP_A- | 1 | 0.455799 | 0.504 | 0.358 | 0.123 | 0.548 |
| 1787940 | | | | | | |
| SNP_A- | 1 | 0.479754 | 0.519 | 0.194 | 0.377 | 0.562 |
| 4224892 | | | | | | |
| SNP_A- | 1 | 0.722157 | 0.347 | 0.666 | 0.241 | 0.635 |
| 8470071 | | | | | | |
| SNP_A- | 1 | 0.607431 | 0.276 | 0.643 | 0.157 | 0.45 |
| 1803248 | | | | | | |
| SNP_A- | 1 | 0.607431 | 0.935 | 0.995 | 0.842 | 0.8 |
| 8565161 | | | | | | |
| SNP_A- | 1 | 0.607431 | 0.615 | 0.493 | 0.895 | 0.371 |
| 8351277 | | | | | | |
| SNP_A- | 1 | 0.257671 | 0.0466 | 0.522 | 0.0817 | 0.356 |
| 8668443 | | | | | | |
| SNP_A- | 0.353652 | 0.176826 | 0.94 | 0.00712 | 0.329 | 0.00302 |
| 8421072 | | | | | | |
| SNP_A- | 1 | 0.270544 | 0.306 | 0.737 | 0.275 | 0.0325 |
| 1842166 | | | | | | |
| SNP_A- | 1 | 0.399696 | 0.369 | 0.045 | 0.0699 | 0.298 |
| 4220021 | | | | | | |
| SNP_A- | 1 | 0.767302 | 0.218 | 0.5 | 0.231 | 0.434 |
| 8299340 | | | | | | |
| SNP_A- | 1 | 0.410203 | 0.444 | 0.605 | 0.418 | 0.885 |
| 2152929 | | | | | | |
| SNP_A- | 1 | 0.479754 | 0.537 | 0.955 | 0.854 | 0.833 |
| 1953240 | | | | | | |
| SNP_A- | 1 | 0.18575 | 0.774 | 0.463 | 0.372 | 0.244 |
| 2002771 | | | | | | |
| SNP_A- | 1 | 0.18575 | 0.651 | 0.402 | 0.678 | 0.267 |
| 2047393 | | | | | | |
| SNP_A- | 1 | 0.607431 | 0.236 | 0.649 | 0.983 | 0.983 |
| 8672704 | | | | | | |
| SNP_A- | 1 | 0.607431 | 0.959 | 0.39 | 0.774 | 0.701 |
| 8677651 | | | | | | |
| SNP_A- | 1 | 0.607431 | 0.619 | 0.773 | 0.304 | 0.95 |
| 8493887 | | | | | | |
| SNP_A- | 1 | 0.607431 | 0.142 | 0.206 | 0.462 | 0.31 |
| 8490285 | | | | | | |
| SNP_A- | 1 | 0.630877 | 0.286 | 0.568 | 0.514 | 0.348 |
| 2166983 | | | | | | |
| SNP_A- | 1 | 0.607431 | 0.66 | 0.381 | 0.419 | 0.594 |
| 8658724 | | | | | | |
| SNP_A- | 1 | 0.734952 | 0.338 | 0.838 | 0.194 | 0.911 |
| 8378831 | | | | | | |
| SNP_A- | 1 | 0.655907 | 0.141 | 0.336 | 0.503 | 0.571 |
| 8296527 | | | | | | |
| SNP_A- | 1 | 0.362855 | 0.249 | 0.28 | 0.0678 | 0.316 |
| 1972641 | | | | | | |
| SNP_A- | 1 | 0.734952 | 0.792 | 0.583 | 0.718 | 0.601 |
| 8405569 | | | | | | |
| SNP_A- | 1 | 0.464791 | 0.539 | 0.0568 | 0.086 | 0.318 |
| 2171537 | | | | | | |
| SNP_A- | 1 | 0.270544 | 0.286 | 0.0101 | 0.203 | 0.0122 |
| 4252168 | | | | | | |
| SNP_A- | 1 | 0.270544 | 0.743 | 0.262 | 0.766 | 0.0178 |
| 8453740 | | | | | | |
| SNP_A- | 1 | 0.655907 | 0.53 | 0.516 | 0.915 | 0.541 |
| 4252121 | | | | | | |
| SNP_A- | 1 | 0.725998 | 0.0703 | 0.794 | 0.148 | 0.561 |
| 2000347 | | | | | | |
| SNP_A- | 1 | 0.778751 | 0.175 | 0.821 | 0.372 | 0.927 |
| 8510071 | | | | | | |
| SNP_A- | 1 | 0.479754 | 0.0313 | 0.395 | 0.17 | 0.261 |
| 2083150 | | | | | | |
| SNP_A- | 1 | 0.479754 | 0.0313 | 0.395 | 0.17 | 0.261 |
| 4235811 | | | | | | |
| SNP_A- | 1 | 0.490341 | 0.727 | 0.293 | 0.485 | 0.612 |
| 8485648 | | | | | | |
| SNP_A- | 1 | 0.677226 | 0.346 | 0.164 | 0.9 | 0.0206 |
| 8356840 | | | | | | |
| SNP_A- | 1 | 0.479754 | 0.552 | 0.0448 | 0.104 | 0.308 |
| 1834789 | | | | | | |
| SNP_A- | 1 | 0.811523 | 0.756 | 0.0781 | 0.822 | 0.19 |
| 8642499 | | | | | | |
| SNP_A- | 1 | 0.374319 | 0.558 | 0.254 | 0.947 | 0.61 |
| 4205314 | | | | | | |
| SNP_A- | 1 | 0.744268 | 0.523 | 0.888 | 0.464 | 0.838 |
| 2152506 | | | | | | |
| SNP_A- | 1 | 0.410203 | 0.023 | 0.591 | 0.422 | 0.52 |
| 8432970 | | | | | | |
| SNP_A- | 1 | 0.73431 | 0.634 | 0.19 | 0.619 | 0.156 |
| 8548394 | | | | | | |
| SNP_A- | 1 | 0.754805 | 0.614 | 0.922 | 0.484 | 0.815 |
| 8681923 | | | | | | |
| SNP_A- | 1 | 0.754805 | 0.315 | 0.904 | 0.48 | 0.877 |
| 8630612 | | | | | | |
| SNP_A- | 1 | 0.767302 | 0.186 | 0.656 | 0.983 | 0.662 |
| 4204345 | | | | | | |
| SNP_A- | 1 | 0.754805 | 0.305 | 0.379 | 0.246 | 0.444 |
| 8398578 | | | | | | |
| SNP_A- | 1 | 0.835697 | 0.333 | 0.861 | 0.318 | 0.921 |
| 2243420 | | | | | | |
| SNP_A- | 1 | 0.767302 | 0.442 | 0.763 | 0.374 | 0.691 |
| 2052179 | | | | | | |
| SNP_A- | 1 | 0.767302 | 0.442 | 0.763 | 0.374 | 0.691 |
| 2059271 | | | | | | |
| SNP_A- | 1 | 0.767302 | 0.51 | 0.186 | 0.591 | 0.248 |
| 2206221 | | | | | | |
| SNP_A- | 1 | 0.767302 | 0.542 | 0.631 | 0.449 | 0.72 |
| 2262428 | | | | | | |
| SNP_A- | 1 | 0.767302 | 0.441 | 0.762 | 0.344 | 0.678 |
| 1864375 | | | | | | |
| SNP_A- | 1 | 0.792807 | 0.933 | 0.563 | 0.19 | 0.607 |
| 1976890 | | | | | | |
| SNP_A- | 1 | 0.797762 | 0.00187 | 0.994 | 0.00157 | 0.958 |
| 4287784 | | | | | | |
| SNP_A- | 1 | 0.797762 | 0.00233 | 0.949 | 0.00157 | 0.958 |
| 1942320 | | | | | | |
| SNP_A- | 1 | 0.801006 | 0.0636 | 0.195 | 0.0765 | 0.521 |
| 8456608 | | | | | | |
| SNP_A- | 1 | 0.820371 | 0.874 | 0.944 | 0.858 | 0.822 |
| 8627377 | | | | | | |
| SNP_A- | 1 | 0.811523 | 0.526 | 0.936 | 0.326 | 0.939 |
| 8366937 | | | | | | |
| SNP_A- | 1 | 0.811057 | 0.00155 | 0.955 | 0.00123 | 0.993 |
| 4273665 | | | | | | |
| SNP_A- | 1 | 0.78573 | 0.493 | 0.786 | 0.864 | 0.306 |
| 4195397 | | | | | | |
| SNP_A- | 1 | 0.891826 | 0.337 | 0.316 | 0.178 | 0.615 |
| 2113673 | | | | | | |
| AFFX- | 1 | 0.891826 | 0.337 | 0.316 | 0.178 | 0.615 |
| SNP_6891433 | | | | | | |
| SNP_A- | 1 | 0.787986 | 0.0782 | 0.978 | 0.193 | 0.989 |
| 1965187 | | | | | | |
| SNP_A- | 1 | 0.816056 | 0.72 | 0.408 | 0.946 | 0.909 |
| 1985390 | | | | | | |
| SNP_A- | 1 | 0.833383 | 0.742 | 0.753 | 0.648 | 0.845 |
| 2199372 | | | | | | |
| SNP_A- | 1 | 0.833383 | 0.195 | 0.93 | 0.415 | 0.972 |
| 2223920 | | | | | | |
| SNP_A- | 1 | 0.816056 | 0.00154 | 0.922 | 0.000753 | 0.978 |
| 2075251 | | | | | | |
| SNP_A- | 1 | 0.820675 | 0.759 | 0.78 | 0.708 | 0.524 |
| 1965505 | | | | | | |
| SNP_A- | 1 | 0.850285 | 0.63 | 0.948 | 0.688 | 0.991 |
| 8603804 | | | | | | |
| SNP_A- | 1 | 0.82615 | 0.46 | 0.72 | 0.579 | 0.756 |
| 1962473 | | | | | | |
| SNP_A- | 1 | 0.490341 | 0.276 | 0.386 | 0.155 | 0.502 |
| 8613839 | | | | | | |
| SNP_A- | 1 | 0.479754 | 0.586 | 0.96 | 0.234 | 0.228 |
| 1957079 | | | | | | |
| SNP_A- | 1 | 0.479754 | 0.267 | 0.21 | 0.832 | 0.059 |
| 8432286 | | | | | | |
| SNP_A- | 1 | 0.830132 | 0.457 | 0.847 | 0.252 | 0.588 |
| 4288330 | | | | | | |
| SNP_A- | 1 | 0.830132 | 0.457 | 0.847 | 0.252 | 0.588 |
| 4300393 | | | | | | |
| SNP_A- | 1 | 0.860641 | 0.0717 | 0.255 | 0.102 | 0.382 |
| 8407616 | | | | | | |
| SNP_A- | 1 | 0.850285 | 0.846 | 0.142 | 0.626 | 0.359 |
| 1949138 | | | | | | |
| SNP_A- | 1 | 0.885676 | 0.931 | 0.335 | 0.547 | 0.625 |
| 1908453 | | | | | | |
| SNP_A- | 1 | 0.885676 | 0.816 | 0.887 | 0.933 | 0.84 |
| 8596473 | | | | | | |
| SNP_A- | 1 | 0.891826 | 0.672 | 0.706 | 0.552 | 0.863 |
| 2031097 | | | | | | |
| SNP_A- | 1 | 0.494809 | 0.166 | 0.669 | 0.056 | 0.754 |
| 2144434 | | | | | | |
| SNP_A- | 1 | 0.455799 | 0.236 | 0.00806 | 0.173 | 0.0119 |
| 2110070 | | | | | | |
| SNP_A- | 1 | 0.876162 | 0.478 | 0.246 | 0.748 | 0.279 |
| 1902860 | | | | | | |
| SNP_A- | 1 | 0.906124 | 0.34 | 0.535 | 0.331 | 0.0634 |
| 1868624 | | | | | | |
| SNP_A- | 1 | 0.886006 | 0.465 | 0.748 | 0.481 | 0.875 |
| 2153320 | | | | | | |
| SNP_A- | 1 | 0.922488 | 0.189 | 0.509 | 0.549 | 0.732 |
| 8569796 | | | | | | |
| SNP_A- | 1 | 0.902609 | 0.84 | 0.727 | 0.285 | 0.644 |
| 8352538 | | | | | | |
| SNP_A- | 1 | 0.92824 | 0.209 | 0.312 | 0.158 | 0.518 |
| 8479123 | | | | | | |
| SNP_A- | 1 | 0.937909 | 0.101 | 0.465 | 0.0801 | 0.279 |
| 8582717 | | | | | | |
| SNP_A- | 1 | 0.940195 | 0.728 | 0.375 | 0.38 | 0.362 |
| 8660563 | | | | | | |
| SNP_A- | 1 | 0.947838 | 0.347 | 0.362 | 0.952 | 0.408 |
| 8532464 | | | | | | |
| SNP_A- | 1 | 0.922717 | 0.56 | 0.686 | 0.233 | 0.763 |
| 8611802 | | | | | | |
| SNP_A- | 1 | 0.479754 | 0.707 | 0.403 | 0.153 | 0.831 |
| 8669637 | | | | | | |
| SNP_A- | 1 | 0.964806 | 0.606 | 0.88 | 0.881 | 0.961 |
| 8662057 | | | | | | |
| SNP_A- | 1 | 0.455799 | 0.304 | 0.268 | 0.913 | 0.213 |
| 1925576 | | | | | | |
| SNP_A- | 1 | 0.939565 | 0.244 | 0.342 | 0.564 | 0.167 |
| 8399794 | | | | | | |
| SNP_A- | 1 | 0.955841 | 0.677 | 0.401 | 0.14 | 0.199 |
| 2054062 | | | | | | |

TABLE 15

| | | | | | | | | | isc_pval | |
|---|---|---|---|---|---|---|---|---|---|---|
| dbSNP ID | Genes near locus | Cluster | Chr | Position | MAF | pval | pval_fdr | isc_pval | fdr | Correlation |
| rs12082124 | DEPDC1, LRRC7, | 1 | 1 | 69458450 | 0.0247 | 7.84e−06 | 0.368 | 0.0001003 | 0.607 | Positive |
| rs1409981 | PTGER3 | 2 | 1 | 71125458 | 0.305 | 6.93e−06 | 0.368 | 7.78e−05 | 0.607 | Negative |
| rs1018615 | ETV3, FCRL5, | 3 | 1 | 155572157 | 0.038 | 3.4e−06 | 0.273 | 9.1e−06 | 0.271 | Positive |
| rs1856326 | SLC30A10 | 4 | 1 | 217912907 | 0.237 | 0.00025 | 0.669 | 4.09e−06 | 0.186 | Negative |
| rs10779374 | SLC30A10 | 4 | 1 | 217916196 | 0.243 | 0.00037 | 0.69 | 3.15e−06 | 0.186 | Positive |
| rs10495133 | SLC30A10 | 4 | 1 | 217914460 | 0.249 | 0.00038 | 0.69 | 4.15e−06 | 0.186 | Negative |
| rs10863478 | SLC30A10 | 4 | 1 | 217905980 | 0.254 | 0.00039 | 0.69 | 2.62e−06 | 0.186 | Positive |
| rs11118383 | SLC30A10 | 4 | 1 | 217910815 | 0.248 | 0.00039 | 0.69 | 4.26e−06 | 0.186 | Negative |
| rs1856327 | SLC30A10 | 4 | 1 | 217912636 | 0.248 | 0.00039 | 0.69 | 4.26e−06 | 0.186 | Negative |
| rs10779373 | SLC30A10 | 4 | 1 | 217914398 | 0.248 | 0.00039 | 0.69 | 4.26e−06 | 0.186 | Positive |
| rs10779368 | SLC30A10 | 4 | 1 | 217907040 | 0.246 | 0.00056 | 0.722 | 4.26e−06 | 0.186 | Positive |
| rs1416000 | SLC30A10 | 4 | 1 | 217909214 | 0.247 | 0.00056 | 0.722 | 4.26e−06 | 0.186 | Negative |
| rs1415282 | SLC30A10 | 4 | 1 | 217909523 | 0.247 | 0.00056 | 0.722 | 7.58e−06 | 0.258 | Negative |
| rs13013085 | ST13, TRIB2, | 5 | 2 | 12593877 | 0.207 | 4.89e−05 | 0.487 | 0.0001788 | 0.656 | Negative |
| rs1545255 | ITSN2, NCOA1, | 6 | 2 | 24592914 | 0.21 | 1.05e−05 | 0.391 | 0.002098 | 0.836 | Positive |
| rs2165738 | ITSN2, NCOA1, | 6 | 2 | 24546313 | 0.271 | 4.78e−05 | 0.487 | 0.00259 | 0.850 | Negative |
| rs4665719 | CENPO, ADCY3 | 7 | 2 | 24871364 | 0.239 | 2.06e−06 | 0.241 | 0.0002435 | 0.722 | Positive |
| rs7567997 | CENPO, ADCY3 | 7 | 2 | 24950456 | 0.416 | 6.77e−06 | 0.368 | 0.0002896 | 0.735 | Positive |
| rs6733224 | CENPO, ADCY3 | 7 | 2 | 24984411 | 0.413 | 1.68e−05 | 0.451 | 0.0003063 | 0.744 | Negative |
| rs2033653 | CENPO, ADCY3 | 7 | 2 | 24956950 | 0.408 | 1.7e−05 | 0.451 | 0.0003578 | 0.755 | Negative |
| rs2384058 | CENPO, ADCY3 | 7 | 2 | 24953832 | 0.408 | 2.43e−05 | 0.461 | 0.001118 | 0.812 | Positive |
| rs11675457 | CENPO, ADCY3 | 7 | 2 | 24933274 | 0.393 | 2.47e−05 | 0.461 | 0.00257 | 0.850 | Positive |
| rs10198275 | CENPO, ADCY3 | 7 | 2 | 24984046 | 0.412 | 2.76e−05 | 0.461 | 0.0004973 | 0.767 | Positive |
| rs6545814 | CENPO, ADCY3 | 7 | 2 | 24984820 | 0.412 | 2.76e−05 | 0.461 | 0.0004973 | 0.767 | Positive |
| rs6545800 | CENPO, ADCY3 | 7 | 2 | 24972389 | 0.412 | 3.51e−05 | 0.465 | 0.0006473 | 0.767 | Negative |
| rs10200566 | CENPO, ADCY3 | 7 | 2 | 24983966 | 0.433 | 3.9e−05 | 0.465 | 0.0003531 | 0.755 | Negative |
| rs2384061 | CENPO, ADCY3 | 7 | 2 | 24989124 | 0.386 | 3.97e−05 | 0.465 | 0.00181 | 0.833 | Positive |
| rs11900505 | CENPO, ADCY3 | 7 | 2 | 24985490 | 0.412 | 4.14e−05 | 0.469 | 0.0005097 | 0.767 | Positive |
| rs2033655 | CENPO, ADCY3 | 7 | 2 | 24954596 | 0.396 | 4.99e−05 | 0.487 | 0.004194 | 0.886 | Positive |
| rs1865689 | CENPO, ADCY3 | 7 | 2 | 24961701 | 0.399 | 5.05e−05 | 0.487 | 0.001615 | 0.826 | Negative |
| rs12477891 | ASB3, NRXN1, | 8 | 2 | 51881093 | 0.0484 | 2.27e−06 | 0.241 | 0.0001402 | 0.612 | Negative |
| rs16823406 | GTDC1 | 9 | 2 | 144498321 | 0.0142 | 5.66e−05 | 0.494 | 0.0007962 | 0.788 | Negative |
| rs2568816 | GPD2 | 10 | 2 | 157099822 | 0.289 | 4.95e−05 | 0.487 | 0.001545 | 0.821 | Positive |
| rs16842126 | ACVR1 | 11 | 2 | 158389887 | 0.0137 | 0.00118 | 0.81 | 4.73e−07 | 0.177 | Positive |
| rs11900000 | SPAG16 | 12 | 2 | 214668085 | 0.0631 | 0.00215 | 0.835 | 3.04e−05 | 0.480 | Negative |
| rs1472929 | ARL4C, SPP2, | 13 | 2 | 235017645 | 0.0756 | 1.54e−06 | 0.241 | 8.55e−05 | 0.607 | Positive |
| rs1876715 | ARL4C, SPP2, | 13 | 2 | 235029590 | 0.074 | 1.76e−06 | 0.241 | 0.0001107 | 0.607 | Negative |
| rs6743014 | ARL4C, SPP2, | 13 | 2 | 235015475 | 0.0775 | 1.34e−05 | 0.451 | 0.0006123 | 0.767 | Positive |
| rs7641662 | GRM7, LMCD1, | 14 | 3 | 7805879 | 0.0118 | 3.23e−05 | 0.465 | 0.009134 | 0.928 | Positive |
| rs6791277 | SGOL1, VENTXP7, ZNF385D | 15 | 3 | 21227105 | 0.0145 | 1.77e−06 | 0.241 | 9.61e−05 | 0.607 | Negative |
| rs7648626 | SGOL1, VENTXP7, ZNF385D | 15 | 3 | 21196407 | 0.0274 | 2.96e−05 | 0.461 | 2.35e−05 | 0.456 | Negative |
| rs6550568 | SGOL1, VENTXP7, ZNF385D | 15 | 3 | 21196353 | 0.0281 | 3.76e−05 | 0.465 | 2.35e−05 | 0.456 | Negative |
| rs3774598 | CACNA1D | 16 | 3 | 53797359 | 0.0114 | 0.00081 | 0.757 | 1.46e−05 | 0.363 | Negative |
| rs9821040 | LSAMP | 17 | 3 | 117233816 | 0.185 | 3.24e−05 | 0.465 | 0.0009233 | 0.801 | Positive |
| rs275697 | AGTR1 | 18 | 3 | 149836430 | 0.0536 | 7.1e−05 | 0.539 | 5.12e−06 | 0.192 | Positive |
| rs4689946 | MSX1, STX18, | 19 | 4 | 4871714 | 0.168 | 6e−06 | 0.368 | 0.0006735 | 0.779 | Positive |
| rs1907991 | MSX1, STX18, | 19 | 4 | 4864223 | 0.167 | 3.61e−05 | 0.465 | 0.001974 | 0.833 | Negative |
| rs10517528 | UBE2K | 20 | 4 | 39424918 | 0.189 | 2.5e−05 | 0.461 | 0.000854 | 0.793 | Negative |
| rs11723204 | PRKG2 | 21 | 4 | 82273150 | 0.0276 | 0.00535 | 0.869 | 8.7e−06 | 0.271 | Positive |
| rs12651081 | AFF1 | 22 | 4 | 88103790 | 0.362 | 0.00461 | 0.869 | 4.47e−06 | 0.186 | Positive |
| rs1447993 | AFF1 | 22 | 4 | 88076615 | 0.185 | 0.00553 | 0.869 | 9.33e−06 | 0.271 | Positive |
| rs6836128 | AFF1 | 22 | 4 | 88082794 | 0.182 | 0.0126 | 0.91 | 2.29e−05 | 0.456 | Positive |
| rs17024266 | PDHA2, UNC5C, | 23 | 4 | 96760067 | 0.0343 | 7.96e−08 | 0.0596 | 1.62e−07 | 0.121 | Negative |
| rs17024261 | PDHA2, UNC5C, | 23 | 4 | 96755517 | 0.0327 | 2.38e−05 | 0.461 | 3.03e−05 | 0.480 | Negative |
| rs6814329 | PDHA2, UNC5C, | 23 | 4 | 96861645 | 0.035 | 5.42e−05 | 0.494 | 0.0001138 | 0.607 | Positive |
| rs17050999 | MAML3, SCOC, | 24 | 4 | 141044042 | 0.0304 | 0.00017 | 0.614 | 1.9e−05 | 0.410 | Negative |
| rs7654189 | PALLD | 25 | 4 | 169962988 | 0.34 | 7.62e−06 | 0.368 | 7.57e−05 | 0.607 | Negative |
| rs869396 | PALLD | 25 | 4 | 169924575 | 0.442 | 1.81e−05 | 0.451 | 0.03075 | 0.965 | Negative |
| rs11726774 | PALLD | 25 | 4 | 169963645 | 0.464 | 3.97e−05 | 0.465 | 0.004151 | 0.886 | Positive |
| rs17708289 | PALLD | 25 | 4 | 169928846 | 0.266 | 5.64e−05 | 0.494 | 0.001761 | 0.830 | Positive |
| rs7679982 | PALLD | 25 | 4 | 169929444 | 0.267 | 5.64e−05 | 0.494 | 0.001761 | 0.830 | Positive |
| rs16879248 | FASTKD3, | 26 | 5 | 7917532 | 0.0289 | 1.76e−05 | 0.451 | 0.0001785 | 0.656 | Negative |
| rs16895353 | CD180, MAST4, PPIA, | 27 | 5 | 65966059 | 0.0179 | 2.04e−05 | 0.461 | 0.000156 | 0.631 | Negative |
| rs1977059 | FARS2, | 28 | 6 | 5665825 | 0.0457 | 0.0997 | 0.966 | 2.38e−05 | 0.456 | Negative |
| rs4716037 | ARPC3, MYLIP, | 29 | 6 | 16165496 | 0.354 | 1.61e−05 | 0.451 | 0.001384 | 0.820 | Positive |
| rs6917825 | ID4, RNF144B, RPL21P28, | 30 | 6 | 19002215 | 0.15 | 0.00016 | 0.601 | 1.73e−05 | 0.400 | Negative |
| rs4716312 | ID4, RNF144B, RPL21P28, | 30 | 6 | 18969221 | 0.15 | 0.00032 | 0.67 | 2.49e−05 | 0.465 | Positive |
| rs1360771 | ID4, RNF144B, RPL21P28, | 30 | 6 | 18969437 | 0.145 | 0.00038 | 0.69 | 3.01e−05 | 0.480 | Negative |
| rs849877 | HDGFL1, PRL, | 31 | 6 | 22406678 | 0.393 | 5.18e−05 | 0.49 | 0.003525 | 0.876 | Negative |
| rs1205925 | HDGFL1, PRL, | 31 | 6 | 22469455 | 0.467 | 4.32e−05 | 0.482 | 0.001221 | 0.816 | Negative |
| rs6926543 | FYN | 32 | 6 | 112042375 | 0.366 | 5.51e−05 | 0.494 | 1.76e−05 | 0.400 | Positive |
| rs794258 | FAM184A | 33 | 6 | 119479680 | 0.0284 | 0.00235 | 0.836 | 3.08e−05 | 0.480 | Negative |
| rs7740792 | UTRN | 34 | 6 | 144656559 | 0.0114 | 9.84e−06 | 0.388 | 0.0002812 | 0.735 | Negative |
| rs3757020 | MAP3K4 | 35 | 6 | 161410210 | 0.0244 | 0.0209 | 0.936 | 2.93e−05 | 0.480 | Negative |
| rs12524741 | RPS6KA2 | 36 | 6 | 166836302 | 0.0716 | 4.63e−05 | 0.487 | 1.18e−05 | 0.305 | Positive |
| rs6934309 | RPS6KA2 | 36 | 6 | 166837330 | 0.0714 | 9.41e−05 | 0.563 | 1.13e−05 | 0.301 | Positive |
| rs2345970 | TCP10L2, UNC93A, | 37 | 6 | 167521338 | 0.252 | 0.00039 | 0.69 | 3.25e−06 | 0.186 | Negative |
| rs886739 | AUTS2, WBSCR17, | 38 | 7 | 70213501 | 0.305 | 3.98e−05 | 0.465 | 9.03e−05 | 0.607 | Positive |
| rs17170877 | CNTNAP2 | 39 | 7 | 147516396 | 0.485 | 5.6e−05 | 0.494 | 0.01202 | 0.940 | Positive |
| rs7840084 | SGCZ, | 40 | 8 | 14079214 | 0.147 | 1.93e−05 | 0.461 | 0.0003575 | 0.755 | Positive |
| rs10088053 | ASPH, NKAIN3, | 41 | 8 | 62928256 | 0.0672 | 3.64e−06 | 0.273 | 0.0001702 | 0.637 | Negative |
| rs10963396 | SMARCA2 | 42 | 9 | 1801657 | 0.0251 | 1.63e−05 | 0.451 | 8.74e−05 | 0.607 | Negative |
| rs10116883 | SMARCA2 | 42 | 9 | 1826746 | 0.0222 | 1.99e−05 | 0.461 | 0.007552 | 0.922 | Negative |
| rs953188 | TLE1 | 43 | 9 | 82910311 | 0.111 | 1.49e−05 | 0.451 | 8.4e−07 | 0.186 | Positive |
| rs997020 | TLE1 | 43 | 9 | 82948468 | 0.112 | 1.62e−05 | 0.451 | 4.9e−06 | 0.192 | Negative |
| rs10867699 | TLE1 | 43 | 9 | 82911335 | 0.143 | 3.84e−05 | 0.465 | 3.57e−06 | 0.186 | Positive |
| rs2809841 | TLE1 | 43 | 9 | 82979213 | 0.138 | 7.14e−05 | 0.539 | 1.12e−05 | 0.301 | Positive |
| rs17086403 | FRMD3 | 44 | 9 | 85325008 | 0.0609 | 3.63e−05 | 0.465 | 0.003067 | 0.861 | Negative |
| rs901683 | MARCH8 | 45 | 10 | 45286428 | 0.0633 | 1.18e−05 | 0.42 | 0.0002523 | 0.726 | Positive |
| rs10824983 | PCDH15, PRKRIR, | 46 | 10 | 54716153 | 0.421 | 4.93e−05 | 0.487 | 0.007762 | 0.923 | Positive |
| rs11212408 | SLC35F2 | 47 | 11 | 107222310 | 0.371 | 0.00457 | 0.869 | 3.37e−05 | 0.495 | Positive |
| rs10492108 | DDX12 | 48 | 12 | 9422322 | 0.177 | 2.78e−05 | 0.461 | 0.005041 | 0.892 | Positive |
| rs7302181 | DDX12 | 48 | 12 | 9369404 | 0.193 | 2.84e−05 | 0.461 | 0.004073 | 0.886 | Negative |
| rs11050596 | DDX12 | 48 | 12 | 9422128 | 0.193 | 4.56e−05 | 0.487 | 0.005877 | 0.903 | Negative |
| rs2720185 | LRIG3, XRCC6BP1, | 49 | 12 | 56765747 | 0.0122 | 0.00581 | 0.869 | 2.65e−06 | 0.186 | Negative |
| rs12825850 | SLC9A7 | 50 | 12 | 96773971 | 0.142 | 2.92e−05 | 0.461 | 0.01166 | 0.938 | Negative |
| rs10507737 | PCDH9 | 51 | 13 | 65929499 | 0.209 | 4.09e−05 | 0.469 | 0.0002645 | 0.734 | Negative |
| rs4148536 | ABCC4 | 52 | 13 | 94515499 | 0.0655 | 9.56e−05 | 0.563 | 1.06e−06 | 0.186 | Negative |
| rs17093751 | ACTR10, SLC35F4, | 53 | 14 | 57375098 | 0.0137 | 1.56e−06 | 0.241 | 0.0006033 | 0.767 | Positive |
| rs17094008 | ACTR10, SLC35F4, | 53 | 14 | 57497859 | 0.054 | 2.58e−05 | 0.461 | 0.006142 | 0.906 | Positive |
| rs1092014 | ACTR10, SLC35F4, | 53 | 14 | 57272128 | 0.0122 | 2.73e−05 | 0.461 | 0.01513 | 0.948 | Negative |
| rs2145489 | ACTR10, SLC35F4, | 53 | 14 | 57374152 | 0.0548 | 3.16e−05 | 0.465 | 0.0004234 | 0.767 | Positive |
| rs1956681 | ACTR10, SLC35F4, | 53 | 14 | 57309830 | 0.0228 | 4.56e−05 | 0.487 | 0.02015 | 0.956 | Positive |
| rs234605 | PAPOLA, VRK1, | 54 | 14 | 96141802 | 0.423 | 2.47e−05 | 0.461 | 0.000126 | 0.607 | Positive |
| rs1400412 | SEMA6D | 55 | 15 | 44672449 | 0.0479 | 0.0001 | 0.565 | 2.8e−05 | 0.480 | Positive |
| rs2719715 | TEKT5 | 56 | 16 | 10680325 | 0.171 | 0.00028 | 0.67 | 2.92e−05 | 0.480 | Negative |
| rs10221110 | TEKT5 | 56 | 16 | 10680908 | 0.171 | 0.00028 | 0.67 | 2.92e−05 | 0.480 | Negative |
| rs12925749 | TEKT5 | 56 | 16 | 10677661 | 0.112 | 0.00096 | 0.791 | 1.92e−05 | 0.410 | Negative |
| rs10500575 | RPSA, ZFHX3, | 57 | 16 | 72446965 | 0.281 | 5.68e−05 | 0.494 | 0.0007026 | 0.786 | Negative |
| rs13330604 | DYNLRB2 | 58 | 16 | 78483602 | 0.164 | 7.87e−06 | 0.368 | 0.001172 | 0.812 | Negative |
| rs8072580 | TANC2 | 59 | 17 | 58504151 | 0.0509 | 0.00185 | 0.812 | 1.55e−05 | 0.374 | Positive |
| rs372889 | IL12RB1 | 60 | 19 | 18034603 | 0.486 | 9.85e−06 | 0.388 | 0.004928 | 0.892 | Negative |
| rs10421285 | ZNF470, ZNF71 | 61 | 19 | 61777581 | 0.364 | 4.45e−05 | 0.487 | 0.001248 | 0.816 | Negative |
| rs11084454 | ZNF470, ZNF71 | 61 | 19 | 61784980 | 0.37 | 3.22e−05 | 0.465 | 0.0008836 | 0.798 | Negative |
| rs741252 | ZNF470, ZNF71, | 61 | 19 | 61792033 | 0.368 | 3.38e−05 | 0.465 | 0.0008836 | 0.798 | Positive |
| rs4801343 | ZNF470, ZNF71, | 61 | 19 | 61787066 | 0.367 | 3.71e−05 | 0.465 | 0.000967 | 0.811 | Negative |
| rs6084145 | NRSN2, SOX12, | 62 | 20 | 273102 | 0.154 | 0.00198 | 0.823 | 3.22e−05 | 0.490 | Negative |
| rs7267965 | MANBAL, | 63 | 20 | 35327104 | 0.0236 | 2.84e−05 | 0.461 | 6.03e−05 | 0.607 | Negative |
| rs7262172 | —, ADA, WISP2, | 64 | 20 | 42750069 | 0.255 | 3.42e−05 | 0.465 | 0.01303 | 0.940 | Positive |
| rs13056461 | C22orf34, FAM19A5, | 65 | 22 | 47541093 | 0.351 | 0.00023 | 0.669 | 3.28e−05 | 0.490 | Positive |
| rs3119588 | ? | 66 | — | 785 | 0.254 | 0.00061 | 0.723 | 9.4e−06 | 0.271 | Negative |
| rs5943590 | IL1RAPL1, | 67 | X | 28977674 | 0.241 | 2.58e−06 | 0.241 | 5.8e−06 | 0.207 | Negative |
| rs2651175 | IL1RAPL1 | 67 | X | 29021974 | 0.204 | 5.08e−05 | 0.487 | 0.0002087 | 0.677 | Negative |
| rs7060905 | COL4A6 | 68 | X | 107557678 | 0.0104 | 2.79e−05 | 0.461 | 2.37e−06 | 0.186 | Negative |

TABLE 16

| Gene | | |
|---|---|---|
| Symbol | Cluster | Description |
| LRRC7 | 1 | leucine rich repeat containing 7 |
| ST13 | 5 | suppression of tumorigenicity 13 (colon |
| | | carcinoma) (Hsp70 interacting protein) |
| ITSN2 | 6 | intersectin 2 |
| ADCY3 | 7 | adenylate cyclase 3 |
| NRXN1 | 8 | neurexin 1 |
| ACVR1 | 11 | activin A receptor |
| ARL4C | 13 | ADP-ribosylation factor-like 4C |
| CACNA1D | 16 | calcium channel |
| LSAMP | 17 | limbic system-associated membrane protein |
| STX18 | 19 | syntaxin 18 |
| UNC5C | 23 | unc-5 homolog C (C. elegans) |
| PALLD | 25 | palladin |
| MAST4 | 27 | microtubule associated serine/threonine kinase |
| | | family member 4 |
| PPIA | 27 | peptidylprolyl isomerase A (cyclophilin A) |
| ARPC3 | 29 | actin related protein 2/3 complex |
| MYLIP | 29 | myosin regulatory light chain interacting protein |
| ID4 | 30 | inhibitor of DNA binding 4 |
| FYN | 32 | FYN oncogene related to SRC |
| UTRN | 34 | utrophin |
| MAP3K4 | 35 | mitogen-activated protein kinase kinase kinase 4 |
| TCP10L2 | 37 | t-complex 10-like 2 (mouse) |
| CNTNAP2 | 39 | contactin associated protein-like 2 |
| SGCZ | 40 | sarcoglycan zeta |
| FRMD3 | 44 | FERM domain containing 3 |
| PCDH15 | 46 | protocadherin 15 |
| SLC9A7 | 50 | solute carrier family 9 (sodium/hydrogen |
| | | exchanger) |
| PCDH9 | 51 | protocadherin 9 |
| ACTR10 | 53 | actin-related protein 10 homolog (S. cerevisiae) |
| SEMA6D | 55 | sema domain |
| ZFHX3 | 57 | zinc finger homeobox 3 |
| DYNLRB2 | 58 | dynein |
| TANC2 | 59 | tetratricopeptide repeat |
| NRSN2 | 62 | neurensin 2 |

### REFERENCES

1. Wichter T, Matheja P, Eckardt L, Kies P, Schafers K, Schulze-Bahr E, Haverkamp W, Borggrefe M, Schober O, Breithardt G, Schafers M. Cardiac autonomic dysfunction in Brugada syndrome. Circulation. 2002; 105(6):702-706.
2. Shah S H, Pitt G S. Genetics of cardiac repolarization. Nat Genet, 2009; 41(4):388-389.
3. Alcalai R, Seidman J G, Seidman C E. Genetic basis of hypertrophic cardiomyopathy: from bench to the clinics. J Cardiovasc Electrophysiol. 2008; 19(1):104-110.
4. Leonardo E D, Hinck L, Masu M, Keino-Masu K, Ackerman S L, Tessier-Lavigne M. Vertebrate homologues of C. elegans UNC-5 are candidate netrin receptors. Nature. 1997; 386027):833-838.
5. Korobova F, Svitkina T. Arp2/3 complex is important for filopodia formation, growth cone motility, and neuritogenesis in neuronal cells. Mol Biol Cell, 2008; 19(4):1561-1574.
6. Ni X, Ji C, Cao G, Cheng H, Guo L, Gu S, Ying K, Zhao R C, Mao Y. Molecular cloning and characterization of the protein 4.10 gene, a novel member of the protein 4.1 family with focal expression in ovary. J Hum Genet. 2003; 48(2):101-106.
7. Wingrove J. Unpublished Data. 2010, see Appendix A
8. Miura Y, Tam Ido A, Morinaga T, Hashimoto T, Tamaoki Cloning and characterization of an ATBF1 isoform that expresses in a neuronal differentiation-dependent manner. J Biol Chem. 1995; 270(45):26840-26848.
9. Gomez Del Pulgar Valdes-Mora F, Bandres E, Perez-Palacios R, Espina C, Cejas P, Garcia-Cabezas M A, Nistal M, Casado E, Gonzalez-Baron M, Garcia-Foncillas J, Lacal J C. Cdc42 is highly expressed in colorectal adenocarcinoma and downregulates ID4 through an epigenetic mechanism. Int J Oncol. 2008; 33(1):185-193.
10. Benjamin E J, Rice K M, Arking D E, Pfeufer A, van Noord C, Smith A V, Schnabel R B, Bis J C, Boerwinkie E, Sinner M F, Dehghan A, Lubitz S A, D'Agostino R B, Sr., Lumley T, Ehret G B, Heeringa J, Aspelund T, Newton-Cheh C, Larson M G, Marciante K D, Soliman E Z, Rivadeneira F, Wang T J, Eiriksdottir G, Levy D, Psaty B M, Li M, Chamberlain A M, Hofman A, Vasan R S, Harris T B, Rotter J I, Kao W H, Agarwal S K, Stricker B H, Wang K, Launer L J, Smith N L, Chakravarti A, Uitterlinden A G, Wolf P A, Sotoodehnia N, Kottgen A, van Duijn C M, Meitinger T, Mueller M, Perz S, Steinbeck G, Wichmann H E, Lunetta K L, Heckbert S R, Gudnason V, Alonso A, Kaab S, Ellinor P T, Witteman J C. Variants in ZFHX3 are associated with atrial fibrillation in individuals of European ancestry, Nat. Genet. 2009; 41(8):879-881.
11. Gudbjartsson D F, Holm H, Gretarsdottir S, Thorleifsson G, Walters G B, Thorgeirsson G, Gulcher J, Mathiesen E B, Njolstad I, Nyrnes A, Wilsgaard T, Hald E M, Hveem K, Stoltenberg C, Kucera G, Stubblefield T, Carter S, Roden D, Ng M C, Baum L, So W Y, Wong K S, Chan J C, Gieger C, Wichmann H E, Gschwendtner A, Dichgans M, Kuhlenbaumer G, Berger K, Ringelstein E B, Bevan S, Markus H S, Kostulas K, Hillert J, Sveinbjornsdottir S, Valdimarsson E M, Lochen M L, Ma R C, Darbar D, Kong A, Arnar D O, Thorsteinsdottir U, Stefansson K. A sequence variant in ZFHX3 on 16q22 associates with atrial fibrillation and ischemic stroke. Nat Genet. 2009; 41(8):876-878.
12. Boukhelifa M, Moza M, Johansson T, Rachlin A, Parast M, Huttelmaier S, Roy P, Jockusch B M, Carpen O, Karlsson R, Otey C A. The proline-rich protein palladin is a binding partner for profilin. FEBS J. 2006; 273(1):26-33.
13. Calhoun C C, Lu Y C, Song J, Chiu R. Knockdown endogenous CypA with siRNA U2OS cells results in disruption of F-actin structure and alters tumor phenotype. Mol Cell Biochem, 2009; 320(1-2):35-43.
14. Olsson P A, Korhonen L, Mercer E A, Lindholm D. MIR is a novel ERM-like protein that interacts with myosin regulatory light chain and inhibits neurite outgrowth. J Biol. Chem. 1999; 274(51):36288-36292.
15. Qu X, Wei H, Zhai Y, Que H, Chen Q, Tang F, Wu Y, Xing G, Zhu Y, Liu S, Fan M, He F. Identification, characterization, and functional study of the two novel human members of the semaphorin gene family, J Biol Chem. 2002; 277(38):35574-35585.
16. Toyofuku T, Zhang H, Kumanogoh A, Takegahara N, Suto F, Kamei J, Aoki K, Yabuki M, Hori M, Fujisawa H, Kikutani H. Dual roles of Sema6D in cardiac morphogenesis through region-specific association of its receptor, Plexin-A1, with off-track and vascular endothelial growth factor receptor type 2. Genes Dev. 2004; 18(4):435-447.
17. Susalka S J, Nikulina K, Salata M W, Vaughan P S, King S M, Vaughan K T, Pfister K K. The roadblock light chain binds a novel region of the cytoplasmic Dynein intermediate chain. J Biol Chem. 2002277(36):32939-32946.
18. Eckley D M, Schroer T A. Interactions between the evolutionarily conserved, actin-related protein, Arp11, actin, and Arp1. Mol Biol Cell. 2003; 14(7):2645-2654.
19. Nakanishi K, Ida M, Suzuki H, Kitano C, Yamamoto A, Mari N, Araki M, Taketani S. Molecular characterization of a transport vesicle protein Neurensin-2, a homologue of Neurensin-1, expressed in neural cells. Brain Res. 2006; 1081(1):1-8.
20. Hatsuzawa K, Hirose H, Tani K, Yamamoto A, Scheller R H, Tagaya M. Syntaxin 18, a SNAP receptor that functions in the endoplasmic reticulum, intermediate compartment, and cis-Golgi vesicle trafficking. J Biol Chem. 2000; 275(18):13713-13720.
21. Wei S M, Xie C G, Abe Y, Cai J T. ADP-ribosylation factor like 7 (ARL7) interacts with alpha-tublin and modulates intracellular vesicular transport. Biochem Biophys Res Commun. 2009; 384(3):352-356,
22. Kagami. T, Chen S, Memar P, Choi M, Foster L J, Numata M. Identification and biochemical characterization of the SLC9A7 interactome. Mol Membr Biol. 2008; 25(5):436-447.
23. Poliak S, Gollan L, Martinez R, Custer A, Einheber S, Salzer J L, Trimmer J S, Shrager P, Peles E. Caspr2, a new member of the neurexin superfamily, is localized at the juxtaparanodes of myelinated axons and associates with K+ channels. Neuron. 1999; 24(4):1037-1047.
24. Nguyen T, Sudhof T C. Binding properties of neuroligin 1 and neurexin 1beta reveal function as heterophilic cell adhesion molecules. J Biol Chem. 1997; 27241):26032-26039.
25. Walikonis R S, Oguni A, Khorosheva E M, Jeng C J, Asuncion F J, Kennedy M B. Densin-180 forms a ternary complex with the (alpha)-subunit of Ca2+/calmodulin-dependent protein kinase II and (alpha)-actinin. J Neurosci. 2001; 21(2):423-433,
26. Sano K, Tanihara H, Heimark R L, Obata S, Davidson M, St John T, Taketani S, Suzuki S. Protocadherins: a large family of cadherin-related molecules in central nervous system. EMBO J. 1993; 12(6):2249-2256,
27. Pimenta A F, Fischer I, Levitt P. cDNA cloning and structural analysis of the human limbic-system-associated membrane protein (LAW). Gene. 1996; 170(2):189-195.
28. Lim S H, Kwon S K, Lee M K, Moon J, Jeong D G, Park E, Kim S J, Park B C, Lee S C, Ryu S E, Yu D Y, Chung B H, Kim F, Myung P K, Lee J R. Synapse formation regulated by protein tyrosine phosphatase receptor T through interaction with cell adhesion molecules and Fyn. EMBO J. 2009; 28(22):3564-3578.
29. Marangi P A, Wieland S T, Fuhrer C. Laminin-1 redistributes postsynaptic proteins and requires rapsyn, tyrosine phosphorylation, and Src and Fyn to stably cluster acetylcholine receptors. J Cell Biol. 2002; 157(5):883-895.
30. Wayman G A, Impey S, Storm D R. Ca2+ inhibition of type III adenylyl cyclase in vivo. J Biol Chem. 1995; 270(37):21480-21486.
31. Barrios-Rodiles M, Brown K R, Ozdamar B, Bose R, Liu Z, Donovan R S, Shinjo F, Liu Y, Dembowy J, Taylor I W, Luga V, Przulj N, Robinson M, Suzuki H, Hayashizaki Y, Jurisica I, Wrana J L. High-throughput mapping of a dynamic signaling network in mammalian cells. Science. 2005; 307(5715):1621-1625.
32. Laporte S A, Oakley R H, Zhang J, Holt J A, Ferguson S S, Caron M G, Barak L S. The beta2-adrenergic receptor/betaarrestin complex recruits the clathrin adaptor AP-2 during endocytosis. Proc Natl Acad Sci USA. 1999; 96(7):3712-3717,
33. Pucharcos C, Estivill X, de la Luna S. Intersectin 2, a new multinodular protein involved in clathrin-mediated endocytosis, FEBS Lett. 2000; 478(1-2):43-51.
34. Seifert M, Ampofo C, Mehraein Y, Reichrath J, Welter C. Expression analysis of human intersectin 2 gene (ITSN2) minor splice variants showing differential expression in normal human brain. Oncol Rep. 2007; 17(5):1207-1211.
35. Fan G H, Yang W, Sai J, Richmond A. Hsc/Hsp70 interacting protein (hip) associates with CXCR2 and regulates the receptor signaling and trafficking. J Biol Chem. 2002; 277(8):6590-6597,
36. Antzelevitch C, Pollevick G D, Cordeiro J M, Casis O, Sanguinetti M C, Aizawa Y, Guerchicoff A, Pfeiffer R, Oliva A, Wollnik B, Gelber P, Bonaros E P, Jr., Burashnikov E, Wu Y, Sargent J D, Schickel S, Oberheiden R, Bhatia A, Hsu L F, Haissaguerre M, Schimpf R, Borggrefe M, Wolpert C. Loss-of-function mutations in the cardiac calcium channel underlie a new clinical entity characterized by ST-segment elevation, short QT intervals, and sudden cardiac death. Circulation. 2007; 115(4):442-449.
37. Nakamura M, Sunagawa M, Kosugi T, Sperelakis N. Actin filament disruption inhibits L-type Ca(2+) channel current in cultured vascular smooth muscle cells. Am J Physiol Cell Physiol. 2000; 279(2):C480-487,
38. Sadeghi A, Doyle A D, Johnson B D, Regulation of the cardiac L-type Ca2+ channel by the actin-binding proteins alpha-actinin and dystrophin. Am J Physiol Cell Physiol. 2002; 282(6):C1502-1511.
39. Waite A, Tinsley C L, Locke M, Blake D J. The neurobiology of the dystrophin-associated glycoprotein complex. Ann Med. 2009; 41(5):344-359.

1. A method for predicting the likelihood of a sudden cardiac event (SCE) in a subject, comprising:
obtaining a first dataset associated with a sample obtained from the subject, wherein the first dataset comprises data for a single nucleotide polymorphism (SNP) marker selected from Table 15; and
analyzing the first dataset to determine the presence or absence of data for the SNP marker, wherein the presence of the SNP marker data is positively correlated or negatively correlated with the likelihood of SCE in the subject.

2. The method of claim 1, wherein the SNP marker is rs17024266.

3. The method of claim 1, wherein the first dataset comprises data for at least two, three, four, five, six, seven, eight, nine, ten, eleven, twelve, thirteen, fourteen, fifteen, sixteen, seventeen, eighteen, nineteen, twenty or more SNP markers selected from Table 15, and further comprising analyzing the first dataset to determine the presence or absence of data for the at least two, three, four, five, six, seven, eight, nine, ten, eleven, twelve, thirteen, fourteen, fifteen, sixteen, seventeen, eighteen, nineteen, twenty or more SNP markers selected from Table 15.

4. The method of claim 3, further comprising determining the likelihood of SCE in the subject according to the relative number of positively correlated and negatively correlated SNP marker data present in the first dataset.

5. The method of claim 1, father comprising determining the likelihood that the subject would benefit from implantation of an internal cardioverter defibrillator (ICD) based on the analysis.

6. The method of claim 1, wherein the SCE is a ventricular arrhythmia.

7. The method of claim 1, wherein the SNP marker comprises at least one SNP marker selected from the group consisting of: rs17024266, rs1472929, rs17093751, rs6791277, rs4665719, rs12477891, rs5943590, rs1018615, and rs10088053.

8. The method of claim 1, wherein the likelihood of SCE in the subject is increased in the subject compared to a control.

9. The method of claim 8, wherein the control is a second dataset associated with a control sample, wherein the second dataset comprises data for a control wild-type marker at a specified locus rather than the SNP marker at that locus.

10. The method of claim 1, wherein the likelihood of SCE in the subject is not increased in the subject compared to a control.

11. The method of claim 1, further comprising selecting a therapeutic regimen based on the analysis.

12. The method of claim 1, wherein the data is genotyping data.

13. The method of claim 1, wherein the method is implemented on one or more computers.

14. The method of claim 1, wherein the first dataset is obtained stored on a storage memory.

15. The method of claim 1, wherein obtaining the first dataset associated with the sample comprises obtaining the sample and processing the sample to experimentally determine the first dataset.

16. The method of claim 1, wherein obtaining the first dataset associated with the sample comprises receiving the first dataset directly or indirectly from a third party that has processed the sample to experimentally determine the first dataset.

17. The method of claim 1, wherein the data is obtained from a nucleotide-based assay.

18. The method of claim 1, wherein the subject is a human subject.

19. The method of claim 1, further comprising assessing a clinical factor in the subject; and combining the assessment with the analysis of the first dataset to predict the likelihood of SCE in the subject.

20. The method of claim 19, wherein the clinical factor comprises at least one clinical factor selected from the group consisting of age, gender, race, implant indication, prior pacing status, ICD presence, cardiac resynchronization therapy defibrillator (CRT-D) presence, total number of devices, device type, defibrillation thresholds performed, number of programming zones, heart failure (HF) etiology, HF onset, left ventricular ejection fraction (LVEF) at implant, New York Heart Association (NYHA) class, months from most recent myocardial infarction (MI) at implant, prior arrhythmia event in setting of MI or arthroscopic chondral osseous autograft transplantation (Cor procedure), diabetes status, Blood Urea Nitrogen (BUN), Cr, renal disease history, rhythm parameters to determine sinus v. non-sinus, heart rate, QRS duration prior to implant, left bundle branch block, systolic blood pressure, history of hypertension, smoking status, pulmonary disease, body mass index (BMI), family history of sudden cardiac death, B-type natriuretic peptide (BNP) levels, prior cardiac surgeries, medications, microvolt-level T-wave alternans (MTWA) result, and inducibility at electro-physiologic study (EPS).

21. A method for determining the likelihood of SCE in a subject, comprising:
obtaining a sample from the subject, wherein the sample comprises a SNP marker selected from Table 15;
contacting the sample with a reagent;
generating a complex between the reagent and the SNP marker;
detecting the complex to obtain a dataset associated with the sample, wherein the dataset comprises data for the SNP marker; and
analyzing the dataset to determine the presence or absence of the SNP marker, wherein the presence of the marker is positively correlated or negatively correlated with the likelihood of SCE in the subject.

22. A computer-implemented method for predicting the likelihood of SCE in a subject, comprising:
storing, in a storage memory, a dataset associated with a first sample obtained from the subject, wherein the dataset comprises data for a SNP marker selected from Table 15; and
analyzing, by a computer processor, the dataset to determine the presence or absence of the SNP marker, wherein the presence of the SNP marker is positively correlated or negatively correlated with the likelihood of SCE in the subject.

23. A system for predicting the likelihood of SCE in a subject, the system comprising:
a storage memory for storing a dataset associated with a sample obtained from the subject, wherein the dataset comprises data for a SNP marker selected from Table 15; and
a processor communicatively coupled to the storage memory for analyzing the dataset to determine the presence or absence of the SNP marker, wherein the presence of the SNP marker is positively correlated or negatively correlated with the likelihood of SCE in the subject.

24. A computer-readable storage medium storing computer-executable program code, the program code comprising:
program code for storing a dataset associated with a sample obtained from a subject, wherein the dataset comprises data for a SNP marker selected from Table 15; and
program code for analyzing the dataset to determine the presence or absence of the SNP marker, wherein the presence of the SNP marker is positively correlated or negatively correlated with the likelihood of SCE in the subject.

25. A kit for use in predicting the likelihood of SCE in a subject, comprising:
a set of reagents comprising a plurality of reagents for determining from a sample obtained from the subject data for a SNP marker selected from Table 15; and
instructions for using the plurality of reagents to determine data from the sample.

26. The kit of claim 25, wherein the instructions comprise instructions for conducting a nucleotide-based assay.

27. A kit for use in predicting the likelihood of SCE in a subject, comprising:
a set of reagents consisting essentially of a plurality of reagents for determining from a sample obtained from the subject data for a SNP marker selected from Table 15; and
instructions for using the plurality of reagents to determine data from the sample.

28. The kit of claim 27, wherein the instructions comprise instructions for conducting a nucleotide-based assay.
